(19) United States
(12) Patent Application Publication
(10) Pub. No.: US 2007/0026512 A1
(43) Pub. Date: Feb. 1, 2007

(54) Atomic structure of the catalytic domain for use in designing and identifying inhibitors of zap-70 kinase

(76) Inventor: Martin Geiser, Therwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/528,709

(22) PCT Filed: Sep. 25, 2003
(86) PCT No.: PCT/EP03/10686

§ 371 Date: Mar. 22, 2005

(30) Foreign Application Priority Data
Sep. 26, 2002 (US) 60413704

(51) Int. Cl. C12N 9/12; G06F 19/00; A61K 38/54

(52) U.S. Cl. 435/194; 514/2; 702/19

(57) ABSTRACT

The present invention relates to the three dimensional structure of ZAP-70 protein tyrosine kinase and describes methods of making a crystal of ZAP-70 and purification of the catalytic domain of ZAP-70 for use in crystallization. The invention also relates to the use of the three dimensional structure of the catalytic domain of ZAP-70 for identifying and designing ligands which inhibit the biological function of ZAP-70.

### FIELD OF THE INVENTION

The present invention relates to ZAP-70 protein tyrosine kinase, in particular, the three-dimensional structure of the catalytic domain of ZAP-70 protein tyrosine kinase. The invention also relates to the crystalline forms of liganded or unliganded human ZAP-70 catalytic domain. Further, the invention describes methods of making of a crystal comprising ZAP-70 and purification of the catalytic domain of ZAP-70 for use in crystallization. The invention also relates to the use of the three-dimensional structure of the catalytic domain of ZAP-70 kinase for identifying and designing ligands or low molecular weight compounds which inhibit the biological function of ZAP-70.

### BACKGROUND OF THE INVENTION

The protein tyrosine kinase ZAP-70 (zeta chain-associated protein of 70 kDa) plays a pivotal role in T cell activation. T cells are involved in transplant rejection, autoimmune diseases and the initiation of inflammatory responses. Activation of T cells requires engagement of the antigen-specific T cell receptor (TCR), resulting in early membrane proximal events which lead to the activation of a number of signal transduction pathways.

One of the early events of T cell activation is the phosphorylation of the TCR zeta chain and the specific association and activation of the Syk family protein tyrosine kinase ZAP-70 with the TCR via its two SH2 domains. Zeta chain-binding together with trans-phosphorylation by the src family kinase Lck leads to the activation of ZAP-70. ZAP-70 phosphorylates its specific substrate LAT (linker for activation of T cells), an adaptor molecule, which then recruits a number of downstream effector molecules. This eventually leads to the activation of early T cell genes, production of cytokines and cellular proliferation. There is ample evidence that interference with ZAP-70/LAT-mediated signaling leads to functional T cell inactivation.

Defects in ZAP-70 are the cause of selective T cell defect (STD), an autosomal recessive form of severe combined immunodeficiency characterized by a selective absence of CD-8-type T cells.

The crystal structure of the tandem SH2 domains of human ZAP-70 in complex with a peptide derived from the TCR zeta chain has been previously described by Hatada et al. (1995). This three-dimensional crystal structure of the SH2 domain has also been the subject of U.S. Pat. No. 6,251,620. The disclosure of this SH2 domain has lead to efforts directed at blocking the ZAP-70 SH2 domain/zeta chain interaction. However, to date no orally active inhibitor of ZAP-70 has been described.

The present invention focuses on the three dimensional structure of the catalytic domain of ZAP-70 for inhibition of ZAP-70 since catalytic activity at the ATP-binding site can be directly inhibited. Before the disclosure of the present invention, there was no three-dimensional crystal structure of the catalytic domain of ZAP-70. With the three-dimensional structure of the catalytic domain of the human ZAP-70, identifying and designing inhibitors of ZAP-70 based on the three-dimensional structure of the catalytic domain is now possible.

### SUMMARY OF THE INVENTION

It is an object of the present invention to provide the three-dimensional structure of ZAP-70 kinase catalytic domain thereby enabling identification and design of ligands or low molecular weight molecules that specifically inhibit ZAP-70 kinase.

The present invention relates to:

(i) a crystal of the ZAP-70 kinase comprising the catalytic domain of ZAP-70 kinase with or without a ligand or low molecular weight compound

(ii) a method of making a crystal of ZAP-70 kinase comprising the ZAP-70 kinase catalytic domain

(iii) methods of using said ZAP-70 kinase crystal comprising the catalytic domain and its structural coordinates.

The three-dimensional structural information revealed from the crystal of the the catalytic domain of ZAP-70 kinase can be used for structure-based drug discovery for screening, identifying and designing inhibitors of ZAP-70 kinase.

### DETAILED DESCRIPTION OF THE INVENTION

The full-length sequence of human ZAP-70 kinase is known and set forth in Genbank Accession number L05148 and SwissProt Accession number P43403, which are incorporated herein by reference.

The present invention provides ZAP-70 kinase catalytic domain in crystallized form. In particular, it provides a crystal comprising the catalytic domain of ZAP-70 kinase and a ligand bound to ZAP-70 as a complex.

In one embodiment of the present invention, a crystal of the catalytic domain of ZAP-70 kinase comprising a unit cell dimension of a=35.77±5 Ångstroms b=57.56±5 Ångstroms c=80.20±5 Ångstroms, α=68.97±5 degrees β=89.83±5 degrees γ=89.95±5 degrees is provided. Depending on the particular conditions for crystallization, the parameters characterising the unit cell may vary with a limited range, for example, a, b, c each vary by up to 5 Angstroms and α, β, γ each vary by up to 5 degrees. The space group of the present invention is P1 primitive triclinic.

The term “unit cell” according to the invention refers to the basic shape block. The entire volume of a crystal may be constructed by regular assembly of such blocks. Each unit cell comprises a complete representation of the unit of pattern, the repetition of which builds up the crystal.

The term “space group” according to the invention refers to the arrangement of symmetry elements of a crystal.

In another embodiment of the invention, a crystal of ZAP-70 kinase comprising the catalytic domain of ZAP-70 kinase in complex with a ligand is provided wherein said crystal has a three-dimensional structure characterized by the atomic structure coordinates of Table 1.

In a further embodiment of the invention, said catalytic domain of ZAP-70 kinase comprises the sequence of SEQ ID. No. 2, fragment or homologue thereof.

In yet another embodiment of the invention, said catalytic domain of ZAP-70 kinase comprises at least the ATP-binding site.

Further provided by this invention is a crystal comprising the catalytic domain of ZAP-70 bound to at least one ligand or low molecular weight compound.

The term “ligand” according to the invention, refers to a molecule or group of molecules that bind to one or more specific sites of ZAP-70, preferably to the catalytic domain of ZAP-70 and most preferably to the ATP binding-site of said catalytic domain. Ligands according to the invention are preferably low molecular weight molecules.

The term “low molecular weight compound” according to the invention refers to preferably organic compounds generally having a molecular weight less than about 1000, more preferably less than about 500. Most preferably, said low molecular weight compounds or ligands inhibit ZAP-70 biological activity.

In context of a ZAP-70 inhibitor, the terms “peptide” or “peptide derivative” are intended to embrace a “peptidomimetic” or “peptide analogue” which complement the three-dimensional structure of the binding site of ZAP-70 kinase or can be designed with improved physical or chemical properties to bind with the three-dimensional binding site of the ZAP-70 kinase catalytic domain as provided in the present invention.

The term “mutant” refers to differences in the wild-type sequence of ZAP-70 kinase set forth in Genbank Accession number L05148 or SwissProt Accession number P43403 by deletion, insertion or preferably replacement of one or more selected amino acids.

According to the present invention, the term “mutant” also refers to a polypeptide, whose amino acid sequence differs from the wild-type sequence given in SEQ ID No.2 by deletion, insertion or preferably replacement of one or more selected amino acids. For example, a ZAP-70 mutant of the catalytic domain of the present invention is preferably at least 50% homologous to SEQ ID No. 2, more preferably at least 80% homologous to SEQ ID No. 2 most preferably at least 90% homologous to SEQ ID No. 2.

A “fragment” of ZAP-70 catalytic domain according to the invention comprises more than 50% of the full-length sequence of the ZAP-70 catalytic domain according to SEQ ID No. 2, more preferably at least 80% of the full-length sequence of the ZAP-70 catalytic domain according to SEQ ID No. 2, most preferably at least 90% of the full-length sequence of the ZAP-70 catalytic domain according to SEQ ID No. 2.

In one embodiment of the invention, a ZAP-70 mutant of the catalytic domain may be crystallizable with or without at least one ligand.

In another embodiment of the invention, a ZAP-70 fragment of the catalytic domain may be crystallizable with or without at least one ligand.

In yet another embodiment of the invention, a method is provided wherein the catalytic domain of ZAP-70, a fragment or homologue thereof is bound to at least one ligand at any step prior to crystallization.

According to the present invention, ZAP-70 crystals are stable for at least one month, if kept under suitable conditions. Hepes pH 7.2 is identified as being suitable for the concentration of ZAP-70 without precipitation. Initially during purification, high concentrations of glycerol (30-50% v/v) are preferred, below which rapid precipitation occurs at protein concentrations in excess of 5-10 mg/ml. During the final concentration steps 1% v/v ethylene glycol can substitute for glycerol allowing concentrations in excess of 30 mg/ml to be reached. An additional cation-exchange step is also recommended to remove incorrectly folded or unstable ZAP-70 which interferes with the concentration and crystallisation process.

The purified protein ZAP-70 catalytic domain of SEQ. ID No.2, homologue or fragment thereof is advantageously obtainable according to method of the present invention by initial expression of the full-length ZAP-70 SEQ ID No.1 flanked by protease recognition sequences. This facilitates efficient proteolytic release of the desired domain. This method is preferable to standard methods known in the art whereby the desired domain typically is isolated from the full-length protein and then expressed.

Purification of an N-terminally tagged full-length ZAP-70 using a nickel-chelating affinity column yields protein of limited purity which cannot be easily purified by additional “standard” chromatographic procedures such as ion-exchange or size-exclusion chromatography. According to the present invention, affinity chromatography using γ-aminophenyl-ATP sepharose is the preferred means of purification leading to a high purity the ZAP-70 protein. Identification of the desired ZAP-70 catalytic domain is preferably by immunochemical method, for example, Western-blotting.

In one embodiment of the invention, a method for making a crystal of a ZAP-70 kinase is provided comprising the following steps:

(i) purification of the full-length ZAP-70 kinase (SEQ ID No.1)

(ii) proteolytic domain definition

(iii) expression of the full-length ZAP-70 kinase of SEQ ID No.1 flanked by protease recognition sequences to facilitate proteolytic release of the desired domain of ZAP-70

(iv) expression of the full-length ZAP-kinase from step (iii) in a suitable host cell

(v) controlled proteolysis of the desired domain at protease recognition sites

(vi) rapid purification of the desired ZAP-70 domain.

In a preferred embodiment of the invention, said method for making a crystal comprises the desired domain of ZAP-70 kinase domain comprising the catalytic domain of ZAP-70 kinase of SEQ ID No.2, a fragment or homologue thereof.

According to the invention, ZAP-70 may be prepared by isolation from natural sources, e.g. cultured human cells or preferably by recombinant heterologous expression. Expression of recombinant ZAP-70 is achievable in eukaryotic or prokaryotic systems. For example, recombinant human ZAP-70 may be expressed in insect cells, such as Sf9 cells, using a suitable recombinant baculovirus system or in bacteria.

The kinase may be expressed as a fusion protein, e.g. a glutathione-S-transferase (GST) or histidine-tagged fusion protein. If desired, the fusion partner is removed before crystallization. The heterologously produced ZAP-70 to be used for crystallization is biologically active. Such ability may be determined by morphological, biochemical or viability analysis well-known in the art.

Methods for the preparation of ZAP-70 mutants are commonly known in the art. For example, ZAP-70 mutants may be prepared by expression of ZAP-70 DNA previously modified in its coding region by oligo-nucleotide directed mutagenesis.

In the present invention, purified ZAP-70 is preferably at least 90% homogeneous. Protein homogeneity is determinable according to analytical methods well-known in the art, e.g. sequence analysis, electrophoresis, spectroscopic or chromatographic techniques. The purified protein is enzymatically active. Appropriate assays for determining ZAP-70 kinase activity towards a suitable substrate, e.g. a natural substrate or a synthetic substrate which is known in the art.

In one embodiment of the invention, prior to crystallization ZAP-70 may be reacted with a low molecular weight compound or ligand which is capable of suitably binding to the ZAP-70 catalytic domain site. Preferred is a compound inhibiting ZAP-70 activity. Kinase inhibition is determinable employing assays known in the art. Suitable inhibitors include ATP-competitive kinase inhibitors which act on the catalytic domain to inhibit ZAP-70 activity.

Various methods of cystallization can be used in the claimed invention including vapor diffusion, dialysis or batch crystallization. In vapor diffusion crystallization, a small volume (i.e., a few microliters) of protein solution is mixed with a solution containing a precipitant. This mixed volume is suspended over a well containing a small amount, i.e. about 1 ml, of precipitant. Vapor diffusion from the drop to the well will result in crystal formation in the drop.

The dialysis method of crystallization utilizes a semipermeable size-exclusion membrane that retains the protein but allows small molecules (i.e. buffers and precipitants) to diffuse in and out. In dialysis, rather than concentrating the protein and the precipitant by evaporation, the precipitant is allowed to slowly diffuse through the membrane and reduce the solubility of the protein while keeping the protein concentration fixed.

The batch method generally involves the slow addition of a precipitant to an aqueous solution of protein until the solution just becomes turbid, at this point the container can be sealed and left undisturbed for a period of time until crystallization occurs. In the batch technique the precipitant and the target molecule solution are simply mixed. Supersaturation is achieved directly rather than by diffusion. Often the batch technique is performed under oil. The oil prevents evaporation and extremely small drops can be used. For this, the term “microbatch” is used. A modification of this technique is not to use paraffin oil (which prevents evaporation completely) but rather use silicone oil or a mixture of silicone and paraffin oils so that a slow evaporation is possible.

The claimed invention can encompass any and all methods of crystallization. One skilled in the art can choose any of such methods and vary the parameters such that the chosen method results in the desired crystals.

One preferred method of crystallization of ZAP-70 involves mixing a ZAP-70 solution with a “reservoir buffer”, with a lower concentration of the precipitating agent necessary for crystal formation. For crystal formation, the concentration of the precipitating agent has to be increased, e.g. by addition of precipitating agent, for example by titration, or by allowing the concentration of precipitating agent to balance by diffusion between the crystallization buffer and a reservoir buffer. Under suitable conditions such diffusion of precipitating agent occurs along the gradient of precipitating agent, e.g. from the reservoir buffer having a higher concentration of precipitating agent into the crystallization buffer having a lower concentration of precipitating agent. Diffusion may be achieved e.g. by vapour diffusion techniques allowing diffusion of water in the common gas phase. Known techniques are e.g. vapour diffusion methods, such as the “hanging drop” or the “sitting drop” method. In the vapour diffusion method a drop of crystallization buffer containing the protein is hanging above or sitting beside a much larger pool of reservoir buffer. Alternatively, the balancing of the precipitating agent can be achieved through a semipermeable membrane that separates the crystallization buffer from the reservoir buffer and prevents dilution of the protein into the reservoir buffer.

Formation of ZAP-70 kinase catalytic domain crystals can be achieved under various conditions which are essentially determined by the following parameters: pH, presence of salts and additives, precipitating agent, protein concentration and temperature. The pH may range, for example, from about 4.0 to 9.0.

The present invention also relates to a computer readable medium having stored a model of the ZAP-70 catalytic domain crystal structure. In a preferred embodiment, said model is built from all or part of the X-ray diffraction data shown in the atomic coordinates of Table 1.

The present invention provides the structure coordinates of human ZAP-70 catalytic domain. The term “structure coordinates” or “atomic coordinates” refers to mathematical coordinates derived from the mathematical equations related to the pattern obtained on diffraction of a monochromatic beam of X-rays by the atoms (scattering centers) of a crystal comprising a ZAP-70 catalytic domain. The diffraction data are used to calculate an electron density map of the repeating unit of the crystal. The electron density maps are used to establish the positions of the individual atoms within the unit cell of the crystal.

Structural coordinates of a crystalline composition of this invention may be stored in a machine-readable form on a machine-readable storage medium, e.g. a computer hard drive, diskette, DAT tape, etc., for display as a three-dimensional shape or for other uses involving computer-assisted manipulation of, or computation based on, the structural coordinates or the three-dimensional structures they define. For example, data defining the three dimensional structure of a protein of the ZAP family, or portions or structurally similar homologues of such proteins, may be stored in a machine-readable storage medium, and may be displayed as a graphical three-dimensional representation of the protein structure, typically using a computer capable of reading the data from said storage medium and programmed with instructions for creating the representation from such data.

In one embodiment of the invention, a method is provided for determining the three-dimensional structure of the catalytic domain of ZAP-70 comprising:

(i) crystallization of ZAP-70 kinase comprising the catalytic domain of ZAP-70 (SEQ ID No.2), fragment or homologue thereof

(ii) collecting x-ray diffraction data in the form of atomic coordinates for said crystal

(iii) utilizing the atomic coordinates of Table 1 in whole or in part to determine the three-dimensional structure of the catalytic domain of ZAP-70, fragment, or homologue thereof.

In another embodiment of the invention, a method is provided for determining the three-dimensional structure of a complex comprising the catalytic domain of ZAP-70 kinase (SEQ ID No.2), fragment or homologue thereof bound to at least one ligand comprising:

(i) obtaining x-ray diffraction data for a crystal of the complex

(ii) utilizing the atomic coordinates of Table 1 in whole or in part to determine the three-dimensional structure of the complex.

According to the present invention, a three-dimensional ZAP-70 model is obtainable from a ZAP-70 crystal comprising the catalytic domain of ZAP-70, fragment or homologue thereof. Such a model can be built or refined from all or part of the ZAP-70 kinase structure data of the present invention using the x-ray diffraction coordinates, particularly the atomic structure coordinates of Table 1.

The knowledge obtained from the three-dimensional model of the catalytic binding site of ZAP-70 can be used in various ways. For example, it can be used to identify chemical entities, for example, small organic and bioorganic molecules such as peptidomimetics and synthetic organic molecules that bind to ZAP-70 and preferably block or prevent a ZAP-70 mediated or associated process or event, or that act as ZAP-70 agonists. Using the three-dimensional structure of the ZAP-70 catalytic domain, the skilled artisan constructs a model of the ZAP-70. For example, every atom can be depicted as a sphere of the appropriate van der Waals radius, and a detailed surface map of the ZAP-70 catalytic domain can be constructed.

Chemical entities that have a surface that mimics the accessible surface of the catalytic binding site of ZAP-70 can be constructed by those skilled in the art. By way of example, the skilled artisan can screen three-dimensional structural databases of compounds to identify those compounds that position appropriate functional groups in similar three dimensional structural arrangement, then build combinatorial chemistry libraries around such chemical entities to identify those with high affinity to the catalytic binding site of ZAP-70.

In one embodiment of the invention, a method is provided for identifying a ligand or low molecular weight compound that binds to the catalytic domain of ZAP-70 kinase comprising the steps of:

(i) using the three-dimensional structure of the catalytic domain derived in whole or in part from the set of atomic coordinates in Table 1 to select a potential ligand or low molecular weigh compound that binds to the catalytic domain of ZAP-70

(ii) selecting those ligands or low molecular weight compounds that bind to the catalytic domain of ZAP-70.

In another embodiment of the invention, a method is provided for identifying a ligand or low molecular weight compound that binds to the catalytic domain of ZAP-70 kinase wherein the catalytic domain of ZAP-70 comprises at least the ATP binding site of said domain,

In yet another embodiment of the invention, a method is provided for identifying ligands which inhibit the biological activity of ZAP-70 kinase.

Ligands or small molecular compounds can be identified from screening compound databases or libraries and using a computational means to form a fitting operation to a binding site on the catalytic domain of ZAP-70 kinase. The three dimensional structure of the catalytic domain of ZAP-70 as provided in the present invention in whole or in part by the structural coordinates of Table 1, can be used together with various docking programs.

The potential inhibitory or binding effect of a chemical entity on ZAP-70 may be analyzed prior to its actual synthesis and testing by the use of computer-modeling techniques. If the theoretical structure of the given chemical entity suggests insufficient interaction and association between it and ZAP-70, the need for synthesis and testing of the chemical entity is obviated. However, if computer modeling indicates a strong interaction, the molecule may then be synthesized and tested for its ability to bind to ZAP-70. Thus, expensive and time-consuming synthesis of inoperative compounds may be avoided.

An inhibitory or other binding compound of ZAP-70 may be computationally evaluated and designed by means of a series of steps in which chemical entities or fragments are screened and selected for their ability to associate with the individual binding sites of ZAP-70. Thus, one skilled in the art may use one of several methods to screen chemical entities or fragments for their ability to associate with ZAP-70. This process may begin by visual inspection of, for example, the binding site on a computer screen based on the structural coordinates of Table 1 in whole or in part. Selected fragments or chemical entities may then be positioned in a variety of orientations, or “docked,” within the catalytic binding site of ZAP-70. Docking may be accomplished using software such as Quanta and SyLyl, followed by energy minimization and molecular dynamics with standard molecular mechanics force fields, such as CHARMM and AMBER. Specialized computer programs may be of use for selecting interesting fragments or chemical entities. These programs include, for example, GRID, available from Oxford University, Oxford, UK; 5 MCSS or CATALYST, available from Molecular Simulations, Burlington, Mass.; AUTODOCK, available from Scripps Research Institute, La Jolla, Calif.; DOCK, available from University of California, San Francisco, Calif., and XSITE, available from University College of London, UK.

Using molecular replacement to exploit a set of coordinates such as those of Table 1 of the invention, the structure of a crystalline ZAP-kinase catalytic domain or portion thereof can for example, be bound to one or more ligands or low molecular weight compounds to form a complex.

The term “molecular replacement” refers to a method that involves generating a preliminary structural model of a crystal whose structural coordinates are unknown, by orienting and positioning a molecule whose structural coordinates are known, e.g., the ZAP-70 kinase catalytic domain coordinates within the unit cell of the unknown crystal, so as to best account for the observed diffraction pattern of the unknown crystal. Phases can then be calculated from this model, and combined with the observed amplitudes to give an approximated Fourier synthesis of the structure whose coordinates are unknown. This in turn can be subject to any of the several forms of refinement to provide a final accurate structure of the unknown crystal. Using the structural coordinates provided by this invention, molecular replacement may be used to determine the structural coordinates of a crystalline co complex, unknown ligand, mutant, or homolog, or of a different crystalline form of ZAP-70 kinase. Additionally, the claimed crystal and its coordinates may be used to determine the structural coordinates of a chemical entity that associates with ZAP-70.

“Homology modeling” according to the invention involves constructing a model of an unknown structure using structural coordinates of one or more related proteins, protein domains and/or one subdomains such as the catalytic domain of ZAP-70 kinase. Homology modeling may be conducted by fitting common or homologous portions of the protein or peptide whose three dimensional structure is to be solved to the three dimensional structure of homologous structural elements. Homology modeling can include rebuilding part or all of a three dimensional structure with replace of amino acids or other components by those of the related structure to be solved.

Molecular replacement according to the present invention, uses a molecule having a known structure. The three-dimensional structure of the catalytic domain of ZAP-70 provided in whole or in part in Table 1 in a machine-readable form on a data-carrier can be used as a starting point to model the structure of an unknown crystalline sample. This technique is based on the principle that two molecules which have similar structures, orientations and positions in the unit cell diffract similarly. Molecular replacement involves positioning the known structure in the unit cell in the same location and orientation as the unknown structure. Once positioned, the atoms of the known structure in the unit cell are used to calculate the structure factors that would result from a hypothetical diffraction experiment. This involves rotating the known structure in the six dimensions (three angular and three spatial dimensions) until alignment of the known structure with the experimental data is achieved. This approximate structure can be fine-tuned to yield a more accurate and often higher resolution structure using various refinement techniques. For instance, the resultant model for the structure defined by the experimental data may be subjected to rigid body refinement in which the model is subjected to limited additional rotation in the six dimensions yielding positioning shifts of under about 5%. The refined model may then be further refined using other known refinement methods. The present invention also enables homologues and mutants of ZAP-70 catalytic domain and the solving of their crystal structure. Based on the three-dimensional structure of ZAP-70 catalytic domain as provided in the present invention and using the atomic coordinates of Table 1 in whole or in part, the effects of site-specific mutations can be predicted. More specifically, the structural information provided herein permits the identification of desirable sites for amino acid modification, particularly amino acid mutation resulting in substitutional, insertional or deletional variants. Such variants may be designed to have special properties, particularly properties distinct from wild-type ZAP-70 catalytic domain, such as altered catalytic activity. Substitutions, deletions and insertions may be combined to arrive at a desired variant. Such variants can be prepared by methods well-known in the art, e.g. starting from wild-type ZAP-70 catalytic domain, or by de novo synthesis.).

ZAP-70 catalytic domain may also crystallize in a form different from the one disclosed herein. The structural information provided, for example, in SEQ ID No. 2 and Table 1 in whole or in part, is also useful for solving the structure of other crystal forms. Furthermore, it may serve to solve the structure of a ZAP-70 catalytic domain mutant, a ZAP-70 catalytic domain co-complex or a sufficiently homologous protein.

The ZAP-70 catalytic domain structural information provided herein is useful for the design of ligands or small molecule compounds which are capable of selectively interacting with ZAP-70 catalytic domain and thereby specifically modulating the biological activity of ZAP-70. Furthermore, this information can be used to design and prepare ZAP-70 mutants, e.g. mutants with altered catalytic activity, model the three-dimensional structure and solve the crystal structure of proteins, such as ZAP-70 catalytic domain homologues, ZAP-70 catalytic domain mutants or ZAP-70 catalytic domain co-complexes, involving e.g. molecular replacement.

The present invention provides a method for designing a ligand or low molecular weight compound capable of binding with ZAP-70 catalytic domain, said method comprising:

(i) using the atomic coordinates of Table 1 in whole or in part to determine the three-dimensional structure of the ZAP-70 catalytic domain

(ii) probing said three-dimensional structural of the ZAP-70 catalytic domain with candidate ligands or low molecular weight compounds to determine which bind to the catalytic domain of ZAP-70

(iii) selecting those ligands or low molecular weight compounds which bind to the catalytic domain of ZAP-70

(iv) optionally, modifying those ligands or low molecular weight compounds which bind to maximize physical binding properties such as solubility, affinity, specificity or potency.

Preferred is a method for designing a ZAP-70 inhibitor which interacts at the catalytic binding site. The present invention also relates to the chemical entity or ligand identified by such method. One approach enabled by this invention is the use of the structural coordinates of ZAP-70 catalytic domain to design chemical entities that bind to or associate with ZAP-70 kinase and alter the physical properties of the chemical entities in different ways. Thus, properties such as, for example, solubility, affinity, specificity, potency, on/off rates, or other binding characteristics may all be altered and/or maximized. One may design desired chemical entities by probing an ZAP-70 crystal comprising the catalytic domain with a library of different entities to determine optimal sites for interaction between candidate chemical entities and ZAP-70. For example, high-resolution x-ray diffraction data collected from crystals saturated with solvent allows the determination of where each type of solvent molecule adheres. Small molecules that bind tightly to those sites can then be designed and synthesized and tested for the desired activity. Once the desired activity is obtained, the molecules can be further altered to maximize desirable properties.

The invention also contemplates computational screening of small-molecule databases or designing of chemical entities that can bind in whole or in part to ZAP-70 catalytic domain. They may also be used to solve the crystal structure of mutants, co-complexes, or the crystalline form of any other molecule homologous to, or capable of associating with, at least a portion of ZAP-kinase. One method that may be employed for this purpose is molecular replacement. An unknown crystal structure, which may be any unknown structure, such as, for example, another crystal form of ZAP-70 kinase catalytic domain, an ZAP-70 kinase catalytic domain mutant or peptide, or a co-complex with ZAP-70 kinase, or any other unknown crystal of a chemical entity that associates with ZAP-70 that is of interest, may be determined using the whole of part of the structural coordinates set forth in Table 1. This method provides an accurate structural form for the unknown crystal far more quickly and efficiently than attempting to determine such information without the invention herein.

In one preferred embodiment of the invention, candidate ligands are screened in silico. The information obtained can thus be used to obtain maximally effective inhibitors or agonists of ZAP-70. The design of chemical entities that inhibit or agonize ZAP-70 generally involves consideration of at least two factors. First, the chemical entity must be capable of physically or structurally associating with ZAP-70, preferably at the catalytic site of ZAP-70. The association may be any physical, structural, or chemical association, such as, for example, covalent or noncovalent bonding, or van der Waals, hydrophobic, or electrostatic interactions. Second, the chemical entity must be able to assume a conformation that allows it to associate with ZAP-70, preferentially at the catalytic site of ZAP-70. Although not all portions of the chemical entity will necessarily participate in the association with ZAP-70, those non-participating portions may still influence the overall conformation of the molecule. This in turn may have a significant impact on the desirability of the chemical entity. Such confirmational requirements include the overall three-dimensional structure and orientation of the chemical entity in relation to all or a portion of the binding site.

Once a compound has been designed or selected by the above methods, the efficiency with which that compound may bind to ZAP-70 may be tested and modified for the maximum desired characteristic(s) using computational or experimental evaluation. Various parameters can be maximized depending on the desired result. These include, but are not limited to, specificity, affinity, on/off rates, hydrophobicity, solubility, and other characteristics readily identifiable by the skilled artisan.

The present invention also relates to identification of compounds which modulate ZAP-70. Preferred are compounds which inhibit ZAP-70 activity and are potentially useful for the treatment of diseases and conditions such as those which involve T cell and lymphocyte activation.

The present invention enables the use of molecular design techniques, particularly the rational drug design approach, to prepare new or improved chemical entities and compounds, including ZAP-70 inhibitors, capable of irreversibly or reversibly, modulating ZAP-70 activity. Improved entities or compounds means that these entities or compounds are superior to the “original” or parent compound they are derived from with regard to a property relevant to therapeutic use including suitability for in vivo administration, e.g. cellular uptake, solubility, stability against (enzymatic) degradation, binding affinity or specificity, and the like. For example, on the basis on the information provided herein it is possible to specially design ZAP-70 inhibitors which covalently, or preferably non-covalently, bind to ZAP-70. Such inhibitors may act in a competitive or uncompetitive manner, bind at or close to the active site of ZAP-70 or act allosterically.

In the design of ZAP-70 modulators the following aspects should be considered: (i) if the candidate compound is capable of physically and structurally associating with ZAP-70 kinase catalytic domain, and/or (ii) if the compound is capable of assuming a conformation allowing it to associate with ZAP-kinase catalytic domain. Advantageously, computer modelling techniques are used in the process of assessing these abilities for the modulator as a whole, or a fragment thereof—in order to minimize efforts in the synthesis or testing of insuccessful candidate compounds. Specialized computer software is well-known in the art.

Another design approach is to probe a ZAP-70 catalytic domain crystal with a variety of different chemical entities to determine optimal sites for interaction beween candidate ZAP-70 inhibitors and the target enzyme. Yet another possibility which arises from the present invention is to screen computationally small molecule data bases for chemical entities or compounds that are capable of binding, in whole or in part, to ZAP-70 catalytic domain. The quality of fit to the binding site may be judged e.g. by shape complementarity or by estimated interaction energy. Knowledge of the three-dimensional arrangement of the modifications can be then utilized for the design of new ZAP-70 ligands or low molecular weight compounds such as selective inhibitors.

Chemical entities that are capable of associating with the ZAP family member may inhibit its interaction with naturally occurring ligands of the protein and may inhibit biological functions mediated by such interaction. In the case of ZAP-70, such biological functions include activation of T cells during an immune response. Such chemical entities are potential drug candidates.

Compounds of the structures selected or designed by any of the foregoing means may be tested for their ability to bind to a ZAP family protein, inhibit the binding of a ZAP family protein to a natural or non-natural ligand therefor, and/or inhibit a biological function mediated by a ZAP family member.

The following examples serve to illustrate the present invention but should not be construed as a limitation thereof. The invention particularly relates to the specific embodiments described in these examples. Compounds first identified by any of the methods described herein are also encompassed by this invention.

### EXAMPLES

### Example 1

### Initial Purification of Full-Length ZAP Kinase

The full length amino acid sequence of the ZAP-70 kinase is given in SEQ ID No. 1. N-terminally His₆-tagged full-length ZAP kinase is expressed in Sf9 cells, this differs from the wild-type sequence in that the N-terminal is modified by insertion of a hexa-histidine sequence in between the N-terminal methionine and proline (position 2), such that this proline is now in position 8. Cell pellets are harvested and frozen at −80° C. until required. A 39 g wet cell pellet is suspended in 350 ml ice-cold buffer A (50 mM sodium phosphate pH 8, containing 12 EDTA-free Complete™ protease inhibitor tablets, 10 mM β-mercaptoethanol, 10% v/v glycerol, 0.1 mM MnCl₂, 10 mM imidazole and 300 mM NaCl). The cells are lysed for 3 minutes on ice using a Heidolph-Diax tissue-grinder followed by 10 strokes in a glass-teflon homogeniser. The resultant lysate is centrifuged for 45 min at 43,000 g at 4° C. and subsequently filtered through successive glass-fibre, 1.2 μM and 0.43 μM filter membranes. This clarified supernatant is loaded at a flow-rate of 2 ml/min onto an XK16/20 chromatography column (Amersham Biosciences) containing 20 ml Ni—NTA-agarose (Qiagen) affinity resin equilibrated with buffer A. Once all the material has been loaded, the column is washed (at a flow-rate of 4 ml/min) with buffer A until the UV-absorbance of the flow-through material has once again returned to baseline levels. At this point (using the same flow-rate), buffer B (25 mM Tris-HCl pH8, 10% v/v glycerol, 50 mM NaCl and 250 mM imidazole) is applied to the column and the peak of protein which elutes is collected. This material is loaded directly onto a 16 ml column of γ-aminophenyl-ATP sepharose equilibrated with buffer C (25 mM Tris pH 8.0 containing: 30% v/v glycerol, 1 mM DTT, 1 mM MgCl₂ and 50 mM NaCl) at a flow-rate of 2 ml/min. The column is eluted by applying a gradient of 0-1M NaCl in buffer C over 7 column volumes. The eluted peak is concentrated by ultrafiltration using a 30,000 Mᵣ cut-off membrane (Amicon) to approximately 7 ml and further purified using a Superdex 75 (XK16/60) size-exclusion column equilibrated with buffer C (but without 50 mM NaCl). The fractions containing ZAP 70 monomer are collected and pooled prior to concentration to 2.5 mg/ml and subsequent limited proteolytic digestion.

Full-length ZAP-70 is eluted from the NTA-agarose column at a purity of approximately 60% as determined by reducing SDS-PAGE. Subsequent chromatography on a 16 ml column of γ-aminophenyl-ATP sepharose gives a rather broad peak of much higher purity which can be concentrated. Detailed analysis of this peak reveals that the earlier eluting fractions contain predominantly aggregated material and that a discrete peak towards the end of the profile contains most of the monomeric ZAP of high purity. Size-exclusion chromatography gives a major peak comprising ˜90% of the protein, the remainder eluting slightly earlier in a position where dimeric or aggregated protein would be expected to elute. At this stage the purity of the preparation is in excess of 90% and suitable for use in the limited proteolytic definition of minimal kinase domains. At this stage, highest purity is very important so as to minimise the number of additional sequences present following proteolytic digestion.

### Example 2

### Proteolytic Catalytic Domain Definition

ZAP-70 is incubated for 20 h at room temperature at a 1:100 concentration ratio with the following proteases: thermolysin, carboxypeptidase A, thrombin, Arg C, Glu C, Factor Xa, Carboxypeptidase Y, chymotrpsin, Lys C, Asp N, elastase, trypsin and subtilisin (1:1 ratio). Following incubation samples are removed, subjected to reducing SDS-PAGE electrophoresis (Novex 4-20% gels, Invitrogen) and compared with non-digested controls. In the cases where faster migrating bands are observed, which are of sufficient size (˜30 kDa) to contain the catalytic domain, samples are re-run on SDS-PAGE and Western-blotted using an anti-His₆ antibody (Sigma, H-1029). This is in order to identify N-terminal and C-terminal ZAP-70 fragments (the His₆-tag is at the N-terminus, consequently forms showing anti-His₆ immunoreactivity are truncated at the C-terminus and are of no interest). Non-immunoreactive fragments are subjected to SDS-PAGE and transferred electrophoretically to PVDF membranes, the bands visualised, excised and their N-terminal sequences analysed.

Following digestion with thermolysin, trypsin, Glu C, Asp N and elastase, faster migrating bands are observed which are of sufficient size (˜30 kDa) to contain the catalytic domain. When Western-blotted against the (His)₆ N-terminal tag, non-immunoreactive fragments are produced by trypsin, elastase and thermolysin digestion, and are thereby defined as being C-terminal in origin. These fragments are sequenced. Trypsin and Thermolysin digestion both give a fragment with Isoleucine 299 as N-terminus. Elastase produces an arginine298 fragment and thermolysin an additional leucine277 fragment. Based upon these results, ZAP catalytic domains beginning with leucine277 and arginine298 are identified as being potentially suitable for crystallisation.

### Example 3

### Cloning of PreScission™ I and II Constructs

Previous attempts to purify a number of catalytic domain constructs proved difficult due to low levels of expression of soluble protein and instability of these proteins. This instability might be, in part, due to incorrect in-vivo folding of the isolated domains and in part due to non-optimal construct length. Therefore, the full-length ZAP-70 is expressed, flanked by the PreScission™ protease recognition sequences to facilitate efficient proteolytic release of the desired domain. Based on the results obtained from the limited proteolysis of the full-length version of ZAP-70, two constructs were made: PreScission™I, a C-terminally His₆-tagged ZAP-70 with a PreScission site inserted immediately prior to leucine 277 (residue 285 in construct) and PreScission II™, which contained two such sites, one upstream of arginine298 (residue 306 in construct) and one upstream of the C-terminal His₆ tag, so that this could be removed simultaneously. With the oligonucleotide MG474 and RS366 (see: SEQ ID No.3 and 4 respectively) and the plasmid NPL2173 encoding the wild type full-length ZAP-70 gene (Genbank Accession Nr. L05148), a DNA fragment is amplified which upon integration into the original NPL2173 allows the introduction of the PreScission™ cleavage site between alanine297 and arginine298. Another cleavage site is added after alanine619, preceeding the (His)₆-tag. The integration of the PCR fragment is done as described earlier (Geiser et al. Biotechnology 2001). The resulting plasmid is sequenced and the correct clone called pXI347 (PlasNova NPL003792). Similarly the plasmid pXI345 (NPL003793) is constructed by integrating the PCR fragment obtained from the NPL2173 plasmid template with the oligonucleotides MG475 and MG479 (SEQ ID No 5 and SEQ ID No 6) In pXI345, the PreScission™ cleavage site is integrated between threonine282 and leucine277 and the PreScission™ cleavage site in front of the (His)₆ tag is removed. The two plasmids are then introduced by transfection together with a linearized baculovirus DNA into insect cells. The numbering of amino acids is based on the sequences differs from that of Genbank Accession number L05148, by virtue of the inclusion of the purification tag and the inserted protease recognition sequences.

Based on the results of the limited proteolysis of the full sized ZAP-70 introduced after positions 276, 297 and after amino acid position 619, PreScission™ cleavage sites. The plasmids encoding the new mutants of ZAP-70 are called pXI345 and pXI347 respectively. These plasmids encode the protein ZAP PreScission™ I and ZAP PreScission™ II respectively. As a result of the mutations, complete and highly specific proteolysis of the full-length proteins can be carried out with a high quantitative recovery of the catalytic domain of ZAP-70.

### Example 4

### Expression and medium-scale fermentation of PreScission II ZAP in Baculovirus

Sf21 cells propagated in Excell 401 medium with 10% fetal calf serum are transfected with 500 ng of each recombinant transfer vector and 5 μl of linear AcNPV virus DNA (BacPAK 6) by lipofection using Bacfectin as transfection reagent (both BD/Clontech, Palo Alto, Calif.). After five days of incubation, the transfection supernatants are harvested and subjected to plaque assay, to derive a homogenous viral population. The isolated virus plaque picks are further amplified by infection of Sf21 cells grown in suspension in Excell 401 plus 1% FCS in roller culture, until full working virus stocks of both viruses are developed. These are again titered by plaque assays.

Large scale productions are carried out using the Wave bioreactor (Wave Biotech AG, Tagelswangen, Switzerland) at 10 I working volume. Sf9 cells growing in SF900 II medium (Gibco/Life Technologies) are inoculated in the Wave bag and allowed to grow for three consecutive days, reaching maximal cell densities of approx. 5×10⁶ cells ml. During the cultivation and infection process the airflow, the rocking rate and the rocking angle of the Wave reactor thermoplate are monitored and adjusted. The Sf9 cells are infected at cell densities of 1.6-4.9×10⁶ cells/ml and different multiplicities of infection (m.o.i.) between 0.5, 1 and 2 m.o.i. Simultaneously with virus addition, yeastolate (Gibco/Life Technologies) is fed to the cultures at a final concentration of 4 g/l. Cell density as well as cell viability is carefully recorded during the infection period.

Plaque assays of the amplified working virus stocks give rise to titers of 5.6×10⁷ pfu/ml for the ZAP70 PreScission™ I construct and 1.7×10⁸ pfu/ml for the ZAP-70 PreScission™ II construct. Both are subsequently used for 10 litre large-scale production of the two PreScission™ ZAP constructs. Both proteins are well-expressed and at least partially soluble; however, although LC-MS of NTA superflow purified PreScission™ II showed several peaks with masses of 72,404, 72,478, 72,557 and 72,635 which were thought to correspond to non-, mono-, di- and tri-phosphorylated full-length kinase, the PreScission™ I material gave such a heterogeneous LC-MS spectrum that it was impossible to assign masses, therefore all expression efforts were concentrated towards production of the PreScission™ II construct.

### Example 5

### Method for Optimising Harvesting of the Sf9 Cultures

The baculovirus expression system is a lytic system; as the infection proceeds, cells die and lyse, losing their contents into the medium. In the case of the expression of proteins such as ZAP, which are expressed intracellularly, there is a small “window” of expression in which maximal protein expression occurs before this is lost through cell lysis. Typically Western blotting will be used in conjunction with a time course of infection as well as variation of the multiplicity of infection (m.o.i.) to determine this time-point. However, in the case of ZAP PreScission™ II, the observation is made that increasing levels of expression are also accompanied by increasing levels of insoluble protein. Therefore, rapid small-scale purifications lysing 1 g quantities of cells give an “on-line” readout of “purifiable” ZAP.

1 g cell pellets are removed at different time points during fermentation and lysed as described previously (but using 15 ml lysis buffer, containing 1 Complete™ EDTA-free protease inhibitor tablet). Due to the scale-down, a 0.5 ml Ni—NTA-agarose column is used and the whole process from lysis through to analytical RP-HPLC estimate of the content of isolated protein is reduced to 34 hours. With an optimised harvest time of 48 h, the yield of soluble ZAP can be increased 4-fold to approximately 0.75 mg purifiable ZAP/g cells or −7.5 mg/litre culture.

### Example 6

### Staurosporin Binding to the ZAP Kinase Catalytic Domain

ZAP-70 kinase catalytic domain (R₂₉₈-A₆₁₉; SEQ ID No.2) defined by limited proteolysis is recloned as the full-length ZAP-70 kinase with a C-terminal His₆-affinity tag, but flanked by two PreScission™ protease sites. Expression is carried out using SF9 cells grown in 10 litre Wave™ bioreactors (0.45 m.o.i; 48 h). Cells are lysed in ice-cold buffer A (50 mM NaPO₄, 10% v/v glycerol, 10 mM β-mercaptoethanol, 300 mM NaCl, 10 mM Imidazole; pH 8.0) containing Complete™ EDTA-free protease inhibitor. The clarified lysate is passed over a 20 ml Ni—NTA-agarose column, the column washed with buffer A and then eluted with buffer B (25 mM Tris, 10% v/v glycerol, 50 mM NaCl, 250 mM Imidazole; pH 8.0). All chromatography steps are either carried out on-ice or using jacketed, cooled columns. The protein is immediately desalted into 25 mM Tris pH8.0, containing 1 mM EDTA, 1 mM DTT, 30% v/v glycerol and 150 mM NaCl, (using a 50 ml sephadex G-25 desalting column; HiPrep™ 26/10 Amersham Biosciences) concentrated and staurosporin added to 2-Molar excess (by addition of the correct volume of staurosporin dissolved in DMSO to 2 mg/ml).

The protein solution (60 ml) is frozen at −80° C. until required then concentrated using a 30,000 Mᵣ cut-off ultrafiltration membrane (Amicon) down to 10-15 ml prior to size-exclusion chromatography using an XK26/90 column packed with Superdex 75™ and equilibrated with 25 mM Tris pH 8, 1 mM EDTA, 1 mM DTT, 150 mM NaCl and 30% v/v glycerol. Fractions are collected, those corresponding to the monomeric material (ca. 40 ml) are pooled and incubated with PreScission™ protease to excise the catalytic domain from the rest of the molecule. Typically 40 ml of solution, containing 42 mg ZAP PreScission™ II is digested for 135 min at room temperature with 420 μl PreScission™ protease solution. The cleavage reaction is monitored by reversed phase HPLC so that the reaction can be stopped as quickly as possible by immediately desalting into buffer C (20 mM NaPO₄, 5 mM DTT, 10 mM NaCl, 1 mM MgCl₂: pH 7.2). Typically as 3×14 ml portions, applied at a flow-rate of 5 ml/minute to a HiLoad™ 26/10 column. The desalted protein, containing a mixture of monophosphorylated (25%) and non-phosphorylated (75%) ZAP is applied to an HR10/8 cation-exchange column (Mono S™ Amersham Biosciences) equilibrated in buffer C. The column is loaded at a flow-rate of 1 ml/minute and eluted at two ml/min using a 0-250 mM NaCl gradient over 512 ml.

Two major peaks are eluted, the monophosphorylated protein eluting at approximately 80 mM NaCl, the non-phosphorylated protein, slightly later at approximately 100 mM NaCl. The non- and mono-phosphorylated ZAP kinase catalytic domain peaks are collected separately, desalted (HiLoad 26/10 column) into 20 mM HEPES pH 7.2 (containing 5 mM DTT, 1 mM MgCl₂, 150 mM NaCl and 1% v/v ethyleneglycol). Staurosporine is again added to 10 Molar excess to both forms which are subsequently concentrated to 1040 mg/ml for crystallisation. This desalting step is critical, because it removes the glycerol required for chromatography (without which, the protein precipitates) and replaces it with a low concentration of ethylene glycol which sufficiently stabilises the protein through the concentration step, but doesn't interfere with the subsequent crystallization. 30% V/v glycerol, on the other hand, is not suitable for crystallisation as it has too large an influence on the evaporative sitting-drop process. In the case of the non-phosphorylated protein, half of the eluted peak, 36 ml is concentrated to 130 μl and a final concentration of 36 mg/ml.

By optimisation of the fermentation and harvest conditions, the level of ZAP expression is increased such that the purity of ZAP PreScission™ II being eluted from the NTA column is in excess of 75% and could be adequately purified by an additional size-exclusion chromatography step prior to cleavage. Following cleavage with PreScission™ protease, two bands are observed of similar mass representing the kinase and N-terminal portions of the molecule. Cation-exchange chromatography is applied to separate the two forms. The N-terminal portion passed through the column under the conditions used. The two peaks that are eluted from the column represented two different phosphorylated forms; the monophosphorylated protein eluting at approximately 80 mM NaCl and the non-phosphorylated protein, slightly later at approximately 100 mM NaCl. These different forms are collected separately for crystallisation. The yield of the mono-phosphorylated form is approximately 4-fold lower than the non-phosphorylated form, therefore most crystallography efforts concentrated on the non-phosphorylated form.

### Example 7

### Crystallisation of PreScission II ZAP

9 mg/ml ZAP-70 non-phosphorylated catalytic kinase domain in 20 mM Hepes, 5 mM DTT, 1 mM MgCl₂, 150 mM NaCl, 30% Glycerol, and staurosporine, are used for initial crystallization screening using 96 well sitting drop crystallography plates. The first promising microcrystals are obtained with crystallization screen at 10° C. Optimization of these crystallisation conditions is carried out by hanging drop vapor diffusion. The crystallisation screen at 10° C. has the following crystallisation conditions: 0.1 M Tris pH 7.5, 0.1 M KCl, 18% PEG 5000 monomethylether. Optimisation of this crystallisation condition along with optimisation of the formulation of the protein preparation results in larger crystals. Hanging drop vapor diffusion is used for optimisation. Diffracting single crystals are obtained with 29 mg/ml Zap70 nonphosphorylated kinase domain in 20 mM Hepes, 5 mM DTT, 1 mM MgCl₂, 150 mM NaCl, 1% Ethylene glycol, and staurosporine. The optimal growth condition is: 0.1 M Tris HCl pH 7.5, 0.2 M KCl, 20% PEG 5000 monomethylether at a crystallisation temperature of 10° C. Crystals appear after 1-2 days and optimal crystal size is reached after 1-2 weeks. Some single crystals grow, but most single fragments are obtained by breaking apart clusters.

### Example 8

### Protein Production and Crystallization of the Human ZAP-70 Protein Kinase Catalytic Domain

SEQ ID No.2 of the human ZAP-70 protein kinase catalytic domain (non-phosphorylated) is used for crystallization The construct comprises ZAP-70 residues 298 to 619 plus two additional residues from the PreScission™ cleavage site at the N-terminus and 6 residues from the PreScission™ cleavage site at the C-terminus. Small, but well-diffracting single crystals are obtained with a preparation of 29 mg/ml protein concentration and 1% Ethylene glycol. Optimal growth conditions are 20% PEG 5000 monomethylether, 0.1 M Tris HCl pH 7.5, 200 mM KCl. Crystals appear after 1-2 days and optimal crystal size is reached after 1-2 weeks. Some single crystals form but most single fragments are obtained by breaking apart clusters.

Cryo-Protection

Crystals are transferred from the drop into a solution consisting of the crystallization solution (well solution) plus 20% (v/v) of glycerol and 1 mM inhibitor. Crystals mounted in a 0.05 μm cryo loop are soaked in this cryo buffer for about 10-15 sec and then dipped into liquid propane.

Data Collection

A crystal is frozen in liquid propane and diffraction data are collected at 80K with a MAR CCD camera at the SLS in Villingen, Switzerland. A wavelength of 0.9803 A is used. 340 images are collected with 1.0° oscillation each, using an exposure time of 6 sec per frame and a crystal-to-detector distance of 140 mm. Raw diffraction data are processed and scaled with the HKL program suite version 1.96.6 (Otwinowski and Minor, 1996). Crystal data and data statistics are shown in Table 2.

Structure Determination and Refinement

The structure of the catalytic Zap70 kinase domain is determined by molecular replacement, using the coordinates of the LCK kinase domain x-ray structure (pdb: 3LCK, Yamaguchi and Hendrickson, 1996) as search model. Residues 245 to 501 of LCK are used and the phospho-Tyr residue 394 is removed for sequence similarity comparisons.

Molecular replacement is performed with the auto molecular replacement script in CCP4, using data to a maximal resolution of 4.0 Å. A 80% fraction completeness of the model with 50% fraction similarity to the input structure and 2 molecules in the asymmetric unit is expected. A clear solution is found and after an initial refinement in CNX using the standard script refine.inp the structure has an R-factor of 41.7% (R-free 43.5%). Inspection of the σ_{A}-weighted Fo-Fc electron density map with the program 0 version 7.0 (Jones et al., 1991) reveals a strong density for most of the C-alpha trace and most of the side chains. There are several insertions and deletions and a number of loops which are found outside the electron density and have to be corrected manually.

The model of the human ZAP-70 protein kinase catalytic domain is built and adjusted to fit the density where necessary. Insertions, mutations and deletions are made accordingly. The numbering was changed using the numbering of the SwissProt Accession number P43403 entry for ZAP-70. The structure is refined by a number of cycles of torsion angle dynamics and energy minimization, interspersed by model rebuilding steps. For refinement, the “refine.inp” script of CNX 2000 is used, with the following (non-default) option: Bulk solvent correction (based on the mask method).

Cross-validation is used throughout refinement using a test set comprising 5% of the reflections. Water molecules are identified with the CNX script water_pick.inp, and selected based on difference peak height (greater than 3.0σ), hydrogen-bonding and distance criteria. NCS restrain is used for the two molecules in the asymmetric unit.

The quality of the final refined model is assessed with the programs CNX 2000 (Brünger, 1998) (see: FIG. 7). Pictures are made in O (Jones et al., 1991) or WebLab ViewerLite 3.5 (Molecular Simulations, Inc.).

Results

Small single crystals of the human Zap70 kinase domain are obtained with PEGMME at pH 7.5. The crystals grow in space group P1 with 2 monomers per asymmetric unit. The structure is determined by molecular replacement. The final model includes two kinase domains (residue 331 to 603), one molecule of staurosporine per kinase domain and 261 water molecules. It has a good geometry with a rms deviation of 0.011 Å on bond lengths and 1.3° on bond angles. The final R-factor was 0.182 (Rfree=0.209) for all reflections between 19.26 Å and 1.90 Å.

Overall Structure

The crystal structure is extremely well defined, the full c-alpha trace between residue 331 and 603 is defined. All loops and important residues have good electron density. The B-factor distribution is as expected. The staurosporine-binding site and all interactions with the kinase domain can be described in detail. The quality of the model is good, the final R-factor is 18.2% (R-free 20.9%).

The ZAP-70 kinase domain folds into the typical kinase domain fold. Most of the residues are well defined by the electron density with a few exceptions: The N-terminal residues 296 to 330 and the C-terminal residues 604 to 625 are completely disordered and therefore not visible in the x-ray structure. There are a few side chains at the surface of the molecules that do not show a well-defined electron density. A number of side chains clearly show alternating side chain positions. Those were not refined; there is only one side chain with alternating conformation close to the staurosporin-binding site, which could be of importance: SER 478.

There is an electron density of the linker region between β5 and αD. This region has an unusual sequence (Ala417-Gly418-Gly419-Gly420-Pro421) and forms part of the staurosporine binding site. This region is well ordered and the electron density is well defined. The two important contacts (Glu 415O-N1, Ala 417N -O5).

Staurosporine-Binding Site

The binding site for staurosporine in mostly shaped by the hydrophobic side chains of the following residues: Leu344, Phe349, Val352, Val399, Met414, Met416, Ala417, Leu468. Deep in the binding pocket, two polar interactions contribute to the staurosporine binding: the main chain carbonyl oxygen of residue Glu415 binds to nitrogen N1 of staurosporine and the main chain peptide nitrogen of residue Ala417 interacts with the carbonyl oxygen 05 of staurosporine. At the other end of the staurosporine molecule there are a few polar interactions between the sugar moiety and the protein. The staurosporine methoxy group forms only van der Waals contacts, but the -N-Met group interacts with the protein through a hydrogen bridge network that is formed by a number of solvent molecules and the side chains of residue Lys424, His423, Arg465 (also involving the carboxyl oxygen of the peptide bond), Asp479, Asn466.

An alignment (ClustalW) with some of the well-known kinase structures show that the sequence variability is rather high. Nevertheless, if the c-alpha traces get superimposed, usually a relatively good fit is noticed (53-59%).

From the alignment, many kinases have insertions or deletions compared to Zap70 which makes is extremely difficult to model the structure in every detail. Nevertheless, the ATP-binding site has a relatively conserved structure and is easier to model than most of the rest of the kinase domain.

Phosphorylation Sites

Phosphorylation of residues in the activation segment causes conformational changes in the catalytic kinase domain that lead to the correct positioning of substrate binding residues and catalytic residues, and relief of steric blocking to enable access of substrate to the catalytic site.

The kinase domain crystallized here, is not phosphorylated. However, Zap70 contains a number of tyrosine residues, which can be phosphorylated in vivo and contribute to the regulation of the kinase activity and adaptor molecule binding. Due to the fact, that we are targeting the ATP binding site, it should not be a huge drawback that we only have the structure of the non-activated/phosphorylated kinase domain.

The phosphorylation sites 474, 492, 493, 597, and 598 are defined in the x-ray structure. Tyrosine 492 and 493 are both located in the activation loop. Transphosphorylation of Tyrosine 493 by Lck leads to activation of Zap70. (Chan, et al 1995, Wange et al 1995, Mège, et al 1996) This tyrosine residue corresponds to Tyrosine 1163 of the insulin receptor kinase domain which causes upon transphosphorylation, a major conformational change of the activation loop (Hubbard 1997). It is thus likely that the structure of the corresponding activation loop of ZAP-70 phosphorylated at Tyrosine 493 will be different than the one shown here. The role of Tyrosine 492 is less clear. A negative regulatory function has been proposed (Chan, et al 1995). Tyrosine 474 is required for association with the Shc adapter, which couples T cell receptor signaling to the Ras pathway (Pacini, et al 1998). The surface exposed Tyrosine 474 is located at the beginning of the β8 segment, close to the following activation segment. Tyrosine 597 and 598 located at the surface of the protein near to the C-terminus are supposed to be involved in regulating the functional activity of ZAP-70 possibly by inhibitory proteins (Zeitlmann, et al 1998).

TABLE 1
Atomic Coordinates of ZAP-70 catalytic Domain with Staurosporine

| | | | |
|---|---|---|---|
| REMARK | 3 | PROGRAM: CNX 2000.1 | |
| REMARK | 3 | AUTHORS: Brunger, Adams, Clore, Delano, | |
| REMARK | 3 | Gros, Grosse-Kunstleve, Jiang, | |
| REMARK | 3 | Kuszewski, Nilges, Pannu, Read, | |
| REMARK | 3 | Rice, Simonson, Warren | |
| REMARK | 3 | and | |
| REMARK | 3 | Molecular Simulations Inc., | |
| REMARK | 3 | (Badger, Berard, Kumar, Szalma, | |
| REMARK | 3 | Yip). | |
| REMARK | 3 | | |
| REMARK | 3 | DATA USED IN REFINEMENT. | |
| REMARK | 3 | RESOLUTION RANGE HIGH (ANGSTROMS): | 1.90 |
| REMARK | 3 | RESOLUTION RANGE LOW (ANGSTROMS): | 19.26 |
| REMARK | 3 | DATA CUTOFF (SIGMA(F)): | 0.0 |
| REMARK | 3 | DATA CUTOFF HIGH (ABS(F)): | 933984.58 |
| REMARK | 3 | DATA CUTOFF LOW (ABS(F)): | 0.000000 |
| REMARK | 3 | COMPLETENESS (WORKING +TEST) (%): | 96.7 |
| REMARK | 3 | NUMBER OF REFLECTIONS: | 45463 |
| REMARK | 3 | | |
| REMARK | 3 | FIT TO DATA USED IN REFINEMENT. | |
| REMARK | 3 | CROSS-VALIDATION METHOD: | THROUGHOUT |
| REMARK | 3 | FREE R VALUE TEST SET SELECTION: | RANDOM |
| REMARK | 3 | R VALUE (WORKING SET): | 0.182 |
| REMARK | 3 | FREE R VALUE: | 0.209 |
| REMARK | 3 | FREE R VALUE TEST SET SIZE (%): | 5.0 |
| REMARK | 3 | FREE R VALUE TEST SET COUNT: | 2284 |
| REMARK | 3 | ESTIMATED ERROR OF FREE R VALUE: | 0.004 |
| REMARK | 3 | | |
| REMARK | 3 | FIT IN THE HIGHEST RESOLUTION BIN. | |
| REMARK | 3 | TOTAL NUMBER OF BINS USED: | 6 |
| REMARK | 3 | BIN RESOLUTION RANGE HIGH (A): | 1.90 |
| REMARK | 3 | BIN RESOLUTION RANGE LOW (A): | 2.02 |
| REMARK | 3 | BIN COMPLETENESS (WORKING +TEST) (%): | 93.7 |
| REMARK | 3 | REFLECTIONS IN BIN (WORKING SET): | 6918 |
| REMARK | 3 | BIN R VALUE (WORKING SET): | 0.195 |
| REMARK | 3 | BIN FREE R VALUE: | 0.241 |
| REMARK | 3 | BIN FREE R VALUE TEST SET SIZE (%): | 5.2 |
| REMARK | 3 | BIN FREE R VALUE TEST SET COUNT: | 378 |
| REMARK | 3 | ESTIMATED ERROR OF BIN FREE R VALUE: | 0.012 |
| REMARK | 3 | | |
| REMARK | 3 | NUMBER OF NON-HYDROGEN ATOMS USED IN | |
| | | REFINEMENT. | |
| REMARK | 3 | PROTEIN ATOMS: | 4739 |
| REMARK | 3 | NUCLEIC ACID ATOMS: | 0 |
| REMARK | 3 | HETEROGEN ATOMS: | 0 |
| REMARK | 3 | SOLVENT ATOMS: | 0 |
| REMARK | 3 | | |
| REMARK | 3 | B VALUES. | |
| REMARK | 3 | FROM WILSON PLOT (A**2) | 24.1 |
| REMARK | 3 | MEAN B VALUE (OVERALL, A**2) | 35.0 |
| REMARK | 3 | OVERALL ANISOTROPIC B VALUE. | |
| REMARK | 3 | Bil (A**2): | 0.16 |
| REMARK | 3 | B22 (A**2): | −1.31 |
| REMARK | 3 | B33 (A**2): | 1.15 |
| REMARK | 3 | B12 (A**2): | 0.00 |
| REMARK | 3 | B13 (A**2): | 2.08 |
| REMARK | 3 | B23 (A**2): | −1.25 |
| REMARK | 3 | | |
| REMARK | 3 | BULK SOLVENT MODELING. | |
| REMARK | 3 | METHOD USED: | FLAT MODEL |
| REMARK | 3 | KSOL: | 0.38494 |
| REMARK | 3 | BSOL: | 52.9849 (A**2) |
| REMARK | 3 | | |
| REMARK | 3 | ESTIMATED COORDINATE ERROR. | |
| REMARK | 3 | ESD FROM LUZZATI PLOT (A): | 0.19 |
| REMARK | 3 | ESD FROM SIGMAA (A): | 0.01 |
| REMARK | 3 | LOW RESOLUTION CUTOFF (A): | 5.00 |
| REMARK | 3 | | |
| REMARK | 3 | CROSS-VALIDATED ESTIMATED COORDINATE | |
| | | ERROR. | |
| REMARK | 3 | ESD FROM C-V LUZZATI PLOT (A): | 0.22 |
| REMARK | 3 | ESD FROM C-V SIGMAA (A): | 0.11 |
| REMARK | 3 | | |
| REMARK | 3 | RMS DEVIATIONS FROM IDEAL VALUES. | |
| REMARK | 3 | BOND LENGTHS (A): | 0.011 |
| REMARK | 3 | BOND ANGLES (DEGREES): | 1.3 |
| REMARK | 3 | DIHEDRAL ANGLES (DEGREES): | 21.4 |
| REMARK | 3 | IMPROPER ANGLES (DEGREES): | 0.91 |
| REMARK | 3 | | |
| REMARK | 3 | ISOTROPIC THERMAL MODEL: | RESTRAINED |
| REMARK | 3 | | |
| REMARK | 3 | ISOTROPIC THERMAL FACTOR RESTRAINTS. | RMS SIGMA |
| REMARK | 3 | MAIN-CHAIN BOND (A**2): | 3.56 ; 1.50 |
| REMARK | 3 | MAIN-CHAIN ANGLE (A**2): | 4.55 ; 2.00 |
| REMARK | 3 | SIDE-CHAIN BOND (A**2): | 4.82 ; 2.00 |
| REMARK | 3 | SIDE-CHAIN ANGLE (A**2): | 6.37 ; 2.50 |
| REMARK | 3 | | |
| REMARK | 3 | NCS MODEL: NONE | |
| REMARK | 3 | | |
| REMARK | 3 | NCS RESTRAINTS. | RMS SIGMA/WEIGHT |
| REMARK | 3 | GROUP 1 POSITIONAL (A) | NULL ; NULL |
| REMARK | 3 | GROUP 1 B-FACTOR (A**2) | NULL ; NULL |
| REMARK | 3 | | |
| REMARK | 3 | PARAMETER FILE 1: | MSI_CNX_TOPPAR/proteinrep.param |
| REMARK | 3 | PARAMETER FILE 2: | MSI_10 CNX_TOPPAR/waterrep.param |
| REMARK | 3 | PARAMETER FILE 3: | stu.param |
| REMARK | 3 | TOPOLOGY FILE 1: | MSI_CNX_TOPPAR/protein.top |
| REMARK | 3 | TOPOLOGY FILE 2: | MSI_.CNX_TOPPAR/water.top |
| REMARK | 3 | TOPOLOGY FILE 3: | stu.toppar |
| REMARK | 3 | | |

| | | |
|---|---|---|
| REMARK | 3 | OTHER REFINEMENT REMARKS: NULL |
| SEQRES | 1 | A 273 PHE LEU LYS ARG ASP ASN LEU LEU ILE ALA ASP ILE GLU |
| SEQRES | 2 | A 273 LEU GLY CYS GLY ASN PHE GLY SER VAL ARG GLN GLY VAL |
| SEQRES | 3 | A 273 TYR ARG MET ARG LYS LYS GLN ILE ASP VAL ALA ILE LYS |
| SEQRES | 4 | A 273 VAL LEU LYS GLN GLY THR GLU LYS ALA ASP THR GLU GLU |
| SEQRES | 5 | A 273 MET MET ARG GLU ALA GLN ILE MET HIS GLN LEU ASP ASN |
| SEQRES | 6 | A 273 PRO TYR ILE VAL ARG LEU ILE GLY VAL CYS GLN ALA GLU |
| SEQRES | 7 | A 273 ALA LEU MET LEU VAL MET GLU MET ALA GLY GLY GLY PRO |
| SEQRES | 8 | A 273 LEU HIS LYS PHE LEU VAL GLY LYS ARG GLU GLU ILE PRO |
| SEQRES | 9 | A 273 VAL SER ASN VAL ALA GLU LEU LEU HIS GLN VAL SER MET |
| SEQRES | 10 | A 273 GLY MET LYS TYR LEU GLU GLU LYS ASN PHE VAL HIS ARG |
| SEQRES | 11 | A 273 ASP LEU ALA ALA ARG ASN VAL LEU LEU VAL ASN ARG HIS |
| SEQRES | 12 | A 273 TYR ALA LYS ILE SER ASP PHE GLY LEU SER LYS ALA LEU |
| SEQRES | 13 | A 273 GLY ALA ASP ASP SER TYR TYR THR ALA ARG SER ALA GLY |
| SEQRES | 14 | A 273 LYS TRP PRO LEU LYS TRP TYR ALA PRO GLU CYS ILE ASN |
| SEQRES | 15 | A 273 PHE ARG LYS PHE SER SER ARG SER ASP VAL TRP SER TYR |
| SEQRES | 16 | A 273 GLY VAL THR MET TRP GLU ALA LEU SER TYR GLY GLN LYS |
| SEQRES | 17 | A 273 PRO TYR LYS LYS MET LYS GLY PRO GLU VAL MET ALA PHE |
| SEQRES | 18 | A 273 ILE GLU GLN GLY LYS ARG MET GLU CYS PRO PRO GLU CYS |
| SEQRES | 19 | A 273 PRO PRO GLU LEU TYR ALA LEU MET SER ASP CYS TRP ILE |
| SEQRES | 20 | A 273 TYR LYS TRP GLU ASP ARG PRO ASP PHE LEU THR VAL GLU |
| SEQRES | 21 | A 273 GLN ARG MET ARG ALA CYS TYR TYR SER LEU ALA SER LYS |
| SEQRES | 1 | B 273 PHE LEU LYS ARG ASP ASN LEU LEU ILE ALA ASP ILE GLU |
| SEQRES | 2 | B 273 LEU GLY CYS GLY ASN PHE GLY SER VAL ARG GLN GLY VAL |
| SEQRES | 3 | B 273 TYR ARG MET ARG LYS LYS GLN ILE ASP VAL ALA ILE LYS |
| SEQRES | 4 | B 273 VAL LEU LYS GLN GLY THR GLU LYS ALA ASP THR GLU GLU |
| SEQRES | 5 | B 273 MET MET ARG GLU ALA GLN ILE MET HIS GLN LEU ASP ASN |
| SEQRES | 6 | B 273 PRO TYR ILE VAL ARG LEU ILE GLY VAL CYS GLN ALA GLU |
| SEQRES | 7 | B 273 ALA LEU MET LEU VAL MET GLU MET ALA GLY GLY GLY PRO |
| SEQRES | 8 | B 273 LEU HIS LYS PHE LEU VAL GLY LYS ARG GLU GLU ILE PRO |
| SEQRES | 9 | B 273 VAL SER ASN VAL ALA GLU LEU LEU HIS GLN VAL SER MET |
| SEQRES | 10 | B 273 GLY MET LYS TYR LEU GLU GLU LYS ASN PHE VAL HIS ARG |
| SEQRES | 11 | B 273 ASP LEU ALA ALA ARG ASN VAL LEU LEU VAL ASN ARG HIS |
| SEQRES | 12 | B 273 TYR ALA LYS ILE SER ASP PHE GLY LEU SER LYS ALA LEU |
| SEQRES | 13 | B 273 GLY ALA ASP ASP SER TYR TYR THR ALA ARG SER ALA GLY |
| SEQRES | 14 | B 273 LYS TRP PRO LEU LYS TRP TYR ALA PRO GLU CYS ILE ASN |
| SEQRES | 15 | B 273 PHE ARG LYS PHE SER SER ARG SER ASP VAL TRP SER TYR |
| SEQRES | 16 | B 273 GLY VAL THR MET TRP GLU ALA LEU SER TYR GLY GLN LYS |
| SEQRES | 17 | B 273 PRO TYR LYS LYS MET LYS GLY PRO GLU VAL MET ALA PHE |
| SEQRES | 18 | B 273 ILE GLU GLN GLY LYS ARG MET GLU CYS PRO PRO GLU CYS |
| SEQRES | 19 | B 273 PRO PRO GLU LEU TYR ALA LEU MET SER ASP CYS TRP ILE |
| SEQRES | 20 | B 273 TYR LYS TRP GLU ASP ARG PRO ASP PHE LEU THR VAL GLU |
| SEQRES | 21 | B 273 GLN ARG MET ARG ALA CYS TYR TYR SER LEU ALA SER LYS |
| SEQRES | 1 | C 1 STU |
| SEQRES | 1 | D 1 STU |
| SEQRES | 1 | S 261 TIP TIP TIP TIP TIP TIP TIP TIP TIP TIP TIP TIP TIP |
| SEQRES | 2 | S 261 TIP TIP TIP TIP TIP TIP TIP TIP TIP TIP TIP TIP TIP |
| SEQRES | 3 | S 261 TIP TIP TIP TIP TIP TIP TIP TIP TIP TIP TIP TIP TIP |
| SEQRES | 4 | S 261 TIP TIP TIP TIP TIP TIP TIP TIP TIP TIP TIP TIP TIP |
| SEQRES | 5 | S 261 TIP TIP TIP TIP TIP TIP TIP TIP TIP TIP TIP TIP TIP |
| SEQRES | 6 | S 261 TIP TIP TIP TIP TIP TIP TIP TIP TIP TIP TIP TIP TIP |
| SEQRES | 7 | S 261 TIP TIP TIP TIP TIP TIP TIP TIP TIP TIP TIP TIP TIP |
| SEQRES | 8 | S 261 TIP TIP TIP TIP TIP TIP TIP TIP TIP TIP TIP TIP TIP |
| SEQRES | 9 | S 261 TIP TIP TIP TIP TIP TIP TIP TIP TIP TIP TIP TIP TIP |
| SEQRES | 10 | S 261 TIP TIP TIP TIP TIP TIP TIP TIP TIP TIP TIP TIP TIP |
| SEQRES | 11 | S 261 TIP TIP TIP TIP TIP TIP TIP TIP TIP TIP TIP TIP TIP |
| SEQRES | 12 | S 261 TIP TIP TIP TIP TIP TIP TIP TIP TIP TIP TIP TIP TIP |
| SEQRES | 13 | S 261 TIP TIP TIP TIP TIP TIP TIP TIP TIP TIP TIP TIP TIP |
| SEQRES | 14 | S 261 TIP TIP TIP TIP TIP TIP TIP TIP TIP TIP TIP TIP TIP |
| SEQRES | 15 | S 261 TIP TIP TIP TIP TIP TIP TIP TIP TIP TIP TIP TIP TIP |
| SEQRES | 16 | S 261 TIP TIP TIP TIP TIP TIP TIP TIP TIP TIP TIP TIP TIP |
| SEQRES | 17 | S 261 TIP TIP TIP TIP TIP TIP TIP TIP TIP TIP TIP TIP TIP |
| SEQRES | 18 | S 261 TIP TIP TIP TIP TIP TIP TIP TIP TIP TIP TIP TIP TIP |
| SEQRES | 19 | S 261 TIP TIP TIP TIP TIP TIP TIP TIP TIP TIP TIP TIP TIP |
| SEQRES | 20 | S 261 TIP TIP TIP TIP TIP TIP TIP TIP TIP TIP TIP TIP TIP |
| SEQRES | 21 | S 261 TIP |
| CRYST | 1 | 35.768 57.562 80.211 68.97 89.83 89.95 P 1 5 |
| ORIGX | 1 | 1.000000 0.000000 0.000000 0.00000 |
| ORIGX | 2 | 0.000000 1.000000 0.000000 0.00000 |
| ORIGX | 3 | 0.000000 0.000000 1.000000 0.00000 |
| SCALE | 1 | 0.027958 −0.000024 −0.000079 0.00000 |
| SCALE | 2 | 0.000000 0.017373 −0.006679 0.00000 |
| SCALE | 3 | 0.000000 0.000000 0.013357 0.00000 |

| | | | | | | | | | | | |
|---|---|---|---|---|---|---|---|---|---|---|---|
| ATOM | 1 | CB | PHE | A | 331 | −0.844 | −23.839 | −13.860 | 1.00 | 47.80 | C |
| ATOM | 2 | CG | PHE | A | 331 | −0.845 | −24.256 | −12.414 | 1.00 | 49.69 | C |
| ATOM | 3 | CD1 | PHE | A | 331 | −2.033 | −24.319 | −11.699 | 1.00 | 53.17 | C |
| ATOM | 4 | CD2 | PHE | A | 331 | 0.342 | −24.562 | −11.764 | 1.00 | 47.25 | C |
| ATOM | 5 | CE1 | PHE | A | 331 | −2.041 | −24.679 | −10.359 | 1.00 | 48.16 | C |
| ATOM | 6 | CE2 | PHE | A | 331 | 0.344 | −24.924 | −10.421 | 1.00 | 51.80 | C |
| ATOM | 7 | CZ | PHE | A | 331 | −0.849 | −24.982 | −9.720 | 1.00 | 49.42 | C |
| ATOM | 8 | C | PHE | A | 331 | −0.689 | −21.905 | −15.439 | 1.00 | 47.08 | C |
| ATOM | 9 | O | PHE | A | 331 | −1.461 | −21.795 | −16.393 | 1.00 | 48.72 | O |
| ATOM | 10 | N | PHE | A | 331 | −2.670 | −22.155 | −13.939 | 1.00 | 46.43 | N |
| ATOM | 11 | CA | PHE | A | 331 | −1.196 | −22.366 | −14.074 | 1.00 | 43.11 | C |
| ATOM | 12 | N | LEU | A | 332 | 0.610 | −21.631 | −15.514 | 1.00 | 40.15 | N |
| ATOM | 13 | CA | LEU | A | 332 | 1.245 | −21.166 | −16.746 | 1.00 | 42.89 | C |
| ATOM | 14 | CB | LEU | A | 332 | 1.949 | −19.824 | −16.490 | 1.00 | 42.08 | C |
| ATOM | 15 | CG | LEU | A | 332 | 1.135 | −18.662 | −15.894 | 1.00 | 41.32 | C |
| ATOM | 16 | CD1 | LEU | A | 332 | 2.088 | −17.561 | −15.415 | 1.00 | 36.46 | C |
| ATOM | 17 | CD2 | LEU | A | 332 | 0.146 | −18.116 | −16.931 | 1.00 | 40.99 | C |
| ATOM | 18 | C | LEU | A | 332 | 2.273 | −22.195 | −17.207 | 1.00 | 41.99 | C |
| ATOM | 19 | O | LEU | A | 332 | 2.746 | −23.003 | −16.416 | 1.00 | 41.81 | O |
| ATOM | 20 | N | LYS | A | 333 | 2.615 | −22.171 | −18.492 | 1.00 | 42.98 | N |
| ATOM | 21 | CA | LYS | A | 333 | 3.614 | −23.091 | −19.018 | 1.00 | 44.97 | C |
| ATOM | 22 | CB | LYS | A | 333 | 3.495 | −23.221 | −20.537 | 1.00 | 45.64 | C |
| ATOM | 23 | CG | LYS | A | 333 | 2.201 | −23.857 | −20.971 | 1.00 | 51.28 | C |
| ATOM | 24 | CD | LYS | A | 333 | 2.310 | −24.340 | −22.408 | 1.00 | 57.41 | C |
| ATOM | 25 | CE | LYS | A | 333 | 1.041 | −25.067 | −22.838 | 1.00 | 58.44 | C |
| ATOM | 26 | NZ | LYS | A | 333 | 1.189 | −25.656 | −24.206 | 1.00 | 62.81 | N |
| ATOM | 27 | C | LYS | A | 333 | 4.998 | 22.577 | −18.673 | 1.00 | 43.59 | C |
| ATOM | 28 | O | LYS | A | 333 | 5.321 | −21.420 | −18.952 | 1.00 | 39.19 | O |
| ATOM | 29 | N | ARG | A | 334 | 5.813 | −23.455 | −18.097 | 1.00 | 37.84 | N |
| ATOM | 30 | CA | ARG | A | 334 | 7.167 | −23.109 | −17.698 | 1.00 | 42.87 | C |
| ATOM | 31 | CB | ARG | A | 334 | 7.831 | −24.309 | −17.015 | 1.00 | 46.05 | C |
| ATOM | 32 | CG | ARG | A | 334 | 9.264 | −24.063 | −16.553 | 1.00 | 41.63 | C |
| ATOM | 33 | CD | ARG | A | 334 | 9.303 | −23.119 | −15.368 | 1.00 | 37.46 | C |
| ATOM | 34 | NE | ARG | A | 334 | 10.669 | −22.817 | −14.947 | 1.00 | 37.67 | N |
| ATOM | 35 | CZ | ARG | A | 334 | 11.471 | −21.972 | 15.580 | 1.00 | 41.87 | C |
| ATOM | 36 | NH1 | ARG | A | 334 | 11.038 | −21.340 | −16.671 | 1.00 | 38.44 | N |
| ATOM | 37 | NH2 | ARG | A | 334 | 12.699 | −21.747 | −15.121 | 1.00 | 41.45 | N |
| ATOM | 38 | C | ARG | A | 334 | 7.999 | −22.691 | −18.905 | 1.00 | 42.10 | C |
| ATOM | 39 | O | ARG | A | 334 | 8.981 | −21.961 | −18.769 | 1.00 | 39.41 | O |
| ATOM | 40 | N | ASP | A | 335 | 7.587 | −23.169 | −20.077 | 1.00 | 43.76 | N |
| ATOM | 41 | CA | ASP | A | 335 | 8.274 | −22.895 | −21.340 | 1.00 | 44.45 | C |
| ATOM | 42 | CB | ASP | A | 335 | 7.718 | −23.822 | −22.423 | 1.00 | 52.46 | C |
| ATOM | 43 | CG | ASP | A | 335 | 7.632 | −25.261 | −31.962 | 1.00 | 55.02 | C |
| ATOM | 44 | OD1 | ASP | A | 335 | 8.688 | −25.925 | −21.897 | 1.00 | 60.98 | O |
| ATOM | 45 | OD2 | ASP | A | 335 | 6.512 | −25.723 | −21.643 | 1.00 | 62.85 | O |
| ATOM | 46 | C | ASP | A | 335 | 8.110 | −21.455 | −21.789 | 1.00 | 43.50 | C |
| ATOM | 47 | O | ASP | A | 335 | 8.904 | −20.951 | −22.594 | 1.00 | 41.39 | O |
| ATOM | 48 | N | ASN | A | 336 | 7.079 | −20.783 | −21.287 | 1.00 | 30.71 | N |
| ATOM | 49 | CA | ASN | A | 336 | 6.837 | −19.407 | −21.673 | 1.00 | 33.27 | C |
| ATOM | 50 | CB | ASN | A | 336 | 5.341 | −19.129 | −31.723 | 1.00 | 36.84 | C |
| ATOM | 51 | CG | ASN | A | 336 | 4.647 | −19.915 | −22.809 | 1.00 | 49.26 | C |
| ATOM | 52 | OD1 | ASN | A | 336 | 5.193 | −20.109 | −23.899 | 1.00 | 49.53 | O |
| ATOM | 53 | ND2 | ASN | A | 336 | 3.426 | −20.351 | −22.527 | 1.00 | 48.43 | N |
| ATOM | 54 | C | ASN | A | 336 | 7.494 | −18.421 | −20.731 | 1.00 | 28.53 | C |
| ATOM | 55 | O | ASN | A | 336 | 7.363 | −17.210 | −20.895 | 1.00 | 30.15 | O |
| ATOM | 56 | N | LEU | A | 337 | 8.203 | −18.948 | −19.749 | 1.00 | 32.36 | N |
| ATOM | 57 | CA | LEU | A | 337 | 8.855 | −18.115 | −18.750 | 1.00 | 34.27 | C |
| ATOM | 58 | CB | LEU | A | 337 | 8.365 | −18.518 | −17.350 | 1.00 | 30.14 | C |
| ATOM | 59 | CG | LEU | A | 337 | 8.920 | −17.781 | −16.125 | 1.00 | 28.85 | C |
| ATOM | 60 | CD1 | LEU | A | 337 | 8.290 | −16.416 | −16.039 | 1.00 | 27.73 | C |
| ATOM | 61 | CD2 | LEU | A | 337 | 8.586 | −18.559 | −14.850 | 1.00 | 35.78 | C |
| ATOM | 62 | C | LEU | A | 337 | 10.380 | −18.202 | −18.800 | 1.00 | 31.60 | C |
| ATOM | 63 | O | LEU | A | 337 | 10.956 | −19.279 | −18.875 | 1.00 | 32.32 | O |
| ATOM | 64 | N | LEU | A | 338 | 11.032 | −17.051 | −18.741 | 1.00 | 25.65 | N |
| ATOM | 65 | CA | LEU | A | 338 | 12.486 | −17.006 | −18.731 | 1.00 | 24.94 | C |
| ATOM | 66 | CB | LEU | A | 338 | 13.020 | −16.257 | −19.977 | 1.00 | 28.20 | C |
| ATOM | 67 | CG | LEU | A | 338 | 14.554 | −16.272 | −20.089 | 1.00 | 30.23 | C |
| ATOM | 68 | CD1 | LEU | A | 338 | 15.028 | −17.702 | −20.306 | 1.00 | 33.75 | C |
| ATOM | 69 | CD2 | LEU | A | 338 | 15.012 | −15.400 | −21.245 | 1.00 | 31.10 | C |
| ATOM | 70 | C | LEU | A | 338 | 12.855 | −16.235 | −17.465 | 1.00 | 29.76 | C |
| ATOM | 71 | O | LEU | A | 338 | 12.624 | −15.030 | −17.384 | 1.00 | 30.99 | O |
| ATOM | 72 | N | ILE | A | 339 | 13.401 | −16.933 | −16.471 | 1.00 | 30.96 | N |
| ATOM | 73 | CA | ILE | A | 339 | 13.763 | −16.266 | −15.219 | 1.00 | 29.19 | C |
| ATOM | 74 | CB | ILE | A | 339 | 13.645 | −17.230 | −14.011 | 1.00 | 33.77 | C |
| ATOM | 75 | CG2 | ILE | A | 339 | 13.921 | −16.458 | −13.704 | 1.00 | 30.52 | C |
| ATOM | 76 | CG1 | ILE | A | 339 | 12.267 | −17.901 | 14.025 | 1.00 | 32.27 | C |
| ATOM | 77 | CD1 | ILE | A | 339 | 12.057 | −18.955 | −12.960 | 1.00 | 38.69 | C |
| ATOM | 78 | C | ILE | A | 339 | 15.185 | −15.743 | −15.277 | 1.00 | 31.14 | C |
| ATOM | 79 | O | ILE | A | 339 | 16.119 | −16.500 | −15.534 | 1.00 | 34.19 | O |
| ATOM | 80 | N | ALA | A | 340 | 15.347 | −14.448 | −15.029 | 1.00 | 29.75 | N |
| ATOM | 81 | CA | ALA | A | 340 | 16.676 | −13.843 | −15.055 | 1.00 | 34.31 | C |
| ATOM | 82 | CB | ALA | A | 340 | 16.569 | −12.342 | −15.334 | 1.00 | 32.66 | C |
| ATOM | 83 | C | ALA | A | 340 | 17.406 | −14.090 | −13.736 | 1.00 | 36.59 | C |
| ATOM | 84 | O | ALA | A | 340 | 16.788 | −14.463 | −12.724 | 1.00 | 34.25 | O |
| ATOM | 85 | N | ASP | A | 341 | 18.718 | −13.901 | −13.745 | 1.00 | 34.25 | N |
| ATOM | 86 | CA | ASP | A | 341 | 19.514 | −14.093 | −12.542 | 1.00 | 37.73 | C |
| ATOM | 87 | CA | ASP | A | 341 | 20.924 | −14.582 | −12.911 | 1.00 | 45.65 | C |
| ATOM | 88 | CG | ASP | A | 341 | 21.639 | −15.259 | −11.738 | 1.00 | 56.30 | C |
| ATOM | 89 | OD1 | ASP | A | 341 | 21.455 | −14.811 | −10.588 | 1.00 | 59.09 | O |
| ATOM | 90 | OD2 | ASP | A | 341 | 22.399 | −16.234 | −11.966 | 1.00 | 62.33 | O |
| ATOM | 91 | C | ASP | A | 341 | 19.594 | −12.739 | −11.850 | 1.00 | 43.58 | C |
| ATOM | 92 | O | ASP | A | 341 | 20.683 | −12.245 | −11.562 | 1.00 | 44.94 | O |
| ATOM | 93 | N | ILE | A | 342 | 18.434 | −12.134 | −11.595 | 1.00 | 36.82 | N |
| ATOM | 94 | CA | ILE | A | 342 | 18.349 | −10.829 | −10.954 | 1.00 | 35.58 | C |
| ATOM | 95 | CB | ILE | A | 342 | 17.894 | −9.763 | −11.969 | 1.00 | 38.49 | C |
| ATOM | 96 | CG2 | ILE | A | 342 | 17.708 | −8.407 | −11.281 | 1.00 | 43.25 | C |
| ATOM | 97 | CG1 | ILE | A | 342 | 18.912 | −9.675 | −13.106 | 1.00 | 36.31 | C |
| ATOM | 98 | CD1 | ILE | A | 342 | 18.468 | −8.769 | −14.239 | 1.00 | 46.83 | C |
| ATOM | 99 | C | ILE | A | 342 | 17.327 | −10.911 | −9.825 | 1.00 | 37.22 | C |
| ATOM | 100 | O | ILE | A | 342 | 16.214 | −11.364 | −10.037 | 1.00 | 31.02 | O |
| ATOM | 101 | N | GLU | A | 343 | 17.710 | −10.475 | −8.633 | 1.00 | 33.59 | N |
| ATOM | 102 | CA | GLU | A | 343 | 16.816 | −10.526 | −7.485 | 1.00 | 36.18 | C |
| ATOM | 103 | CB | GLU | A | 343 | 17.566 | −11.064 | −6.264 | 1.00 | 36.52 | C |
| ATOM | 104 | CG | GLU | A | 343 | 16.713 | −11.123 | −5.003 | 1.00 | 40.70 | C |
| ATOM | 105 | CD | GLU | A | 343 | 17.356 | −11.929 | −3.889 | 1.00 | 48.49 | C |
| ATOM | 106 | OE1 | GLU | A | 343 | 17.391 | −13.175 | −3.992 | 1.00 | 50.24 | O |
| ATOM | 107 | OE2 | GLU | A | 343 | 17.833 | −11.314 | −2.915 | 1.00 | 51.47 | O |
| ATOM | 108 | C | GLU | A | 343 | 16.263 | −9.143 | −7.180 | 1.00 | 37.22 | C |
| ATOM | 109 | O | GLU | A | 343 | 17.031 | −8.236 | −6.890 | 1.00 | 32.38 | O |
| ATOM | 110 | N | LEU | A | 344 | 14.937 | −8.989 | −7.246 | 1.00 | 25.47 | N |
| ATOM | 111 | CA | LEU | A | 344 | 14.317 | −7.693 | −6.969 | 1.00 | 23.40 | C |
| ATOM | 112 | CB | LEU | A | 344 | 12.916 | −7.594 | −7.644 | 1.00 | 23.43 | C |
| ATOM | 113 | CG | LEU | A | 344 | 12.969 | −7.779 | −9.161 | 1.00 | 32.75 | C |
| ATOM | 114 | CD1 | LEU | A | 344 | 11.538 | −7.940 | −9.704 | 1.00 | 27.03 | C |
| ATOM | 115 | CD2 | LEU | A | 344 | 13.684 | −6.585 | −9.819 | 1.00 | 32.92 | C |
| ATOM | 116 | C | LEU | A | 344 | 14.185 | −7.494 | −5.471 | 1.00 | 25.30 | C |
| ATOM | 117 | O | LEU | A | 344 | 14.236 | −6.365 | −4.982 | 1.00 | 29.77 | O |
| ATOM | 118 | N | GLY | A | 345 | 13.997 | −8.591 | −4.736 | 1.00 | 25.68 | N |
| ATOM | 119 | CA | GLY | A | 345 | 13.884 | −8.500 | −3.296 | 1.00 | 27.74 | C |
| ATOM | 120 | C | GLY | A | 345 | 13.777 | −9.886 | −2.700 | 1.00 | 29.98 | C |
| ATOM | 121 | O | GLY | A | 345 | 13.835 | −10.872 | −3.418 | 1.00 | 31.85 | O |
| ATOM | 122 | N | CYS | A | 346 | 13.638 | −9.976 | −1.382 | 1.00 | 29.57 | N |
| ATOM | 123 | CA | CYS | A | 346 | 13.498 | −11.286 | −0.755 | 1.00 | 32.14 | C |
| ATOM | 124 | CB | CYS | A | 346 | 14.867 | −11.885 | −0.465 | 1.00 | 38.98 | C |
| ATOM | 125 | SG | CYS | A | 346 | 15.741 | −10.909 | 0.749 | 1.00 | 43.33 | S |
| ATOM | 126 | C | CYS | A | 346 | 12.719 | −11.167 | 0.544 | 1.00 | 36.85 | C |
| ATOM | 127 | O | CYS | A | 346 | 12.395 | −10.063 | 0.978 | 1.00 | 38.86 | O |
| ATOM | 128 | N | GLY | A | 347 | 12.428 | −12.316 | 1.142 | 1.00 | 36.28 | N |
| ATOM | 129 | CA | GLY | A | 347 | 11.704 | −12.369 | 2.402 | 1.00 | 35.52 | C |
| ATOM | 130 | C | GLY | A | 347 | 11.923 | −13.746 | 2.997 | 1.00 | 35.81 | C |
| ATOM | 131 | O | GLY | A | 347 | 12.671 | −14.541 | 2.453 | 1.00 | 32.94 | O |
| ATOM | 132 | N | ASN | A | 348 | 11.272 | −14.040 | 4.116 | 1.00 | 39.18 | N |
| ATOM | 133 | CA | ASN | A | 348 | 11.406 | −15.350 | 4.749 | 1.00 | 42.21 | C |
| ATOM | 134 | CB | ASN | A | 348 | 10.644 | −15.387 | 6.080 | 1.00 | 48.61 | C |
| ATOM | 135 | CG | ASN | A | 348 | 11.277 | −14.514 | 7.131 | 1.00 | 53.37 | C |
| ATOM | 136 | OD1 | ASN | A | 348 | 12.422 | −14.702 | 7.492 | 1.00 | 56.08 | O |
| ATOM | 137 | ND2 | ASN | A | 348 | 10.516 | −13.546 | 7.635 | 1.00 | 59.19 | N |
| ATOM | 138 | C | ASN | A | 348 | 10.881 | −16.464 | 3.852 | 1.00 | 39.31 | C |
| ATOM | 139 | O | ASN | A | 348 | 11.275 | −17.619 | 3.996 | 1.00 | 39.22 | O |
| ATOM | 140 | N | PHE | A | 349 | 9.980 | −16.116 | 2.933 | 1.00 | 35.86 | N |
| ATOM | 141 | CA | PHE | A | 349 | 9.404 | −17.095 | 2.016 | 1.00 | 32.64 | C |
| ATOM | 142 | CB | PHE | A | 349 | 8.143 | −16.518 | 1.352 | 1.00 | 37.33 | C |
| ATOM | 143 | CG | PHE | A | 349 | 8.416 | −15.318 | 0.467 | 1.00 | 33.86 | C |
| ATOM | 144 | CD1 | PHE | A | 349 | 9.019 | −15.475 | −0.777 | 1.00 | 38.44 | C |
| ATOM | 145 | CD2 | PHE | A | 349 | 8.121 | −14.031 | 0.905 | 1.00 | 37.11 | C |
| ATOM | 146 | CE1 | PHE | A | 349 | 8.427 | −14.360 | −1.573 | 1.00 | 37.32 | C |
| ATOM | 147 | CE2 | PHE | A | 349 | 8.427 | −12.917 | 0.123 | 1.00 | 38.27 | C |
| ATOM | 148 | CZ | PHE | A | 349 | 9.036 | −13.082 | −1.120 | 1.00 | 36.15 | C |
| ATOM | 149 | C | PHE | A | 349 | 10.394 | −17.491 | 0.919 | 1.00 | 33.34 | C |
| ATOM | 150 | O | PHE | A | 349 | 10.333 | −18.586 | 0.367 | 1.00 | 36.04 | O |
| ATOM | 151 | N | GLY | A | 350 | 11.287 | −16.575 | 0.574 | 1.00 | 36.72 | N |
| ATOM | 152 | CA | GLY | A | 350 | 12.234 | −16.848 | −0.490 | 1.00 | 29.36 | C |
| ATOM | 153 | C | GLY | A | 350 | 12.627 | −15.562 | −1.166 | 1.00 | 32.07 | C |
| ATOM | 154 | O | GLY | A | 350 | 12.959 | −14.591 | 0.506 | 1.00 | 37.38 | O |
| ATOM | 155 | N | SER | A | 351 | 12.595 | −15.526 | −2.493 | 1.00 | 34.36 | N |
| ATOM | 156 | CA | SER | A | 351 | 12.993 | −14.304 | −3.151 | 1.00 | 33.56 | C |
| ATOM | 157 | CB | SER | A | 351 | 14.439 | −14.414 | −3.605 | 1.00 | 39.89 | C |
| ATOM | 158 | OG | SER | A | 351 | 14.564 | −15.336 | −4.654 | 1.00 | 38.83 | O |
| ATOM | 159 | C | SER | A | 351 | 12.093 | −13.929 | −4.318 | 1.00 | 25.34 | C |
| ATOM | 160 | O | SER | A | 351 | 11.246 | −14.703 | −4.741 | 1.00 | 30.57 | O |
| ATOM | 161 | N | VAL | A | 352 | 12.282 | −12.719 | −4.812 | 1.00 | 25.32 | N |
| ATOM | 162 | CA | VAL | A | 352 | 11.484 | −12.243 | −5.934 | 1.00 | 27.67 | C |
| ATOM | 163 | CB | VAL | A | 352 | 10.777 | −10.928 | −5.596 | 1.00 | 26.27 | C |
| ATOM | 164 | CG1 | VAL | A | 352 | 9.988 | −10.443 | −6.807 | 1.00 | 28.62 | C |
| ATOM | 165 | CG2 | VAL | A | 352 | 9.846 | −11.136 | −4.387 | 1.00 | 25.93 | C |
| ATOM | 166 | C | VAL | A | 352 | 12.504 | −12.002 | −7.022 | 1.00 | 24.75 | C |
| ATOM | 167 | O | VAL | A | 352 | 13.416 | −11.177 | −6.862 | 1.00 | 26.64 | O |
| ATOM | 168 | N | ARG | A | 353 | 12.334 | −12.720 | −8.124 | 1.00 | 21.24 | N |
| ATOM | 169 | CA | ARG | A | 353 | 13.259 | −12.640 | −9.253 | 1.00 | 24.10 | C |
| ATOM | 170 | CB | ARG | A | 353 | 13.643 | −14.052 | −9.721 | 1.00 | 24.70 | C |
| ATOM | 171 | CG | ARG | A | 353 | 14.430 | −14.913 | −8.722 | 1.00 | 41.94 | C |
| ATOM | 172 | CD | ARG | A | 353 | 15.851 | −14.401 | −8.516 | 1.00 | 48.67 | C |
| ATOM | 173 | NE | ARG | A | 353 | 16.805 | −15.494 | −8.339 | 1.00 | 56.82 | N |
| ATOM | 174 | CZ | ARG | A | 353 | 17.287 | −16.243 | −9.330 | 1.00 | 59.51 | C |
| ATOM | 175 | NH1 | ARG | A | 353 | 16.913 | −16.024 | −10.585 | 1.00 | 54.48 | N |
| ATOM | 176 | NH2 | ARG | A | 353 | 18.140 | −17.226 | −9.065 | 1.00 | 58.82 | N |
| ATOM | 177 | C | ARG | A | 353 | 12.625 | −11.910 | −10.430 | 1.00 | 30.05 | C |
| ATOM | 178 | O | ARG | A | 353 | 11.403 | −11.914 | −10.605 | 1.00 | 32.28 | O |
| ATOM | 179 | N | GLN | A | 354 | 13.461 | −11.288 | −11.245 | 1.00 | 26.89 | N |
| ATOM | 180 | CA | GLN | A | 354 | 12.954 | −10.605 | −12.429 | 1.00 | 21.13 | C |
| ATOM | 181 | CB | GLN | A | 354 | 13.886 | −9.449 | −12.813 | 1.00 | 27.45 | C |
| ATOM | 182 | CG | GLN | A | 354 | 13.394 | −8.651 | −14.005 | 1.00 | 33.20 | C |
| ATOM | 183 | CD | GLN | A | 354 | 14.196 | −7.386 | −14.228 | 1.00 | 35.81 | C |
| ATOM | 184 | OE1 | GLN | A | 354 | 14.929 | −6.932 | −13.344 | 1.00 | 32.36 | O |
| ATOM | 185 | NE2 | GLN | A | 354 | 14.048 | −6.798 | −15.410 | 1.00 | 38.36 | N |
| ATOM | 186 | C | GLN | A | 354 | 12.954 | −11.650 | −13.537 | 1.00 | 26.03 | C |
| ATOM | 187 | O | GLN | A | 354 | 13.773 | −12.580 | −13.516 | 1.00 | 27.62 | O |
| ATOM | 188 | N | GLY | A | 355 | 12.044 | −11.498 | −14.498 | 1.00 | 23.59 | N |
| ATOM | 189 | CA | GLY | A | 355 | 12.005 | −12.421 | −15.621 | 1.00 | 23.56 | C |
| ATOM | 190 | C | GLY | A | 355 | 11.167 | −11.834 | −16.740 | 1.00 | 28.28 | C |
| ATOM | 191 | O | GLY | A | 355 | 10.786 | −10.665 | −16.702 | 1.00 | 25.55 | O |
| ATOM | 192 | N | VAL | A | 356 | 10.901 | −12.664 | −17.755 | 1.00 | 28.64 | N |
| ATOM | 193 | CA | VAL | A | 356 | 10.074 | −12.264 | −18.883 | 1.00 | 29.66 | C |
| ATOM | 194 | CB | VAL | A | 356 | 10.921 | −12.045 | −20.174 | 1.00 | 34.30 | C |
| ATOM | 195 | CG1 | VAL | A | 356 | 10.005 | −11.659 | −21.351 | 1.00 | 32.44 | C |
| ATOM | 196 | CG2 | VAL | A | 356 | 11.931 | −10.977 | −19.919 | 1.00 | 29.39 | C |
| ATOM | 197 | C | VAL | A | 356 | 9.108 | −13.402 | −19.127 | 1.00 | 23.76 | C |
| ATOM | 198 | O | VAL | A | 356 | 9.485 | −14.569 | −19.033 | 1.00 | 26.47 | O |
| ATOM | 199 | N | TYR | A | 357 | 7.859 | −13.064 | −19.438 | 1.00 | 28.83 | N |
| ATOM | 200 | CA | TYR | A | 357 | 6.847 | −14.082 | −19.703 | 1.00 | 29.55 | C |
| ATOM | 201 | CB | TYR | A | 357 | 5.697 | −14.003 | −18.693 | 1.00 | 32.75 | C |
| ATOM | 202 | CG | TYR | A | 357 | 4.603 | −14.999 | −18.981 | 1.00 | 31.78 | C |
| ATOM | 203 | CD1 | TYR | A | 357 | 4.766 | −16.355 | −18.702 | 1.00 | 33.06 | C |
| ATOM | 204 | CE1 | TYR | A | 357 | 3.761 | −17.287 | −19.010 | 1.00 | 38.12 | C |
| ATOM | 205 | CD2 | TYR | A | 357 | 3.47 | −14.587 | −19.575 | 1.00 | 38.71 | C |
| ATOM | 206 | CE2 | TYR | A | 357 | 2.410 | −15.504 | −19.887 | 1.00 | 39.65 | C |
| ATOM | 207 | CZ | TYR | A | 357 | 2.587 | −16.844 | −19.601 | 1.00 | 39.40 | C |
| ATOM | 208 | OH | TYR | A | 357 | 1.577 | −17.722 | −19.904 | 1.00 | 48.05 | O |
| ATOM | 209 | C | TYR | A | 357 | 6.291 | −13.846 | −21.098 | 1.00 | 33.01 | C |
| ATOM | 210 | O | TYR | A | 357 | 6.038 | −12.699 | −21.484 | 1.00 | 34.42 | O |
| ATOM | 211 | N | ARG | A | 358 | 6.093 | −14.925 | −21.845 | 1.00 | 40.49 | N |
| ATOM | 212 | CA | ARG | A | 358 | 5.582 | −14.797 | −23.201 | 1.00 | 43.14 | C |
| ATOM | 213 | CB | ARG | A | 358 | 6.303 | −15.787 | −24.128 | 1.00 | 42.64 | C |
| ATOM | 214 | CG | ARG | A | 358 | 5.804 | −15.762 | −25.580 | 1.00 | 47.94 | C |
| ATOM | 215 | CD | ARG | A | 358 | 6.599 | −16.738 | −26.435 | 1.00 | 46.49 | C |
| ATOM | 216 | NE | ARG | A | 358 | 7.470 | −19.024 | 26.159 | 1.00 | 47.27 | N |
| ATOM | 217 | CZ | ARG | A | 358 | 7.470 | −19.024 | −26.159 | 1.00 | 47.27 | C |
| ATOM | 218 | NH1 | ARG | A | 358 | 8.496 | −18.736 | −26.947 | 1.00 | 48.46 | N |
| ATOM | 219 | NH2 | ARG | A | 358 | 7.365 | −20.239 | −25.636 | 1.00 | 49.08 | N |
| ATOM | 220 | C | ARG | A | 358 | 4.083 | −15.023 | −23.299 | 1.00 | 37.81 | C |
| ATOM | 221 | O | ARG | A | 358 | 3.602 | −16.102 | −22.983 | 1.00 | 39.13 | O |
| ATOM | 222 | N | MET | A | 359 | 3.361 | −13.984 | −23.712 | 1.00 | 48.72 | N |
| ATOM | 223 | CA | MET | A | 359 | 1.917 | −14.072 | −23.926 | 1.00 | 52.67 | C |
| ATOM | 224 | CB | MET | A | 359 | 1.185 | −12.898 | −23.284 | 1.00 | 56.85 | C |
| ATOM | 225 | CG | MET | A | 359 | 1.487 | −12.704 | −21.817 | 1.00 | 59.91 | C |
| ATOM | 226 | SD | MET | A | 359 | 0.442 | −11.450 | −21.068 | 1.00 | 68.40 | S |
| ATOM | 227 | CE | MET | A | 359 | 1.094 | −9.949 | −21.845 | 1.00 | 66.35 | C |
| ATOM | 228 | C | MET | A | 359 | 1.810 | −13.963 | −25.443 | 1.00 | 57.95 | C |
| ATOM | 229 | O | MET | A | 359 | 2.127 | −13.913 | −26.006 | 1.00 | 66.19 | O |
| ATOM | 230 | N | ARG | A | 360 | 1.393 | −15.043 | −26.097 | 1.00 | 60.89 | N |
| ATOM | 231 | CA | ARG | A | 360 | 1.282 | −15.079 | −27.561 | 1.00 | 63.37 | C |
| ATOM | 232 | CB | ARG | A | 360 | 0.383 | −16.246 | −27.994 | 1.00 | 64.54 | C |
| ATOM | 233 | CG | ARG | A | 360 | 0.960 | −17.616 | −27.623 | 1.00 | 65.69 | C |
| ATOM | 234 | CD | ARG | A | 360 | 2.353 | −17.786 | −28.231 | 1.00 | 65.62 | C |
| ATOM | 235 | NE | ARG | A | 360 | 3.255 | −18.567 | −27.384 | 1.00 | 65.60 | N |
| ATOM | 236 | CZ | ARG | A | 360 | 3.082 | −19.851 | −27.070 | 1.00 | 63.16 | C |
| ATOM | 237 | NH1 | ARG | A | 360 | 2.027 | −20.525 | −27.527 | 1.00 | 62.71 | N |
| ATOM | 238 | NH2 | ARG | A | 360 | 3.977 | −20.470 | −26.309 | 1.00 | 62.89 | N |
| ATOM | 239 | C | ARG | A | 360 | 0.788 | −13.774 | −28.175 | 1.00 | 63.27 | C |
| ATOM | 240 | O | ARG | A | 360 | −0.417 | −13.601 | −28.401 | 1.00 | 68.94 | O |
| ATOM | 241 | N | LYS | A | 361 | 1.743 | −12.878 | 28.448 | 1.00 | 59.53 | N |
| ATOM | 242 | CA | LYS | A | 361 | 1.511 | −11.549 | −29.022 | 1.00 | 56.77 | C |
| ATOM | 243 | CB | LYS | A | 361 | 0.219 | −10.938 | −28.461 | 1.00 | 60.05 | C |
| ATOM | 244 | CG | LYS | A | 361 | 0.226 | −10.722 | −26.950 | 1.00 | 60.48 | C |
| ATOM | 245 | CD | LYS | A | 361 | −1.174 | −10.402 | −26.409 | 1.00 | 62.81 | C |
| ATOM | 246 | CE | LYS | A | 361 | −1.771 | −9.144 | −27.036 | 1.00 | 64.09 | C |
| ATOM | 247 | NZ | LYS | A | 361 | −3.154 | −8.872 | −26.526 | 1.00 | 65.39 | N |
| ATOM | 248 | C | LYS | A | 361 | 2.701 | −10.647 | −28.659 | 1.00 | 61.19 | C |
| ATOM | 249 | O | LYS | A | 361 | 3.151 | −9.813 | −29.464 | 1.00 | 60.48 | O |
| ATOM | 250 | N | LYS | A | 362 | 3.204 | −10.819 | −27.436 | 1.00 | 52.69 | N |
| ATOM | 251 | CA | LYS | A | 362 | 4.334 | −10.035 | −26.949 | 1.00 | 54.92 | C |
| ATOM | 252 | CB | LYS | A | 362 | 3.901 | −8.588 | −26.737 | 1.00 | 56.85 | C |
| ATOM | 253 | CG | LYS | A | 362 | 2.785 | −8.432 | −25.728 | 1.00 | 53.56 | C |
| ATOM | 254 | CD | LYS | A | 362 | 2.334 | −6.986 | −25.680 | 1.00 | 58.19 | C |
| ATOM | 255 | CE | LYS | A | 362 | 1.452 | −6.738 | −24.479 | 1.00 | 60.02 | C |
| ATOM | 256 | NZ | LYS | A | 362 | 0.281 | −7.656 | −24.462 | 1.00 | 59.99 | C |
| ATOM | 257 | C | LYS | A | 362 | 4.900 | −10.601 | −25.642 | 1.00 | 48.81 | N |
| ATOM | 258 | O | LYS | A | 362 | 4.298 | −11.460 | −25.015 | 1.00 | 48.05 | C |
| ATOM | 259 | N | GLN | A | 362 | 6.069 | −10.119 | −25.241 | 1.00 | 51.22 | O |
| ATOM | 260 | CA | GLN | A | 363 | 6.682 | −10.594 | −24.007 | 1.00 | 49.77 | N |
| ATOM | 261 | CB | GLN | A | 363 | 8.095 | −11.132 | −24.267 | 1.00 | 54.46 | C |
| ATOM | 262 | CG | GLN | A | 363 | 8.154 | −12.259 | −25.286 | 1.00 | 52.95 | C |
| ATOM | 263 | CD | GLN | A | 363 | 9.545 | −12.865 | −25.397 | 1.00 | 54.97 | C |
| ATOM | 264 | OE1 | GLN | A | 363 | 10.550 | −12.189 | −25.172 | 1.00 | 58.63 | O |
| ATOM | 265 | NE2 | GLN | A | 363 | 9.606 | −14.135 | −25.764 | 1.00 | 57.23 | N |
| ATOM | 266 | C | GLN | A | 363 | 6.737 | −9.459 | −23.014 | 1.00 | 46.14 | C |
| ATOM | 267 | O | GLN | A | 363 | 7.198 | −8.366 | −23.329 | 1.00 | 52.39 | O |
| ATOM | 268 | N | GLN | A | 363 | 6.269 | −9.716 | −21.799 | 1.00 | 38.56 | N |
| ATOM | 269 | CA | ILE | A | 364 | 6.266 | −8.671 | −20.796 | 1.00 | 38.92 | C |
| ATOM | 270 | CB | ILE | A | 364 | 4.865 | −8.450 | −20.233 | 1.00 | 46.04 | C |
| ATOM | 271 | CG2 | ILE | A | 364 | 3.907 | −8.072 | −21.353 | 1.00 | 42.34 | C |
| ATOM | 272 | CG1 | ILE | A | 364 | 4.388 | −9.715 | −19.542 | 1.00 | 38.48 | C |
| ATOM | 273 | CD1 | ILE | A | 364 | 3.262 | −9.453 | −18.591 | 1.00 | 46.96 | C |
| ATOM | 274 | C | ILE | A | 364 | 7.199 | −8.974 | −19.643 | 1.00 | 35.15 | C |
| ATOM | 275 | O | ILE | A | 364 | 7.473 | −10.142 | −19.337 | 1.00 | 29.70 | O |
| ATOM | 276 | N | ASP | A | 365 | 7.685 | −7.922 | −19.005 | 1.00 | 32.92 | N |
| ATOM | 277 | CA | ASP | A | 365 | 8.577 | −8.086 | −17.872 | 1.00 | 31.39 | C |
| ATOM | 278 | CB | ASP | A | 365 | 9.253 | −6.762 | −17.522 | 1.00 | 35.51 | C |
| ATOM | 279 | CG | ASP | A | 365 | 10.142 | −6.278 | −18.685 | 1.00 | 40.87 | C |
| ATOM | 280 | OD1 | ASP | A | 365 | 10.983 | −7.066 | −19.158 | 1.00 | 44.73 | O |
| ATOM | 281 | OD2 | ASP | A | 365 | 9.997 | −5.102 | −19.089 | 1.00 | 47.04 | O |
| ATOM | 282 | C | ASP | A | 365 | 7.728 | −8.520 | −16.663 | 1.00 | 31.78 | C |
| ATOM | 283 | O | ASP | A | 365 | 6.656 | 7.977 | 16.422 | 1.00 | 31.99 | O |
| ATOM | 284 | N | VAL | A | 366 | 8.214 | −9.484 | −15.889 | 1.00 | 25.27 | N |
| ATOM | 285 | CA | VAL | A | 366 | 7.472 | −9.945 | −14.722 | 1.00 | 24.77 | C |
| ATOM | 286 | CB | VAL | A | 366 | 6.831 | −11.346 | 14.959 | 1.00 | 21.57 | C |
| ATOM | 287 | CG1 | VAL | A | 366 | 5.761 | −11.282 | −16.055 | 1.00 | 23.23 | C |
| ATOM | 288 | CG2 | VAL | A | 366 | 7.923 | −12.389 | −15.281 | 1.00 | 24.77 | C |
| ATOM | 289 | C | VAL | A | 366 | 8.365 | −10.061 | −13.486 | 1.00 | 26.20 | C |
| ATOM | 290 | O | VAL | A | 366 | 9.576 | −10.016 | −13.580 | 1.00 | 26.40 | O |
| ATOM | 291 | N | ALA | A | 367 | 7.742 | −10.156 | −12.315 | 1.00 | 23.36 | N |
| ATOM | 292 | CA | ALA | A | 367 | 8.471 | −10.354 | −11.074 | 1.00 | 27.38 | C |
| ATOM | 293 | CB | ALA | A | 367 | 8.166 | −9.227 | −10.067 | 1.00 | 25.34 | C |
| ATOM | 294 | C | ALA | A | 367 | 7.911 | −11.716 | −10.615 | 1.00 | 29.93 | C |
| ATOM | 295 | O | ALA | A | 367 | 6.703 | −11.961 | −10.667 | 1.00 | 29.39 | O |
| ATOM | 296 | N | ILE | A | 368 | 8.799 | −12.603 | −10.203 | 1.00 | 26.23 | N |
| ATOM | 297 | CA | ILE | A | 368 | 8.418 | −13.962 | −9.814 | 1.00 | 26.26 | C |
| ATOM | 298 | CB | ILE | A | 368 | 9.182 | −15.008 | −10.677 | 1.00 | 29.25 | C |
| ATOM | 299 | CG2 | ILE | A | 368 | 8.711 | −16.425 | −10.355 | 1.00 | 27.48 | C |
| ATOM | 300 | CG1 | ILE | A | 368 | 8.974 | −14.706 | −12.168 | 1.00 | 26.91 | C |
| ATOM | 301 | CD1 | ILE | A | 368 | 10.261 | −14.543 | −12.965 | 1.00 | 35.91 | C |
| ATOM | 302 | C | ILE | A | 368 | 8.744 | −14.257 | −8.352 | 1.00 | 26.03 | C |
| ATOM | 303 | O | ILE | A | 368 | 9.893 | −14.229 | −7.959 | 1.00 | 24.27 | O |
| ATOM | 304 | N | LYS | A | 369 | 7.720 | −14.539 | −7.561 | 1.00 | 25.90 | N |
| ATOM | 305 | CA | LYS | A | 369 | 7.953 | −14.862 | −6.156 | 1.00 | 26.26 | C |
| ATOM | 306 | CB | LYS | A | 369 | 6.691 | −14.547 | −5.344 | 1.00 | 25.23 | C |
| ATOM | 307 | CG | LYS | A | 369 | 6.822 | −14.795 | −3.870 | 1.00 | 31.86 | C |
| ATOM | 308 | CD | LYS | A | 369 | 5.509 | −14.423 | −3.158 | 1.00 | 34.03 | C |
| ATOM | 309 | CE | LYS | A | 369 | 5.438 | −14.876 | −1.716 | 1.00 | 39.65 | C |
| ATOM | 310 | NZ | LYS | A | 369 | 4.354 | −14.402 | −0.910 | 1.00 | 31.37 | N |
| ATOM | 311 | C | LYS | A | 369 | 8.298 | −16.361 | −6.140 | 1.00 | 27.61 | C |
| ATOM | 312 | O | LYS | A | 369 | 7.474 | −17.219 | −6.492 | 1.00 | 29.29 | O |
| ATOM | 313 | N | VAL | A | 370 | 9.532 | −16.674 | −5.764 | 1.00 | 26.60 | N |
| ATOM | 314 | CA | VAL | A | 370 | 9.990 | −18.065 | −5.766 | 1.00 | 28.07 | C |
| ATOM | 315 | CB | VAL | A | 370 | 11.355 | −18.200 | −6.481 | 1.00 | 33.07 | C |
| ATOM | 316 | CG1 | VAL | A | 370 | 11.721 | −19.667 | −6.633 | 1.00 | 38.83 | C |
| ATOM | 317 | CG2 | VAL | A | 370 | 11.315 | −17.506 | −7.841 | 1.00 | 30.80 | C |
| ATOM | 318 | C | VAL | A | 370 | 10.154 | −18.575 | −4.340 | 1.00 | 33.33 | C |
| ATOM | 319 | O | VAL | A | 370 | 10.920 | −18.021 | −3.558 | 1.00 | 31.84 | O |
| ATOM | 320 | N | LEU | A | 371 | 9.433 | −19.627 | −3.996 | 1.00 | 32.52 | N |
| ATOM | 321 | CA | LEU | A | 371 | 9.555 | −20.176 | −2.653 | 1.00 | 37.90 | C |
| ATOM | 322 | CB | LEU | A | 371 | 8.383 | −21.111 | −2.357 | 1.00 | 40.40 | C |
| ATOM | 323 | CG | LEU | A | 371 | 7.003 | −20.462 | −2.304 | 1.00 | 38.75 | C |
| ATOM | 324 | CD1 | LEU | A | 371 | 5.960 | −21.529 | −2.018 | 1.00 | 42.86 | C |
| ATOM | 325 | CD2 | LEU | A | 371 | 6.987 | −19.389 | −1.236 | 1.00 | 40.03 | C |
| ATOM | 326 | C | LEU | A | 371 | 10.877 | −20.936 | −2.523 | 1.00 | 41.99 | C |
| ATOM | 327 | O | LEU | A | 371 | 11.250 | −21.709 | −3.413 | 1.00 | 37.51 | O |
| ATOM | 328 | N | LYS | A | 372 | 11.517 | −20.717 | −1.411 | 1.00 | 40.63 | N |
| ATOM | 329 | CA | LYS | A | 372 | 12.847 | −21.374 | −1.151 | 1.00 | 47.85 | C |
| ATOM | 330 | CB | LYS | A | 372 | 13.430 | −20.879 | 0.176 | 1.00 | 49.15 | C |
| ATOM | 331 | CG | LYS | A | 372 | 12.538 | −21.128 | 1.390 | 1.00 | 52.08 | C |
| ATOM | 332 | CD | LYS | A | 372 | 13.153 | −20.548 | 2.655 | 1.00 | 53.51 | C |
| ATOM | 333 | CE | LYS | A | 372 | 12.283 | −20.840 | 3.875 | 1.00 | 55.66 | C |
| ATOM | 334 | NZ | LYS | A | 372 | 12.885 | −20.313 | 5.137 | 1.00 | 59.56 | N |
| ATOM | 335 | C | LYS | A | 372 | 12.691 | −22.894 | −1.121 | 1.00 | 52.35 | C |
| ATOM | 336 | O | LYS | A | 372 | 11.591 | −23.413 | −0.941 | 1.00 | 50.37 | O |
| ATOM | 337 | N | GLN | A | 373 | 13.799 | −23.605 | −1.312 | 1.00 | 59.78 | N |
| ATOM | 338 | CA | GLN | A | 373 | 13.780 | −25.067 | −1.304 | 1.00 | 59.32 | C |
| ATOM | 339 | CB | GLN | A | 373 | 15.115 | −25.606 | −1.835 | 1.00 | 63.62 | C |
| ATOM | 340 | CG | GLN | A | 373 | 15.155 | −27.116 | −2.051 | 1.00 | 66.24 | C |
| ATOM | 341 | CD | GLN | A | 373 | 14.191 | −27.586 | −3.134 | 1.00 | 71.01 | C |
| ATOM | 342 | OE1 | GLN | A | 373 | 12.970 | −27.465 | −2.995 | 1.00 | 69.21 | O |
| ATOM | 343 | NE2 | GLN | A | 373 | 14.739 | −28.129 | −4.219 | 1.00 | 68.75 | N |
| ATOM | 344 | C | GLN | A | 373 | 13.529 | −25.584 | 0.116 | 1.00 | 60.87 | C |
| ATOM | 345 | O | GLN | A | 373 | 14.088 | −25.061 | 1.085 | 1.00 | 58.13 | O |
| ATOM | 346 | N | GLY | A | 374 | 12.673 | −26.595 | 0.238 | 1.00 | 60.47 | N |
| ATOM | 347 | CA | GLY | A | 374 | 12.378 | −27.150 | 1.548 | 1.00 | 63.49 | C |
| ATOM | 348 | C | GLY | A | 374 | 11.112 | −26.589 | 2.169 | 1.00 | 65.45 | C |
| ATOM | 349 | O | GLY | A | 374 | 10.744 | −26.960 | 3.287 | 1.00 | 64.91 | O |
| ATOM | 350 | N | THR | A | 375 | 10.448 | −25.694 | 1.440 | 1.00 | 64.08 | N |
| ATOM | 351 | CA | THR | A | 375 | 9.211 | −25.073 | 1.910 | 1.00 | 62.35 | C |
| ATOM | 352 | CB | THR | A | 375 | 8.624 | −24.117 | 0.837 | 1.00 | 61.07 | C |
| ATOM | 353 | OG1 | THR | A | 375 | 9.499 | −22.997 | 0.658 | 1.00 | 58.51 | O |
| ATOM | 354 | CG2 | THR | A | 375 | 7.251 | −23.619 | 1.255 | 1.00 | 60.44 | C |
| ATOM | 355 | C | THR | A | 375 | 8.164 | −26.128 | 2.254 | 1.00 | 60.26 | C |
| ATOM | 356 | O | THR | A | 375 | 7.812 | −26.960 | 1.420 | 1.00 | 61.11 | O |
| ATOM | 357 | N | GLU | A | 376 | 7.669 | −26.085 | 3.486 | 1.00 | 61.48 | N |
| ATOM | 358 | CA | GLU | A | 376 | 6.659 | −27.038 | 3.937 | 1.00 | 63.66 | C |
| ATOM | 359 | CB | GLU | A | 376 | 6.395 | −26.854 | 5.431 | 1.00 | 65.34 | C |
| ATOM | 360 | CG | GLU | A | 376 | 5.410 | −27.863 | 6.011 | 1.00 | 70.83 | C |
| ATOM | 361 | CD | GLU | A | 376 | 5.443 | −27.905 | 7.531 | 1.00 | 74.17 | C |
| ATOM | 362 | OE1 | GLU | A | 376 | 5.151 | −26.868 | 8.174 | 1.00 | 71.51 | O |
| ATOM | 363 | OE2 | GLU | A | 376 | 5.765 | −28.983 | 8.083 | 1.00 | 74.47 | 0 |
| ATOM | 364 | C | GLU | A | 376 | 5.365 | −26.847 | 3.157 | 1.00 | 64.69 | C |
| ATOM | 365 | O | GLU | A | 376 | 5.071 | −25.745 | 2.693 | 1.00 | 62.88 | O |
| ATOM | 366 | N | LYS | A | 377 | 4.595 | −27.921 | 3.015 | 1.00 | 60.06 | N |
| ATOM | 367 | CA | LYS | A | 377 | 3.339 | −27.860 | 2.282 | 1.00 | 61.26 | C |
| ATOM | 368 | CB | LYS | A | 377 | 2.637 | −29.219 | 2.317 | 1.00 | 66.02 | C |
| ATOM | 369 | CG | LYS | A | 377 | 1.309 | −29.238 | 1.575 | 1.00 | 65.91 | C |
| ATOM | 370 | CD | LYS | A | 377 | 0.916 | −30.656 | 1.195 | 1.00 | 71.13 | C |
| ATOM | 371 | CE | LYS | A | 377 | −0.343 | −30.679 | 0.335 | 1.00 | 69.84 | C |
| ATOM | 372 | NZ | LYS | A | 377 | −0.745 | −32.070 | −0.037 | 1.00 | 70.61 | N |
| ATOM | 373 | C | LYS | A | 377 | 2.405 | −26.794 | 2.833 | 1.00 | 60.41 | C |
| ATOM | 374 | O | LYS | A | 377 | 1.535 | −26.293 | 2.121 | 1.00 | 58.25 | O |
| ATOM | 375 | C | ALA | A | 378 | 2.586 | −26.456 | 4.104 | 1.00 | 60.36 | N |
| ATOM | 376 | CA | ALA | A | 378 | 1.757 | −25.450 | 4.756 | 1.00 | 60.42 | C |
| ATOM | 377 | CB | ALA | A | 378 | 2.126 | −25.353 | 6.232 | 1.00 | 60.96 | C |
| ATOM | 378 | C | ALA | A | 378 | 1.914 | −24.083 | 4.090 | 1.00 | 58.57 | C |
| ATOM | 379 | O | ALA | A | 378 | 0.928 | −23.406 | 3.792 | 1.00 | 58.22 | O |
| ATOM | 380 | N | ASP | A | 379 | 3.159 | −23.690 | 3.849 | 1.00 | 58.14 | N |
| ATOM | 381 | CA | ASP | A | 379 | 3.446 | −22.398 | 3.237 | 1.00 | 59.61 | C |
| ATOM | 382 | CB | ASP | A | 379 | 4.889 | −21.999 | 3.513 | 1.00 | 61.20 | C |
| ATOM | 383 | CG | ASP | A | 379 | 5.387 | −22.533 | 4.841 | 1.00 | 69.25 | C |
| ATOM | 384 | OD1 | ASP | A | 379 | 4.801 | −22.184 | 5.892 | 1.00 | 70.24 | O |
| ATOM | 385 | OD2 | ASP | A | 379 | 6.367 | −23.313 | 4.831 | 1.00 | 70.38 | O |
| ATOM | 386 | C | ASP | A | 379 | 3.198 | −22.428 | 1.730 | 1.00 | 57.10 | C |
| ATOM | 387 | O | ASP | A | 379 | 2.944 | −21.394 | 1.118 | 1.00 | 54.39 | O |
| ATOM | 388 | N | THR | A | 380 | 3.279 | −23.616 | 1.137 | 1.00 | 51.67 | N |
| ATOM | 389 | CA | THR | A | 380 | 3.023 | −23.766 | −0.287 | 1.00 | 52.52 | C |
| ATOM | 390 | CB | THR | A | 380 | 3.350 | −25.193 | −0.755 | 1.00 | 53.17 | C |
| ATOM | 391 | OG1 | THR | A | 380 | 4.769 | −25.392 | −0.709 | 1.00 | 55.35 | O |
| ATOM | 392 | CG2 | THR | A | 380 | 2.858 | −25.411 | −2.168 | 1.00 | 57.49 | C |
| ATOM | 393 | C | THR | A | 380 | 1.536 | −23.488 | −0.506 | 1.00 | 53.07 | C |
| ATOM | 394 | O | THR | A | 380 | 1.142 | −22.808 | −1.449 | 1.00 | 46.30 | O |
| ATOM | 395 | N | GLU | A | 381 | 0.714 | −24.029 | 0.386 | 1.00 | 48.31 | N |
| ATOM | 396 | CA | GLU | A | 381 | −0.726 | −23.844 | 0.310 | 1.00 | 49.46 | C |
| ATOM | 397 | CB | GLU | A | 381 | −1.421 | −24.722 | 1.355 | 1.00 | 55.95 | C |
| ATOM | 398 | CG | GLU | A | 381 | −2.487 | −25.651 | 0.794 | 1.00 | 57.87 | C |
| ATOM | 399 | CD | GLU | A | 381 | −1.909 | −26.852 | 0.064 | 1.00 | 66.85 | C |
| ATOM | 400 | OE1 | GLU | A | 381 | −1.256 | −26.663 | −0.988 | 1.00 | 67.42 | O |
| ATOM | 401 | OE2 | GLU | A | 381 | −2.112 | −27.992 | 0.547 | 1.00 | 68.23 | O |
| ATOM | 402 | C | GLU | A | 381 | −1.025 | −22.371 | 0.585 | 1.00 | 47.48 | C |
| ATOM | 403 | O | GLU | A | 381 | −1.985 | −21.810 | 0.061 | 1.00 | 44.28 | O |
| ATOM | 404 | N | GLU | A | 382 | −0.181 | −21.752 | 1.404 | 1.00 | 42.74 | N |
| ATOM | 405 | CA | GLU | A | 382 | −0.332 | −20.346 | 1.764 | 1.00 | 44.98 | C |
| ATOM | 406 | CB | GLU | A | 382 | 0.697 | −19.989 | 2.828 | 1.00 | 50.35 | C |
| ATOM | 407 | CG | GLU | A | 382 | 0.552 | −18.600 | 3.407 | 1.00 | 54.73 | C |
| ATOM | 408 | CD | GLU | A | 382 | 1.479 | −18.376 | 4.593 | 1.00 | 61.59 | C |
| ATOM | 409 | OE1 | GLU | A | 382 | 2.719 | −18.344 | 4.400 | 1.00 | 67.89 | O |
| ATOM | 410 | OE2 | GLU | A | 382 | 0.963 | −18.241 | 5.726 | 1.00 | 66.58 | O |
| ATOM | 411 | C | GLU | A | 382 | −0.150 | −19.453 | 0.531 | 1.00 | 43.95 | C |
| ATOM | 412 | O | GLU | A | 382 | −0.893 | −18.490 | 0.331 | 1.00 | 37.04 | O |
| ATOM | 413 | N | MET | A | 383 | 0.847 | −19.772 | −0.288 | 1.00 | 38.03 | N |
| ATOM | 414 | CA | MET | A | 383 | 1.063 | −18.984 | −1.490 | 1.00 | 37.01 | C |
| ATOM | 415 | CB | MET | A | 383 | 2.425 | −19.295 | −2.124 | 1.00 | 37.76 | C |
| ATOM | 416 | CG | MET | A | 383 | 2.854 | −18.195 | −3.108 | 1.00 | 44.12 | C |
| ATOM | 417 | SD | MET | A | 383 | 4.495 | −18.400 | −3.806 | 1.00 | 45.79 | S |
| ATOM | 418 | CE | MET | A | 383 | 4.148 | −19.720 | −4.849 | 1.00 | 27.93 | C |
| ATOM | 419 | C | MET | A | 383 | −0.054 | −19.244 | −2.490 | 1.00 | 33.92 | C |
| ATOM | 420 | O | MET | A | 383 | −0.365 | −18.389 | −3.311 | 1.00 | 28.10 | O |
| ATOM | 421 | N | MET | A | 384 | 0.672 | −20.423 | −2.432 | 1.00 | 30.29 | N |
| ATOM | 422 | CA | MET | A | 384 | −1.770 | −20.707 | −3.346 | 1.00 | 35.41 | C |
| ATOM | 423 | CB | MET | A | 384 | −2.129 | −22.190 | −3.338 | 1.00 | 37.28 | C |
| ATOM | 424 | CG | MET | A | 384 | −1.098 | −23.082 | −4.013 | 1.00 | 44.29 | C |
| ATOM | 425 | SD | MET | A | 384 | −0.753 | −22.611 | −5.733 | 1.00 | 47.70 | S |
| ATOM | 426 | CE | MET | A | 384 | −2.394 | −22.740 | −6.491 | 1.00 | 43.44 | C |
| ATOM | 427 | C | MET | A | 384 | −2.988 | −19.871 | −2.970 | 1.00 | 32.30 | C |
| ATOM | 428 | O | MET | A | 384 | −3.746 | −19.436 | −3.838 | 1.00 | 32.40 | O |
| ATOM | 429 | N | ARG | A | 385 | −3.186 | −19.642 | −1.680 | 1.00 | 36.42 | N |
| ATOM | 430 | CA | ARG | A | 385 | −4.305 | −18.814 | −1.250 | 1.00 | 36.70 | C |
| ATOM | 431 | CB | ARG | A | 385 | −4.470 | −18.850 | 0.270 | 1.00 | 41.37 | C |
| ATOM | 432 | CG | ARG | A | 385 | −5.158 | −20.106 | 0.794 | 1.00 | 46.84 | C |
| ATOM | 433 | CD | ARG | A | 385 | −5.569 | −19.933 | 2.261 | 1.00 | 45.50 | C |
| ATOM | 434 | NE | ARG | A | 385 | −4.432 | −20.021 | 3.171 | 1.00 | 49.04 | N |
| ATOM | 435 | CZ | ARG | A | 385 | −3.842 | −21.165 | 3.508 | 1.00 | 48.50 | C |
| ATOM | 436 | NH1 | ARG | A | 385 | −4.291 | −22.309 | 3.007 | 1.00 | 48.28 | N |
| ATOM | 437 | NH2 | ARG | A | 385 | −2.806 | −21.167 | 4.334 | 1.00 | 48.88 | N |
| ATOM | 438 | C | ARG | A | 385 | −4.048 | −17.381 | −1.712 | 1.00 | 32.36 | C |
| ATOM | 439 | O | ARG | A | 385 | −4.970 | −16.666 | −2.088 | 1.00 | 34.82 | O |
| ATOM | 440 | N | GLU | A | 386 | −2.780 | −16.976 | −1.692 | 1.00 | 29.93 | N |
| ATOM | 441 | CA | GLU | A | 386 | −2.780 | −15.633 | −2.125 | 1.00 | 29.40 | C |
| ATOM | 442 | CB | GLU | A | 386 | −0.893 | −15.413 | −1.881 | 1.00 | 28.50 | C |
| ATOM | 443 | CG | GLU | A | 386 | −0.333 | −14.053 | −2.289 | 1.00 | 33.73 | C |
| ATOM | 444 | CD | GLU | A | 386 | 1.178 | −13.959 | −2.037 | 1.00 | 35.02 | C |
| ATOM | 445 | OE1 | GLU | A | 386 | 1.750 | −14.956 | −1.548 | 1.00 | 36.68 | O |
| ATOM | 446 | OE2 | GLU | A | 386 | 1.797 | −12.905 | −2.343 | 1.00 | 36.70 | O |
| ATOM | 447 | C | GLU | A | 386 | −2.713 | −15.481 | −3.615 | 1.00 | 27.47 | C |
| ATOM | 448 | O | GLU | A | 386 | −3.215 | −14.437 | −4.060 | 1.00 | 28.61 | O |
| ATOM | 449 | N | ALA | A | 387 | −2.412 | −16.525 | −4.377 | 1.00 | 30.23 | N |
| ATOM | 450 | CA | ALA | A | 387 | −2.673 | −16.488 | −5.806 | 1.00 | 33.30 | C |
| ATOM | 451 | CB | ALA | A | 387 | −2.097 | −17.735 | −6.484 | 1.00 | 32.95 | C |
| ATOM | 452 | C | ALA | A | 387 | −4.194 | −16.386 | −6.031 | 1.00 | 30.92 | C |
| ATOM | 453 | O | ALA | A | 387 | −4.638 | −15.601 | −6.843 | 1.00 | 33.56 | O |
| ATOM | 454 | N | GLN | A | 388 | −4.975 | −17.163 | −5.286 | 1.00 | 31.73 | N |
| ATOM | 455 | CA | GLN | A | 388 | −6.440 | −17.119 | −6.438 | 1.00 | 36.88 | C |
| ATOM | 456 | CB | GLN | A | 388 | −7.108 | −18.109 | −4.461 | 1.00 | 36.88 | C |
| ATOM | 457 | CG | GLN | A | 388 | −6.725 | −19.564 | −4.694 | 1.00 | 48.53 | C |
| ATOM | 458 | CD | GLN | A | 388 | −7.273 | −20.496 | −3.619 | 1.00 | 55.51 | C |
| ATOM | 459 | OE1 | GLN | A | 388 | −8.480 | −20.524 | −3.360 | 1.00 | 58.01 | O |
| ATOM | 460 | NE2 | GLN | A | 388 | −6.383 | =21.263 | −2.982 | 1.00 | 52.09 | N |
| ATOM | 461 | C | GLN | A | 388 | −7.001 | −15.711 | −5.201 | 1.00 | 33.60 | C |
| ATOM | 462 | O | GLN | A | 388 | −7.904 | −15.271 | −5.902 | 1.00 | O | |
| ATOM | 463 | N | ILE | A | 389 | −6.469 | −15.004 | −4.204 | 1.00 | 30.12 | N |
| ATOM | 464 | CA | ILE | A | 389 | −6.914 | −13.643 | −3.905 | 1.00 | 25.06 | C |
| ATOM | 465 | CB | ILE | A | 389 | −6.305 | −13.132 | −2.569 | 1.00 | 28.76 | C |
| ATOM | 466 | CG2 | ILE | A | 389 | −6.507 | −11.626 | −2.426 | 1.00 | 29.47 | C |
| ATOM | 467 | CG1 | ILE | A | 389 | −6.964 | −13.868 | −1.398 | 1.00 | 37.06 | C |
| ATOM | 468 | CD1 | ILE | A | 389 | −6.359 | −13.540 | −0.058 | 1.00 | 34.67 | C |
| ATOM | 469 | C | ILE | A | 389 | −6.524 | −12.696 | −5.022 | 1.00 | 26.55 | C |
| ATOM | 470 | O | ILE | A | 389 | −7.326 | −11.901 | −5.494 | 1.00 | 30.71 | O |
| ATOM | 471 | N | MET | A | 390 | −5.278 | −12.779 | −5.461 | 1.00 | 27.33 | N |
| ATOM | 472 | CA | MET | A | 390 | −4.845 | −11.910 | −6.529 | 1.00 | 24.55 | C |
| ATOM | 473 | CB | MET | A | 390 | −3.353 | −12.085 | −6.809 | 1.00 | 24.16 | C |
| ATOM | 474 | CG | MET | A | 390 | −2.457 | −11.523 | −5.714 | 1.00 | 24.68 | C |
| ATOM | 475 | SD | MET | A | 390 | −0.694 | −11.770 | −6.180 | 1.00 | 31.52 | S |
| ATOM | 476 | CE | MET | A | 390 | −0.450 | −10.325 | −7.210 | 1.00 | 31.50 | C |
| ATOM | 477 | C | MET | A | 390 | −5.638 | −12.215 | −7.807 | 1.00 | 29.46 | C |
| ATOM | 478 | O | MET | A | 390 | −5.960 | −11.302 | −8.558 | 1.00 | 30.11 | O |
| ATOM | 479 | N | HIS | A | 391 | −5.945 | −13.493 | −8.024 | 1.00 | 30.83 | N |
| ATOM | 480 | CA | HIS | A | 391 | −6.689 | −13.890 | −9.230 | 1.00 | 37.84 | C |
| ATOM | 481 | CB | HIS | A | 391 | −6.738 | −15.420 | −9.340 | 1.00 | 43.52 | C |
| ATOM | 482 | CG | HIS | A | 391 | −7.283 | −15.921 | −10.645 | 1.00 | 49.94 | C |
| ATOM | 483 | CD2 | HIS | A | 391 | −6.657 | −16.380 | −11.756 | 1.00 | 51.86 | C |
| ATOM | 484 | ND1 | HIS | A | 391 | −8.633 | −15.956 | −10.924 | 1.00 | 55.85 | N |
| ATOM | 485 | CE1 | HIS | A | 391 | −8.816 | −16.416 | −12.149 | 1.00 | 15.35 | C |
| ATOM | 486 | NE2 | HIS | A | 391 | −7.634 | −16.681 | −12.675 | 1.00 | 57.80 | N |
| ATOM | 487 | C | HIS | A | 391 | −8.106 | −13.321 | −9.231 | 1.00 | 42.10 | C |
| ATOM | 488 | O | HIS | A | 391 | −8.778 | −13.315 | −10.262 | 1.00 | 47.79 | O |
| ATOM | 489 | N | GLN | A | 392 | −8.559 | −12.839 | −8.077 | 1.00 | 39.91 | N |
| ATOM | 490 | CA | GLN | A | 392 | −9.898 | −12.259 | −7.965 | 1.00 | 41.34 | C |
| ATOM | 491 | CB | GLN | A | 392 | −10.543 | −12.659 | −6.633 | 1.00 | 43.83 | C |
| ATOM | 492 | CG | GLN | A | 392 | −10.631 | −14.161 | −6.437 | 1.00 | 47.90 | C |
| ATOM | 493 | CD | GLN | A | 392 | −11.399 | −14.548 | −5.197 | 1.00 | 51.89 | C |
| ATOM | 494 | OE1 | GLN | A | 392 | −12.612 | −14.361 | −5.124 | 1.00 | 58.95 | O |
| ATOM | 495 | NE2 | GLN | A | 392 | −10.699 | −15.095 | −4.210 | 1.00 | 51.91 | N |
| ATOM | 496 | C | GLN | A | 392 | −9.860 | −10.742 | −8.067 | 1.00 | 46.24 | C |
| ATOM | 497 | O | GLN | A | 392 | −10.888 | −10.081 | −7.932 | 1.00 | 43.30 | O |
| ATOM | 498 | N | LEU | A | 393 | −8.676 | −10.190 | −8.317 | 1.00 | 43.90 | N |
| ATOM | 499 | CA | LEU | A | 393 | −8.511 | −8.741 | −8.416 | 1.00 | 43.71 | C |
| ATOM | 500 | CB | LEU | A | 393 | −7.350 | −8.287 | −7.530 | 1.00 | 46.31 | C |
| ATOM | 501 | CG | LEU | A | 393 | −7.356 | −8.772 | −6.080 | 1.00 | 47.53 | C |
| ATOM | 502 | CD1 | LEU | A | 393 | −6.097 | −8.274 | −5.384 | 1.00 | 44.78 | C |
| ATOM | 503 | CD2 | LEU | A | 393 | −8.601 | −8.278 | −5.364 | 1.00 | 38.33 | C |
| ATOM | 504 | C | LEU | A | 393 | −8.269 | −8.257 | −9.843 | 1.00 | 45.51 | C |
| ATOM | 505 | O | LEU | A | 393 | −7.485 | −8.847 | −10.597 | 1.00 | 50.21 | O |
| ATOM | 506 | N | ASP | A | 394 | −8.924 | −7.167 | −10.220 | 1.00 | 41.60 | N |
| ATOM | 507 | CA | ASP | A | 394 | −8.759 | 6.638 | −11.565 | 1.00 | 38.62 | C |
| ATOM | 508 | CB | ASP | A | 394 | −9.874 | −7.175 | −12.473 | 1.00 | 45.48 | C |
| ATOM | 509 | CG | ASP | A | 394 | −9.733 | −6.718 | −13.917 | 1.00 | 53.93 | C |
| ATOM | 510 | OD1 | ASP | A | 394 | −10.609 | −7.075 | −14.740 | 1.00 | 54.65 | O |
| ATOM | 511 | OD2 | ASP | A | 394 | −8.756 | −6.009 | −14.235 | 1.00 | 49.40 | O |
| ATOM | 512 | C | ASP | A | 394 | −8.821 | −5.132 | −11.499 | 1.00 | 41.64 | C |
| ATOM | 513 | O | ASP | A | 394 | −9.870 | −4.523 | −11.744 | 1.00 | 41.10 | O |
| ATOM | 514 | N | ASN | A | 395 | −7.692 | −4.519 | −11.169 | 1.00 | 38.06 | N |
| ATOM | 515 | CA | ASN | A | 395 | −7.654 | −3.075 | 11.057 | 1.00 | 29.93 | C |
| ATOM | 516 | CB | ASN | A | 395 | −7.902 | −2.678 | −9.592 | 1.00 | 27.46 | C |
| ATOM | 517 | CG | ASN | A | 395 | −8.027 | −1.187 | −9.397 | 1.00 | 34.86 | C |
| ATOM | 518 | OD1 | ASN | A | 395 | −9.106 | −0.602 | −9.581 | 1.00 | 38.20 | O |
| ATOM | 519 | ND2 | ASN | A | 395 | −6.923 | −0.552 | −9.025 | 1.00 | 25.87 | N |
| ATOM | 520 | C | ASN | A | 395 | −6.285 | −2.610 | −11.530 | 1.00 | 30.95 | C |
| ATOM | 521 | O | ASN | A | 395 | −5.258 | −3.260 | −11.269 | 1.00 | 28.17 | O |
| ATOM | 522 | N | PRO | A | 396 | −6.248 | −1.467 | −12.222 | 1.00 | 28.97 | N |
| ATOM | 523 | CD | PRO | A | 396 | −7.395 | −0.706 | −12.748 | 1.00 | 39.56 | C |
| ATOM | 524 | CA | PRO | A | 396 | −4.996 | −0.919 | −12.733 | 1.00 | 28.79 | C |
| ATOM | 525 | CB | PRO | A | 396 | −5.464 | 0.277 | −13.556 | 1.00 | 37.29 | C |
| ATOM | 526 | CG | PRO | A | 396 | −6.842 | −0.149 | −14.019 | 1.00 | 42.13 | C |
| ATOM | 527 | C | PRO | A | 396 | −3.997 | −0.494 | −11.656 | 1.00 | 28.11 | C |
| ATOM | 528 | O | PRO | A | 396 | −2.821 | −0.352 | −11.939 | 1.00 | 27.45 | O |
| ATOM | 529 | N | TYR | A | 397 | −4.471 | −0.298 | −10.422 | 1.00 | 24.07 | N |
| ATOM | 530 | CA | TYR | A | 397 | −3.576 | 0.171 | −9.346 | 1.00 | 22.72 | C |
| ATOM | 531 | CB | TYR | A | 397 | −4.250 | 1.346 | −8.618 | 1.00 | 21.97 | C |
| ATOM | 532 | CG | TYR | A | 397 | −4.655 | 2.442 | −9.593 | 1.00 | 25.66 | C |
| ATOM | 533 | CD1 | TYR | A | 397 | −5.994 | 2.640 | −9.946 | 1.00 | 29.62 | C |
| ATOM | 534 | CE1 | TYR | A | 397 | −6.356 | 3.604 | −10.886 | 1.00 | 34.32 | C |
| ATOM | 535 | CD2 | TYR | A | 397 | −3.689 | 3.238 | −10.206 | 1.00 | 25.78 | C |
| ATOM | 536 | CE2 | TYR | A | 397 | −4.044 | 4.206 | −11.152 | 1.00 | 36.01 | C |
| ATOM | 537 | CZ | TYR | A | 397 | −5.376 | 4.380 | −11.490 | 1.00 | 37.11 | C |
| ATOM | 538 | OH | TYR | A | 397 | −5.694 | 4.380 | −11.490 | 1.00 | 37.11 | O |
| ATOM | 539 | C | TYR | A | 397 | −3.127 | −0.896 | −8.368 | 1.00 | 21.63 | C |
| ATOM | 540 | O | TYR | A | 397 | −2.730 | −0.615 | −7.213 | 1.00 | 23.29 | O |
| ATOM | 541 | N | ILE | A | 398 | −3.193 | −2.125 | −8.832 | 1.00 | 22.86 | N |
| ATOM | 542 | CA | ILE | A | 398 | −2.770 | −3.266 | −8.069 | 1.00 | 29.14 | C |
| ATOM | 543 | CB | ILE | A | 398 | −4.005 | −4.065 | −7.641 | 1.00 | 34.44 | C |
| ATOM | 544 | CG2 | ILE | A | 398 | −3.602 | −5.424 | −7.156 | 1.00 | 37.06 | C |
| ATOM | 545 | CG1 | ILE | A | 398 | −4.768 | −3.273 | −6.575 | 1.00 | 28.40 | C |
| ATOM | 546 | CD1 | ILE | A | 398 | −6.166 | −3.759 | −6.305 | 1.00 | 41.40 | C |
| ATOM | 547 | C | ILE | A | 398 | −1.858 | −4.102 | −8.975 | 1.00 | 33.99 | C |
| ATOM | 548 | O | ILE | A | 3998 | −2.096 | −4.193 | −10.183 | 1.00 | 34.40 | O |
| ATOM | 549 | N | VAL | A | 399 | −0.805 | −4.672 | −8.402 | 1.00 | 27.19 | N |
| ATOM | 550 | CA | VAL | A | 399 | 0.109 | −5.540 | −9.155 | 1.00 | 26.99 | C |
| ATOM | 551 | CB | VAL | A | 399 | 1.298 | −6.006 | −8.302 | 1.00 | 29.43 | C |
| ATOM | 552 | CG1 | VAL | A | 399 | 2.078 | −7.146 | −9.026 | 1.00 | 26.20 | C |
| ATOM | 553 | CG2 | VAL | A | 399 | 2.200 | −4.832 | −8.031 | 1.00 | 30.03 | C |
| ATOM | 554 | C | VAL | A | 399 | −0.704 | −6.759 | −9.539 | 1.00 | 33.87 | C |
| ATOM | 555 | O | VAL | A | 399 | −1.162 | −7.498 | −8.670 | 1.00 | 35.03 | O |
| ATOM | 556 | N | ARG | A | 400 | −0.849 | −6.986 | −10.837 | 1.00 | 26.00 | N |
| ATOM | 557 | CA | ARG | A | 400 | −1.652 | −8.098 | −11.315 | 1.00 | 30.60 | C |
| ATOM | 558 | CB | ARG | A | 400 | −2.172 | −7.785 | −12.728 | 1.00 | 38.54 | C |
| ATOM | 559 | CG | ARG | A | 400 | −3.035 | −6.521 | −12.854 | 1.00 | 46.40 | C |
| ATOM | 560 | CD | ARG | A | 400 | −3.410 | −6.240 | −14.331 | 1.00 | 46.44 | C |
| ATOM | 561 | NE | ARG | A | 400 | −4.269 | −5.066 | −14.505 | 1.00 | 52.85 | N |
| ATOM | 562 | CZ | ARG | A | 400 | −5.554 | −5.032 | −14.163 | 1.00 | 56.01 | C |
| ATOM | 563 | NH1 | ARG | A | 400 | −6.117 | −6.110 | −13.633 | 1.00 | 52.93 | N |
| ATOM | 564 | NH2 | ARG | A | 400 | −6.276 | −3.928 | −14.346 | 1.00 | 53.59 | N |
| ATOM | 565 | C | ARG | A | 400 | −0.946 | −9.444 | −11.324 | 1.00 | 27.57 | C |
| ATOM | 566 | O | ARG | A | 400 | 0.257 | −9.548 | −11.572 | 1.00 | 27.90 | O |
| ATOM | 567 | N | LEU | A | 401 | −1.703 | −10.500 | −11.077 | 1.00 | 28.32 | N |
| ATOM | 568 | CA | LEU | A | 401 | −1.147 | −11.838 | −11.096 | 1.00 | 24.81 | C |
| ATOM | 569 | CB | LEU | A | 401 | −1.974 | −12.777 | −10.238 | 1.00 | 32.19 | C |
| ATOM | 570 | CG | LEU | A | 401 | −1.534 | −14.239 | −10.271 | 1.00 | 29.13 | C |
| ATOM | 571 | CD1 | LEU | A | 401 | −0.159 | −14.414 | −9.638 | 1.00 | 37.61 | C |
| ATOM | 572 | CD2 | LEU | A | 401 | −2.578 | −15.046 | −9.516 | 1.00 | 42.15 | C |
| ATOM | 573 | C | LEU | A | 401 | −1.194 | −12.352 | −12.525 | 1.00 | 32.86 | C |
| ATOM | 574 | O | LEU | A | 401 | −2.248 | −12.281 | −13.176 | 1.00 | 30.25 | O |
| ATOM | 575 | N | ILE | A | 402 | −0.060 | −12.831 | −13.026 | 1.00 | 26.16 | N |
| ATOM | 576 | CA | ILE | A | 402 | −0.031 | −13.405 | −14.364 | 1.00 | 28.41 | C |
| ATOM | 577 | CB | ILE | A | 402 | 1.394 | −13.397 | −14.987 | 1.00 | 29.05 | C |
| ATOM | 578 | CG2 | ILE | A | 402 | 1.360 | −14.080 | −16.360 | 1.00 | 33.43 | C |
| ATOM | 579 | CG1 | ILE | A | 402 | 1.898 | −11.957 | −15.135 | 1.00 | 28.86 | C |
| ATOM | 580 | CD1 | ILE | A | 402 | 0.925 | −11.026 | −15.810 | 1.00 | 33.60 | C |
| ATOM | 581 | C | ILE | A | 402 | −0.508 | −14.853 | −14.187 | 1.00 | 31.32 | C/ |
| ATOM | 582 | O | ILE | A | 402 | −1.402 | −15.330 | −14.890 | 1.00 | 29.93 | O |
| ATOM | 583 | N | GLY | A | 403 | 0.070 | −15.548 | −13.224 | 1.00 | 29.11 | N |
| ATOM | 584 | CA | GLY | A | 403 | −0.335 | −16.924 | −12.971 | 1.00 | 28.71 | C |
| ATOM | 585 | C | GLY | A | 403 | 0.639 | −17.626 | −12.060 | 1.00 | 28.89 | C |
| ATOM | 586 | O | GLY | A | 403 | 1.605 | −17.020 | −11.612 | 1.00 | 29.79 | O |
| ATOM | 587 | N | VAL | A | 404 | 0.387 | −18.900 | −11.787 | 1.00 | 27.61 | N |
| ATOM | 588 | CA | VAL | A | 404 | 1.259 | −19.697 | −10.938 | 1.00 | 31.08 | C |
| ATOM | 589 | CB | VAL | A | 404 | 0.436 | −20.508 | −9.911 | 1.00 | 36.46 | C |
| ATOM | 590 | CG1 | VAL | A | 404 | 1.360 | −21.378 | −9.052 | 1.00 | 42.77 | C |
| ATOM | 591 | CG2 | VAL | A | 404 | −0.370 | −19.564 | −9.051 | 1.00 | 37.69 | C |
| ATOM | 592 | C | VAL | A | 404 | 2.019 | −20.671 | −11.283 | 1.00 | 29.99 | C |
| ATOM | 593 | O | VAL | A | 404 | 1.480 | −21.154 | −12.802 | 1.00 | 32.94 | O |
| ATOM | 594 | N | CYS | A | 405 | 3.262 | −20.969 | −11.461 | 1.00 | 31.70 | N |
| ATOM | 595 | CA | CYS | A | 405 | 4.065 | −21.908 | −12.243 | 1.00 | 37.20 | C |
| ATOM | 596 | CB | CYS | A | 405 | 5.131 | −21.150 | −13.042 | 1.00 | 40.22 | C |
| ATOM | 597 | SG | CYS | A | 405 | 5.968 | −22.154 | −14.282 | 1.00 | 46.07 | S |
| ATOM | 598 | C | CYS | A | 405 | 4.717 | −22.939 | −11.324 | 1.00 | 40.66 | C |
| ATOM | 599 | O | CYS | A | 405 | 5.428 | −22.587 | −10.388 | 1.00 | 39.23 | O |
| ATOM | 600 | N | GLN | A | 406 | 4.453 | −24.213 | −11.583 | 1.00 | 40.69 | N |
| ATOM | 601 | CA | GLN | A | 406 | 5.011 | −25.282 | −10.762 | 1.00 | 50.34 | C |
| ATOM | 602 | CB | GLN | A | 406 | 3.900 | −26.245 | −10.326 | 1.00 | 53.46 | C |
| ATOM | 603 | CG | GLN | A | 406 | 4.373 | −27.347 | −9.486 | 1.00 | 59.61 | C |
| ATOM | 604 | CD | GLN | A | 406 | 5.156 | −27.032 | −8.240 | 1.00 | 55.65 | C |
| ATOM | 605 | OE1 | GLN | A | 406 | 6.329 | −26.665 | −8.316 | 1.00 | 61.62 | O |
| ATOM | 606 | NE2 | GLN | A | 406 | 4.504 | −27.099 | −7.088 | 1.00 | 58.42 | N |
| ATOM | 607 | C | GLN | A | 406 | 6.090 | −26.044 | −11.519 | 1.00 | 53.49 | C |
| ATOM | 608 | O | GLN | A | 406 | 5.792 | −26.902 | −12.345 | 1.00 | 56.41 | O |
| ATOM | 609 | N | ALA | A | 407 | 7.342 | −25.718 | −11.234 | 1.00 | 54.94 | N |
| ATOM | 610 | CA | ALA | A | 407 | 8.468 | −26.378 | −11.879 | 1.00 | 59.69 | C |
| ATOM | 611 | CB | ALA | A | 407 | 9.105 | −25.437 | −12.902 | 1.00 | 59.62 | C |
| ATOM | 612 | C | ALA | A | 407 | 9.484 | −26.782 | −10.811 | 1.00 | 57.77 | C |
| ATOM | 613 | O | ALA | A | 407 | 9.173 | −27.578 | −9.923 | 1.00 | 57.65 | O |
| ATOM | 614 | N | GLU | A | 408 | 10.692 | −26.232 | −10.895 | 1.00 | 54.86 | N |
| ATOM | 615 | CA | GLU | A | 408 | 11.730 | −26.541 | −9.921 | 1.00 | 56.21 | C |
| ATOM | 616 | CB | GLU | A | 408 | 13.002 | −25.738 | −10.220 | 1.00 | 60.73 | C |
| ATOM | 617 | CG | GLU | A | 408 | 12.777 | −24.237 | −10.370 | 1.00 | 60.37 | C |
| ATOM | 618 | CD | GLU | A | 408 | 12.597 | −23.801 | −11.818 | 1.00 | 61.95 | C |
| ATOM | 619 | OE1 | GLU | A | 408 | 11.749 | −24.377 | −12.535 | 1.00 | 61.59 | O |
| ATOM | 620 | OE2 | GLU | A | 408 | 13.308 | −22.866 | −12.238 | 1.00 | 60.74 | O |
| ATOM | 621 | C | GLU | A | 408 | 11.216 | −26.210 | −8.524 | 1.00 | 57.49 | C |
| ATOM | 622 | O | GLU | A | 408 | 11.766 | −26.663 | −7.517 | 1.00 | 60.01 | O |
| ATOM | 623 | N | ALA | A | 409 | 10.155 | −25.411 | −8.480 | 1.00 | 56.44 | N |
| ATOM | 624 | CA | ALA | A | 409 | 9.519 | −25.007 | −7.231 | 1.00 | 52.91 | C |
| ATOM | 625 | CB | ALA | A | 409 | 10.480 | −24.178 | −6.392 | 1.00 | 58.00 | C |
| ATOM | 626 | C | ALA | A | 409 | 8.276 | −24.196 | −7.589 | 1.00 | 49.70 | C |
| ATOM | 627 | O | ALA | A | 409 | 8.029 | −23.822 | −8.750 | 1.00 | 46.56. | O |
| ATOM | 628 | N | LEU | A | 410 | 7.420 | −23.942 | −6.601 | 1.00 | 48.39 | N |
| ATOM | 629 | CA | LEU | A | 410 | 6.189 | −23.192 | −6.830 | 1.00 | 42.60 | C |
| ATOM | 630 | CB | LEU | A | 410 | 5.210 | −23.433 | −5.681 | 1.00 | 42.05 | C |
| ATOM | 631 | CG | LEU | A | 410 | 3.794 | −22.949 | −5.968 | 1.00 | 39.06 | C |
| ATOM | 632 | CD1 | LEU | A | 410 | 3.256 | −23.669 | −7.199 | 1.00 | 42.72 | C |
| ATOM | 633 | CD2 | LEU | A | 410 | 2.912 | −23.207 | −4.752 | 1.00 | 46.20 | C |
| ATOM | 634 | C | LEU | A | 410 | 6.516 | −21.711 | −6.941 | 1.00 | 34.88 | C |
| ATOM | 635 | O | LEU | A | 410 | 7.246 | −21.714 | −6.118 | 1.00 | 32.97 | O |
| ATOM | 636 | N | MET | A | 411 | 5.969 | −21.052 | −7.956 | 1.00 | 32.78 | N |
| ATOM | 637 | CA | MET | A | 411 | 6.269 | −19.647 | −8.171 | 1.00 | 27.80 | C |
| ATOM | 638 | CB | MET | A | 411 | 7.272 | −19.506 | −9.326 | 1.00 | 29.67 | C |
| ATOM | 639 | CG | MET | A | 411 | 8.546 | −20.333 | −9.200 | 1.00 | 37.85 | C |
| ATOM | 640 | SD | MET | A | 411 | 9.647 | −20.153 | −10.644 | 1.00 | 40.33 | S |
| ATOM | 641 | CE | MET | A | 411 | 8.672 | −20.909 | −11.859 | 1.00 | 38.19 | C |
| ATOM | 642 | C | MET | A | 411 | 5.024 | −18.846 | −8.521 | 1.00 | 29.61 | C |
| ATOM | 643 | O | MET | A | 411 | 4.126 | −19.354 | −9.171 | 1.00 | 32.53 | O |
| ATOM | 644 | N | LEU | A | 412 | 4.975 | −17.593 | −8.083 | 1.00 | 26.11 | N |
| ATOM | 645 | CA | LEU | A | 412 | 3.864 | −16.704 | −8.426 | 1.00 | 23.60 | C |
| ATOM | 646 | CB | LEU | A | 412 | 3.340 | −15.957 | −7.197 | 1.00 | 30.66 | C |
| ATOM | 647 | CG | LEU | A | 412 | 2.475 | −16.741 | −6.224 | 1.00 | 43.14 | C |
| ATOM | 648 | CD1 | LEU | A | 412 | 1.856 | −15.774 | −5.208 | 1.00 | 39.76 | C |
| ATOM | 649 | CD2 | LEU | A | 412 | 1.389 | −17.457 | −7.018 | 1.00 | 42.58 | C |
| ATOM | 650 | C | LEU | A | 412 | 4.450 | −15.688 | −9.391 | 1.00 | 33.31 | C |
| ATOM | 651 | O | LEU | A | 412 | 5.348 | −14.947 | −9.004 | 1.00 | 24.93 | O |
| ATOM | 652 | N | VAL | A | 413 | 3.928 | −15.663 | −10.626 | 1.00 | 26.19 | N |
| ATOM | 653 | CA | VAL | A | 413 | 4.385 | −14.739 | −11.674 | 1.00 | 24.01 | C |
| ATOM | 654 | CB | VAL | A | 413 | 4.374 | −15.425 | −13.070 | 1.00 | 25.63 | C |
| ATOM | 655 | CG1 | VAL | A | 413 | 4.951 | −14.465 | −14.143 | 1.00 | 22.85 | C |
| ATOM | 656 | CG2 | VAL | A | 413 | 5.192 | −16.718 | −13.010 | 1.00 | 26.67 | C |
| ATOM | 657 | C | VAL | A | 413 | 3.458 | −13.540 | −11.700 | 1.00 | 26.19 | C |
| ATOM | 658 | O | VAL | A | 413 | 2.240 | −13.673 | −11.925 | 1.00 | 28.56 | O |
| ATOM | 659 | N | MET | A | 414 | 4.052 | −12.368 | −11.490 | 1.00 | 22.49 | N |
| ATOM | 660 | CA | MET | A | 414 | 3.324 | −11.108 | −11.418 | 1.00 | 22.14 | C |
| ATOM | 661 | CB | MET | A | 414 | 3.422 | −10.545 | −9.987 | 1.00 | 25.30 | C |
| ATOM | 662 | CG | MET | A | 414 | 2.767 | −11.409 | −8.917 | 1.00 | 28.88 | C |
| ATOM | 663 | SD | MET | A | 414 | 3.377 | −10.874 | −7.264 | 1.00 | 30.85 | S |
| ATOM | 664 | CE | MET | A | 414 | 4.890 | −11.872 | −7.109 | 1.00 | 32.45 | C |
| ATOM | 665 | C | MET | A | 414 | 3.871 | −10.049 | −12.336 | 1.00 | 34.63 | C |
| ATOM | 666 | O | MET | A | 414 | 5.017 | −10.131 | −12.796 | 1.00 | 25.46 | O |
| ATOM | 667 | N | GLU | A | 415 | 3.075 | −9.012 | −12.568 | 1.00 | 25.97 | N |
| ATOM | 668 | CA | GLU | A | 415 | 3.548 | −7.913 | −13.395 | 1.00 | 25.91 | C |
| ATOM | 669 | CB | GLU | A | 415 | 2.470 | −6.842 | −13.568 | 1.00 | 37.31 | C |
| ATOM | 670 | CG | GLU | A | 415 | 1.181 | −7.321 | −14.191 | 1.00 | 41.51 | C |
| ATOM | 671 | CD | GLU | A | 415 | 0.151 | −6.190 | −14.258 | 1.00 | 56.50 | C |
| ATOM | 672 | OE1 | GLU | A | 415 | −0.056 | −5.493 | −13.222 | 1.00 | 39.84 | O |
| AOTM | 673 | OE2 | GLU | A | 415 | −0.444 | −5.998 | −15.346 | 1.00 | 55.15 | O |
| ATOM | 674 | C | GLU | A | 415 | 4.711 | 07.273 | −12.651 | 1.00 | 26.45 | C |
| ATOM | 675 | O | GLU | A | 415 | 4.751 | −7.287 | −12.651 | 1.00 | 26.92 | O |
| ATOM | 676 | N | MET | A | 416 | 5.657 | −6.713 | −13.400 | 1.00 | 27.12 | N |
| ATOM | 677 | CA | MET | A | 416 | 6.784 | −6.047 | −12.778 | 1.00 | 26.18 | C |
| ATOM | 678 | CB | MET | A | 416 | 8.078 | −6.047 | −12.778 | 1.00 | 29.98 | C |
| ATOM | 679 | CG | MET | A | 416 | 9.308 | −5.885 | −12.729 | 1.00 | 32.43 | C |
| ATOM | 680 | SD | MET | A | 416 | 10.835 | −6.141 | −13.631 | 1.00 | 40.73 | S |
| ATOM | 681 | CE | MET | A | 416 | 11.931 | −4.939 | −12.809 | 1.00 | 40.48 | C |
| ATOM | 682 | C | MET | A | 416 | 6.577 | −4.539 | −12.781 | 1.00 | 30.68 | C |
| ATOM | 683 | O | MET | A | 416 | 6.117 | −3.981 | −13.766 | 1.00 | 29.07 | O |
| ATOM | 684 | N | ALA | A | 417 | 6.916 | −3.893 | −11.670 | 1.00 | 28.26 | N |
| ATOM | 685 | CA | ALA | A | 417 | 6.843 | −2.436 | −11.561 | 1.00 | 31.40 | C |
| ATOM | 686 | CB | ALA | A | 417 | 5.886 | −2.023 | −11.253 | 1.00 | 31.37 | C |
| ATOM | 687 | C | ALA | A | 417 | 8.286 | −2.023 | −11.253 | 1.00 | 31.10 | C |
| ATOM | 688 | O | ALA | A | 417 | 8.672 | −1.865 | −10.087 | 1.00 | 31.97 | O |
| ATOM | 689 | N | GLY | A | 418 | 9.064 | −1.876 | −12.330 | 1.00 | 35.10 | N |
| ATOM | 690 | CA | GLY | A | 418 | 10.485 | 1.554 | −12.258 | 1.00 | 34.74 | C |
| ATOM | 691 | C | GLY | A | 418 | 10.936 | −0.340 | −11.474 | 1.00 | 32.22 | C |
| ATOM | 692 | O | GLY | A | 418 | 12.120 | −0.205 | −11.147 | 1.00 | 33.36 | O |
| ATOM | 693 | N | GLY | A | 419 | 10.002 | 0.559 | −11.183 | 1.00 | 26.94 | N |
| ATOM | 694 | CA | GLY | A | 419 | 10.354 | 1.740 | −10.421 | 1.00 | 27.31 | C |
| ATOM | 695 | C | GLY | A | 419 | 10.674 | 1.386 | −8.978 | 1.00 | 27.31 | C |
| ATOM | 696 | O | GLY | A | 419 | 11.321 | 2.150 | −8.287 | 1.00 | 31.61 | O |
| ATOM | 697 | N | GLY | A | 420 | 10.196 | 0.235 | −8.506 | 1.00 | 32.29 | N |
| ATOM | 698 | CA | GLY | A | 420 | 10.469 | −0.166 | −7.139 | 1.00 | 27.34 | C |
| ATOM | 699 | C | GLY | A | 420 | 9.632 | 0.511 | −6.054 | 1.00 | 27.22 | C |
| ATOM | 700 | O | GLY | A | 420 | 8.709 | 1.263 | −6.369 | 1.00 | 25.34 | O |
| ATOM | 701 | N | PRO | A | 421 | 9.952 | 0.280 | −4.770 | 1.00 | 27.85 | N |
| ATOM | 702 | CD | PRO | A | 421 | 11.122 | −0.509 | −4.330 | 1.00 | 29.71 | C |
| ATOM | 703 | CA | PRO | A | 421 | 9.247 | 0.843 | −3.612 | 1.00 | 25.99 | C |
| ATOM | 704 | CB | PRO | A | 421 | 10.049 | 0.310 | −2.411 | 1.00 | 33.47 | C |
| ATOM | 705 | CG | PRO | A | 421 | 10.755 | −0.872 | −2.921 | 1.00 | 33.83 | C |
| ATOM | 706 | C | PRO | A | 421 | 9.178 | 2.358 | −3.574 | 1.00 | 32.23 | C |
| ATOM | 707 | O | PRO | A | 421 | 10.126 | 3.052 | 03.845 | 1.00 | 25.70 | O |
| ATOM | 708 | N | LEU | A | 422 | 8.002 | 2.856 | −3.196 | 1.00 | 24.75 | N |
| ATOM | 709 | CA | LEU | A | 422 | 7.726 | 4.280 | −3.094 | 1.00 | 24.74 | C |
| ATOM | 710 | CB | LEU | A | 422 | 6.258 | 4.475 | −2.705 | 1.00 | 25.86 | C |
| ATOM | 711 | CG | LEU | A | 422 | 5.736 | 5.899 | −2.536 | 1.00 | 27.46 | C |
| ATOM | 712 | CD1 | LEU | A | 422 | 5.749 | 6.599 | −3.879 | 1.00 | 29.48 | C |
| ATOM | 713 | CD2 | LEU | A | 422 | 4.322 | 5.851 | −1.989 | 1.00 | 26.17 | C |
| ATOM | 714 | C | LEU | A | 422 | 8.607 | 5.010 | −2.078 | 1.00 | 24.09 | C |
| ATOM | 715 | O | LEU | A | 422 | 9.065 | 6.128 | −2.347 | 1.00 | 27.09 | O |
| ATOM | 716 | N | HIS | A | 423 | 8.821 | 4.423 | −0.908 | 1.00 | 23.84 | N |
| ATOM | 717 | CA | HIS | A | 423 | 9.627 | 5.097 | 0.097 | 1.00 | 31.59 | C |
| ATOM | 718 | CB | HIS | A | 423 | 9.677 | 4.309 | 1.410 | 1.00 | 33.34 | C |
| ATOM | 719 | CG | HIS | A | 423 | 10.255 | 2.935 | 1.274 | 1.00 | 32.56 | C |
| ATOM | 720 | CD2 | HIS | A | 423 | 10.263 | 2.066 | 0.236 | 1.00 | 33.97 | C |
| ATOM | 721 | ND1 | HIS | A | 423 | 10.947 | 2.318 | 2.296 | 1.00 | 40.55 | N |
| ATOM | 722 | CE1 | HIS | A | 423 | 11.355 | 1.128 | 1.894 | 1.00 | 33.85 | C |
| ATOM | 723 | NE2 | HIS | A | 423 | 10.952 | 0.950 | 0.647 | 1.00 | 37.19 | N |
| ATOM | 724 | C | HIS | A | 423 | 11.048 | 5.352 | −0.417 | 1.00 | 35.72 | C |
| ATOM | 725 | O | HIS | A | 423 | 11.583 | 6.444 | −0.235 | 1.00 | 38.98 | O |
| ATOM | 726 | N | LYS | A | 424 | 11.647 | 4.356 | −1.064 | 1.00 | 26.67 | N |
| ATOM | 727 | CA | LYS | A | 424 | 13.000 | 4.559 | −1.576 | 1.00 | 30.40 | C |
| ATOM | 728 | CB | LYS | A | 424 | 13.625 | 3.217 | −1.950 | 1.00 | 30.75 | C |
| ATOM | 729 | CG | LYS | A | 424 | 13.884 | 2.327 | −0.747 | 1.00 | 32.42 | C |
| ATOM | 730 | CD | LYS | A | 424 | 14.486 | 0.996 | −1.191 | 1.00 | 42.37 | C |
| ATOM | 731 | CE | LYS | A | 424 | 14.635 | 0.035 | −0.020 | 1.00 | 47.25 | C |
| ATOM | 732 | NZ | LYS | A | 424 | 15.606 | −1.054 | −0.360 | 1.00 | 54.40 | N |
| ATOM | 733 | C | LYS | A | 424 | 13.025 | 5.500 | −2.767 | 1.00 | 30.87 | C |
| ATOM | 734 | O | LYS | A | 424 | 13.976 | 6.259 | −2.944 | 1.00 | 27.95 | O |
| ATOM | 735 | N | PHE | A | 425 | 11.975 | 5.479 | −3.586 | 1.00 | 27.56 | N |
| ATOM | 736 | CA | PHE | A | 425 | 11.919 | 6.345 | −4.755 | 1.00 | 27.56 | C |
| ATOM | 737 | CB | PHE | A | 425 | 10.693 | 6.006 | −5.612 | 1.00 | 33.85 | C |
| ATOM | 738 | CG | PHE | A | 425 | 10.536 | 6.885 | −6.816 | 1.00 | 33.08 | C |
| ATOM | 739 | CD1 | PHE | A | 425 | 11.259 | 6.634 | −7.976 | 1.00 | 43.47 | C |
| ATOM | 740 | CD2 | PHE | A | 425 | 9.692 | 7.986 | −6.778 | 1.00 | 38.10 | C |
| ATOM | 741 | CE1 | PHE | A | 425 | 11.146 | 7.476 | −9.083 | 1.00 | 42.11 | C |
| ATOM | 742 | CE2 | PHE | A | 425 | 9.567 | 8.832 | −7.874 | 1.00 | 47.88 | C |
| ATOM | 743 | CZ | PHE | A | 425 | 10.297 | 8.574 | −9.033 | 1.00 | 44.70 | C |
| ATOM | 744 | C | PHE | A | 425 | 11.861 | 7.838 | −4.391 | 1.00 | 29.42 | C |
| ATOM | 745 | O | PHE | A | 425 | 12.421 | 8.681 | −5.097 | 1.00 | 27.18 | O |
| ATOM | 746 | N | LEU | A | 426 | 11.198 | 8.174 | −3.295 | 1.00 | 26.78 | N |
| ATOM | 747 | CA | LEU | A | 426 | 11.053 | 9.581 | −2.919 | 1.00 | 23.65 | C |
| ATOM | 748 | CB | LEU | A | 426 | 9.750 | 9.793 | −2.142 | 1.00 | 23.99 | C |
| ATOM | 749 | CG | LEU | A | 426 | 8.484 | 9.543 | −2.972 | 1.00 | 23.37 | C |
| ATOM | 750 | CD1 | LEU | A | 426 | 7.234 | 9.646 | −2.081 | 1.00 | 23.77 | C |
| ATOM | 751 | CD2 | LEU | A | 426 | 8.429 | 10.547 | −4.096 | 1.00 | 27.33 | C |
| ATOM | 752 | C | LEU | A | 426 | 12.210 | 10.161 | −2.100 | 1.00 | 29.71 | C |
| ATOM | 753 | O | LEU | A | 426 | 12.345 | 11.379 | −2.011 | 1.00 | 24.55 | O |
| ATOM | 754 | N | VAL | A | 427 | 13.021 | 9.296 | −1.501 | 1.00 | 25.47 | N |
| ATOM | 755 | CA | VAL | A | 427 | 14.130 | 9.781 | −0.661 | 1.00 | 28.99 | C |
| ATOM | 756 | CB | VAL | A | 427 | 14.996 | 8.607 | −0.132 | 1.00 | 34.76 | C |
| ATOM | 757 | CG1 | VAL | A | 427 | 16.250 | 9.148 | 0.578 | 1.00 | 37.93 | C |
| ATOM | 758 | CG2 | VAL | A | 427 | 14.186 | 7.762 | 0.852 | 1.00 | 30.76 | C |
| ATOM | 759 | C | VAL | A | 427 | 15.027 | 10.780 | −1.403 | 1.00 | 34.84 | C |
| ATOM | 760 | O | VAL | A | 427 | 15.605 | 10.464 | −2.445 | 1.00 | 37.55 | O |
| ATOM | 761 | N | GLY | A | 428 | 15.117 | 11.993 | −0.862 | 1.00 | 43.08 | N |
| ATOM | 762 | CA | GLY | A | 428 | 15.941 | 13.030 | −1.469 | 1.00 | 41.65 | C |
| ATOM | 763 | C | GLY | A | 428 | 15.437 | 13.634 | −2.768 | 1.00 | 47.74 | C |
| ATOM | 764 | O | GLY | A | 428 | 16.196 | 14.303 | −3.479 | 1.00 | 42.77 | O |
| ATOM | 765 | N | LYS | A | 429 | 14.163 | 13.430 | −3.090 | 1.00 | 37.30 | N |
| ATOM | 766 | CA | LYS | A | 429 | 13.624 | 13.972 | −4.328 | 1.00 | 41.14 | C |
| ATOM | 767 | CB | LYS | A | 429 | 13.024 | 12.840 | −5.161 | 1.00 | 46.03 | C |
| ATOM | 768 | CG | LYS | A | 429 | 14.064 | 11.799 | −5.568 | 1.00 | 46.82 | C |
| ATOM | 769 | CD | LYS | A | 429 | 13.607 | 11.007 | −6.775 | 1.00 | 54.33 | C |
| ATOM | 770 | CE | LYS | A | 429 | 14.575 | 9.879 | −7.099 | 1.00 | 52.59 | C |
| ATOM | 771 | NZ | LYS | A | 429 | 14.028 | 9.018 | −8.182 | 1.00 | 61.02 | N |
| ATOM | 772 | C | LYS | A | 429 | 12.596 | 15.079 | −4.098 | 1.00 | 43.46 | C |
| ATOM | 773 | O | LYS | A | 429 | 11.741 | 15.347 | −4.952 | 1.00 | 46.64 | O |
| ATOM | 774 | N | ARG | A | 430 | 12.696 | 15.736 | −2.950 | 1.00 | 38.34 | N |
| ATOM | 775 | CA | ARG | A | 430 | 11.765 | 16.798 | −2.604 | 1.00 | 51.71 | C |
| ATOM | 776 | CB | ARG | A | 430 | 12.050 | 17.298 | −1.189 | 1.00 | 53.36 | C |
| ATOM | 777 | CG | ARG | A | 430 | 10.922 | 18.125 | −0.591 | 1.00 | 62.96 | C |
| ATOM | 778 | CD | ARG | A | 430 | 11.039 | 18.217 | 0.928 | 1.00 | 63.14 | C |
| ATOM | 779 | NE | ARG | A | 430 | 9.729 | 18.100 | 1.576 | 1.00 | 52.24 | N |
| ATOM | 780 | CZ | ARG | A | 430 | 9.526 | 17.461 | 2.724 | 1.00 | 68.34 | C |
| ATOM | 781 | NH1 | ARG | A | 430 | 10.545 | 16.890 | 3.355 | 1.00 | 63.91 | N |
| ATOM | 782 | NH2 | ARG | A | 430 | 8.293 | 17.358 | 3.231 | 1.00 | 65.90 | N |
| ATOM | 783 | C | ARG | A | 430 | 11.818 | 17.957 | −3.591 | 1.00 | 54.65 | C |
| ATOM | 784 | O | ARG | A | 430 | 10.867 | 18.723 | −3.716 | 1.00 | 53.09 | O |
| ATOM | 785 | N | GLU | A | 431 | 12.932 | 18.078 | −4.307 | 1.00 | 55.81 | N |
| ATOM | 786 | CA | GLU | A | 431 | 13.077 | 19.158 | −5.268 | 1.00 | 54.84 | C |
| ATOM | 787 | CB | GLU | A | 431 | 14.428 | 19.855 | −5.092 | 1.00 | 58.53 | C |
| ATOM | 788 | CG | GLU | A | 431 | 14.491 | 20.755 | −3.869 | 1.00 | 61.42 | C |
| ATOM | 789 | CD | GLU | A | 431 | 15.662 | 21.716 | −3.910 | 1.00 | 67.37 | C |
| ATOM | 790 | OE1 | GLU | A | 431 | 15.715 | 22.555 | −4.841 | 1.00 | 71.67 | O |
| ATOM | 791 | OE2 | GLU | A | 431 | 16.530 | 21.638 | −3.013 | 1.00 | 68.85 | O |
| ATOM | 792 | C | GLU | A | 431 | 12.913 | 18.709 | −6.705 | 1.00 | 55.28 | C |
| ATOM | 793 | O | GLU | A | 431 | 12.995 | 19.521 | −7.622 | 1.00 | 58.15 | O |
| ATOM | 794 | N | GLU | A | 432 | 12.678 | 17.420 | −6.912 | 1.00 | 50.44 | N |
| ATOM | 795 | CA | GLU | A | 432 | 12.498 | 16.923 | −8.263 | 1.00 | 48.35 | C |
| ATOM | 796 | CB | GLU | A | 432 | 13.596 | 15.918 | −8.623 | 1.00 | 55.95 | C |
| ATOM | 797 | CG | GLU | A | 432 | 13.285 | 14.477 | −8.287 | 1.00 | 55.95 | C |
| ATOM | 798 | CD | GLU | A | 432 | 14.264 | 13.513 | −8.934 | 1.00 | 62.60 | C |
| ATOM | 799 | OE1 | GLU | A | 432 | 15.463 | 13.571 | −8.594 | 1.00 | 66.37 | O |
| ATOM | 800 | OE2 | GLU | A | 432 | 13.837 | 12.700 | −9.786 | 65.64 | O | |
| ATOM | 801 | C | GLU | A | 432 | 11.125 | 16.285 | −8.432 | 1.00 | 47.94 | C |
| ATOM | 802 | O | GLU | A | 432 | 10.661 | 16.082 | −9.548 | 1.00 | 47.37 | O |
| ATOM | 803 | N | ILE | A | 433 | 10.474 | 15.968 | −7.315 | 1.00 | 39.35 | N |
| ATOM | 804 | CA | ILE | A | 433 | 9.143 | 15.364 | −7.361 | 1.00 | 35.26 | C |
| ATOM | 805 | CB | ILE | A | 433 | 9.148 | 13.942 | −6.758 | 1.00 | 40.43 | C |
| ATOM | 806 | CG2 | ILE | A | 433 | 7.742 | 13.332 | −6.819 | 1.00 | 32.65 | C |
| ATOM | 807 | CG1 | ILE | A | 433 | 10.120 | 13.056 | −7.527 | 1.00 | 39.75 | C |
| ATOM | 808 | CD1 | ILE | A | 433 | 9.720 | 12.837 | −8.964 | 1.00 | 41.51 | C |
| ATOM | 809 | C | ILE | A | 433 | 8.252 | 16.258 | −6.521 | 1.00 | 36.84 | C |
| ATOM | 810 | O | ILE | A | 433 | 8.276 | 16.182 | −5.291 | 1.00 | 32.85 | O |
| ATOM | 811 | N | PRO | A | 434 | 7.467 | 17.134 | −7.172 | 1.00 | 33.02 | N |
| ATOM | 812 | CD | PRO | A | 434 | 7.510 | 17.400 | −8.618 | 1.00 | 41.68 | C |
| ATOM | 813 | CA | PRO | A | 434 | 6.554 | 18.074 | −6.512 | 1.00 | 36.57 | C |
| ATOM | 814 | CB | PRO | A | 434 | 5.925 | 18.812 | −7.693 | 1.00 | 37.02 | C |
| ATOM | 815 | CG | PRO | A | 434 | 7.033 | 18.833 | −8.679 | 1.00 | 47.39 | C |
| ATOM | 816 | C | PRO | A | 434 | 5.512 | 17.377 | −5.648 | 1.00 | 31.45 | C |
| ATOM | 817 | O | PRO | A | 434 | 5.227 | 16.199 | −5.848 | 1.00 | 28.84 | O |
| ATOM | 818 | N | VAL | A | 435 | 4.944 | 18.121 | −4.702 | 1.00 | 32.88 | N |
| ATOM | 819 | CA | VAL | A | 435 | 3.919 | 17.568 | −3.821 | 1.00 | 34.12 | C |
| ATOM | 820 | CB | VAL | A | 435 | 3.457 | 18.619 | −2.777 | 1.00 | 32.16 | C |
| ATOM | 821 | CG1 | VAL | A | 435 | 2.335 | 18.041 | −1.904 | 1.00 | 32.92 | C |
| ATOM | 822 | CG2 | VAL | A | 435 | 4.629 | 19.012 | −1.901 | 1.00 | 39.50 | C |
| ATOM | 823 | C | VAL | A | 435 | 2.731 | 17.093 | −4.642 | 1.00 | 34.03 | C |
| ATOM | 824 | O | VAL | A | 435 | 2.096 | 16.076 | −4.299 | 1.00 | 26.73 | O |
| ATOM | 825 | N | SER | A | 436 | 2.424 | 17.821 | −5.721 | 1.00 | 27.34 | N |
| ATOM | 826 | CA | SER | A | 436 | 1.321 | 17.445 | −6.595 | 1.00 | 26.52 | C |
| ATOM | 827 | CB | SER | A | 436 | 1.152 | 18.482 | −7.752 | 1.00 | 34.16 | C |
| ATOM | 828 | OG | SER | A | 436 | 2.339 | 18.588 | −8.494 | 1.00 | 36.95 | O |
| ATOM | 829 | C | SER | A | 436 | 1.517 | 16.038 | −7.187 | 1.00 | 29.26 | C |
| ATOM | 830 | O | SER | A | 436 | 0.548 | 15.298 | −7.359 | 1.00 | 28.55 | O |
| ATOM | 831 | N | ASN | A | 437 | 2.763 | 15.676 | −7.486 | 1.00 | 24.99 | N |
| ATOM | 832 | CA | ASN | A | 437 | 3.085 | 14.372 | −8.060 | 1.00 | 24.95 | C |
| ATOM | 833 | CB | ASN | A | 437 | 4.499 | 14.432 | −8.649 | 1.00 | 30.76 | C |
| ATOM | 834 | CG | ASN | A | 437 | 4.891 | 13.192 | −9.462 | 1.00 | 33.40 | C |
| ATOM | 835 | OD1 | ASN | A | 437 | 5.922 | 13.209 | −10.140 | 1.00 | 31.35 | O |
| ATOM | 836 | ND2 | ASN | A | 437 | 4.107 | 12.116 | −9.381 | 1.00 | 33.64 | N |
| ATOM | 837 | C | ASN | A | 437 | 2.957 | 13.330 | −6.923 | 1.00 | 24.92 | C |
| ATOM | 838 | O | ASN | A | 437 | 2.472 | 12.224 | −7.133 | 1.00 | 26.65 | O |
| ATOM | 839 | N | VAL | A | 438 | 3.371 | 13.695 | −5.719 | 1.00 | 25.75 | N |
| ATOM | 840 | CA | VAL | A | 438 | 3.201 | 12.755 | −4.612 | 1.00 | 24.63 | C |
| ATOM | 841 | CB | VAL | A | 438 | 3.819 | 13.291 | −3.321 | 1.00 | 28.75 | C |
| ATOM | 842 | CG1 | VAL | A | 438 | 3.496 | 12.348 | −2.162 | 1.00 | 25.61 | C |
| ATOM | 843 | CG2 | VAL | A | 438 | 5.338 | 13.426 | −3.500 | 1.00 | 27.56 | C |
| ATOM | 844 | C | VAL | A | 438 | 1.697 | 12.523 | −4.409 | 1.00 | 24.68 | C |
| ATOM | 845 | O | VAL | A | 438 | 1.277 | 11.391 | −4.258 | 1.00 | 22.54 | O |
| ATOM | 846 | N | ALA | A | 439 | 0.888 | 13.591 | −4.441 | 1.00 | 22.86 | N |
| ATOM | 847 | CA | ALA | A | 439 | −0.567 | 13.437 | −4.280 | 1.00 | 25.85 | C |
| ATOM | 848 | CB | ALA | A | 439 | −1.263 | 14.843 | −4.217 | 1.00 | 25.16 | C |
| ATOM | 849 | C | ALA | A | 439 | −1.188 | 12.589 | −5.395 | 1.00 | 22.87 | C |
| ATOM | 850 | O | ALA | A | 439 | −2.146 | 11.861 | −5.154 | 1.00 | 23.17 | O |
| ATOM | 851 | N | GLU | A | 440 | −0.647 | 12.685 | −6.614 | 1.00 | 21.21 | N |
| ATOM | 852 | CA | GLU | A | 440 | −1.157 | 11.900 | −7.728 | 1.00 | 25.32 | C |
| ATOM | 853 | CB | GLU | A | 440 | −0.380 | 12.246 | −9.014 | 1.00 | 30.55 | C |
| ATOM | 854 | CG | GLU | A | 440 | −0.636 | 11.301 | −10.184 | 1.00 | 29.05 | C |
| ATOM | 855 | CD | GLU | A | 440 | 0.286 | 11.582 | −11.368 | 1.00 | 40.39 | C |
| ATOM | 856 | OE1 | GLU | A | 440 | 1.448 | 11.984 | −11.145 | 1.00 | 35.18 | O |
| ATOM | 857 | OE2 | GLU | A | 440 | −0.151 | 11.385 | −12.523 | 1.00 | 36.07 | O |
| ATOM | 858 | C | GLU | A | 440 | −0.998 | 10.404 | −7.406 | 1.00 | 31.15 | C |
| ATOM | 859 | O | GLU | A | 440 | −1.923 | 9.614 | −7.585 | 1.00 | 23.98 | O |
| ATOM | 860 | N | LEU | A | 441 | 0.181 | 10.035 | −6.913 | 1.00 | 24.24 | N |
| ATOM | 861 | CA | LEU | A | 441 | 0.500 | 8.646 | −6.573 | 1.00 | 21.78 | C |
| ATOM | 862 | CB | LEU | A | 441 | 1.991 | 8.541 | −6.222 | 1.00 | 22.41 | C |
| ATOM | 863 | CG | LEU | A | 441 | 2.937 | 8.916 | −7.366 | 1.00 | 22.95 | C |
| ATOM | 864 | CD1 | LEU | A | 441 | 4.389 | 8.882 | −6.901 | 1.00 | 27.25 | C |
| ATOM | 865 | CD2 | LEU | A | 441 | 2.720 | 7.939 | −8.521 | 1.00 | 19.84 | C |
| ATOM | 866 | C | LEU | A | 441 | −0.354 | 8.186 | −5.377 | 1.00 | 18.04 | C |
| ATOM | 867 | O | LEU | A | 441 | −0.872 | 7.067 | −5.375 | 1.00 | 23.44 | O |
| ATOM | 868 | N | LEU | A | 442 | −0.476 | 9.040 | −4.369 | 1.00 | 20.94 | N |
| ATOM | 869 | CA | LEU | A | 442 | −1.310 | 8.653 | −3.219 | 1.00 | 24.86 | C |
| ATOM | 870 | CB | LEU | A | 442 | −1.233 | 9.698 | −2.115 | 1.00 | 22.54 | C |
| ATOM | 871 | CG | LEU | A | 442 | 0.100 | 9.753 | −1.362 | 1.00 | 22.80 | C |
| ATOM | 872 | CD1 | LEU | A | 442 | 0.125 | 10.921 | −0..55 | 1.00 | 22.67 | C |
| ATOM | 873 | CD2 | LEU | A | 442 | 0.315 | 8.398 | −0.661 | 1.00 | 23.13 | C |
| ATOM | 874 | C | LEU | A | 442 | −2.761 | 8.469 | −3.667 | 1.00 | 25.24 | C |
| ATOM | 875 | O | LEU | A | 442 | −3.454 | 7.581 | −3.187 | 1.00 | 22.68 | O |
| ATOM | 876 | N | HIS | A | 443 | −3.228 | 9.290 | −4.605 | 1.00 | 23.39 | N |
| ATOM | 877 | CA | HIS | A | 443 | −4.607 | 9.115 | −5.069 | 1.00 | 22.12 | C |
| ATOM | 878 | CB | HIS | A | 443 | −5.024 | 10.280 | −5.985 | 1.00 | 27.77 | C |
| ATOM | 879 | CG | HIS | A | 443 | −6.388 | 10.101 | −6.573 | 1.00 | 28.68 | C |
| ATOM | 880 | CD2 | HIS | A | 443 | −7.565 | 9.739 | −6.004 | 1.00 | 27.01 | C |
| ATOM | 881 | ND1 | HIS | A | 443 | −6.628 | 10.196 | −7.929 | 1.00 | 24.79 | N |
| ATOM | 882 | CE1 | HIS | A | 443 | −7.893 | 9.897 | −8.171 | 1.00 | 24.33 | C |
| ATOM | 883 | NE2 | HIS | A | 443 | −8.484 | 9.613 | −7.021 | 1.00 | 22.99 | N |
| ATOM | 884 | C | HIS | A | 443 | −4.781 | 7.762 | −5.781 | 1.00 | 22.67 | C |
| ATOM | 885 | O | HIS | A | 443 | −5.792 | 7.077 | −5.600 | 1.00 | 21.81 | O |
| ATOM | 886 | N | GLN | A | 444 | −3.791 | 7.354 | −6.569 | 1.00 | 22.84 | N |
| ATOM | 887 | CA | GLN | A | 444 | −3.855 | 6.076 | −7.251 | 1.00 | 18.98 | C |
| ATOM | 888 | CB | GLN | A | 444 | −2.637 | 5.909 | −8.152 | 1.00 | 23.77 | C |
| ATOM | 889 | CG | GLN | A | 444 | −2.679 | 6.923 | −9.303 | 1.00 | 23.82 | C |
| ATOM | 890 | CD | GLN | A | 444 | −1.445 | 6.867 | −10.130 | 1.00 | 29.64 | C |
| ATOM | 891 | OE1 | GLN | A | 444 | −0.424 | 6.344 | −9.697 | 1.00 | 28.87 | O |
| ATOM | 892 | NE2 | GLN | A | 444 | −1.521 | 7.419 | −11.342 | 1.00 | 28.75 | N |
| ATOM | 893 | C | GLN | A | 444 | −3.911 | 4.958 | −6.218 | 1.00 | 21.47 | C |
| ATOM | 894 | O | GLN | A | 444 | −4.682 | 4.029 | −6.354 | 1.00 | 22.83 | O |
| ATOM | 895 | N | VAL | A | 445 | −3.114 | 5.094 | −5.162 | 1.00 | 21.95 | N |
| ATOM | 896 | CA | VAL | A | 445 | −3.162 | 4.075 | −4.119 | 1.00 | 19.79 | C |
| ATOM | 897 | CB | VAL | A | 445 | −2.131 | 4.359 | −3.001 | 1.00 | 21.17 | C |
| ATOM | 898 | CG1 | VAL | A | 445 | −2.309 | 3.326 | −1.862 | 1.00 | 22.83 | C |
| ATOM | 899 | CG2 | VAL | A | 445 | −0.732 | 4.274 | −3.575 | 1.00 | 19.35 | C |
| ATOM | 900 | C | VAL | A | 445 | −4.576 | 4.070 | −3.510 | 1.00 | 20.72 | C |
| ATOM | 901 | O | VAL | A | 445 | −5.107 | 3.002 | −3.225 | 1.00 | 21.91 | O |
| ATOM | 902 | N | SER | A | 446 | −5.184 | 5.244 | −3.316 | 1.00 | 18.98 | N |
| ATOM | 903 | CA | SER | A | 446 | −6.543 | 5.260 | −2.734 | 1.00 | 19.98 | C |
| ATOM | 904 | CB | SER | A | 446 | −6.988 | 6.704 | −2.353 | 1.00 | 21.69 | C |
| ATOM | 905 | OG | SER | A | 446 | −7.327 | 7.481 | −3.468 | 1.00 | 24.84 | O |
| ATOM | 906 | C | SER | A | 446 | −7.573 | 4.627 | −3.662 | 1.00 | 20.82 | C |
| ATOM | 907 | O | SER | A | 446 | −8.553 | 4.037 | −3.187 | 1.00 | 21.15 | O |
| ATOM | 908 | N | MET | A | 447 | −7.366 | 4.714 | −4.974 | 1.00 | 19.01 | N |
| ATOM | 909 | CA | MET | A | 447 | −8.294 | 4.078 | −5.909 | 1.00 | 22.58 | C |
| ATOM | 910 | CB | MET | A | 447 | −8.071 | 4.588 | −7.347 | 1.00 | 23.29 | C |
| ATOM | 911 | CG | MET | A | 447 | −8.540 | 6.039 | −7.513 | 1.00 | 27.40 | C |
| ATOM | 912 | SD | MET | A | 447 | −8.500 | 6.538 | −9.288 | 1.00 | 29.46 | S |
| ATOM | 913 | CE | MET | A | 447 | −6.769 | 6.928 | −9.496 | 1.00 | 28.17 | C |
| ATOM | 914 | C | MET | A | 447 | −8.133 | 2.558 | −5.838 | 1.00 | 25.02 | C |
| ATOM | 915 | O | MET | A | 447 | −9.114 | 1.821 | −5.859 | 1.00 | 22.91 | O |
| ATOM | 916 | N | GLY | A | 448 | −6.903 | 2.076 | −5.729 | 1.00 | 20.29 | N |
| ATOM | 917 | CA | GLY | A | 448 | −6.712 | 0.644 | −5.625 | 1.00 | 22.77 | C |
| ATOM | 918 | C | GLY | A | 448 | −7.337 | 0.135 | −4.327 | 1.00 | 23.66 | C |
| ATOM | 919 | O | GLY | A | 448 | −7.963 | −0.934 | −4.331 | 1.00 | 24.13 | 0 |
| ATOM | 920 | N | MET | A | 449 | −7.192 | 0.892 | −3.236 | 1.00 | 22.40 | N |
| ATOM | 921 | CA | MET | A | 449 | −7.756 | 0.450 | −1.939 | 1.00 | 21.23 | C |
| ATOM | 922 | CB | MET | A | 449 | −7.139 | 1.232 | −0.775 | 1.00 | 20.57 | C |
| ATOM | 923 | CG | MET | A | 449 | −5.611 | 1.012 | 0.550 | 1.00 | 19.53 | C |
| ATOM | 924 | SD | MET | A | 449 | −5.170 | 0.771 | 0.382 | 1.00 | 25.09 | S |
| ATOM | 925 | CE | MET | A | 449 | −6.110 | −1.175 | 1.087 | 1.00 | 26.33 | C |
| ATOM | 926 | C | MET | A | 449 | −9.282 | 0.566 | −1.888 | 1.00 | 22.12 | C |
| ATOM | 927 | O | MET | A | 449 | −9.964 | 0.236 | −1.245 | 1.00 | 22.36 | O |
| ATOM | 928 | N | LYS | A | 450 | −9.832 | 1.554 | −2.578 | 1.00 | 21.36 | N |
| ATOM | 929 | CA | LYS | A | 450 | −11.276 | 1.705 | −2.642 | 1.00 | 24.95 | C |
| ATOM | 930 | CB | LYS | A | 450 | −11.616 | 2.949 | −3.457 | 1.00 | 24.95 | C |
| ATOM | 931 | CG | LYS | A | 450 | −13.113 | 3.108 | −3.668 | 1.00 | 34.59 | C |
| ATOM | 932 | CD | LYS | A | 450 | −13.388 | 4.170 | −4.678 | 1.00 | 35.54 | C |
| ATOM | 933 | CE | LYS | A | 450 | −14.854 | 4.088 | −5.081 | 1.00 | 43.28 | C |
| ATOM | 934 | NZ | LYS | A | 450 | −15.200 | 5.226 | −5.962 | 1.00 | 51.23 | N |
| ATOM | 935 | C | LYS | A | 450 | −11.811 | 0.436 | −3.328 | 1.00 | 20.54 | C |
| ATOM | 936 | O | LYS | A | 450 | −12.829 | −0.138 | −2.926 | 1.00 | 23.77 | O |
| ATOM | 937 | N | TYR | A | 451 | −11.093 | −0.035 | −4.339 | 1.00 | 22.16 | N |
| ATOM | 938 | CA | TYR | A | 451 | −11.490 | −1.254 | −5.053 | 1.00 | 25.84 | C |
| ATOM | 939 | CB | TYR | A | 451 | −10.606 | −1.466 | −6.295 | 1.00 | 30.33 | C |
| ATOM | 940 | CG | TYR | A | 451 | −10.851 | −2.776 | 7.036 | 1.00 | 26.97 | C |
| ATOM | 941 | CD1 | TYR | A | 451 | −11.957 | −2.942 | −7.887 | 1.00 | 32.31 | C |
| ATOM | 942 | CE1 | TYR | A | 451 | −12.156 | −4.161 | −8.577 | 1.00 | 35.26 | C |
| ATOM | 943 | CD2 | TYR | A | 451 | −9.975 | −3.841 | −6.887 | 1.00 | 29.55 | C |
| ATOM | 944 | CE2 | TYR | A | 451 | −10.171 | −5.039 | −7.538 | 1.00 | 38.20 | C |
| ATOM | 945 | CZ | TYR | A | 451 | −11.250 | −5.202 | −8.384 | 1.00 | 34.79 | C |
| ATOM | 946 | OH | TYR | A | 451 | −11.351 | −6.416 | −9.028 | 1.00 | 33.34 | O |
| ATOM | 947 | C | TYR | A | 451 | −11.413 | −2.477 | −4.156 | 1.00 | 25.12 | C |
| ATOM | 948 | O | TYR | A | 451 | −12.349 | −3.279 | −4.111 | 1.00 | 21.15 | O |
| ATOM | 949 | N | LEU | A | 452 | −10.317 | −2.621 | −3.411 | 1.00 | 23.09 | N |
| ATOM | 950 | CA | LEU | A | 452 | −10.190 | −3.769 | −2.528 | 1.00 | 22.43 | C |
| ATOM | 951 | CB | LEU | A | 452 | −8.812 | −3.775 | −1.805 | 1.00 | 24.62 | C |
| ATOM | 952 | CG | LEU | A | 452 | −7.633 | −4.080 | −2.723 | 1.00 | 29.88 | C |
| ATOM | 953 | CD1 | LEU | A | 452 | −6.355 | −3.885 | −1.901 | 1.00 | 40.57 | C |
| ATOM | 954 | CD2 | LEU | A | 452 | −7.706 | −5.499 | −3.261 | 1.00 | 37.63 | C |
| ATOM | 955 | C | LEU | A | 452 | −11.289 | −3.770 | −1.487 | 1.00 | 23.29 | C |
| ATOM | 956 | O | LEU | A | 452 | −11.835 | −4.835 | −1.160 | 1.00 | 24.20 | O |
| ATOM | 957 | N | GLU | A | 453 | −11.615 | −2.586 | −0.966 | 1.00 | 21.59 | N |
| ATOM | 958 | CA | GLU | A | 453 | −12.644 | −2.465 | 0.044 | 1.00 | 23.71 | C |
| ATOM | 959 | CB | GLU | A | 453 | −12.707 | −1.020 | 0.530 | 1.00 | 26.43 | C |
| ATOM | 960 | CG | GLU | A | 453 | −13.831 | −0.730 | 1.517 | 1.00 | 27.65 | C |
| ATOM | 961 | CD | GLU | A | 453 | −13.831 | 0.727 | 1.990 | 1.00 | 34.87 | C |
| ATOM | 962 | OE1 | GLU | A | 453 | −14.451 | 1.582 | 1.298 | 1.00 | 33.02 | O |
| ATOM | 963 | OE2 | GLU | A | 453 | −13.200 | 1.012 | 3.042 | 1.00 | 30.22 | C |
| ATOM | 964 | C | GLU | A | 453 | −14.014 | −2.901 | −0.520 | 1.00 | 27.31 | C |
| ATOM | 965 | O | GLU | A | 453 | −14.763 | −3.656 | 0.129 | 1.00 | 25.17 | O |
| ATOM | 966 | N | GLU | A | 454 | −14.348 | −2.407 | −1.706 | 1.00 | 22.10 | N |
| ATOM | 967 | CA | GLU | A | 454 | −15.640 | −2.769 | −2.310 | 1.00 | 25.66 | C |
| ATOM | 968 | CB | GLU | A | 454 | −15.871 | −1.997 | −3.619 | 1.00 | 31.24 | C |
| ATOM | 969 | CG | GLU | A | 454 | −14.964 | −2.392 | −4.754 | 1.00 | 42.57 | C |
| ATOM | 970 | CD | GLU | A | 454 | −15.261 | −1.624 | −6.046 | 1.00 | 58.39 | C |
| ATOM | 971 | OE1 | GLU | A | 454 | −15.273 | −0.363 | −6.015 | 1.00 | 51.95 | O |
| ATOM | 972 | OE2 | GLU | A | 454 | −15.472 | −2.287 | −7.094 | 1.00 | 58.17 | O |
| ATOM | 973 | C | GLU | A | 454 | −15.721 | −4.263 | −2.581 | 1.00 | 31.04 | C |
| ATOM | 974 | O | GLU | A | 454 | −16.810 | −4.837 | −2.583 | 1.00 | 30.48 | O |
| ATOM | 975 | N | LYS | A | 455 | −14.583 | −4.915 | −2.799 | 1.00 | 24.65 | N |
| ATOM | 976 | CA | LYS | A | 455 | −14.580 | −6.358 | −3.056 | 1.00 | 26.01 | C |
| ATOM | 977 | CB | LYS | A | 455 | −13.474 | 6.742 | −4.058 | 1.00 | 26.87 | C |
| ATOM | 978 | CG | LYS | A | 455 | −13.600 | −6.047 | −5.435 | 1.00 | 32.48 | C |
| ATOM | 979 | CD | LYS | A | 455 | −14.919 | −6.413 | −6.093 | 1.00 | 34.46 | C |
| ATOM | 980 | CE | LYS | A | 455 | −15.166 | −5.643 | −7.397 | 1.00 | 39.80 | C |
| ATOM | 981 | NZ | LYS | A | 455 | −16.498 | −6.028 | −7.995 | 1.00 | 35.47 | N |
| ATOM | 982 | C | LYS | A | 455 | −14.402 | −7.158 | −1.764 | 1.00 | 25.88 | C |
| ATOM | 983 | O | LYS | A | 455 | −14.205 | −8.367 | −1.799 | 1.00 | 33.28 | O |
| ATOM | 984 | N | ASN | A | 456 | −14.484 | −6.474 | −0.632 | 1.00 | 26.80 | N |
| ATOM | 985 | CA | ASN | A | 456 | −14.348 | −7.110 | 0.679 | 1.00 | 33.33 | C |
| ATOM | 986 | CB | ASN | A | 456 | −15.537 | −8.042 | 0.956 | 1.00 | 33.66 | C |
| ATOM | 987 | CG | ASN | A | 456 | −16.833 | −7.293 | 1.019 | 1.00 | 41.21 | C |
| ATOM | 988 | OD1 | ASN | A | 456 | −17.005 | −6.397 | 1.847 | 1.00 | 38.41 | O |
| ATOM | 989 | ND2 | ASN | A | 456 | −17.763 | −7.640 | 0.128 | 1.00 | 43.69 | N |
| ATOM | 990 | C | ASN | A | 456 | −13.048 | −7.865 | 0.932 | 1.00 | 32.96 | C |
| ATOM | 991 | O | ASN | A | 456 | −13.065 | −8.982 | 1.447 | 1.00 | 29.31 | O |
| ATOM | 992 | N | PHE | A | 457 | −11.925 | −7.265 | 0.539 | 1.00 | 25.61 | N |
| ATOM | 993 | CA | PHE | A | 457 | −10.633 | −7.871 | 0.827 | 1.00 | 23.17 | C |
| ATOM | 994 | CB | PHE | A | 457 | −9.823 | −8.104 | −0.457 | 1.00 | 24.54 | C |
| ATOM | 995 | CG | PHE | A | 457 | −10.302 | −9.259 | −1.274 | 1.00 | 27.01 | C |
| ATOM | 996 | CD1 | PHE | A | 457 | −11.141 | −9.064 | −2.372 | 1.00 | 38.40 | C |
| ATOM | 997 | CD2 | PHE | A | 457 | −9.912 | −10.546 | −0.952 | 1.00 | 30.92 | C |
| ATOM | 998 | CE1 | PHE | A | 457 | −11.573 | −10.154 | −3.131 | 1.00 | 40.99 | C |
| ATOM | 999 | CE2 | PHE | A | 457 | −10.338 | −11.633 | −1.702 | 1.00 | 39.35 | C |
| ATOM | 1000 | CZ | PHE | A | 457 | −11.169 | −11.435 | −2.792 | 1.00 | 32.26 | C |
| ATOM | 1001 | C | PHE | A | 457 | −9.876 | −6.876 | 1.724 | 1.00 | 22.95 | C |
| ATOM | 1002 | O | PHE | A | 457 | −10.006 | −5.666 | 1.538 | 1.00 | 26.81 | O |
| ATOM | 1003 | N | VAL | A | 458 | −9.095 | −7.404 | 2.677 | 1.00 | 22.07 | N |
| ATOM | 1004 | CA | VAL | A | 458 | −8.271 | −6.587 | 3.574 | 1.00 | 18.40 | C |
| ATOM | 1005 | CB | VAL | A | 458 | −8.476 | −6.999 | 5.067 | 1.00 | 24.28 | C |
| ATOM | 1006 | CG1 | VAL | A | 458 | −7.490 | −6.239 | 5.978 | 1.00 | 19.09 | C |
| ATOM | 1007 | CG2 | VAL | A | 458 | −9.885 | −6.680 | 5.496 | 1.00 | 24.91 | C |
| ATOM | 1008 | C | VAL | A | 458 | −6.811 | −6.870 | 3.172 | 1.00 | 21.95 | C |
| ATOM | 1009 | O | VAL | A | 458 | −6.412 | −8.007 | 3.081 | 1.00 | 22.78 | O |
| ATOM | 1010 | N | HIS | A | 459 | −6.017 | −5.836 | 2.953 | 1.00 | 23.11 | N |
| ATOM | 1011 | CA | HIS | A | 459 | −4.620 | −6.058 | 2.514 | 1.00 | 22.90 | C |
| ATOM | 1012 | CB | HIS | A | 459 | −4.106 | −4.777 | 1.821 | 1.00 | 25.33 | C |
| ATOM | 1013 | CG | HIS | A | 459 | −2.735 | −4.915 | 1.233 | 1.00 | 27.05 | C |
| ATOM | 1014 | CD2 | HIS | A | 459 | −2.319 | −4.984 | −0.055 | 1.00 | 35.51 | C |
| ATOM | 1015 | ND1 | HIS | A | 459 | −1.598 | −4.995 | 2.004 | 1.00 | 30.45 | N |
| ATOM | 1016 | CE1 | HIS | A | 459 | −0.539 | −5.097 | 1.218 | 1.00 | 30.89 | C |
| ATOM | 1017 | NE2 | HIS | A | 459 | −0.950 | −5.091 | −0.037 | 1.00 | 27.20 | N |
| ATOM | 1018 | C | HIS | A | 459 | −3.726 | −6.406 | 3.702 | 1.00 | 22.15 | C |
| ATOM | 1019 | O | HIS | A | 459 | −2.926 | −7.373 | 3.669 | 1.00 | 26.50 | O |
| ATOM | 1020 | N | ARG | A | 460 | −3.870 | −5.592 | 4.743 | 1.00 | 22.61 | N |
| ATOM | 1021 | CA | ARG | A | 460 | −3.160 | −5.697 | 6.007 | 1.00 | 21.98 | C |
| ATOM | 1022 | CB | ARG | A | 460 | −3.202 | −7.138 | 6.526 | 1.00 | 27.77 | C |
| ATOM | 1023 | CG | ARG | A | 460 | −2.704 | −7.226 | 7.957 | 1.00 | 39.24 | C |
| ATOM | 1024 | CD | ARG | A | 460 | −2.722 | −8.632 | 8.504 | 1.00 | 31.08 | C |
| ATOM | 1025 | NE | ARG | A | 460 | −1.706 | −8.745 | 9.539 | 1.00 | 33.21 | N |
| ATOM | 1026 | CZ | ARG | A | 460 | −1.459 | −9.857 | 10.236 | 1.00 | 39.79 | C |
| ATOM | 1027 | NH1 | ARG | A | 460 | −2.161 | −10.955 | 10.014 | 1.00 | 32.10 | N |
| ATOM | 1028 | NH2 | ARG | A | 460 | −0.492 | −9.866 | 11.146 | 1.00 | 40.69 | N |
| ATOM | 1029 | C | ARG | A | 460 | −1.706 | −5.211 | 6.022 | 1.00 | 28.94 | C |
| ATOM | 1030 | O | ARG | A | 460 | −1.177 | −4.874 | 7.079 | 1.00 | 36.64 | O |
| ATOM | 1031 | N | ASP | A | 461 | −1.072 | −5.145 | 4.861 | 1.00 | 24.64 | N |
| ATOM | 1032 | CA | ASP | A | 461 | 0.325 | −4.733 | 4.836 | 1.00 | 26.58 | C |
| ATOM | 1033 | CB | ASP | A | 461 | 1.172 | −5.943 | 4.374 | 1.00 | 27.03 | C |
| ATOM | 1034 | CG | ASP | A | 461 | 2.667 | −5.827 | 4.743 | 1.00 | 38.59 | C |
| ATOM | 1035 | OD1 | ASP | A | 461 | 3.050 | −4.953 | 5.551 | 1.00 | 48.65 | O |
| ATOM | 1036 | OD2 | ASP | A | 461 | 3.468 | −6.646 | 4.224 | 1.00 | 49.76 | O |
| ATOM | 1037 | C | ASP | A | 461 | 0.521 | −3.500 | 3.932 | 1.00 | 27.57 | C |
| ATOM | 1038 | O | ASP | A | 461 | 1.524 | −3.370 | 3.237 | 1.00 | 26.49 | O |
| ATOM | 1039 | N | LEU | A | 462 | −0.429 | −2.572 | 3.956 | 1.00 | 22.27 | N |
| ATOM | 1040 | CA | LEU | A | 462 | −0.278 | −1.380 | 3.139 | 1.00 | 21.53 | C |
| ATOM | 1041 | CB | LEU | A | 462 | −1.636 | −0.666 | 2.987 | 1.00 | 22.43 | C |
| ATOM | 1042 | CG | LEU | A | 462 | −1.648 | 0.602 | 2.128 | 1.00 | 30.85 | C |
| ATOM | 1043 | CD1 | LEU | A | 462 | −1.215 | 0.224 | 0.715 | 1.00 | 29.37 | C |
| ATOM | 1044 | CD2 | LEU | A | 462 | −3.042 | 1.270 | 2.133 | 1.00 | 27.57 | C |
| ATOM | 1045 | C | LEU | A | 462 | 0.779 | −0.477 | 3.804 | 1.00 | 22.66 | C |
| ATOM | 1046 | O | LEU | A | 462 | 0.671 | −0.125 | 4.950 | 1.00 | 26.45 | O |
| ATOM | 1047 | N | ALA | A | 463 | 1.832 | −0.159 | 3.063 | 1.00 | 21.02 | N |
| ATOM | 1048 | CA | ALA | A | 463 | 2.926 | 0.688 | 3.572 | 1.00 | 19.38 | C |
| ATOM | 1049 | CB | ALA | A | 463 | 3.865 | −0.138 | 4.481 | 1.00 | 20.15 | C |
| ATOM | 1050 | C | ALA | A | 463 | 3.669 | 1.169 | 2.323 | 1.00 | 17.38 | C |
| ATOM | 1051 | O | ALA | A | 463 | 3.586 | 0.534 | 1.274 | 1.00 | 21.49 | O |
| ATOM | 1052 | N | ALA | A | 464 | 4.383 | 2.277 | 2.438 | 1.00 | 20.81 | N |
| ATOM | 1053 | CA | ALA | A | 464 | 5.062 | 2.797 | 1.271 | 1.00 | 22.24 | C |
| ATOM | 1054 | CB | ALA | A | 464 | 5.810 | 4.094 | 1.651 | 1.00 | 19.74 | C |
| ATOM | 1055 | C | ALA | A | 464 | 6.005 | 1.777 | 0.641 | 1.00 | 23.62 | C |
| ATOM | 1056 | O | ALA | A | 464 | 6.180 | 1.782 | −0.571 | 1.00 | 22.85 | O |
| ATOM | 1057 | N | ARG | A | 465 | 6.611 | 0.915 | 1.457 | 1.00 | 23.55 | N |
| ATOM | 1058 | CA | ARG | A | 465 | 7.528 | −0.112 | 0.962 | 1.00 | 22.78 | C |
| ATOM | 1059 | CB | ARG | A | 465 | 8.203 | −0.852 | 2.133 | 1.00 | 26.22 | C |
| ATOM | 1060 | CG | ARG | A | 465 | 7.254 | −1.662 | 3.026 | 1.00 | 22.40 | C |
| ATOM | 1061 | CD | ARG | A | 465 | 7.980 | −2.238 | 4.272 | 1.00 | 27.73 | C |
| ATOM | 1062 | NE | ARG | A | 465 | 7.030 | −2.873 | 5.180 | 1.00 | 32.59 | N |
| ATOM | 1063 | CZ | ARG | A | 465 | 6.337 | −2.232 | 6.126 | 1.00 | 41.47 | C |
| ATOM | 1064 | NH1 | ARG | A | 465 | 6.491 | −0.928 | 6.320 | 1.00 | 34.18 | N |
| ATOM | 1065 | NH2 | ARG | A | 465 | 5.441 | −2.887 | 6.841 | 1.00 | 40.07 | N |
| ATOM | 1066 | C | ARG | A | 465 | 6.814 | −1.136 | 0.084 | 1.00 | 24.95 | C |
| ATOM | 1067 | O | ARG | A | 465 | 7.461 | −1.867 | −0.673 | 1.00 | 25.58 | O |
| ATOM | 1068 | N | ASN | A | 466 | 5.484 | −1.203 | 0.199 | 1.00 | 24.98 | N |
| ATOM | 1069 | CA | ASN | A | 466 | 4.715 | −2.149 | −0.603 | 1.00 | 23.15 | C |
| ATOM | 1070 | CB | ASN | A | 466 | 3.785 | −2.982 | 0.276 | 1.00 | 20.54 | C |
| ATOM | 1071 | CG | ASN | A | 466 | 4.537 | −3.987 | 1.118 | 1.00 | 29.73 | C |
| ATOM | 1072 | OD1 | ASN | A | 466 | 5.600 | −4.469 | 0.710 | 1.00 | 26.55 | O |
| ATOM | 1073 | ND2 | ASN | A | 466 | 4.000 | −4.310 | 2.302 | 1.00 | 25.54 | N |
| ATOM | 1074 | C | ASN | A | 466 | 3.947 | −1.509 | −1.752 | 1.00 | 23.75 | C |
| ATOM | 1075 | O | ASN | A | 466 | 3.185 | −2.171 | −2.422 | 1.00 | 24.79 | O |
| ATOM | 1076 | N | VAL | A | 467 | 4.170 | −0.220 | −1.959 | 1.00 | 21.31 | N |
| ATOM | 1077 | CA | VAL | A | 467 | 3.561 | 0.462 | −3.086 | 1.00 | 19.34 | C |
| ATOM | 1078 | CB | VAL | A | 467 | 3.127 | 1.872 | −2.699 | 1.00 | 21.47 | C |
| ATOM | 1079 | CG1 | VAL | A | 467 | 2.736 | 2.647 | −3.979 | 1.00 | 22.29 | C |
| ATOM | 1080 | CG2 | VAL | A | 467 | 1.957 | 1.792 | −1.696 | 1.00 | 19.95 | C |
| ATOM | 1081 | C | VAL | A | 467 | 4.733 | 0.538 | −4.076 | 1.00 | 22.01 | C |
| ATOM | 1082 | O | VAL | A | 467 | 5.817 | 1.025 | 3.721 | 1.00 | 22.82 | O |
| ATOM | 1083 | N | LEU | A | 468 | 4.524 | 0.036 | −5.290 | 1.00 | 21.63 | N |
| ATOM | 1084 | CA | LEU | A | 468 | 5.601 | 0.021 | −6.286 | 1.00 | 25.97 | C |
| ATOM | 1085 | CB | LEU | A | 468 | 5.828 | −1.399 | −6.801 | 1.00 | 21.35 | C |
| ATOM | 1086 | CG | LEU | A | 468 | 6.073 | −2.498 | −5.767 | 1.00 | 25.31 | C |
| ATOM | 1087 | CD1 | LEU | A | 468 | 5.995 | −3.859 | −6.430 | 1.00 | 25.16 | C |
| ATOM | 1088 | CD2 | LEU | A | 468 | 7.434 | −2.267 | −5.110 | 1.00 | 27.20 | C |
| ATOM | 1089 | C | LEU | A | 468 | 5.285 | 0.920 | −7.465 | 1.00 | 29.27 | C |
| ATOM | 1090 | O | LEU | A | 468 | 4.143 | 1.006 | −7.884 | 1.00 | 26.29 | O |
| ATOM | 1091 | N | LEU | A | 469 | 6.311 | 1.554 | −8.035 | 1.00 | 24.41 | N |
| ATOM | 1093 | CA | LEU | A | 469 | 6.063 | 2.440 | −9.156 | 1.00 | 25.24 | C |
| ATOM | 1093 | CB | LEU | A | 469 | 6.886 | 3.714 | −8.973 | 1.00 | 28.20 | C |
| ATOM | 1094 | CG | LEU | A | 469 | 6.583 | 4.441 | −7.661 | 1.00 | 40.39 | C |
| ATOM | 1095 | CD1 | LEU | A | 469 | 7.566 | 5.547 | −7.471 | 1.00 | 42.75 | C |
| ATOM | 1096 | CD2 | LEU | A | 469 | 5.180 | 5.007 | −7.681 | 1.00 | 36.92 | C |
| ATOM | 1097 | C | LEU | A | 469 | 6.356 | 1.844 | −10.518 | 1.00 | 25.99 | C |
| ATOM | 1098 | O | LEU | A | 469 | 7.411 | 1.237 | −10.729 | 1.00 | 27.91 | O |
| ATOM | 1099 | N | VAL | A | 470 | 5.416 | 1.985 | −11.445 | 1.00 | 27.37 | N |
| ATOM | 1100 | CA | VAL | A | 470 | 5.662 | 1.526 | −12.795 | 1.00 | 27.70 | C |
| ATOM | 1101 | CB | VAL | A | 470 | 4.369 | 1.352 | −13.589 | 1.00 | 30.57 | C |
| ATOM | 1102 | CG1 | VAL | A | 470 | 4.693 | 1.012 | −15.042 | 1.00 | 35.99 | C |
| ATOM | 1103 | CG2 | VAL | A | 470 | 3.536 | 0.219 | −12.963 | 1.00 | 28.74 | C |
| ATOM | 1104 | C | VAL | A | 470 | 6.517 | 2.663 | −13.384 | 1.00 | 32.73 | C |
| ATOM | 1105 | O | VAL | A | 470 | 7.467 | 2.415 | −14.137 | 1.00 | 35.56 | O |
| ATOM | 1106 | N | ASN | A | 471 | 6.172 | 3.895 | −13.012 | 1.00 | 30.15 | N |
| ATOM | 1107 | CA | ASN | A | 471 | 6.889 | 5.122 | −13.415 | 1.00 | 35.49 | C |
| ATOM | 1108 | CB | ASN | A | 471 | 6.441 | 5.610 | −14.805 | 1.00 | 38.79 | C |
| ATOM | 1109 | CG | ASN | A | 471 | 4.941 | 5.766 | −14.912 | 1.00 | 38.59 | C |
| ATOM | 1110 | OD1 | ASN | A | 471 | 4.246 | 4.841 | −15.320 | 1.00 | 61.19 | O |
| ATOM | 1111 | ND2 | ASN | A | 471 | 4.432 | 6.917 | −14.518 | 1.00 | 37.18 | N |
| ATOM | 1112 | C | ASN | A | 471 | 6.561 | 6.186 | −12.366 | 1.00 | 34.10 | C |
| ATOM | 1113 | O | ASN | A | 471 | 5.766 | 5.925 | −11.458 | 1.00 | 31.54 | O |
| ATOM | 1114 | N | ARG | A | 472 | 7.144 | 7.383 | −12.468 | 1.00 | 31.44 | N |
| ATOM | 1115 | CA | ARG | A | 472 | 6.889 | 8.423 | −11.467 | 1.00 | 34.23 | C |
| ATOM | 1116 | CB | ARG | A | 472 | 7.812 | 9.628 | −11.699 | 1.00 | 36.29 | C |
| ATOM | 1117 | CG | ARG | A | 472 | 7.539 | 10.352 | −13.002 | 1.00 | 38.64 | C |
| ATOM | 1118 | CD | ARG | A | 472 | 8.440 | 11.577 | −13.126 | 1.00 | 42.01 | C |
| ATOM | 1119 | NE | ARG | A | 472 | 7.960 | 12.686 | −12.310 | 1.00 | 42.62 | N |
| ATOM | 1120 | CZ | ARG | A | 472 | 8.611 | 13.835 | −12.142 | 1.00 | 49.56 | C |
| ATOM | 1121 | NH1 | ARG | A | 472 | 9.789 | 14.036 | −12.734 | 1.00 | 41.21 | N |
| ATOM | 1122 | NH2 | ARG | A | 472 | 8.082 | 14.791 | −11.395 | 1.00 | 45.67 | N |
| ATOM | 1123 | C | ARG | A | 472 | 5.442 | 8.907 | −11.393 | 1.00 | 23.39 | C |
| ATOM | 1124 | O | ARG | A | 472 | 5.087 | 9.644 | −10.491 | 1.00 | 29.27 | O |
| ATOM | 1125 | N | HIS | A | 473 | 4.621 | 8.504 | −12.357 | 1.00 | 26.03 | N |
| ATOM | 1126 | CA | HIS | A | 473 | 3.234 | 8.911 | −12.392 | 1.00 | 32.63 | C |
| ATOM | 1127 | CB | HIS | A | 473 | 2.947 | 9.729 | −13.680 | 1.00 | 27.76 | C |
| ATOM | 1128 | CG | HIS | A | 473 | 3.693 | 11.026 | −13.743 | 1.00 | 31.49 | C |
| ATOM | 1129 | CD1 | HIS | A | 473 | 4.728 | 11.426 | −14.522 | 1.00 | 40.44 | C |
| ATOM | 1130 | ND1 | HIS | A | 473 | 3.462 | 12.056 | −12.858 | 1.00 | 33.49 | N |
| ATOM | 1131 | CE1 | HIS | A | 473 | 4.327 | 13.031 | −13.082 | 1.00 | 42.59 | C |
| ATOM | 1132 | NE2 | HIS | A | 473 | 5.108 | 12.673 | −14.086 | 1.00 | 41.30 | N |
| ATOM | 1133 | C | HIS | A | 473 | 2.296 | 7.710 | −12.329 | 1.00 | 28.13 | C |
| ATOM | 1134 | O | HIS | A | 473 | 1.105 | 7.842 | −12.651 | 1.00 | 25.55 | O |
| ATOM | 1135 | N | TYR | A | 474 | 2.813 | 6.547 | −11.928 | 1.00 | 26.97 | N |
| ATOM | 1136 | CA | TYR | A | 474 | 1.957 | 5.357 | −11.889 | 1.00 | 29.51 | C |
| ATOM | 1137 | CB | TYR | A | 474 | 1.991 | 4.652 | −13.250 | 1.00 | 26.22 | C |
| ATOM | 1138 | CG | TYR | A | 474 | 0.982 | 3.515 | −13.472 | 1.00 | 30.97 | C |
| ATOM | 1139 | CD1 | TYR | A | 474 | −0.077 | 3.274 | −12.582 | 1.00 | 28.77 | C |
| ATOM | 1140 | CE1 | TYR | A | 474 | −1.001 | −2.218 | −12.823 | 1.00 | 31.08 | C |
| ATOM | 1141 | CD2 | TYR | A | 474 | 1.091 | 2.689 | −14.596 | 1.00 | 33.81 | C |
| ATOM | 1142 | CE2 | TYR | A | 474 | 0.191 | 1.658 | −14.838 | 1.00 | 34.98 | C |
| ATOM | 1143 | CZ | TYR | A | 474 | −0.853 | 1.418 | −13.960 | 1.00 | 33.21 | C |
| ATOM | 1144 | OH | TYR | A | 474 | −1.723 | 0.375 | −14.256 | 1.00 | 31.45 | O |
| ATOM | 1145 | C | TYR | A | 474 | 2.358 | 4.376 | −10.789 | 1.00 | 25.47 | C |
| ATOM | 1146 | O | TYR | A | 474 | 3.336 | 3.636 | −10.932 | 1.00 | 28.97 | O |
| ATOM | 1147 | N | ALA | A | 475 | 1.571 | 4.374 | −9.710 | 1.00 | 22.78 | N |
| ATOM | 1148 | CA | ALA | A | 475 | 1.837 | 3.495 | −8.577 | 1.00 | 26.62 | C |
| ATOM | 1149 | CB | ALA | A | 475 | 1.696 | 4.297 | −7.265 | 1.00 | 27.18 | C |
| ATOM | 1150 | C | ALA | A | 475 | 0.897 | 2.280 | −8.553 | 1.00 | 28.20 | C |
| ATOM | 1151 | O | ALA | A | 475 | −0.234 | 2.341 | −9.045 | 1.00 | 25.92 | O |
| ATOM | 1152 | N | LYS | A | 476 | 1.380 | 1.169 | −7.990 | 1.00 | 24.41 | N |
| ATOM | 1153 | CA | LYS | A | 476 | 0.569 | −0.039 | −7.855 | 1.00 | 23.63 | C |
| ATOM | 1154 | CB | LYS | A | 476 | 0.957 | −1.092 | −8.912 | 1.00 | 26.51 | C |
| ATOM | 1155 | CG | LYS | A | 476 | 0.418 | −0.795 | −10.305 | 1.00 | 27.53 | C |
| ATOM | 1156 | CD | LYS | A | 476 | 0.985 | −1.811 | −11.310 | 1.00 | 32.34 | C |
| ATOM | 1157 | CE | LYS | A | 476 | 0.273 | −1.748 | −12.657 | 1.00 | 36.56 | C |
| ATOM | 1158 | NZ | LYS | A | 476 | −0.989 | −2.542 | −12.605 | 1.00 | 37.90 | N |
| ATOM | 1159 | C | LYS | A | 476 | 0.772 | −0.657 | −6.477 | 1.00 | 25.52 | C |
| ATOM | 1160 | O | LYS | A | 476 | 1.898 | −0.684 | −5.956 | 1.00 | 25.22 | O |
| ATOM | 1161 | N | ILE | A | 477 | −0.309 | −1.172 | −5.896 | 1.00 | 22.11 | N |
| ATOM | 1162 | CA | ILE | A | 477 | −0.214 | −1.800 | −4.580 | 1.00 | 24.01 | C |
| ATOM | 1163 | CB | ILE | A | 477 | −1.577 | −1.846 | −3.915 | 1.00 | 21.78 | C |
| ATOM | 1164 | CG2 | ILE | A | 477 | −1.499 | −2.642 | −2.596 | 1.00 | 25.26 | C |
| ATOM | 1165 | CG1 | ILE | A | 477 | −2.081 | −0.417 | −3.708 | 1.00 | 20.73 | C |
| ATOM | 1166 | CD1 | ILE | A | 477 | −3.578 | −0.332 | −3.534 | 1.00 | 22.58 | C |
| ATOM | 1167 | C | ILE | A | 477 | 0.267 | −3.223 | −4.766 | 1.00 | 24.46 | C |
| ATOM | 1168 | O | ILE | A | 477 | −0.216 | −3.919 | −5.654 | 1.00 | 22.86 | O |
| ATOM | 1169 | N | SER | A | 478 | 1.214 | −3.666 | −3.932 | 1.00 | 22.32 | N |
| ATOM | 1170 | CA | SER | A | 478 | 1.691 | −5.039 | −4.071 | 1.00 | 22.20 | C |
| ATOM | 1171 | CB | SER | A | 478 | 3.097 | −5.049 | −4.649 | 1.00 | 28.05 | C |
| ATOM | 1172 | OG | SER | A | 478 | 3.965 | −4.509 | −3.674 | 1.00 | 38.71 | O |
| ATOM | 1173 | C | SER | A | 478 | 1.723 | −5.787 | −2.738 | 1.00 | 26.29 | C |
| ATOM | 1174 | O | SER | A | 478 | 1.311 | −5.261 | −1.697 | 1.00 | 25.87 | O |
| ATOM | 1175 | N | ASP | A | 479 | 2.247 | −7.010 | −2.810 | 1.00 | 23.81 | N |
| ATOM | 1176 | CA | ASP | A | 479 | 2.394 | −7.959 | −1.690 | 1.00 | 25.45 | C |
| ATOM | 1177 | CB | ASP | A | 479 | 3.465 | −7.519 | −0.666 | 1.00 | 24.64 | C |
| ATOM | 1178 | CG | ASP | A | 479 | 3.850 | −8.767 | 0.288 | 1.00 | 35.61 | C |
| ATOM | 1179 | OD1 | ASP | A | 479 | 3.141 | −9.710 | 0.289 | 1.00 | 30.47 | O |
| ATOM | 1180 | OD2 | ASP | A | 479 | 4.860 | −8.567 | 1.018 | 1.00 | 31.61 | O |
| ATOM | 1181 | C | ASP | A | 479 | 1.099 | −8.294 | −0.952 | 1.00 | 21.75 | C |
| ATOM | 1182 | O | ASP | A | 479 | 0.819 | −7.751 | 0.144 | 1.00 | 21.47 | O |
| ATOM | 1183 | N | PHE | A | 480 | 0.359 | −9.246 | −1.524 | 1.00 | 24.40 | N |
| ATOM | 1184 | CA | PHE | A | 480 | −0.905 | −9.719 | −0.946 | 1.00 | 27.55 | C |
| ATOM | 1185 | CB | PHE | A | 480 | −1.910 | −9.973 | −2.077 | 1.00 | 22.84 | C |
| ATOM | 1186 | CG | PHE | A | 480 | −2.335 | −8.709 | −2.776 | 1.00 | 25.54 | C |
| ATOM | 1187 | CD1 | PHE | A | 480 | −3.472 | −7.999 | −2.373 | 1.00 | 37.22 | C |
| ATOM | 1188 | CD2 | PHE | A | 480 | −1.531 | −8.165 | −3.774 | 1.00 | 29.75 | C |
| ATOM | 1189 | CE1 | PHE | A | 480 | −3.788 | 6.767 | −2.950 | 1.00 | 36.53 | C |
| ATOM | 1190 | CE2 | PHE | A | 480 | −1.841 | −6.925 | −4.351 | 1.00 | 31.15 | C |
| ATOM | 1191 | CZ | PHE | A | 480 | −2.956 | −6.229 | −3.945 | 1.00 | 30.92 | C |
| ATOM | 1192 | C | PHE | A | 480 | −0.734 | −10.978 | −0.070 | 1.00 | 22.25 | C |
| ATOM | 1193 | O | PHE | A | 480 | −1.693 | −11.697 | 0.201 | 1.00 | 28.80 | O |
| ATOM | 1194 | N | GLY | A | 481 | 0.494 | −11.200 | 0.395 | 1.00 | 26.68 | N |
| ATOM | 1195 | CA | GLY | A | 481 | 0.808 | −12.354 | 1.234 | 1.00 | 25.28 | C |
| ATOM | 1196 | C | GLY | A | 481 | 0.041 | −12.422 | 2.553 | 1.00 | 29.85 | C |
| ATOM | 1197 | O | GLY | A | 481 | −0.094 | −13.489 | 3.125 | 1.00 | 28.84 | O |
| ATOM | 1198 | N | LEU | A | 482 | −0.455 | −11.293 | 3.049 | 1.00 | 21.56 | N |
| ATOM | 1199 | CA | LEU | A | 482 | −1.217 | −11.299 | 4.316 | 1.00 | 23.95 | C |
| ATOM | 1200 | CB | LEU | A | 482 | −0.608 | −10.287 | 5.304 | 1.00 | 25.94 | C |
| ATOM | 1201 | CG | LEU | A | 482 | 0.789 | −10.660 | 5.792 | 1.00 | 31.12 | C |
| ATOM | 1202 | CD1 | LEU | A | 482 | 1.418 | −9.482 | 6.554 | 1.00 | 34.67 | C |
| ATOM | 1203 | CD2 | LEU | A | 482 | 0.680 | −11.884 | 6.675 | 1.00 | 35.08 | C |
| ATOM | 1204 | C | LEU | A | 482 | −2.667 | −10.925 | 4.097 | 1.00 | 21.10 | C |
| ATOM | 1205 | O | LEU | A | 482 | −3.441 | −10.766 | 5.050 | 1.00 | 26.73 | O |
| ATOM | 1206 | N | SER | A | 483 | −3.050 | −10.794 | 2.837 | 1.00 | 21.83 | N |
| ATOM | 1207 | CA | SER | A | 483 | −4.409 | −10.375 | 2.518 | 1.00 | 17.76 | C |
| ATOM | 1208 | CB | SER | A | 483 | −4.465 | −9.958 | 1.042 | 1.00 | 22.28 | C |
| ATOM | 1209 | OG | SER | A | 483 | −3.595 | −8.851 | 0.860 | 1.00 | 29.25 | O |
| ATOM | 1210 | C | SER | A | 483 | −5.440 | −11.475 | 2.785 | 1.00 | 21.06 | C |
| ATOM | 1211 | O | SER | A | 483 | −5.128 | −12.645 | 2.739 | 1.00 | 24.20 | O |
| ATOM | 1212 | N | LYS | A | 484 | −6.681 | −11.071 | 3.034 | 1.00 | 20.71 | N |
| ATOM | 1213 | CA | LYS | A | 484 | −7.738 | −12.041 | 3.291 | 1.00 | 21.00 | C |
| ATOM | 1214 | CB | LYS | A | 484 | −7.862 | −12.318 | 4.791 | 1.00 | 24.78 | C |
| ATOM | 1215 | CG | LYS | A | 484 | −6.650 | −13.105 | 5.362 | 1.00 | 38.83 | C |
| ATOM | 1216 | CD | LYS | A | 484 | −6.770 | −13.393 | 6.859 | 1.00 | 45.27 | C |
| ATOM | 1217 | CE | LYS | A | 484 | −5.677 | −14.378 | 7.302 | 1.00 | 43.33 | C |
| ATOM | 1218 | NZ | LYS | A | 484 | −4.326 | −14.049 | 6.778 | 1.00 | 56.25 | N |
| ATOM | 1219 | C | LYS | A | 484 | −9.076 | −11.545 | 2.768 | 1.00 | 25.54 | C |
| ATOM | 1220 | O | LYS | A | 484 | −9.320 | −10.348 | 2.677 | 1.00 | 26.31 | O |
| ATOM | 1221 | N | ALA | A | 485 | −9.941 | −12.491 | 2.434 | 1.00 | 23.65 | N |
| ATOM | 1222 | CA | ALA | A | 485 | −11.280 | −12.138 | 1.974 | 1.00 | 22.67 | C |
| ATOM | 1223 | CB | ALA | A | 485 | −11.814 | −13.210 | 0.995 | 1.00 | 31.69 | C |
| ATOM | 1224 | C | ALA | A | 485 | −12.144 | −12.086 | 3.249 | 1.00 | 31.19 | C |
| ATOM | 1225 | O | ALA | A | 485 | −12.006 | −12.931 | 4.136 | 1.00 | 29.37 | O |
| ATOM | 1226 | N | LEU | A | 486 | −12.995 | −11.073 | 3.349 | 1.00 | 23.82 | N |
| ATOM | 1227 | CA | LEU | A | 486 | −13.873 | −10.926 | 4.518 | 1.00 | 20.69 | C |
| ATOM | 1228 | CB | LEU | A | 486 | −14.113 | −9.435 | 4.825 | 1.00 | 28.46 | C |
| ATOM | 1229 | CG | LEU | A | 486 | −13.031 | −8.604 | 5.532 | 1.00 | 31.01 | C |
| ATOM | 1230 | CD1 | LEU | A | 486 | −13.418 | −7.137 | 5.591 | 1.00 | 36.09 | C |
| ATOM | 1231 | CD2 | LEU | A | 486 | −12.840 | −9.129 | 6.923 | 1.00 | 37.43 | C |
| ATOM | 1232 | C | LEU | A | 486 | −15.239 | −11.583 | 4.288 | 1.00 | 31.06 | C |
| ATOM | 1233 | O | LEU | A | 486 | −15.911 | −11.953 | 5.235 | 1.00 | 33.37 | O |
| ATOM | 1234 | N | GLY | A | 487 | −15.651 | −11.715 | 3.031 | 1.00 | 28.59 | N |
| ATOM | 1235 | CA | GLY | A | 487 | −16.955 | −12.284 | 2.753 | 1.00 | 33.58 | C |
| ATOM | 1236 | C | GLY | A | 487 | −18.031 | −11.345 | 3.283 | 1.00 | 38.11 | C |
| ATOM | 1237 | O | GLY | A | 487 | −17.980 | −10.144 | 3.077 | 1.00 | 36.30 | O |
| ATOM | 1238 | N | ALA | A | 488 | −18.987 | −11.910 | 4.004 | 1.00 | 38.66 | N |
| ATOM | 1239 | CA | ALA | A | 488 | −20.092 | −11.130 | 4.556 | 1.00 | 40.22 | C |
| ATOM | 1240 | CB | ALA | A | 488 | −21.287 | −12.045 | 4.812 | 1.00 | 46.29 | C |
| ATOM | 1241 | C | ALA | A | 488 | −19.733 | −10.388 | 5.843 | 1.00 | 40.58 | C |
| ATOM | 1242 | O | ALA | A | 488 | −20.438 | −9.532 | 6.295 | 1.00 | 40.10 | O |
| ATOM | 1243 | N | ASP | A | 489 | −18.578 | −10.699 | 6.422 | 1.00 | 36.35 | N |
| ATOM | 1244 | CA | ASP | A | 489 | −18.160 | −10.041 | 7.672 | 1.00 | 35.41 | C |
| ATOM | 1245 | CB | ASP | A | 489 | −17.192 | −10.955 | 8.431 | 1.00 | 37.29 | C |
| ATOM | 1246 | CG | ASP | A | 489 | −17.782 | −12.314 | 8.686 | 1.00 | 49.00 | C |
| ATOM | 1247 | OD1 | ASP | A | 489 | −19.013 | −12.383 | 8.909 | 1.00 | 49.08 | O |
| ATOM | 1248 | OD2 | ASP | A | 489 | −17.018 | −13.305 | 8.680 | 1.00 | 50.85 | O |
| ATOM | 1249 | C | ASP | A | 489 | −17.508 | −8.676 | 7.484 | 1.00 | 30.55 | C |
| ATOM | 1250 | O | ASP | A | 489 | −16.974 | −8.388 | 6.427 | 1.00 | 32.36 | O |
| ATOM | 1251 | N | ASP | A | 490 | −17.538 | −7.845 | 8.529 | 1.00 | 28.02 | N |
| ATOM | 1252 | CA | ASP | A | 490 | −16.923 | −6.527 | 8.441 | 1.00 | 31.41 | C |
| ATOM | 1253 | CB | ASP | A | 490 | −17.768 | −5.486 | 9.178 | 1.00 | 38.68 | C |
| ATOM | 1254 | CG | ASP | A | 490 | −17.934 | −5.812 | 10.630 | 1.00 | 48.46 | C |
| ATOM | 1255 | OD1 | ASP | A | 490 | −18.996 | −5.471 | 11.213 | 1.00 | 57.52 | O |
| ATOM | 1256 | OD2 | ASP | A | 490 | −16.999 | −6.397 | 11.198 | 1.00 | 54.69 | O |
| ATOM | 1257 | C | ASP | A | 490 | −15.492 | −6.523 | 8.983 | 1.00 | 26.85 | C |
| ATOM | 1258 | O | ASP | A | 490 | −14.799 | −5.512 | 8.906 | 1.00 | 24.78 | O |
| ATOM | 1259 | N | SER | A | 491 | −15.046 | −7.652 | 9.521 | 1.00 | 23.91 | N |
| ATOM | 1260 | CA | SER | A | 491 | −13.666 | −7.760 | 10.031 | 1.00 | 22.38 | C |
| ATOM | 1261 | CB | SER | A | 491 | −13.522 | −6.993 | 11.336 | 1.00 | 29.00 | C |
| ATOM | 1262 | OG | SER | A | 491 | −14.280 | −7.634 | 12.371 | 1.00 | 32.97 | O |
| ATOM | 1263 | C | SER | A | 491 | −13.333 | −9.202 | 10.309 | 1.00 | 29.41 | C |
| ATOM | 1264 | O | SER | A | 491 | −14.204 | −10.076 | 10.200 | 1.00 | 25.81 | O |
| ATOM | 1265 | N | TYR | A | 492 | −12.068 | −9.462 | 10.640 | 1.00 | 23.00 | N |
| ATOM | 1266 | CA | TYR | A | 492 | −11.679 | −10.788 | 11.033 | 1.00 | 26.00 | C |
| ATOM | 1267 | CB | TYR | A | 492 | −11.165 | −11.647 | 9.858 | 1.00 | 26.94 | C |
| ATOM | 1268 | CG | TYR | A | 492 | −9.811 | −11.265 | 9.328 | 1.00 | 26.14 | C |
| ATOM | 1269 | CD1 | TYR | A | 492 | −8.639 | −11.830 | 9.855 | 1.00 | 24.16 | C |
| ATOM | 1270 | CE1 | TYR | A | 492 | −7.378 | −11.414 | 9.419 | 1.00 | 28.54 | C |
| ATOM | 1271 | CD2 | TYR | A | 492 | −9.693 | −10.285 | 8.348 | 1.00 | 25.38 | C |
| ATOM | 1272 | CE2 | TYR | A | 492 | −8.424 | −9.859 | 7.902 | 1.00 | 23.56 | C |
| ATOM | 1273 | CZ | TYR | A | 492 | −7.282 | −10.428 | 8.447 | 1.00 | 26.57 | C |
| ATOM | 1274 | OH | TYR | A | 492 | −6.040 | −9.993 | 8.045 | 1.00 | 24.90 | O |
| ATOM | 1275 | C | TYR | A | 492 | −10.630 | −10.613 | 12.122 | 1.00 | 23.17 | C |
| ATOM | 1276 | O | TYR | A | 492 | −10.082 | −9.521 | 12.308 | 1.00 | 22.18 | O |
| ATOM | 1277 | N | TYR | A | 493 | −10.404 | −11.690 | 12.865 | 1.00 | 21.40 | N |
| ATOM | 1278 | CA | TYR | A | 493 | −9.406 | −11.705 | 13.943 | 1.00 | 18.61 | C |
| ATOM | 1279 | CB | TYR | A | 493 | −10.015 | −12.168 | 15.262 | 1.00 | 23.77 | C |
| ATOM | 1280 | CG | TYR | A | 493 | −10.965 | −11.174 | 15.837 | 1.00 | 24.82 | C |
| ATOM | 1281 | CD1 | TYR | A | 493 | −12.229 | −11.002 | 15.282 | 1.00 | 26.28 | C |
| ATOM | 1282 | CE1 | TYR | A | 493 | −13.105 | −10.061 | 15.789 | 1.00 | 33.92 | C |
| ATOM | 1283 | CD2 | TYR | A | 493 | −10.598 | −10.375 | 16.921 | 1.00 | 24.00 | C |
| ATOM | 1284 | CE2 | TYR | A | 493 | −11.468 | −9.431 | 17.439 | 1.00 | 29.62 | C |
| ATOM | 1285 | CZ | TYR | A | 493 | −12.719 | −9.284 | 16.868 | 1.00 | 33.02 | C |
| ATOM | 1286 | OH | TYR | A | 493 | −13.592 | −8.384 | 17.410 | 1.00 | 32.06 | O |
| ATOM | 1287 | C | TYR | A | 493 | −8.394 | −12.708 | 13.459 | 1.00 | 19.01 | C |
| ATOM | 1288 | O | TYR | A | 493 | −8.749 | −13.842 | 13.091 | 1.00 | 22.83 | O |
| ATOM | 1289 | N | THR | A | 494 | −7.141 | −12.272 | 13.400 | 1.00 | 26.38 | N |
| ATOM | 1290 | CA | THR | A | 494 | −6.077 | −13.079 | 12.834 | 1.00 | 32.64 | C |
| ATOM | 1291 | CB | THR | A | 494 | −5.029 | −12.172 | 12.119 | 1.00 | 31.23 | C |
| ATOM | 1292 | OG1 | THR | A | 494 | −4.099 | −12.987 | 11.401 | 1.00 | 41.68 | O |
| ATOM | 1293 | OG2 | THR | A | 494 | −4.270 | −11.355 | 13.123 | 1.00 | 33.57 | C |
| ATOM | 1294 | C | THR | A | 494 | −5.332 | −13.934 | 13.830 | 1.00 | 34.49 | C |
| ATOM | 1295 | O | THR | A | 494 | −5.210 | −13.605 | 15.005 | 1.00 | 28.24 | O |
| ATOM | 1296 | N | ALA | A | 495 | −4.747 | −15.006 | 13.322 | 1.00 | 44.02 | N |
| ATOM | 1297 | CA | ALA | A | 495 | −3.959 | −15.870 | 14.166 | 1.00 | 44.59 | C |
| ATOM | 1298 | CB | ALA | A | 495 | −3.887 | −17.248 | 13.552 | 1.00 | 46.73 | C |
| ATOM | 1299 | C | ALA | A | 495 | −2.562 | −15.276 | 14.307 | 1.00 | 50.58 | C |
| ATOM | 1300 | O | ALA | A | 495 | −2.108 | −14.536 | 13.439 | 1.00 | 49.05 | O |
| ATOM | 1301 | N | ARG | A | 496 | −1.899 | −15.593 | 15.409 | 1.00 | 54.28 | N |
| ATOM | 1302 | CA | ARG | A | 496 | −0.552 | −15.110 | 15.668 | 1.00 | 58.65 | C |
| ATOM | 1303 | CB | ARG | A | 496 | −0.261 | −15.109 | 17.173 | 1.00 | 60.21 | C |
| ATOM | 1304 | CG | ARG | A | 496 | 0.432 | −13.854 | 17.698 | 1.00 | 65.46 | C |
| ATOM | 1305 | CD | ARG | A | 496 | 1.644 | −13.487 | 16.885 | 1.00 | 66.41 | C |
| ATOM | 1306 | NE | ARG | A | 496 | 2.227 | −12.233 | 17.353 | 1.00 | 67.01 | N |
| ATOM | 1307 | CZ | ARG | A | 496 | 3.254 | −11.624 | 16.771 | 1.00 | 70.08 | C |
| ATOM | 1308 | NH1 | ARG | A | 496 | 3.816 | −12.156 | 15.690 | 1.00 | 71.57 | N |
| ATOM | 1309 | NH2 | ARG | A | 496 | 3.722 | −10.486 | 17.268 | 1.00 | 67.36 | N |
| ATOM | 1310 | C | ARG | A | 496 | 0.441 | −16.048 | 14.971 | 1.00 | 60.54 | C |
| ATOM | 1311 | O | ARG | A | 496 | 0.365 | −17.270 | 15.112 | 1.00 | 59.09 | O |
| ATOM | 1312 | N | SER | A | 497 | 1.362 | −15.475 | 14.209 | 1.00 | 58.17 | N |
| ATOM | 1313 | CA | SER | A | 497 | 2.370 | −16.275 | 13.523 | 1.00 | 60.35 | C |
| ATOM | 1314 | CB | SER | A | 497 | 2.276 | −16.074 | 12.010 | 1.00 | 60.39 | C |
| ATOM | 1315 | OG | SER | A | 497 | 2.664 | −14.760 | 11.653 | 1.00 | 64.92 | O |
| ATOM | 1316 | C | SER | A | 497 | 3.736 | −15.821 | 14.016 | 1.00 | 60.92 | C |
| ATOM | 1317 | O | SER | A | 497 | 3.836 | −14.890 | 14.817 | 1.00 | 58.88 | O |
| ATOM | 1318 | N | ALA | A | 498 | 4.781 | −16.480 | 13.531 | 1.00 | 63.41 | N |
| ATOM | 1319 | CA | ALA | A | 498 | 6.146 | −16.143 | 13.911 | 1.00 | 61.88 | C |
| ATOM | 1320 | CB | ALA | A | 498 | 7.102 | −17.263 | 13.482 | 1.00 | 59.84 | C |
| ATOM | 1321 | C | ALA | A | 498 | 6.556 | −14.827 | 13.254 | 1.00 | 60.58 | C |
| ATOM | 1322 | O | ALA | A | 498 | 6.132 | −14.515 | 12.138 | 1.00 | 60.40 | O |
| ATOM | 1323 | N | GLY | A | 499 | 7.381 | −14.057 | 13.950 | 1.00 | 60.46 | N |
| ATOM | 1324 | CA | GLY | A | 499 | 7.833 | −12.798 | 13.393 | 1.00 | 63.38 | C |
| ATOM | 1325 | C | GLY | A | 499 | 7.226 | −11.608 | 14.100 | 1.00 | 61.34 | C |
| ATOM | 1326 | O | GLY | A | 499 | 6.240 | −11.741 | 14.827 | 1.00 | 64.30 | O |
| ATOM | 1327 | N | LYS | A | 500 | 7.817 | −10.440 | 13.886 | 1.00 | 60.00 | N |
| ATOM | 1328 | CA | LYS | A | 500 | 7.338 | −9.216 | 14.511 | 1.00 | 56.90 | C |
| ATOM | 1329 | CB | LYS | A | 500 | 8.502 | −8.246 | 14.717 | 1.00 | 60.17 | C |
| ATOM | 1330 | CG | LYS | A | 500 | 9.535 | −8.705 | 15.729 | 1.00 | 63.00 | C |
| ATOM | 1331 | CD | LYS | A | 500 | 8.991 | −8.599 | 17.148 | 1.00 | 67.02 | C |
| ATOM | 1332 | CE | LYS | A | 500 | 10.049 | −8.992 | 18.174 | 1.00 | 65.56 | C |
| ATOM | 1333 | NZ | LYS | A | 500 | 9.537 | −8.897 | 19.573 | 1.00 | 68.82 | N |
| ATOM | 1334 | C | LYS | A | 500 | 6.272 | −8.539 | 13.654 | 1.00 | 51.76 | C |
| ATOM | 1335 | O | LYS | A | 500 | 6.271 | −8.656 | 12.434 | 1.00 | 48.14 | O |
| ATOM | 1336 | N | TRP | A | 501 | 5.363 | −7.829 | 14.311 | 1.00 | 47.51 | N |
| ATOM | 1337 | CA | TRP | A | 501 | 4.307 | −7.104 | 13.602 | 1.00 | 46.32 | C |
| ATOM | 1338 | CB | TRP | A | 501 | 3.028 | −7.109 | 14.441 | 1.00 | 41.88 | C |
| ATOM | 1339 | CG | TRP | A | 501 | 2.225 | −8.356 | 14.335 | 1.00 | 37.72 | C |
| ATOM | 1340 | CD2 | TRP | A | 501 | 0.855 | −8.518 | 14.721 | 1.00 | 36.73 | C |
| ATOM | 1341 | CE2 | TRP | A | 501 | 0.471 | −9.832 | 14.363 | 1.00 | 39.69 | C |
| ATOM | 1342 | CE3 | TRP | A | 501 | −0.089 | −7.679 | 15.334 | 1.00 | 38.48 | C |
| ATOM | 1343 | CD1 | TRP | A | 501 | 2.613 | −9.547 | 13.789 | 1.00 | 42.00 | C |
| ATOM | 1344 | NE1 | TRP | A | 501 | 1.562 | −10.441 | 13.797 | 1.00 | 38.68 | N |
| ATOM | 1345 | CZ2 | TRP | A | 501 | −0.821 | −10.325 | 14.593 | 1.00 | 35.30 | C |
| ATOM | 1346 | CZ3 | TRP | A | 501 | −1.379 | −8.176 | 15.565 | 1.00 | 32.86 | C |
| ATOM | 1347 | CH2 | TRP | A | 501 | −1.726 | −9.483 | 15.192 | 1.00 | 35.05 | C |
| ATOM | 1348 | C | TRP | A | 501 | 4.765 | −5.658 | 13.385 | 1.00 | 39.20 | C |
| ATOM | 1349 | O | TRP | A | 501 | 5.361 | −5.063 | 14.279 | 1.00 | 42.54 | O |
| ATOM | 1350 | N | PRO | A | 502 | 4.500 | −5.079 | 12.197 | 1.00 | 36.57 | N |
| ATOM | 1351 | CD | PRO | A | 502 | 3.828 | −5.725 | 11.050 | 1.00 | 35.66 | C |
| ATOM | 1352 | CA | PRO | A | 502 | 4.885 | −3.689 | 11.876 | 1.00 | 38.88 | C |
| ATOM | 1353 | CB | PRO | A | 502 | 4.721 | −3.623 | 10.359 | 1.00 | 37.46 | C |
| ATOM | 1354 | CG | PRO | A | 502 | 3.547 | −4.559 | 10.117 | 1.00 | 42.86 | C |
| ATOM | 1355 | C | PRO | A | 502 | 3.908 | −2.772 | 12.604 | 1.00 | 36.51 | C |
| ATOM | 1356 | O | PRO | A | 502 | 3.018 | −2.152 | 11.988 | 1.00 | 28.01 | O |
| ATOM | 1357 | N | LEU | A | 503 | 4.079 | −2.706 | 13.921 | 1.00 | 29.24 | N |
| ATOM | 1358 | CA | LEU | A | 503 | 3.185 | −1.931 | 14.780 | 1.00 | 29.71 | C |
| ATOM | 1359 | CB | LEU | A | 503 | 3.701 | −1.950 | 16.227 | 1.00 | 36.06 | C |
| ATOM | 1360 | CG | LEU | A | 503 | 3.999 | −3.328 | 16.817 | 1.00 | 39.54 | C |
| ATOM | 1361 | CD1 | LEU | A | 503 | 4.596 | −3.172 | 18.215 | 1.00 | 48.04 | C |
| ATOM | 1362 | CD2 | LEU | A | 503 | 2.727 | −4.145 | 16.872 | 1.00 | 43.63 | C |
| ATOM | 1363 | C | LEU | A | 503 | 2.962 | −0.489 | 14.364 | 1.00 | 25.89 | C |
| ATOM | 1364 | O | LEU | A | 503 | 1.875 | 0.057 | 14.580 | 1.00 | 24.95 | O |
| ATOM | 1365 | N | LYS | A | 504 | 3.979 | 0.132 | 13.766 | 1.00 | 24.94 | N |
| ATOM | 1366 | CA | LYS | A | 504 | 3.860 | 1.527 | 13.379 | 1.00 | 25.26 | C |
| ATOM | 1367 | CB | LYS | A | 504 | 5.223 | 2.090 | 12.978 | 1.00 | 28.02 | C |
| ATOM | 1368 | CG | LYS | A | 504 | 6.170 | 2.228 | 14.194 | 1.00 | 31.43 | C |
| ATOM | 1369 | CD | LYS | A | 504 | 7.500 | 2.881 | 13.813 | 1.00 | 31.17 | C |
| ATOM | 1370 | CE | LYS | A | 504 | 8.447 | 2.979 | 15.011 | 1.00 | 36.32 | C |
| ATOM | 1371 | NZ | LYS | A | 504 | 9.635 | 3.805 | 14.649 | 1.00 | 38.54 | N |
| ATOM | 1372 | C | LYS | A | 504 | 2.846 | 1.772 | 12.264 | 1.00 | 26.30 | C |
| ATOM | 1373 | O | LYS | A | 504 | 2.453 | 2.892 | 12.040 | 1.00 | 22.29 | O |
| ATOM | 1374 | N | TRP | A | 505 | 2.430 | 0.719 | 11.581 | 1.00 | 22.68 | N |
| ATOM | 1375 | CA | TRP | A | 505 | 1.437 | 0.866 | 10.515 | 1.00 | 18.95 | C |
| ATOM | 1376 | CB | TRP | A | 505 | 1.890 | 0.096 | 9.275 | 1.00 | 25.33 | C |
| ATOM | 1377 | CG | TRP | A | 505 | 2.892 | 0.834 | 8.461 | 1.00 | 25.92 | C |
| ATOM | 1378 | CD2 | TRP | A | 505 | 4.295 | 0.938 | 8.717 | 1.00 | 24.46 | C |
| ATOM | 1379 | CE2 | TRP | A | 505 | 4.834 | 1.808 | 7.739 | 1.00 | 23.54 | C |
| ATOM | 1380 | CE3 | TRP | A | 505 | 5.155 | 0.393 | 9.681 | 1.00 | 26.35 | C |
| ATOM | 1381 | CD1 | TRP | A | 505 | 2.633 | 1.615 | 7.365 | 1.00 | 19.83 | C |
| ATOM | 1382 | NE1 | TRP | A | 505 | 3.801 | 2.208 | 6.926 | 1.00 | 24.61 | N |
| ATOM | 1383 | CZ2 | TRP | A | 505 | 6.198 | 2.138 | 7.692 | 1.00 | 27.89 | C |
| ATOM | 1384 | CZ3 | TRP | A | 505 | 6.516 | 0.709 | 9.635 | 1.00 | 27.25 | C |
| ATOM | 1385 | CH2 | TRP | A | 505 | 7.024 | 1.581 | 8.642 | 1.00 | 26.23 | C |
| ATOM | 1386 | C | TRP | A | 505 | 0.070 | 0.305 | 10.971 | 1.00 | 22.11 | C |
| ATOM | 1387 | O | TRP | A | 505 | −0.926 | 0.429 | 10.258 | 1.00 | 22.39 | O |
| ATOM | 1388 | N | TYR | A | 506 | 0.027 | −0.266 | 12.167 | 1.00 | 21.52 | N |
| ATOM | 1389 | CA | TYR | A | 506 | −1.231 | −0.889 | 12.650 | 1.00 | 22.86 | C |
| ATOM | 1390 | CB | TYR | A | 506 | −0.859 | −2.165 | 13.422 | 1.00 | 21.24 | C |
| ATOM | 1391 | CG | TYR | A | 506 | −0.540 | −3.393 | 12.593 | 1.00 | 23.75 | C |
| ATOM | 1392 | CD1 | TYR | A | 506 | −0.335 | −3.297 | 11.221 | 1.00 | 25.21 | C |
| ATOM | 1393 | CE1 | TYR | A | 506 | −0.008 | −4.434 | 10.444 | 1.00 | 33.36 | C |
| ATOM | 1394 | CD2 | TYR | A | 506 | −0.424 | −4.659 | 13.184 | 1.00 | 30.33 | C |
| ATOM | 1395 | CE2 | TYR | A | 506 | −0.113 | −5.795 | 12.413 | 1.00 | 33.98 | C |
| ATOM | 1396 | CZ | TYR | A | 506 | 0.100 | −5.674 | 11.053 | 1.00 | 34.14 | C |
| ATOM | 1397 | OH | TYR | A | 506 | 0.459 | −6.784 | 10.310 | 1.00 | 36.03 | O |
| ATOM | 1398 | C | TYR | A | 506 | −2.184 | −0.017 | 13.500 | 1.00 | 25.61 | C |
| ATOM | 1399 | O | TYR | A | 506 | −1.737 | 0.682 | 14.412 | 1.00 | 24.59 | O |
| ATOM | 1400 | N | ALA | A | 507 | −3.490 | −0.076 | 13.209 | 1.00 | 21.31 | N |
| ATOM | 1401 | CA | ALA | A | 507 | −4.511 | 0.664 | 13.950 | 1.00 | 21.80 | C |
| ATOM | 1402 | CB | ALA | A | 507 | −5.894 | 0.502 | 13.254 | 1.00 | 18.75 | C |
| ATOM | 1403 | C | ALA | A | 507 | −4.570 | 0.070 | 15.374 | 1.00 | 26.17 | C |
| ATOM | 1404 | O | ALA | A | 507 | −4.161 | −1.064 | 15.607 | 1.00 | 22.46 | O |
| ATOM | 1405 | N | PRO | A | 508 | −5.107 | 0.831 | 16.332 | 1.00 | 22.10 | N |
| ATOM | 1406 | CD | PRO | A | 508 | −5.596 | 2.214 | 16.186 | 1.00 | 22.87 | C |
| ATOM | 1407 | CA | PRO | A | 508 | −5.213 | 0.370 | 17.722 | 1.00 | 24.97 | C |
| ATOM | 1408 | CB | PRO | A | 508 | −5.979 | 1.499 | 18.403 | 1.00 | 25.74 | C |
| ATOM | 1409 | CG | PRO | A | 508 | −5.555 | 2.711 | 17.600 | 1.00 | 34.90 | C |
| ATOM | 1410 | C | PRO | A | 508 | −5.898 | −0.978 | 17.600 | 1.00 | 28.04 | C |
| ATOM | 1411 | O | PRO | A | 508 | −5.3979 | −1.819 | 18.682 | 1.00 | 24.23 | O |
| ATOM | 1412 | N | GLU | A | 509 | −7.019 | −1.193 | 17.229 | 1.00 | 24.59 | N |
| ATOM | 1413 | CA | GLU | A | 509 | −7.754 | −2.445 | 17.352 | 1.00 | 27.89 | C |
| ATOM | 1414 | CB | GLU | A | 509 | −9.128 | −2.368 | 16.654 | 1.00 | 27.01 | C |
| ATOM | 1415 | CG | GLU | A | 509 | −9.060 | −2.329 | 15.121 | 1.00 | 23.52 | C |
| ATOM | 1416 | CD | GLU | A | 509 | −8.971 | −0.909 | 14.578 | 1.00 | 30.32 | C |
| ATOM | 1417 | OE1 | GLU | A | 509 | −8.712 | 0.038 | 15.366 | 1.00 | 23.83 | O |
| ATOM | 1418 | OE2 | GLU | A | 509 | −9.139 | −0.750 | 13.346 | 1.00 | 24.89 | O |
| ATOM | 1419 | C | GLU | A | 509 | −6.954 | −3.636 | 16.829 | 1.00 | 24.33 | C |
| ATOM | 1420 | O | GLU | A | 509 | −7.196 | −4.767 | 17.246 | 1.00 | 24.26 | O |
| ATOM | 1421 | N | CYS | A | 510 | −6.009 | −3.391 | 15.913 | 1.00 | 20.72 | N |
| ATOM | 1422 | CA | CYS | A | 510 | −5.173 | −4.456 | 15.392 | 1.00 | 18.85 | C |
| ATOM | 1423 | CB | CYS | A | 510 | −4.314 | −3.963 | 14.213 | 1.00 | 20.67 | C |
| ATOM | 1424 | SG | CYS | A | 510 | −5.287 | −3.330 | 12.822 | 1.00 | 25.10 | S |
| ATOM | 1425 | C | CYS | A | 510 | −4.216 | −4.926 | 16.503 | 1.00 | 21.45 | C |
| ATOM | 1426 | O | CYS | A | 510 | −4.026 | −6.104 | 16.720 | 1.00 | 24.61 | O |
| ATOM | 1427 | N | ILE | A | 511 | −3.590 | −3.972 | 17.164 | 1.00 | 22.33 | N |
| ATOM | 1428 | CA | ILE | A | 511 | −2.629 | −4.304 | 18.208 | 1.00 | 27.31 | C |
| ATOM | 1429 | CB | ILE | A | 511 | −1.724 | −3.081 | 18.537 | 1.00 | 27.85 | C |
| ATOM | 1430 | CG2 | ILE | A | 511 | −0.701 | −3.081 | 18.537 | 1.00 | 27.85 | C |
| ATOM | 1431 | CG1 | ILE | A | 511 | −1.004 | −2.638 | 17.255 | 1.00 | 27.31 | C |
| ATOM | 1432 | CD1 | ILE | A | 511 | −0.335 | −1.270 | 17.338 | 1.00 | 34.56 | C |
| ATOM | 1433 | C | ILE | A | 511 | −3.318 | −4.797 | 19.475 | 1.00 | 31.42 | C |
| ATOM | 1434 | O | ILE | A | 511 | −2.887 | −5.791 | 20.071 | 1.00 | 29.83 | O |
| ATOM | 1435 | N | ASN | A | 512 | −4.413 | −4.152 | 19.861 | 1.00 | 24.92 | N |
| ATOM | 1436 | CA | ASN | A | 512 | −5.068 | −4.547 | 21.093 | 1.00 | 27.73 | C |
| ATOM | 1437 | CB | ASN | A | 512 | −5.860 | −3.387 | 21.681 | 1.00 | 24.36 | C |
| ATOM | 1438 | CG | ASN | A | 512 | −4.980 | −2.240 | 22.108 | 1.00 | 34.57 | C |
| ATOM | 1439 | OD1 | ASN | A | 512 | −3.903 | −2.442 | 22.653 | 1.00 | 38.07 | O |
| ATOM | 1440 | ND2 | ASN | A | 512 | −5.447 | =1.024 | 21.872 | 1.00 | 38.61 | N |
| ATOM | 1441 | C | ASN | A | 512 | −5.973 | −5.747 | 20.953 | 1.00 | 30.41 | C |
| ATOM | 1442 | O | ASN | A | 512 | −6.100 | −6.530 | 21.893 | 1.00 | 30.25 | D |
| ATOM | 1443 | N | PHE | A | 513 | −6.622 | −5.898 | 19.804 | 1.00 | 23.64 | N |
| ATOM | 1444 | CA | PHE | A | 513 | −7.529 | −7.031 | 19.648 | 1.00 | 23.12 | C |
| ATOM | 1445 | CB | PHE | A | 513 | −8.965 | −6.544 | 19.642 | 1.00 | 24.06 | C |
| ATOM | 1446 | CG | PHE | A | 513 | −9.365 | −5.836 | 20.901 | 1.00 | 33.74 | C |
| ATOM | 1447 | CD1 | PHE | A | 513 | −9.502 | −4.456 | 20.925 | 1.00 | 33.94 | C |
| ATOM | 1448 | CD2 | PHE | A | 513 | −9.566 | −6.558 | 22.081 | 1.00 | 33.20 | C |
| ATOM | 1449 | CE1 | PHE | A | 513 | −9.829 | −3.790 | 22.119 | 1.00 | 40.69 | C |
| ATOM | 1450 | CE2 | PHE | A | 513 | −9.894 | −5.901 | 23.267 | 1.00 | 39.14 | C |
| ATOM | 1451 | CZ | PHE | A | 513 | −10.021 | −4.517 | 23.282 | 1.00 | 38.17 | C |
| ATOM | 1452 | C | PHE | A | 513 | −7.299 | −7.940 | 18.447 | 1.00 | 26.12 | C |
| ATOM | 1453 | O | PHE | A | 513 | −8.122 | −8.815 | 18.183 | 1.00 | 25.90 | O |
| ATOM | 1454 | N | ARG | A | 514 | −6.200 | −7.719 | 17.715 | 1.00 | 20.92 | N |
| ATOM | 1455 | CA | ARG | A | 514 | −5.855 | −8.511 | 16.548 | 1.00 | 18.78 | C |
| ATOM | 1456 | CB | ARG | A | 514 | −5.504 | −9.966 | 16.974 | 1.00 | 22.53 | C |
| ATOM | 1457 | CG | ARG | A | 514 | −4.403 | −9.980 | 18.017 | 1.00 | 24.16 | C |
| ATOM | 1458 | CD | ARG | A | 514 | −3.883 | −11.364 | 18.353 | 1.00 | 26.16 | C |
| ATOM | 1459 | NE | ARG | A | 514 | −2.727 | −11.229 | 19.247 | 1.00 | 25.10 | N |
| ATOM | 1460 | CZ | ARG | A | 514 | −1.983 | −12.248 | 19.657 | 1.00 | 27.44 | C |
| ATOM | 1461 | NH1 | ARG | A | 514 | −2.269 | −13.477 | 19.264 | 1.00 | 27.42 | N |
| ATOM | 1462 | NH2 | ARG | A | 514 | −0.928 | −12.027 | 20.443 | 1.00 | 29.69 | N |
| ATOM | 1463 | C | ARG | A | 514 | −7.008 | −8.520 | 15.532 | 1.00 | 19.73 | C |
| ATOM | 1464 | O | ARG | A | 514 | −7.173 | −9.487 | 14.790 | 1.00 | 21.31 | O |
| ATOM | 1465 | N | LYS | A | 515 | −7.733 | −7.404 | 15.480 | 1.00 | 22.26 | N |
| ATOM | 1466 | CA | LYS | A | 515 | −8.891 | −7.277 | 14.597 | 1.00 | 19.38 | C |
| ATOM | 1467 | CB | LYS | A | 515 | −9.995 | −6.589 | 15.385 | 1.00 | 23.23 | C |
| ATOM | 1468 | CG | LYS | A | 515 | −11.303 | −6.407 | 14.627 | 1.00 | 24.88 | C |
| ATOM | 1469 | CD | LYS | A | 515 | −12.277 | −5.554 | 15.432 | 1.00 | 21.97 | C |
| ATOM | 1470 | CE | LYS | A | 515 | −13.434 | −5.099 | 14.557 | 1.00 | 37.46 | C |
| ATOM | 1471 | NZ | LYS | A | 515 | −14.341 | −4.148 | 15.261 | 1.00 | 42.53 | N |
| ATOM | 1472 | C | LYS | A | 515 | −8.547 | −6.460 | 13.322 | 1.00 | 21.36 | C |
| ATOM | 1473 | O | LYS | A | 515 | −8.165 | −5.303 | 13.436 | 1.00 | 20.92 | O |
| ATOM | 1474 | N | PHE | A | 516 | −8.740 | −7.059 | 12.143 | 1.00 | 21.89 | N |
| ATOM | 1475 | CA | PHE | A | 516 | −8.421 | −6.387 | 10.870 | 1.00 | 21.28 | C |
| ATOM | 1476 | CB | PHE | A | 516 | −7.346 | −7.180 | 10.127 | 1.00 | 20.51 | C |
| ATOM | 1477 | CG | PHE | A | 516 | −6.025 | −7.215 | 10.878 | 1.00 | 21.58 | C |
| ATOM | 1478 | CD1 | PHE | A | 516 | −5.857 | −8.098 | 11.954 | 1.00 | 23.48 | C |
| ATOM | 1479 | CD2 | PHE | A | 516 | −4.998 | −6.338 | 10.555 | 1.00 | 23.31 | C |
| ATOM | 1480 | CE1 | PHE | A | 516 | −4.672 | −8.099 | 12.698 | 1.00 | 25.88 | C |
| ATOM | 1481 | CE2 | PHE | A | 516 | −3.814 | −6.334 | 11.296 | 1.00 | 24.06 | C |
| ATOM | 1482 | CZ | PHE | A | 516 | −3.654 | −7.220 | 12.370 | 1.00 | 29.09 | C |
| ATOM | 1483 | C | PHE | A | 516 | −9.659 | −6.196 | 10.014 | 1.00 | 24.42 | C |
| ATOM | 1484 | O | PHE | A | 516 | −10.584 | −7.031 | 10.045 | 1.00 | 21.09 | O |
| ATOM | 1485 | N | SER | A | 517 | −9.648 | −5.111 | 9.241 | 1.00 | 20.69 | N |
| ATOM | 1486 | CA | SER | A | 517 | −10.818 | −4.718 | 8.434 | 1.00 | 26.64 | C |
| ATOM | 1487 | CB | SER | A | 517 | −11.826 | −4.042 | 9.379 | 1.00 | 21.54 | C |
| ATOM | 1488 | OG | SER | A | 517 | −11.213 | −2.897 | 10.008 | 1.00 | 22.44 | O |
| ATOM | 1489 | C | SER | A | 517 | −10.377 | −3.679 | 7.393 | 1.00 | 21.26 | C |
| ATOM | 1490 | O | SER | A | 517 | −9.236 | −3.288 | 7.397 | 1.00 | 19.96 | O |
| ATOM | 1491 | N | SER | A | 518 | −11.287 | −3.223 | 6.529 | 1.00 | 19.99 | N |
| ATOM | 1492 | CA | SER | A | 518 | −10.873 | −2.180 | 5.589 | 1.00 | 20.05 | C |
| ATOM | 1493 | CB | SER | A | 518 | −12.002 | −1.884 | 4.584 | 1.00 | 27.16 | C |
| ATOM | 1494 | OG | SER | A | 518 | −12.033 | −2.959 | 3.646 | 1.00 | 30.81 | O |
| ATOM | 1495 | C | SER | A | 518 | −10.473 | −0.946 | 6.366 | 1.00 | 19.52 | C |
| ATOM | 1496 | O | SER | A | 518 | −9.591 | −0.196 | 5.937 | 1.00 | 26.21 | O |
| ATOM | 1497 | N | ARG | A | 519 | −11.119 | −0.725 | 7.500 | 1.00 | 22.21 | N |
| ATOM | 1498 | CA | ARG | A | 519 | −10.780 | 0.414 | 8.311 | 1.00 | 18.10 | C |
| ATOM | 1499 | CB | ARG | A | 519 | −11.842 | 0.646 | 9.371 | 1.00 | 22.77 | C |
| ATOM | 1500 | CG | ARG | A | 519 | −13.023 | 1.415 | 8.764 | 1.00 | 25.96 | C |
| ATOM | 1501 | CD | ARG | A | 519 | −14.180 | 1.425 | 9.709 | 1.00 | 34.85 | C |
| ATOM | 1502 | NE | ARG | A | 519 | −15.279 | 2.180 | 9.131 | 1.00 | 42.62 | N |
| ATOM | 1503 | CZ | ARG | A | 519 | −16.510 | 2.180 | 9.633 | 1.00 | 49.29 | C |
| ATOM | 1504 | NH1 | ARG | A | 519 | −16.798 | 1.484 | 10.727 | 1.00 | 43.62 | N |
| ATOM | 1505 | NH2 | ARG | A | 519 | −17.458 | 2.899 | 9.031 | 1.00 | 50.64 | N |
| ATOM | 1506 | C | ARG | A | 519 | −9.393 | 0.343 | 8.939 | 1.00 | 20.79 | C |
| ATOM | 1507 | O | ARG | A | 519 | −8.808 | 1.403 | 9.221 | 1.00 | 20.53 | O |
| ATOM | 1508 | N | SER | A | 520 | −8.877 | −0.867 | 9.182 | 1.00 | 18.48 | N |
| ATOM | 1509 | CA | SER | A | 520 | −7.508 | −0.904 | 9.705 | 1.00 | 19.17 | C |
| ATOM | 1510 | CB | SER | A | 520 | −7.177 | −2.264 | 10.410 | 1.00 | 17.39 | C |
| ATOM | 1511 | OG | SER | A | 520 | −7.348 | −3.401 | 9.603 | 1.00 | 22.96 | O |
| ATOM | 1512 | C | SER | A | 520 | −6.586 | −0.607 | 8.484 | 1.00 | 18.94 | C |
| ATOM | 1513 | O | SER | A | 520 | −5.506 | −0.023 | 8.631 | 1.00 | 21.65 | O |
| ATOM | 1514 | N | ASP | A | 521 | −7.032 | −0.960 | 7.272 | 1.00 | 19.22 | N |
| ATOM | 1515 | CA | ASP | A | 521 | −6.261 | −0.657 | 6.038 | 1.00 | 23.13 | C |
| ATOM | 1516 | CB | ASP | A | 521 | −6.902 | −1.257 | 4.770 | 1.00 | 27.96 | C |
| ATOM | 1517 | CG | ASP | A | 521 | −6.488 | −2.716 | 4.501 | 1.00 | 23.02 | C |
| ATOM | 1518 | OD1 | ASP | A | 521 | −5.536 | −3.223 | 5.145 | 1.00 | 22.00 | O |
| ATOM | 1519 | OD2 | ASP | A | 521 | −7.113 | −3.347 | 3.598 | 1.00 | 23.07 | O |
| ATOM | 1520 | C | ASP | A | 521 | −6.253 | 0.877 | 5.874 | 1.00 | 18.27 | C |
| ATOM | 1521 | O | ASP | A | 521 | −5.238 | 1.473 | 5.445 | 1.00 | 20.55 | O |
| ATOM | 1522 | N | VAL | A | 522 | −7.366 | 1.525 | 6.246 | 1.00 | 16.95 | N |
| ATOM | 1523 | CA | VAL | A | 522 | −7.421 | 3.004 | 6.168 | 1.00 | 16.30 | C |
| ATOM | 1524 | CB | VAL | A | 522 | −8.833 | 3.534 | 6.537 | 1.00 | 18.70 | C |
| ATOM | 1525 | CG1 | VAL | A | 522 | −8.820 | 5.066 | 6.757 | 1.00 | 19.08 | C |
| ATOM | 1526 | CG2 | VAL | A | 522 | −9.801 | 3.174 | 5.395 | 1.00 | 15.77 | C |
| ATOM | 1527 | C | VAL | A | 522 | −6.361 | 3.620 | 7.093 | 1.00 | 20.10 | C |
| ATOM | 1528 | O | VAL | A | 522 | −5.670 | 4.569 | 6.711 | 1.00 | 19.37 | O |
| ATOM | 1529 | N | TRP | A | 523 | −6.210 | 3.069 | 8.294 | 1.00 | 19.01 | N |
| ATOM | 1530 | CA | TRP | A | 523 | −5.186 | 3.593 | 9.216 | 1.00 | 17.00 | C |
| ATOM | 1531 | CB | TRP | A | 523 | −5.240 | 2.819 | 10.551 | 1.00 | 16.75 | C |
| ATOM | 1532 | CG | TRP | A | 523 | −4.247 | 3.339 | 11.594 | 1.00 | 17.47 | C |
| ATOM | 1533 | CD2 | TRP | A | 523 | −4.561 | 4.078 | 12.781 | 1.00 | 18.40 | C |
| ATOM | 1534 | CE2 | TRP | A | 523 | −3.336 | 4.384 | 13.411 | 1.00 | 19.18 | C |
| ATOM | 1535 | CE3 | TRP | A | 523 | −5.763 | 4.514 | 13.379 | 1.00 | 16.68 | C |
| ATOM | 1536 | CD1 | TRP | A | 523 | −2.889 | 3.226 | 11.559 | 1.00 | 22.45 | C |
| ATOM | 1537 | NE1 | TRP | A | 523 | −2.327 | 3.856 | 12.650 | 1.00 | 17.34 | N |
| ATOM | 1538 | CZ2 | TRP | A | 523 | −3.267 | 5.112 | 14.607 | 1.00 | 20.28 | C |
| ATOM | 1539 | CZ3 | TRP | A | 523 | −5.692 | 5.252 | 14.570 | 1.00 | 18.90 | C |
| ATOM | 1540 | CH2 | TRP | A | 523 | −4.457 | 5.536 | 15.170 | 1.00 | 20.73 | C |
| ATOM | 1541 | C | TRP | A | 523 | −3.799 | 3.464 | 8.573 | 1.00 | 16.27 | C |
| ATOM | 1542 | O | TRP | A | 523 | −2.988 | 4.402 | 8.628 | 1.00 | 19.33 | O |
| ATOM | 1543 | N | SER | A | 524 | −3.528 | 2.315 | 7.952 | 1.00 | 17.09 | N |
| ATOM | 1544 | CA | SER | A | 524 | −2.231 | 2.082 | 7.277 | 1.00 | 22.19 | C |
| ATOM | 1545 | CB | SER | A | 524 | −2.178 | 0.658 | 6.722 | 1.00 | 21.77 | C |
| ATOM | 1546 | OG | SER | A | 524 | −2.247 | −0.299 | 7.751 | 1.00 | 31.82 | O |
| ATOM | 1547 | C | SER | A | 524 | −2.008 | 3.039 | 6.114 | 1.00 | 23.32 | C |
| ATOM | 1548 | O | SER | A | 524 | −0.888 | 3.499 | 5.878 | 1.00 | 17.77 | O |
| ATOM | 1549 | N | TYR | A | 525 | −3.076 | 3.334 | 5.381 | 1.00 | 22.60 | N |
| ATOM | 1550 | CA | TYR | A | 525 | −3.042 | 4.279 | 4.274 | 1.00 | 22.61 | C |
| ATOM | 1551 | CB | TYR | A | 525 | −4.457 | 4.413 | 3.642 | 1.00 | 20.36 | C |
| ATOM | 1552 | CG | TYR | A | 525 | −4.463 | 5.402 | 2.506 | 1.00 | 18.97 | C |
| ATOM | 1553 | CD1 | TYR | A | 525 | −3.886 | 5.077 | 1.285 | 1.00 | 21.53 | C |
| ATOM | 1554 | CE1 | TYR | A | 525 | −3.835 | 6.020 | 0.230 | 1.00 | 20.51 | C |
| ATOM | 1555 | CD2 | TYR | A | 525 | −5.005 | 6.685 | 2.673 | 1.00 | 20.00 | C |
| ATOM | 1556 | CE2 | TYR | A | 525 | −4.961 | 7.620 | 1.642 | 1.00 | 21.87 | C |
| ATOM | 1557 | CZ | TYR | A | 525 | −4.376 | 7.284 | 0.428 | 1.00 | 24.64 | C |
| ATOM | 1558 | OH | TYR | A | 525 | −4.358 | 8.214 | −0.589 | 1.00 | 24.81 | O |
| ATOM | 1559 | C | TYR | A | 525 | −2.590 | 5.634 | 4.815 | 1.00 | 18.79 | C |
| ATOM | 1560 | O | TYR | A | 525 | −1.760 | 6.319 | 4.208 | 1.00 | 22.11 | O |
| ATOM | 1561 | N | GLY | A | 526 | −3.133 | 6.036 | 5.963 | 1.00 | 16.47 | N |
| ATOM | 1562 | CA | GLY | A | 526 | −2.737 | 7.291 | 6.582 | 1.00 | 17.93 | C |
| ATOM | 1563 | C | GLY | A | 526 | −1.233 | 7.343 | 6.852 | 1.00 | 20.82 | C |
| ATOM | 1564 | O | GLY | A | 526 | −0.571 | 8.366 | 6.583 | 1.00 | 20.21 | O |
| ATOM | 1565 | N | VAL | A | 527 | −0.691 | 6.262 | 7.409 | 1.00 | 18.53 | N |
| ATOM | 1566 | CA | VAL | A | 527 | 0.755 | 6.202 | 7.680 | 1.00 | 15.75 | C |
| ATOM | 1567 | CB | VAL | A | 527 | 1.109 | 4.902 | 8.477 | 1.00 | 20.06 | C |
| ATOM | 1568 | CG | VAL | A | 527 | 2.624 | 4.842 | 8.768 | 1.00 | 18.66 | C |
| ATOM | 1569 | CG1 | VAL | A | 527 | 0.292 | 4.842 | 8.768 | 1.00 | 18.66 | C |
| ATOM | 1570 | C | VAL | A | 527 | 1.490 | 6.249 | 6.326 | 1.00 | 16.40 | C |
| ATOM | 1571 | O | VAL | A | 527 | 2.527 | 6.888 | 6.205 | 1.00 | 21.73 | O |
| ATOM | 1572 | N | THR | A | 528 | 0.941 | 5.594 | 5.307 | 1.00 | 19.11 | N |
| ATOM | 1573 | CA | THR | A | 528 | 1.562 | 5.595 | 3.975 | 1.00 | 18.53 | C |
| ATOM | 1574 | CB | THR | A | 528 | 0.794 | 4.663 | 3.020 | 1.00 | 21.20 | C |
| ATOM | 1575 | OG1 | THR | A | 528 | 0.891 | 3.318 | 3.519 | 1.00 | 21.69 | O |
| ATOM | 1576 | CG2 | THR | A | 528 | 1.392 | 4.705 | 1.618 | 1.00 | 22.45 | C |
| ATOM | 1577 | C | THR | A | 528 | 1.587 | 7.032 | 3.416 | 1.00 | 24.70 | C |
| ATOM | 1578 | O | THR | A | 528 | 2.599 | 7.456 | 2.850 | 1.00 | 19.73 | O |
| ATOM | 1579 | N | MET | A | 529 | 0.479 | 7.764 | 3.587 | 1.00 | 19.61 | N |
| ATOM | 1580 | CA | MET | A | 529 | 0.425 | 9.179 | 3.143 | 1.00 | 19.25 | C |
| ATOM | 1581 | CB | MET | A | 529 | −0.902 | 9.848 | 3.576 | 1.00 | 20.84 | C |
| ATOM | 1582 | CG | MET | A | 529 | −2.135 | 9.296 | 2.846 | 1.00 | 22.24 | C |
| ATOM | 1583 | SD | MET | A | 529 | −3.608 | 10.284 | 3.352 | 1.00 | 24.00 | S |
| ATOM | 1584 | CE | MET | A | 529 | −3.207 | 11.921 | 2.622 | 1.00 | 23.54 | C |
| ATOM | 1585 | C | MET | A | 529 | 1.560 | 9.943 | 3.821 | 1.00 | 19.48 | C |
| ATOM | 1586 | O | MET | A | 529 | 2.276 | 10.736 | 3.182 | 1.00 | 20.92 | O |
| ATOM | 1587 | N | TRP | A | 530 | 1.724 | 9.726 | 5.124 | 1.00 | 19.75 | N |
| ATOM | 1588 | CA | TRP | A | 530 | 2.777 | 10.434 | 5.882 | 1.00 | 18.54 | C |
| ATOM | 1589 | CB | TRP | A | 530 | 2.731 | 10.069 | 7.376 | 1.00 | 20.48 | C |
| ATOM | 1590 | CG | TRP | A | 530 | 3.582 | 10.944 | 8.225 | 1.00 | 18.20 | C |
| ATOM | 1591 | CD2 | TRP | A | 530 | 4.969 | 10.745 | 8.527 | 1.00 | 18.21 | C |
| ATOM | 1592 | CE2 | TRP | A | 530 | 5.389 | 11.838 | 9.336 | 1.00 | 19.74 | C |
| ATOM | 1593 | CE3 | TRP | A | 530 | 5.899 | 9.752 | 8.197 | 1.00 | 20.68 | C |
| ATOM | 1594 | CD1 | TRP | A | 530 | 3.214 | 12.116 | 8.842 | 1.00 | 18.28 | C |
| ATOM | 1595 | NE1 | TRP | A | 530 | 4.305 | 12.661 | 9.520 | 1.00 | 22.22 | N |
| ATOM | 1596 | CZ2 | TRP | A | 530 | 6.712 | 11.961 | 9.819 | 1.00 | 25.36 | C |
| ATOM | 1597 | CZ3 | TRP | A | 530 | 7.212 | 9.872 | 8.681 | 1.00 | 22.68 | C |
| ATOM | 1598 | CH2 | TRP | A | 530 | 7.598 | 10.969 | 9.479 | 1.00 | 26.97 | C |
| ATOM | 1599 | C | TRP | A | 530 | 4.171 | 10.152 | 5.349 | 1.00 | 21.37 | C |
| ATOM | 1600 | 0 | TRP | A | 530 | 4.972 | 11.093 | 5.118 | 1.00 | 22.21 | O |
| ATOM | 1601 | N | GLU | A | 531 | 4.475 | 8.870 | 5.161 | 1.00 | 22.41 | N |
| ATOM | 1602 | CA | GLU | A | 531 | 5.773 | 8.443 | 4.634 | 1.00 | 23.01 | C |
| ATOM | 1603 | CB | GLU | A | 531 | 5.800 | 6.938 | 4.345 | 1.00 | 22.19 | C |
| ATOM | 1604 | CG | GLU | A | 531 | 5.636 | 5.990 | 5.540 | 1.00 | 26.83 | C |
| ATOM | 1605 | CD | GLU | A | 531 | 5.699 | 4.530 | 5.079 | 1.00 | 29.43 | C |
| ATOM | 1606 | OE1 | GLU | A | 531 | 4.621 | 3.926 | 4.848 | 1.00 | 23.48 | O |
| ATOM | 1607 | OE2 | GLU | A | 531 | 6.836 | 3.999 | 4.621 | 1.00 | 26.99 | O |
| ATOM | 1608 | C | GLU | A | 531 | 6.035 | 9.114 | 3.304 | 1.00 | 22.15 | C |
| ATOM | 1609 | O | GLU | A | 531 | 7.149 | 9.587 | 3.030 | 1.00 | 22.47 | O |
| ATOM | 1610 | N | ALA | A | 532 | 5.017 | 9.120 | 2.445 | 1.00 | 20.60 | N |
| ATOM | 1611 | CA | ALA | A | 532 | 5.170 | 9.710 | 1.121 | 1.00 | 20.49 | C |
| ATOM | 1612 | CB | ALA | A | 532 | 3.951 | 9.366 | 0.222 | 1.00 | 21.29 | C |
| ATOM | 1613 | C | ALA | A | 532 | 5.376 | 11.209 | 1.143 | 1.00 | 22.68 | C |
| ATOM | 1614 | O | ALA | A | 532 | 6.294 | 11.721 | 0.487 | 1.00 | 21.66 | O |
| ATOM | 1615 | N | LEU | A | 533 | 4.523 | 11.918 | 1.881 | 1.00 | 24.52 | N |
| ATOM | 1616 | CA | LEU | A | 533 | 4.601 | 13.377 | 1.597 | 1.00 | 27.55 | C |
| ATOM | 1617 | CB | LEU | A | 533 | 3.301 | 13.951 | 2.553 | 1.00 | 23.33 | C |
| ATOM | 1618 | CG | LEU | A | 533 | 2.113 | 13.809 | 1.586 | 1.00 | 24.16 | C |
| ATOM | 1619 | CD1 | LEU | A | 533 | 0.784 | 14.069 | 2.314 | 1.00 | 30.47 | C |
| ATOM | 1620 | CD2 | LEU | A | 533 | 2.289 | 14.808 | 0.408 | 1.00 | 26.48 | C |
| ATOM | 1621 | C | LEU | A | 533 | 5.832 | 13.849 | 2.737 | 1.00 | 28.49 | C |
| ATOM | 1622 | O | LEU | A | 533 | 6.213 | 15.021 | 2.661 | 1.00 | 23.09 | O |
| ATOM | 1623 | N | SER | A | 534 | 6.458 | 12.921 | 3.462 | 1.00 | 23.64 | N |
| ATOM | 1624 | CA | SER | A | 534 | 7.686 | 13.216 | 4.218 | 1.00 | 24.10 | C |
| ATOM | 1625 | CB | SER | A | 534 | 7.717 | 12.409 | 5.509 | 1.00 | 24.84 | C |
| ATOM | 1626 | OG | SER | A | 534 | 6.724 | 12.875 | 6.402 | 1.00 | 32.38 | O |
| ATOM | 1627 | C | SER | A | 534 | 8.867 | 12.778 | 3.370 | 1.00 | 23.66 | C |
| ATOM | 1628 | O | SER | A | 534 | 10.004 | 12.749 | 3.839 | 1.00 | 26.17 | O |
| ATOM | 1629 | N | TYR | A | 535 | 8.581 | 12.408 | 2.130 | 1.00 | 23.34 | N |
| ATOM | 1630 | CA | TYR | A | 535 | 9.593 | 11.943 | 1.196 | 1.00 | 28.19 | C |
| ATOM | 1631 | CB | TYR | A | 535 | 10.485 | 13.112 | 0.733 | 1.00 | 28.89 | C |
| ATOM | 1632 | CG | TYR | A | 535 | 9.724 | 14.020 | −0.219 | 1.00 | 29.59 | C |
| ATOM | 1633 | CD1 | TYR | A | 535 | 8.811 | 14.939 | 0.274 | 1.00 | 28.98 | C |
| ATOM | 1634 | CE1 | TYR | A | 535 | 8.026 | 15.712 | −0.584 | 1.00 | 30.80 | C |
| ATOM | 1635 | CD2 | TYR | A | 535 | 9.849 | 13.887 | −1.599 | 1.00 | 26.41 | C |
| ATOM | 1636 | CE2 | TYR | A | 535 | 9.063 | 14.647 | −2.472 | 1.00 | 29.12 | C |
| ATOM | 1637 | CZ | TYR | A | 535 | 8.155 | 15.558 | −1.948 | 1.00 | 23.83 | C |
| ATOM | 1638 | OH | TYR | A | 535 | 7.383 | 16.325 | −2.773 | 1.00 | 30.19 | O |
| ATOM | 1639 | C | TYR | A | 535 | 10.447 | 10.781 | 1.709 | 1.00 | 32.28 | C |
| ATOM | 1640 | O | TYR | A | 535 | 11.672 | 10.845 | 1.691 | 1.00 | 27.48 | O |
| ATOM | 1641 | N | GLY | A | 536 | 9.781 | 9.722 | 2.174 | 1.00 | 26.27 | N |
| ATOM | 1642 | CA | GLY | A | 536 | 10.495 | 8.530 | 2.610 | 1.00 | 25.77 | C |
| ATOM | 1643 | C | GLY | A | 536 | 11.019 | 8.433 | 4.022 | 1.00 | 30.26 | C |
| ATOM | 1644 | O | GLY | A | 536 | 11.754 | 7.499 | 4.353 | 1.00 | 37.11 | O |
| ATOM | 1645 | N | GLN | A | 537 | 10.648 | 9.380 | 4.876 | 1.00 | 30.46 | N |
| ATOM | 1646 | CA | GLN | A | 537 | 11.080 | 9.346 | 6.263 | 1.00 | 28.02 | C |
| ATOM | 1647 | CB | GLN | A | 537 | 10.740 | 10.672 | 6.935 | 1.00 | 34.05 | C |
| ATOM | 1648 | CG | GLN | A | 537 | 11.750 | 11.776 | 6.618 | 1.00 | 44.08 | C |
| ATOM | 1649 | CD | GLN | A | 537 | 11.367 | 13.123 | 7.203 | 1.00 | 46.85 | C |
| ATOM | 1650 | OE1 | GLN | A | 537 | 10.983 | 13.224 | 8.371 | 1.00 | 55.73 | O |
| ATOM | 1651 | NE2 | GLN | A | 537 | 11.477 | 14.170 | 6.394 | 1.00 | 50.81 | N |
| ATOM | 1652 | C | GLN | A | 537 | 10.401 | 8.188 | 7.011 | 1.00 | 29.96 | C |
| ATOM | 1653 | O | GLN | A | 537 | 9.343 | 7.715 | 6.601 | 1.00 | 24.20 | O |
| ATOM | 1654 | N | LYS | A | 538 | 11.028 | 7.722 | 8.094 | 1.00 | 26.82 | N |
| ATOM | 1655 | CA | LYS | A | 538 | 10.446 | 6.638 | 8.870 | 1.00 | 27.30 | C |
| ATOM | 1656 | CB | LYS | A | 538 | 11.533 | 5.871 | 9.635 | 1.00 | 32.27 | C |
| ATOM | 1657 | CG | LYS | A | 538 | 12.495 | 5.132 | 8.724 | 1.00 | 41.48 | C |
| ATOM | 1658 | CD | LYS | A | 538 | 13.536 | 4.359 | 9.529 | 1.00 | 46.01 | C |
| ATOM | 1659 | CE | LYS | A | 538 | 14.278 | 3.365 | 8.644 | 1.00 | 48.76 | C |
| ATOM | 1660 | NZ | LYS | A | 538 | 15.278 | 2.577 | 9.429 | 1.00 | 54.95 | N |
| ATOM | 1661 | C | LYS | A | 538 | 9.431 | 7.175 | 9.875 | 1.00 | 28.24 | C |
| ATOM | 1662 | O | LYS | A | 538 | 9.665 | 8.195 | 10.529 | 1.00 | 28.85 | O |
| ATOM | 1663 | N | PRO | A | 539 | 8.283 | 6.496 | 10.008 | 1.00 | 24.53 | N |
| ATOM | 1664 | CD | PRO | A | 539 | 7.742 | 5.389 | 9.198 | 1.00 | 22.29 | C |
| ATOM | 1665 | CA | PRO | A | 539 | 7.286 | 6.966 | 10.972 | 1.00 | 24.04 | C |
| ATOM | 1666 | CB | PRO | A | 539 | 6.053 | 6.118 | 10.644 | 1.00 | 28.10 | C |
| ATOM | 1667 | CG | PRO | A | 539 | 6.644 | 4.857 | 10.098 | 1.00 | 36.04 | C |
| ATOM | 1668 | C | PRO | A | 539 | 7.737 | 6.759 | 12.421 | 1.00 | 28.35 | C |
| ATOM | 1669 | O | PRO | A | 539 | 8.426 | 5.783 | 12.734 | 1.00 | 25.90 | O |
| ATOM | 1670 | N | TYR | A | 540 | 7.343 | 7.680 | 13.292 | 1.00 | 23.53 | N |
| ATOM | 1671 | CA | TYR | A | 540 | 7.656 | 7.645 | 14.722 | 1.00 | 28.38 | C |
| ATOM | 1672 | CB | TYR | A | 540 | 6.831 | 6.555 | 15.418 | 1.00 | 24.00 | C |
| ATOM | 1673 | CG | TYR | A | 540 | 5.331 | 6.586 | 15.095 | 1.00 | 27.38 | C |
| ATOM | 1674 | CD1 | TYR | A | 540 | 4.477 | 7.452 | 15.766 | 1.00 | 24.16 | C |
| ATOM | 1675 | CE1 | TYR | A | 540 | 3.096 | 7.476 | 15.483 | 1.00 | 20.41 | C |
| ATOM | 1676 | CD2 | TYR | A | 540 | 4.800 | 5.746 | 14.119 | 1.00 | 28.23 | C |
| ATOM | 1677 | CE2 | TYR | A | 540 | 3.417 | 5.767 | 13.800 | 1.00 | 21.95 | C |
| ATOM | 1678 | CZ | TYR | A | 540 | 2.580 | 6.632 | 14.496 | 1.00 | 20.01 | C |
| ATOM | 1679 | OH | TYR | A | 540 | 1.244 | 6.670 | 14.207 | 1.00 | 24.93 | O |
| ATOM | 1680 | C | TYR | A | 540 | 9.136 | 7.346 | 14.903 | 1.00 | 28.93 | C |
| ATOM | 1681 | O | TYR | A | 540 | 9.508 | 6.478 | 15.689 | 1.00 | 30.14 | O |
| ATOM | 1682 | N | LYS | A | 541 | 9.942 | 8.095 | 14.152 | 1.00 | 27.25 | N |
| ATOM | 1683 | CA | LYS | A | 541 | 11.391 | 7.985 | 14.100 | 1.00 | 39.82 | C |
| ATOM | 1684 | CB | LYS | A | 541 | 11.939 | 9.274 | 13.472 | 1.00 | 44.06 | C |
| ATOM | 1685 | CG | LYS | A | 541 | 13.334 | 9.167 | 12.893 | 1.00 | 54.68 | C |
| ATOM | 1686 | CD | LYS | A | 541 | 13.699 | 10.418 | 12.075 | 1.00 | 54.79 | C |
| ATOM | 1687 | CE | LYS | A | 541 | 12.794 | 10.586 | 10.848 | 1.00 | 59.69 | C |
| ATOM | 1688 | NZ | LYS | A | 541 | 13.234 | 11.695 | 9.938 | 1.00 | 56.19 | N |
| ATOM | 1689 | C | LYS | A | 541 | 12.074 | 7.722 | 15.447 | 1.00 | 42.70 | C |
| ATOM | 1690 | O | LYS | A | 541 | 12.849 | 6.777 | 15.590 | 1.00 | 43.82 | O |
| ATOM | 1691 | N | LYS | A | 542 | 11.784 | 8.549 | 16.433 | 1.00 | 44.10 | N |
| ATOM | 1692 | CA | LYS | A | 542 | 12.425 | 8.378 | 17.728 | 1.00 | 51.46 | C |
| ATOM | 1693 | CB | LYS | A | 542 | 12.682 | 9.749 | 18.350 | 1.00 | 51.94 | C |
| ATOM | 1694 | CG | LYS | A | 542 | 11.425 | 10.565 | 18.581 | 1.00 | 56.75 | C |
| ATOM | 1695 | CD | LYS | A | 542 | 11.731 | 11.841 | 19.359 | 1.00 | 59.67 | C |
| ATOM | 1696 | CE | LYS | A | 542 | 10.455 | 12.559 | 19.767 | 1.00 | 60.04 | C |
| ATOM | 1697 | NZ | LYS | A | 542 | 10.684 | 13.819 | 20.538 | 1.00 | 63.82 | N |
| ATOM | 1698 | C | LYS | A | 542 | 11.649 | 7.512 | 18.709 | 1.00 | 55.84 | C |
| ATOM | 1699 | O | LYS | A | 542 | 11.746 | 7.724 | 19.920 | 1.00 | 59.11 | O |
| ATOM | 1700 | N | MET | A | 543 | 10.908 | 6.523 | 18.207 | 1.00 | 41.98 | N |
| ATOM | 1701 | CA | MET | A | 543 | 10.125 | 5.658 | 19.083 | 1.00 | 43.90 | C |
| ATOM | 1702 | CB | MET | A | 543 | 8.645 | 6.029 | 19.019 | 1.00 | 45.70 | C |
| ATOM | 1703 | CG | MET | A | 543 | 8.230 | 7.209 | 19.856 | 1.00 | 53.80 | C |
| ATOM | 1704 | SD | MET | A | 543 | 6.477 | 7.548 | 19.578 | 1.00 | 44.32 | S |
| ATOM | 1705 | CE | MET | A | 543 | 6.609 | 7.548 | 19.578 | 1.00 | 44.97 | C |
| ATOM | 1706 | C | MET | A | 543 | 10.235 | 4.185 | 18.755 | 1.00 | 46.16 | C |
| ATOM | 1707 | O | MET | A | 543 | 10.497 | 3.805 | 17.616 | 1.00 | 49.81 | O |
| ATOM | 1708 | N | LYS | A | 544 | 10.001 | 3.359 | 19.769 | 1.00 | 48.84 | N |
| ATOM | 1709 | CA | LYS | A | 544 | 10.042 | 1.909 | 19.627 | 1.00 | 54.57 | C |
| ATOM | 1710 | CB | LYS | A | 544 | 10.872 | 1.284 | 20.748 | 1.00 | 54.75 | C |
| ATOM | 1711 | CG | LYS | A | 544 | 10.262 | 1.451 | 22.134 | 1.00 | 63.19 | C |
| ATOM | 1712 | CD | LYS | A | 544 | 11.076 | 0.704 | 23.191 | 1.00 | 63.73 | C |
| ATOM | 1713 | CE | LYS | A | 544 | 10.400 | 0.726 | 24.560 | 1.00 | 65.53 | C |
| ATOM | 1714 | NZ | LYS | A | 544 | 11.146 | −0.103 | 25.561 | 1.00 | 62.21 | N |
| ATOM | 1715 | C | LYS | A | 544 | 8.612 | 1.374 | 19.699 | 1.00 | 57.82 | C |
| ATOM | 1716 | O | LYS | A | 544 | 7.655 | 2.112 | 19.464 | 1.00 | 56.73 | O |
| ATOM | 1717 | N | GLY | A | 545 | 8.475 | 0.096 | 20.046 | 1.00 | 53.41 | N |
| ATOM | 1718 | CA | GLY | A | 545 | 7.161 | −0.512 | 20.128 | 1.00 | 51.20 | C |
| ATOM | 1719 | C | GLY | A | 545 | 6.218 | 0.124 | 21.133 | 1.00 | 51.82 | C |
| ATOM | 1720 | O | GLY | A | 545 | 5.359 | 0.915 | 20.752 | 1.00 | 53.88 | O |
| ATOM | 1721 | N | PRO | A | 546 | 6.350 | −0.206 | 22.427 | 1.00 | 52.05 | N |
| ATOM | 1722 | CD | PRO | A | 546 | 7.333 | −1.165 | 22.966 | 1.00 | 55.28 | C |
| ATOM | 1723 | CA | PRO | A | 546 | 5.507 | 0.321 | 23.506 | 1.00 | 51.30 | C |
| ATOM | 1724 | CB | PRO | A | 546 | 6.215 | −0.181 | 24.768 | 1.00 | 52.03 | C |
| ATOM | 1725 | CG | PRO | A | 546 | 6.743 | −1.503 | 24.322 | 1.00 | 51.79 | C |
| ATOM | 1726 | C | PRO | A | 546 | 5.325 | 1.835 | 23.515 | 1.00 | 44.10 | C |
| ATOM | 1727 | O | PRO | A | 546 | 4.296 | 2.342 | 23.983 | 1.00 | 37.86 | O |
| ATOM | 1728 | N | GLU | A | 547 | 6.329 | 2.553 | 23.017 | 1.00 | 39.18 | N |
| ATOM | 1729 | CA | GLU | A | 547 | 6.274 | 4.004 | 22.973 | 1.00 | 45.81 | C |
| ATOM | 1730 | CB | GLU | A | 547 | 7.607 | 4.582 | 22.516 | 1.00 | 44.80 | C |
| ATOM | 1731 | CG | GLU | A | 547 | 8.760 | 4.296 | 23.450 | 1.00 | 60.26 | C |
| ATOM | 1732 | CD | GLU | A | 547 | 10.033 | 4.976 | 23.001 | 1.00 | 56.95 | C |
| ATOM | 1733 | OE1 | GLU | A | 547 | 10.093 | 6.220 | 23.034 | 1.00 | 61.75 | O |
| ATOM | 1734 | OE2 | GLU | A | 547 | 10.970 | 4.247 | 22.608 | 1.00 | 59.24 | O |
| ATOM | 1735 | C | GLU | A | 547 | 5.173 | 4.495 | 22.030 | 1.00 | 31.92 | C |
| ATOM | 1736 | O | GLU | A | 547 | 4.417 | 5.381 | 22.384 | 1.00 | 33.88 | O |
| ATOM | 1737 | N | VAL | A | 548 | 5.113 | 3.922 | 20.834 | 1.00 | 36.76 | N |
| ATOM | 1738 | CA | VAL | A | 548 | 4.097 | 4.331 | 19.849 | 1.00 | 30.35 | C |
| ATOM | 1739 | CB | VAL | A | 548 | 4.316 | 3.610 | 18.513 | 1.00 | 35.20 | C |
| ATOM | 1740 | CG1 | VAL | A | 548 | 3.289 | 4.083 | 17.486 | 1.00 | 31.70 | C |
| ATOM | 1741 | CG2 | VAL | A | 548 | 5.723 | 3.888 | 18.016 | 1.00 | 39.20 | C |
| ATOM | 1742 | C | VAL | A | 548 | 2.678 | 4.060 | 20.344 | 1.00 | 32.59 | C |
| ATOM | 1743 | O | VAL | A | 548 | 1.801 | 4.913 | 20.242 | 1.00 | 32.29 | O |
| ATOM | 1744 | N | MET | A | 549 | 2.448 | 2.874 | 20.989 | 1.00 | 34.32 | N |
| ATOM | 1745 | CA | MET | A | 549 | 1.131 | 2.528 | 21.418 | 1.00 | 33.89 | C |
| ATOM | 1746 | CB | MET | A | 549 | 1.171 | 1.124 | 22.025 | 1.00 | 43.40 | C |
| ATOM | 1747 | CG | MET | A | 549 | −0.202 | 0.486 | 22.120 | 1.00 | 54.00 | C |
| ATOM | 1748 | SD | MET | A | 549 | −1.061 | 0.474 | 20.506 | 1.00 | 73.10 | S |
| ATOM | 1749 | CE | MET | A | 549 | −2.239 | 1.839 | 20.656 | 1.00 | 53.85 | C |
| ATOM | 1750 | C | MET | A | 549 | 0.650 | 3.532 | 22.476 | 1.00 | 31.82 | C |
| ATOM | 1751 | O | MET | A | 549 | −0.486 | 4.000 | 22.436 | 1.00 | 30.85 | O |
| ATOM | 1752 | N | ALA | A | 550 | 1.525 | 3.856 | 23.420 | 1.00 | 30.61 | N |
| ATOM | 1753 | CA | ALA | A | 550 | 1.194 | 4.791 | 24.490 | 1.00 | 32.54 | C |
| ATOM | 1754 | CB | ALA | A | 550 | 2.363 | 4.852 | 25.488 | 1.00 | 37.38 | C |
| ATOM | 1755 | C | ALA | A | 550 | 0.910 | 6.186 | 23.911 | 1.00 | 27.78 | C |
| ATOM | 1756 | O | ALA | A | 550 | 0.004 | 6.894 | 24.362 | 1.00 | 28.75 | O |
| ATOM | 1757 | N | PHE | A | 551 | 1.711 | 6.569 | 22.920 | 1.00 | 26.75 | N |
| ATOM | 1758 | CA | PHE | A | 551 | 1.595 | 7.861 | 22.220 | 1.00 | 29.08 | C |
| ATOM | 1759 | CB | PHE | A | 551 | 2.712 | 7.922 | 21.157 | 1.00 | 21.20 | C |
| ATOM | 1760 | CG | PHE | A | 551 | 2.791 | 9.219 | 20.382 | 1.00 | 25.99 | C |
| ATOM | 1761 | CD1 | PHE | A | 551 | 3.346 | 10.358 | 20.954 | 1.00 | 30.16 | C |
| ATOM | 1762 | CD2 | PHE | A | 551 | 2.370 | 9.265 | 19.041 | 1.00 | 29.19 | C |
| ATOM | 1763 | CE1 | PHE | A | 551 | 3.497 | 11.540 | 20.206 | 1.00 | 26.26 | C |
| ATOM | 1764 | CE2 | PHE | A | 551 | 2.521 | 10.449 | 18.286 | 1.00 | 24.90 | C |
| ATOM | 1765 | CZ | PHE | A | 551 | 3.087 | 11.577 | 18.877 | 1.00 | 28.07 | C |
| ATOM | 1766 | C | PHE | A | 551 | 0.214 | 7.955 | 21.565 | 1.00 | 23.03 | C |
| ATOM | 1767 | O | PHE | A | 551 | −0.516 | 8.922 | 21.748 | 1.00 | 24.86 | O |
| ATOM | 1768 | N | ILE | A | 552 | −0.149 | 6.931 | 20.806 | 1.00 | 23.91 | N |
| ATOM | 1769 | CA | ILE | A | 552 | −1.452 | 6.917 | 20.145 | 1.00 | 23.84 | C |
| ATOM | 1770 | CB | ILE | A | 552 | −1.559 | 5.675 | 19.241 | 1.00 | 24.60 | C |
| ATOM | 1771 | CG2 | ILE | A | 552 | −2.973 | 5.513 | 18.716 | 1.00 | 26.02 | C |
| ATOM | 1772 | CG1 | ILE | A | 552 | −0.476 | 5.757 | 18.155 | 1.00 | 28.63 | C |
| ATOM | 1773 | CD1 | ILE | A | 552 | −0.614 | 6.952 | 17.164 | 1.00 | 28.18 | C |
| ATOM | 1774 | C | ILE | A | 552 | −2.575 | 6.921 | 21.184 | 1.00 | 24.71 | C |
| ATOM | 1775 | O | ILE | A | 552 | −3.565 | 7.653 | 21.048 | 1.00 | 25.03 | O |
| ATOM | 1776 | N | GLU | A | 553 | −2.419 | 6.121 | 22.238 | 1.00 | 26.97 | N |
| ATOM | 1777 | CA | GLU | A | 553 | −3.433 | 6.055 | 23.295 | 1.00 | 27.80 | C |
| ATOM | 1778 | CB | GLU | A | 553 | −3.040 | 5.014 | 24.346 | 1.00 | 39.68 | C |
| ATOM | 1779 | CG | GLU | A | 553 | −3.089 | 3.573 | 23.834 | 1.00 | 47.86 | C |
| ATOM | 1780 | CD | GLU | A | 553 | −4.506 | 3.085 | 23.586 | 1.00 | 47.67 | C |
| ATOM | 1781 | OE1 | GLU | A | 553 | −4.662 | 2.019 | 22.950 | 1.00 | 54.95 | O |
| ATOM | 1782 | OE2 | GLU | A | 553 | −5.460 | 3.758 | 24.032 | 1.00 | 55.56 | O |
| ATOM | 1783 | C | GLU | A | 553 | −3.645 | 7.424 | 23.972 | 1.00 | 27.09 | C |
| ATOM | 1784 | O | GLU | A | 553 | −4.757 | 7.751 | 24.386 | 1.00 | 33.91 | O |
| ATOM | 1785 | N | GLN | A | 554 | −2.577 | 8.206 | 24.088 | 1.00 | 30.97 | N |
| ATOM | 1786 | CA | GLN | A | 554 | −2.639 | 9.545 | 24.678 | 1.00 | 31.06 | C |
| ATOM | 1787 | CB | GLN | A | 554 | −1.232 | 10.093 | 24.909 | 1.00 | 33.93 | C |
| ATOM | 1788 | CG | GLN | A | 554 | −0.454 | 9.441 | 26.030 | 1.00 | 47.13 | C |
| ATOM | 1789 | CD | GLN | A | 554 | 0.949 | 9.978 | 26.069 | 1.00 | 48.57 | C |
| ATOM | 1790 | OE1 | GLN | A | 554 | 1.159 | 11.187 | 25.932 | 1.00 | 58.53 | O |
| ATOM | 1791 | NE2 | GLN | A | 554 | 1.921 | 9.089 | 26.250 | 1.00 | 53.00 | N |
| ATOM | 1792 | C | GLN | A | 554 | −3.364 | 10.534 | 23.767 | 1.00 | 34.15 | C |
| ATOM | 1793 | O | GLN | A | 554 | −3.599 | 11.677 | 24.155 | 1.00 | 29.68 | O |
| ATOM | 1794 | N | GLY | A | 555 | −3.698 | 10.104 | 22.552 | 1.00 | 28.32 | N |
| ATOM | 1795 | CA | GLY | A | 555 | −4.389 | 11.008 | 21.644 | 1.00 | 30.45 | C |
| ATOM | 1796 | C | GLY | A | 555 | −3.449 | 11.876 | 20.805 | 1.00 | 27.04 | C |
| ATOM | 1797 | O | GLY | A | 555 | −3.859 | 12.914 | 20.269 | 1.00 | 28.32 | O |
| ATOM | 1798 | N | LYS | A | 556 | −2.188 | 11.478 | 20.694 | 1.00 | 21.48 | N |
| ATOM | 1799 | CA | LYS | A | 556 | −1.240 | 12.239 | 19.886 | 1.00 | 21.96 | C |
| ATOM | 1800 | CB | LYS | A | 556 | 0.096 | 12.390 | 20.618 | 1.00 | 22.25 | C |
| ATOM | 1801 | CG | LYS | A | 556 | −0.048 | 13.123 | 21.964 | 1.00 | 24.42 | C |
| ATOM | 1802 | CD | LYS | A | 556 | 1.312 | 13.380 | 22.586 | 1.00 | 25.86 | C |
| ATOM | 1803 | CE | LYS | A | 556 | 1.157 | 14.112 | 23.934 | 1.00 | 34.77 | C |
| ATOM | 1804 | NZ | LYS | A | 556 | 2.485 | 14.453 | 24.554 | 1.00 | 33.67 | N |
| ATOM | 1805 | C | LYS | A | 556 | −1.007 | 11.516 | 18.567 | 1.00 | 23.68 | C |
| ATOM | 1806 | O | LYS | A | 556 | −1.140 | 10.291 | 18.492 | 1.00 | 22.12 | O |
| ATOM | 1807 | N | ARG | A | 557 | −0.610 | 12.285 | 17.548 | 1.00 | 19.92 | N |
| ATOM | 1808 | CA | ARG | A | 557 | −0.365 | 11.722 | 16.229 | 1.00 | 20.32 | C |
| ATOM | 1809 | CB | ARG | A | 557 | −1.575 | 11.983 | 15.311 | 1.00 | 20.48 | C |
| ATOM | 1810 | CG | ARG | A | 557 | −2.895 | 11.354 | 15.818 | 1.00 | 21.21 | C |
| ATOM | 1811 | CD | ARG | A | 557 | −2.900 | 9.812 | 15.730 | 1.00 | 19.93 | C |
| ATOM | 1812 | NE | ARG | A | 557 | −4.203 | 9.222 | 16.106 | 1.00 | 19.69 | N |
| ATOM | 1813 | CZ | ARG | A | 557 | −4.580 | 8.900 | 17.343 | 1.00 | 22.33 | C |
| ATOM | 1814 | NH1 | ARG | A | 557 | −3.755 | 9.098 | 18.397 | 1.00 | 21.75 | N |
| ATOM | 1815 | NH2 | ARG | A | 557 | −5.771 | 8.337 | 17.536 | 1.00 | 19.39 | N |
| ATOM | 1816 | C | ARG | A | 557 | 0.890 | 12.335 | 15.616 | 1.00 | 20.01 | C |
| ATOM | 1817 | O | ARG | A | 557 | 1.419 | 13.342 | 16.131 | 1.00 | 21.06 | O |
| ATOM | 1818 | N | MET | A | 558 | 1.371 | 11.729 | 14.526 | 1.00 | 23.13 | N |
| ATOM | 1819 | CA | MET | A | 558 | 2.562 | 12.260 | 13.862 | 1.00 | 24.90 | C |
| ATOM | 1820 | CB | MET | A | 558 | 2.999 | 11.351 | 12.710 | 1.00 | 20.87 | C |
| ATOM | 1821 | CG | MET | A | 558 | 3.673 | 10.070 | 13.153 | 1.00 | 26.00 | C |
| ATOM | 1822 | SD | MET | A | 558 | 4.400 | 9.177 | 11.757 | 1.00 | 25.21 | S |
| ATOM | 1823 | CE | MET | A | 558 | 2.987 | 8.458 | 10.949 | 1.00 | 19.49 | C |
| ATOM | 1824 | C | MET | A | 558 | 2.287 | 13.669 | 13.330 | 1.00 | 27.66 | C |
| ATOM | 1825 | O | MET | A | 558 | 1.164 | 13.992 | 12.898 | 1.00 | 21.51 | O |
| ATOM | 1826 | N | GLU | A | 559 | 3.321 | 14.503 | 13.371 | 1.00 | 24.36 | N |
| ATOM | 1827 | CA | GLU | A | 559 | 3.230 | 15.871 | 12.908 | 1.00 | 26.80 | C |
| ATOM | 1828 | CB | GLU | A | 559 | 4.539 | 16.613 | 13.219 | 1.00 | 33.66 | C |
| ATOM | 1829 | CG | GLU | A | 559 | 5.822 | 15.788 | 12.977 | 1.00 | 43.05 | C |
| ATOM | 1830 | CD | GLU | A | 559 | 6.068 | 14.712 | 14.060 | 1.00 | 54.97 | C |
| ATOM | 1831 | OE1 | GLU | A | 559 | 6.463 | 15.072 | 15.198 | 1.00 | 59.07 | O |
| ATOM | 1832 | OE2 | GLU | A | 559 | 5.867 | 13.506 | 13.744 | 1.00 | 44.76 | O |
| ATOM | 1833 | C | GLU | A | 559 | 2.939 | 15.992 | 11.417 | 1.00 | 27.49 | C |
| ATOM | 1834 | O | GLU | A | 559 | 3.209 | 15.088 | 10.642 | 1.00 | 27.65 | O |
| ATOM | 1835 | N | CYS | A | 560 | 2.387 | 17.130 | 11.026 | 1.00 | 22.31 | N |
| ATOM | 1836 | CA | CYS | A | 560 | 2.122 | 17.377 | 9.616 | 1.00 | 23.70 | C |
| ATOM | 1837 | CB | CYS | A | 560 | 1.293 | 18.655 | 9.468 | 1.00 | 27.79 | C |
| ATOM | 1838 | SG | CYS | A | 560 | 0.980 | 19.104 | 7.751 | 1.00 | 30.84 | S |
| ATOM | 1839 | C | CYS | A | 560 | 3.490 | 17.535 | 8.905 | 1.00 | 29.44 | C |
| ATOM | 1840 | O | CYS | A | 560 | 4.350 | 18.300 | 9.351 | 1.00 | 29.36 | O |
| ATOM | 1841 | N | PRO | A | 561 | 3.733 | 16.769 | 7.825 | 1.00 | 29.57 | N |
| ATOM | 1842 | CD | PRO | A | 561 | 2.944 | 15.629 | 7.312 | 1.00 | 26.67 | C |
| ATOM | 1843 | CA | PRO | A | 561 | 5.018 | 16.890 | 7.116 | 1.00 | 31.79 | C |
| ATOM | 1844 | CB | PRO | A | 561 | 4.865 | 15.932 | 5.933 | 1.00 | 29.22 | C |
| ATOM | 1845 | CG | PRO | A | 561 | 3.976 | 14.837 | 6.496 | 1.00 | 25.42 | C |
| ATOM | 1846 | C | PRO | A | 561 | 5.244 | 18.319 | 6.619 | 1.00 | 29.76 | C |
| ATOM | 1847 | O | PRO | A | 561 | 4.302 | 19.055 | 6.362 | 1.00 | 26.53 | O |
| ATOM | 1848 | N | PRO | A | 562 | 6.502 | 18.728 | 6.473 | 1.00 | 36.98 | N |
| ATOM | 1849 | CD | PRO | A | 562 | 7.740 | 18.045 | 6.884 | 1.00 | 38.45 | C |
| ATOM | 1850 | CA | PRO | A | 562 | 6.775 | 20.085 | 5.994 | 1.00 | 38.46 | C |
| ATOM | 1851 | CB | PRO | A | 562 | 8.302 | 20.140 | 5.984 | 1.00 | 42.62 | C |
| ATOM | 1852 | CG | PRO | A | 562 | 8.674 | 19.202 | 7.102 | 1.00 | 47.24 | C |
| ATOM | 1853 | C | PRO | A | 562 | 6.177 | 20.293 | 4.596 | 1.00 | 36.90 | C |
| ATOM | 1854 | O | PRO | A | 562 | 6.268 | 19.415 | 3.745 | 1.00 | 37.38 | O |
| ATOM | 1855 | N | GLU | A | 563 | 5.550 | 21.445 | 4.363 | 1.00 | 38.19 | N |
| ATOM | 1856 | CA | GLU | A | 563 | 4.968 | 21.740 | 3.048 | 1.00 | 43.57 | C |
| ATOM | 1857 | CB | GLU | A | 563 | 6.025 | 21.554 | 1.944 | 1.00 | 50.62 | C |
| ATOM | 1858 | CG | GLU | A | 563 | 7.282 | 22.401 | 2.118 | 1.00 | 53.80 | C |
| ATOM | 1859 | CD | GLU | A | 563 | 8.502 | 21.788 | 1.429 | 1.00 | 62.44 | C |
| ATOM | 1860 | OE1 | GLU | A | 563 | 8.457 | 21.580 | 0.193 | 1.00 | 63.21 | O |
| ATOM | 1861 | OE2 | GLU | A | 563 | 9.504 | 21.509 | 2.131 | 1.00 | 64.48 | O |
| ATOM | 1862 | C | GLU | A | 563 | 3.720 | 20.924 | 2.698 | 1.00 | 44.24 | C |
| ATOM | 1863 | O | GLU | A | 563 | 3.164 | 21.068 | 1.607 | 1.00 | 48.48 | O |
| ATOM | 1864 | N | CYS | A | 564 | 3.275 | 20.048 | 3.598 | 1.00 | 41.39 | N |
| ATOM | 1865 | CA | CYS | A | 564 | 2.060 | 19.285 | 3.344 | 1.00 | 37.90 | C |
| ATOM | 1866 | CB | CYS | A | 564 | 1.968 | 18.064 | 4.279 | 1.00 | 37.32 | C |
| ATOM | 1867 | SG | CYS | A | 564 | 0.438 | 17.119 | 4.110 | 1.00 | 38.37 | S |
| ATOM | 1868 | C | CYS | A | 564 | 0.884 | 20.221 | 3.602 | 1.00 | 37.50 | C |
| ATOM | 1869 | O | CYS | A | 564 | 0.753 | 20.773 | 4.698 | 1.00 | 38.57 | O |
| ATOM | 1870 | N | PRO | A | 565 | 0.012 | 20.416 | 2.597 | 1.00 | 37.07 | N |
| ATOM | 1871 | CD | PRO | A | 565 | 0.058 | 19.780 | 1.271 | 1.00 | 42.57 | C |
| ATOM | 1872 | CA | PRO | A | 565 | −1.160 | 21.294 | 2.716 | 1.00 | 33.51 | C |
| ATOM | 1873 | CB | PRO | A | 565 | −1.791 | 21.231 | 1.330 | 1.00 | 40.30 | C |
| ATOM | 1874 | CG | PRO | A | 565 | −1.369 | 19.883 | 0.824 | 1.00 | 44.48 | C |
| ATOM | 1875 | C | PRO | A | 565 | −2.114 | 20.827 | 3.792 | 1.00 | 35.24 | C |
| ATOM | 1876 | O | PRO | A | 565 | −2.258 | 19.622 | 4.024 | 1.00 | 32.71 | O |
| ATOM | 1877 | N | PRO | A | 566 | −2.800 | 21.776 | 4.448 | 1.00 | 34.52 | N |
| ATOM | 1878 | CD | PRO | A | 566 | −2.692 | 23.231 | 4.207 | 1.00 | 34.66 | C |
| ATOM | 1879 | CA | PRO | A | 566 | −3.762 | 21.487 | 5.516 | 1.00 | 30.97 | C |
| ATOM | 1880 | CB | PRO | A | 566 | −4.398 | 22.862 | 5.795 | 1.00 | 34.13 | C |
| ATOM | 1881 | CG | PRO | A | 566 | −3.308 | 23.823 | 5.454 | 1.00 | 32.16 | C |
| ATOM | 1882 | C | PRO | A | 566 | −4.814 | 20.438 | 5.135 | 1.00 | 34.26 | C |
| ATOM | 1883 | O | PRO | A | 566 | −5.149 | 19.585 | 5.950 | 1.00 | 28.40 | O |
| ATOM | 1884 | N | GLU | A | 567 | −5.331 | 20.498 | 3.902 | 1.00 | 27.60 | N |
| ATOM | 1885 | CA | GLU | A | 567 | −6.367 | 19.562 | 3.456 | 1.00 | 32.42 | C |
| ATOM | 1886 | CB | GLU | A | 567 | −6.859 | 19.918 | 2.036 | 1.00 | 36.27 | C |
| ATOM | 1887 | CG | GLU | A | 567 | −6.961 | 21.413 | 1.739 | 1.00 | 49.38 | C |
| ATOM | 1888 | CD | GLU | A | 567 | −5.604 | 22.059 | 1.539 | 1.00 | 48.16 | C |
| ATOM | 1889 | OE1 | GLU | A | 567 | −4.880 | 21.658 | 0.602 | 1.00 | 63.97 | O |
| ATOM | 1890 | OE2 | GLU | A | 567 | −5.259 | 22.966 | 2.318 | 1.00 | 48.66 | O |
| ATOM | 1891 | C | GLU | A | 567 | −5.848 | 18.127 | 3.440 | 1.00 | 27.96 | C |
| ATOM | 1892 | O | GLU | A | 567 | −6.553 | 17.189 | 3.829 | 1.00 | 27.68 | O |
| ATOM | 1893 | N | LEU | A | 568 | −4.615 | 17.968 | 2.976 | 1.00 | 24.93 | N |
| ATOM | 1894 | CA | LEU | A | 568 | −4.002 | 16.658 | 2.906 | 1.00 | 28.04 | C |
| ATOM | 1895 | CB | LEU | A | 568 | −2.727 | 16.761 | 2.076 | 1.00 | 31.66 | C |
| ATOM | 1896 | CG | LEU | A | 568 | −2.401 | 15.622 | 1.133 | 1.00 | 34.34 | C |
| ATOM | 1897 | CD1 | LEU | A | 568 | −3.680 | 15.015 | 0.564 | 1.00 | 38.44 | C |
| ATOM | 1898 | CD2 | LEU | A | 568 | −1.519 | 16.182 | 0.013 | 1.00 | 25.83 | C |
| ATOM | 1899 | C | LEU | A | 568 | −3.700 | 16.146 | 4.307 | 1.00 | 30.03 | C |
| ATOM | 1900 | O | LEU | A | 568 | −3.906 | 14.963 | 4.591 | 1.00 | 26.77 | O |
| ATOM | 1901 | N | TYR | A | 569 | −3.199 | 17.016 | 5.187 | 1.00 | 24.67 | N |
| ATOM | 1902 | CA | TYR | A | 569 | −2.947 | 16.354 | 6.538 | 1.00 | 22.81 | C |
| ATOM | 1903 | CB | TYR | A | 569 | −2.176 | 17.572 | 7.375 | 1.00 | 21.70 | C |
| ATOM | 1904 | CG | TYR | A | 569 | −1.824 | 17.040 | 8.753 | 1.00 | 21.77 | C |
| ATOM | 1905 | CD1 | TYR | A | 569 | −0.996 | 15.920 | 8.895 | 1.00 | 23.70 | C |
| ATOM | 1906 | CE1 | TYR | A | 569 | −0.720 | 15.375 | 10.158 | 1.00 | 23.93 | C |
| ATOM | 1907 | CD2 | TYR | A | 569 | −2.371 | 17.614 | 9.912 | 1.00 | 26.51 | C |
| ATOM | 1908 | CE2 | TYR | A | 569 | −2.107 | 17.074 | 11.184 | 1.00 | 29.40 | C |
| ATOM | 1909 | CZ | TYR | A | 569 | −1.288 | 15.960 | 11.297 | 1.00 | 28.24 | C |
| ATOM | 1910 | OH | TYR | A | 569 | −1.060 | 15.430 | 12.544 | 1.00 | 25.64 | O |
| ATOM | 1911 | C | TYR | A | 569 | −4.270 | 16.175 | 7.234 | 1.00 | 23.78 | C |
| ATOM | 1912 | O | TYR | A | 569 | −4.326 | 15.199 | 7.999 | 1.00 | 22.51 | O |
| ATOM | 1913 | N | ALA | A | 570 | −5.339 | 16.936 | 6.983 | 1.00 | 23.61 | N |
| ATOM | 1914 | CA | ALA | A | 570 | −6.621 | 16.634 | 7.632 | 1.00 | 26.10 | C |
| ATOM | 1915 | CB | ALA | A | 570 | −7.710 | 17.646 | 7.202 | 1.00 | 26.46 | C |
| ATOM | 1916 | C | ALA | A | 570 | −7.091 | 15.225 | 7.308 | 1.00 | 24.49 | C |
| ATOM | 1917 | O | ALA | A | 570 | −7.666 | 14.532 | 8.162 | 1.00 | 24.77 | O |
| ATOM | 1918 | N | LEU | A | 571 | −6.855 | 14.806 | 6.074 | 1.00 | 23.99 | N |
| ATOM | 1919 | CA | LEU | A | 571 | −7.263 | 13.499 | 5.589 | 1.00 | 21.89 | C |
| ATOM | 1920 | CB | LEU | A | 571 | −7.092 | 13.432 | 4.065 | 1.00 | 25.02 | C |
| ATOM | 1921 | CG | LEU | A | 571 | −7.346 | 12.049 | 3.472 | 1.00 | 23.70 | C |
| ATOM | 1922 | CD1 | LEU | A | 571 | −8.724 | 11.529 | 3.891 | 1.00 | 26.43 | C |
| ATOM | 1923 | CD2 | LEU | A | 571 | −7.236 | 12.135 | 1.953 | 1.00 | 26.11 | C |
| ATOM | 1924 | C | LEU | A | 571 | −6.406 | 12.422 | 6.245 | 1.00 | 20.20 | C |
| ATOM | 1925 | O | LEU | A | 571 | −6.895 | 11.428 | 6.757 | 1.00 | 21.42 | O |
| ATOM | 1926 | N | MET | A | 572 | −5.105 | 12.652 | 6.205 | 1.00 | 21.26 | N |
| ATOM | 1927 | CA | MET | A | 572 | −4.141 | 11.758 | 6.808 | 1.00 | 24.72 | C |
| ATOM | 1928 | CB | MET | A | 572 | −2.771 | 12.434 | 6.700 | 1.00 | 30.40 | C |
| ATOM | 1929 | CG | MET | A | 572 | −1.685 | 11.742 | 7.418 | 1.00 | 32.31 | C |
| ATOM | 1930 | SD | MET | A | 572 | −0.160 | 12.660 | 7.265 | 1.00 | 26.55 | S |
| ATOM | 1931 | CE | MET | A | 572 | −0.165 | 13.232 | 5.451 | 1.00 | 24.24 | C |
| ATOM | 1932 | C | MET | A | 572 | −4.531 | 11.574 | 8.280 | 1.00 | 22.05 | C |
| ATOM | 1933 | O | MET | A | 572 | −4.607 | 10.448 | 8.782 | 1.00 | 23.12 | O |
| ATOM | 1934 | N | SER | A | 573 | −4.796 | 12.686 | 8.965 | 1.00 | 18.12 | N |
| ATOM | 1935 | CA | SER | A | 573 | −5.158 | 12.630 | 10.390 | 1.00 | 21.72 | C |
| ATOM | 1936 | CB | SER | A | 573 | −5.155 | 14.055 | 10.973 | 1.00 | 21.94 | C |
| ATOM | 1937 | OG | SER | A | 573 | −5.574 | 14.093 | 12.330 | 1.00 | 26.24 | O |
| ATOM | 1938 | C | SER | A | 573 | −6.498 | 11.920 | 10.625 | 1.00 | 26.16 | C |
| ATOM | 1939 | O | SER | A | 573 | −6.660 | 11.195 | 11.599 | 1.00 | 21.94 | O |
| ATOM | 1940 | N | ASP | A | 574 | −7.471 | 12.122 | 9.749 | 1.00 | 21.50 | N |
| ATOM | 1941 | CA | ASP | A | 574 | −8.748 | 11.427 | 9.902 | 1.00 | 22.60 | C |
| ATOM | 1942 | CB | ASP | A | 574 | −9.791 | 11.915 | 8.883 | 1.00 | 20.64 | C |
| ATOM | 1943 | CG | ASP | A | 574 | −10.331 | 13.301 | 9.213 | 1.00 | 23.27 | C |
| ATOM | 1944 | OD1 | ASP | A | 574 | −10.160 | 13.772 | 10.360 | 1.00 | 22.69 | O |
| ATOM | 1945 | OD2 | ASP | A | 574 | −10.966 | 13.888 | 8.314 | 1.00 | 26.93 | O |
| ATOM | 1946 | C | ASP | A | 574 | −8.577 | 9.902 | 9.750 | 1.00 | 22.37 | C |
| ATOM | 1947 | O | ASP | A | 574 | −9.359 | 9.141 | 10.333 | 1.00 | 23.59 | O |
| ATOM | 1948 | N | CYS | A | 575 | −7.583 | 9.453 | 8.976 | 1.00 | 18.61 | N |
| ATOM | 1949 | CA | CYS | A | 575 | −7.339 | 8.014 | 8.820 | 1.00 | 17.32 | C |
| ATOM | 1950 | CB | CYS | A | 575 | −6.264 | 7.728 | 7.762 | 1.00 | 20.93 | C |
| ATOM | 1951 | SG | CYS | A | 575 | −6.765 | 8.124 | 6.035 | 1.00 | 23.46 | S |
| ATOM | 1952 | C | CYS | A | 575 | −6.855 | 7.431 | 10.156 | 1.00 | 21.12 | C |
| ATOM | 1953 | O | CYS | A | 575 | −6.857 | 6.215 | 10.343 | 1.00 | 19.67 | O |
| ATOM | 1954 | N | TRP | A | 576 | −6.423 | 8.322 | 11.047 | 1.00 | 19.87 | N |
| ATOM | 1955 | CA | TRP | A | 576 | −5.934 | 7.931 | 12.370 | 1.00 | 21.52 | C |
| ATOM | 1956 | CB | TRP | A | 576 | −4.611 | 8.626 | 12.709 | 1.00 | 20.08 | C |
| ATOM | 1957 | CG | TRP | A | 576 | −3.521 | 8.406 | 11.707 | 1.00 | 19.69 | C |
| ATOM | 1958 | CD2 | TRP | A | 576 | −2.500 | 9.333 | 11.360 | 1.00 | 16.53 | C |
| ATOM | 1959 | CE2 | TRP | A | 576 | −1.694 | 8.716 | 10.367 | 1.00 | 18.10 | C |
| ATOM | 1960 | CE3 | TRP | A | 576 | −2.180 | 10.634 | 11.796 | 1.00 | 17.50 | C |
| ATOM | 1961 | CD1 | TRP | A | 576 | −3.312 | 7.284 | 10.950 | 1.00 | 20.54 | C |
| ATOM | 1962 | NE1 | TRP | A | 576 | −2.207 | 7.464 | 10.137 | 1.00 | 18.64 | N |
| ATOM | 1963 | CZ2 | TRP | A | 576 | −0.584 | 9.357 | 9.970 | 1.00 | 20.52 | C |
| ATOM | 1964 | CZ3 | TRP | A | 576 | −1.064 | 11.282 | 11.231 | 1.00 | 19.63 | C |
| ATOM | 1965 | CH2 | TRP | A | 576 | −0.280 | 10.638 | 10.231 | 1.00 | 18.57 | C |
| ATOM | 1966 | C | TRP | A | 576 | −6.914 | 8.185 | 13.520 | 1.00 | 21.81 | C |
| ATOM | 1967 | O | TRP | A | 576 | −6.487 | 8.424 | 14.664 | 1.00 | 21.51 | O |
| ATOM | 1968 | N | ILE | A | 577 | −8.211 | 8.166 | 13.200 | 1.00 | 19.65 | N |
| ATOM | 1969 | CA | ILE | A | 577 | −9.247 | 8.325 | 14.224 | 1.00 | 20.80 | C |
| ATOM | 1970 | CB | ILE | A | 577 | −10.637 | 8.526 | 13.566 | 1.00 | 22.18 | C |
| ATOM | 1971 | CG2 | ILE | A | 577 | −11.783 | 8.179 | 14.574 | 1.00 | 27.26 | C |
| ATOM | 1972 | CG1 | ILE | A | 577 | −10.749 | 9.997 | 13.091 | 1.00 | 27.26 | C |
| ATOM | 1973 | CD1 | ILE | A | 577 | −11.997 | 10.298 | 12.254 | 1.00 | 29.41 | C |
| ATOM | 1974 | C | ILE | A | 577 | −9.194 | 7.014 | 15.029 | 1.00 | 18.87 | C |
| ATOM | 1975 | O | ILE | A | 577 | −9.204 | 5.927 | 14.455 | 1.00 | 21.86 | O |
| ATOM | 1976 | N | TYR | A | 578 | −9.099 | 7.143 | 16.349 | 1.00 | 20.88 | N |
| ATOM | 1977 | CA | TYR | A | 578 | −8.958 | 5.973 | 17.210 | 1.00 | 25.32 | C |
| ATOM | 1978 | CB | TYR | A | 578 | −8.815 | 6.429 | 18.672 | 1.00 | 23.94 | C |
| ATOM | 1979 | CG | TYR | A | 578 | −8.350 | 5.327 | 19.619 | 1.00 | 26.11 | C |
| ATOM | 1980 | CD1 | TYR | A | 578 | −7.000 | 5.188 | 19.946 | 1.00 | 31.42 | C |
| ATOM | 1981 | CE1 | TYR | A | 578 | −6.561 | 4.141 | 20.787 | 1.00 | 32.15 | C |
| ATOM | 1982 | CD2 | TYR | A | 578 | −9.256 | 4.401 | 20.158 | 1.00 | 33.33 | C |
| ATOM | 1983 | CE2 | TYR | A | 578 | −8.820 | 3.354 | 20.991 | 1.00 | 33.06 | C |
| ATOM | 1984 | CZ | TYR | A | 578 | −7.468 | 3.232 | 21.295 | 1.00 | 31.77 | C |
| ATOM | 1985 | OH | TYR | A | 578 | −7.016 | 2.178 | 22.088 | 1.00 | 33.01 | O |
| ATOM | 1986 | C | TYR | A | 578 | −10.080 | 4.947 | 17.108 | 1.00 | 27.02 | C |
| ATOM | 1987 | O | TYR | A | 578 | −9.819 | 3.750 | 16.903 | 1.00 | 22.11 | O |
| ATOM | 1988 | N | LYS | A | 579 | −11.313 | 5.418 | 17.310 | 1.00 | 24.58 | N |
| ATOM | 1989 | CA | LYS | A | 579 | −12.500 | 4.583 | 17.290 | 1.00 | 27.83 | C |
| ATOM | 1990 | CB | LYS | A | 579 | −13.727 | 5.372 | 17.776 | 1.00 | 29.76 | C |
| ATOM | 1991 | CG | LYS | A | 579 | −13.639 | 5.869 | 19.229 | 1.00 | 39.10 | C |
| ATOM | 1992 | CD | LYS | A | 579 | −14.979 | 6.478 | 19.709 | 1.00 | 42.19 | C |
| ATOM | 1993 | CE | LYS | A | 579 | −14.918 | 6.879 | 21.192 | 1.00 | 50.93 | C |
| ATOM | 1994 | NZ | LYS | A | 579 | −16.124 | 7.694 | 21.593 | 1.00 | 50.31 | N |
| ATOM | 1995 | C | LYS | A | 579 | −12.774 | 4.050 | 15.904 | 1.00 | 29.39 | C |
| ATOM | 1996 | O | LYS | A | 579 | −12.998 | 4.803 | 14.976 | 1.00 | 27.65 | O |
| ATOM | 1997 | N | TRP | A | 580 | −12.772 | 2.729 | 15.802 | 1.00 | 26.39 | N |
| ATOM | 1998 | CA | TRP | A | 580 | −13.012 | 2.038 | 14.544 | 1.00 | 31.72 | C |
| ATOM | 1999 | CB | TRP | A | 580 | −13.087 | 0.542 | 14.847 | 1.00 | 33.80 | C |
| ATOM | 2000 | CG | TRP | A | 580 | −13.287 | −0.297 | 13.670 | 1.00 | 36.87 | C |
| ATOM | 2001 | CD2 | TRP | A | 580 | −14.500 | −0.948 | 13.670 | 1.00 | 37.12 | C |
| ATOM | 2002 | CE2 | TRP | A | 580 | −14.230 | −0.166 | 12.097 | 1.00 | 42.38 | C |
| ATOM | 2003 | CE3 | TRP | A | 580 | −15.792 | −0.998 | 13.835 | 1.00 | 43.97 | C |
| ATOM | 2004 | CD1 | TRP | A | 580 | −12.355 | −0.624 | 12.728 | 1.00 | 41.08 | C |
| ATOM | 2005 | NE1 | TRP | A | 580 | −12.915 | −1.452 | 11.777 | 1.00 | 37.64 | N |
| ATOM | 2006 | CZ2 | TRP | A | 580 | −15.205 | −2.428 | 11.440 | 1.00 | 40.07 | C |
| ATOM | 2007 | CZ3 | TRP | A | 580 | −16.766 | −1.757 | 13.182 | 1.00 | 45.16 | C |
| ATOM | 2008 | CH2 | TRP | A | 580 | −16.464 | −2.462 | 11.996 | 1.00 | 48.53 | C |
| ATOM | 2009 | C | TRP | A | 580 | −14.297 | 2.492 | 13.847 | 1.00 | 32.01 | C |
| ATOM | 2010 | O | TRP | A | 580 | −14.300 | 2.806 | 12.651 | 1.00 | 29.01 | O |
| ATOM | 2011 | N | GLU | A | 581 | −15.395 | 2.528 | 14.590 | 1.00 | 26.90 | N |
| ATOM | 2012 | CA | GLU | A | 581 | −16.673 | 2.930 | 14.006 | 1.00 | 31.38 | C |
| ATOM | 2013 | CB | GLU | A | 581 | −17.785 | 2.761 | 15.043 | 1.00 | 42.49 | C |
| ATOM | 2014 | CG | GLU | A | 581 | −17.518 | 1.642 | 16.043 | 1.00 | 44.23 | C |
| ATOM | 2015 | CD | GLU | A | 581 | −16.725 | 2.134 | 17.243 | 1.00 | 54.00 | C |
| ATOM | 2016 | OE1 | GLU | A | 581 | −17.263 | 2.985 | 17.997 | 1.00 | 62.87 | O |
| ATOM | 2017 | OE2 | GLU | A | 581 | −15.576 | 1.686 | 17.440 | 1.00 | 44.35 | O |
| ATOM | 2018 | C | GLU | A | 581 | −16.703 | 4.365 | 13.461 | 1.00 | 31.42 | C |
| ATOM | 2019 | O | GLU | A | 581 | −17.501 | 4.671 | 12.585 | 1.00 | 32.84 | O |
| ATOM | 2020 | N | ASP | A | 582 | −15.824 | 5.232 | 13.963 | 1.00 | 29.31 | N |
| ATOM | 2021 | CA | ASP | A | 582 | −15.779 | 6.631 | 13.543 | 1.00 | 26.27 | C |
| ATOM | 2022 | CB | ASP | A | 582 | −15.502 | 7.525 | 14.759 | 1.00 | 30.65 | C |
| ATOM | 2023 | CG | ASP | A | 582 | −16.611 | 7.449 | 15.799 | 1.00 | 38.03 | C |
| ATOM | 2024 | OD1 | ASP | A | 582 | −17.741 | 7.089 | 15.416 | 1.00 | 34.32 | O |
| ATOM | 2025 | OD2 | ASP | A | 582 | −16.357 | 7.744 | 16.989 | 1.00 | 35.77 | O |
| ATOM | 2026 | C | ASP | A | 582 | −14.741 | 6.955 | 12.458 | 1.00 | 28.97 | C |
| ATOM | 2027 | O | ASP | A | 582 | −14.682 | 8.086 | 11.957 | 1.00 | 25.58 | O |
| ATOM | 2028 | N | ARG | A | 583 | −13.926 | 5.970 | 12.107 | 1.00 | 26.36 | N |
| ATOM | 2029 | CA | ARG | A | 583 | −12.883 | 6.195 | 11.108 | 1.00 | 26.57 | C |
| ATOM | 2030 | CB | ARG | A | 583 | −11.756 | 5.153 | 11.313 | 1.00 | 24.45 | C |
| ATOM | 2031 | CG | ARG | A | 583 | −10.396 | 5.437 | 10.597 | 1.00 | 21.69 | C |
| ATOM | 2032 | CD | ARG | A | 583 | −9.414 | 4.259 | 10.869 | 1.00 | 18.39 | C |
| ATOM | 2033 | NE | ARG | A | 583 | −9.289 | 4.015 | 12.315 | 1.00 | 17.34 | N |
| ATOM | 2034 | CZ | ARG | A | 583 | −9.081 | 2.825 | 12.866 | 1.00 | 21.68 | C |
| ATOM | 2035 | NH1 | ARG | A | 583 | −8.963 | 1.744 | 12.113 | 1.00 | 18.82 | N |
| ATOM | 2036 | NH2 | ARG | A | 583 | −9.029 | 2.717 | 14.200 | 1.00 | 22.38 | N |
| ATOM | 2037 | C | ARG | A | 583 | −13.525 | 6.070 | 9.720 | 1.00 | 24.67 | C |
| ATOM | 2038 | O | ARG | A | 583 | −14.382 | 5.223 | 9.485 | 1.00 | 25.78 | O |
| ATOM | 2039 | N | PRO | A | 584 | −13.109 | 6.916 | 8.769 | 1.00 | 23.39 | N |
| ATOM | 2040 | CD | PRO | A | 584 | −12.146 | 8.027 | 8.893 | 1.00 | 25.28 | C |
| ATOM | 2041 | CA | PRO | A | 584 | −13.667 | 6.869 | 7.407 | 1.00 | 25.72 | C |
| ATOM | 2042 | CB | PRO | A | 584 | −13.073 | 8.097 | 6.736 | 1.00 | 26.27 | C |
| ATOM | 2043 | CG | PRO | A | 584 | −11.760 | 8.276 | 7.437 | 1.00 | 22.96 | C |
| ATOM | 2044 | C | PRO | A | 584 | −13.318 | 5.609 | 6.620 | 1.00 | 26.41 | C |
| ATOM | 2045 | O | PRO | A | 584 | −12.329 | 4.940 | 6.930 | 1.00 | 23.65 | O |
| ATOM | 2046 | N | ASP | A | 585 | −14.142 | 5.313 | 5.610 | 1.00 | 20.71 | N |
| ATOM | 2047 | CA | ASP | A | 585 | −13.920 | 4.182 | 4.714 | 1.00 | 20.68 | C |
| ATOM | 2048 | CB | ASP | A | 585 | −15.258 | 3.578 | 4.236 | 1.00 | 31.23 | C |
| ATOM | 2049 | CG | ASP | A | 585 | −16.100 | 3.042 | 5.380 | 1.00 | 41.40 | C |
| ATOM | 2050 | OD1 | ASP | A | 585 | −15.581 | 2.249 | 6.197 | 1.00 | 48.33 | O |
| ATOM | 2051 | OD2 | ASP | A | 585 | −17.295 | 3.419 | 5.453 | 1.00 | 55.79 | O |
| ATOM | 2052 | C | ASP | A | 585 | −13.183 | 4.695 | 3.471 | 1.00 | 19.99 | C |
| ATOM | 2053 | O | ASP | A | 585 | −13.097 | 5.907 | 3.256 | 1.00 | 24.11 | O |
| ATOM | 2054 | N | PHE | A | 586 | −12.692 | 3.777 | 2.636 | 1.00 | 21.55 | N |
| ATOM | 2055 | CA | PHE | A | 586 | −11.960 | 4.193 | 1.439 | 1.00 | 25.44 | C |
| ATOM | 2056 | CB | PHE | A | 586 | −11.231 | 3.013 | 0.780 | 1.00 | 23.78 | C |
| ATOM | 2057 | CG | PHE | A | 586 | −9.909 | 2.689 | 1.445 | 1.00 | 21.80 | C |
| ATOM | 2058 | CD1 | PHE | A | 586 | −8.837 | 3.578 | 1.392 | 1.00 | 26.67 | C |
| ATOM | 2059 | CD2 | PHE | A | 586 | −9.754 | 1.480 | 2.146 | 1.00 | 26.93 | C |
| ATOM | 2060 | CE1 | PHE | A | 586 | −7.606 | 3.261 | 2.026 | 1.00 | 19.00 | C |
| ATOM | 2061 | CE2 | PHE | A | 586 | −8.546 | 1.175 | 2.767 | 1.00 | 22.77 | C |
| ATOM | 2062 | CZ | PHE | A | 586 | −7.482 | 2.056 | 2.701 | 1.00 | 20.88 | C |
| ATOM | 2063 | C | PHE | A | 586 | −12.863 | 4.894 | 0.430 | 1.00 | 25.78 | C |
| ATOM | 2064 | O | PHE | A | 586 | −12.399 | 5.669 | −0.358 | 1.00 | 22.13 | O |
| ATOM | 2065 | N | LEU | A | 587 | −14.168 | 4.268 | 0.487 | 1.00 | 27.35 | N |
| ATOM | 2066 | CA | LEU | A | 587 | −15.082 | 5.342 | −0.410 | 1.00 | 27.54 | C |
| ATOM | 2067 | CB | LEU | A | 587 | −16.525 | 4.916 | −0.107 | 1.00 | 33.35 | C |
| ATOM | 2068 | CG | LEU | A | 587 | −17.649 | 5.418 | −1.010 | 1.00 | 40.50 | C |
| ATOM | 2069 | CD1 | LEU | A | 587 | −17.775 | 6.902 | −0.854 | 1.00 | 43.82 | C |
| ATOM | 2070 | CD2 | LEU | A | 587 | −17.358 | 5.069 | −2.455 | 1.00 | 41.16 | C |
| ATOM | 2071 | C | LEU | A | 587 | −14.879 | 6.856 | −0.178 | 1.00 | 21.74 | C |
| ATOM | 2072 | O | LEU | A | 587 | −14.638 | 7.596 | −1.137 | 1.00 | 29.01 | O |
| ATOM | 2073 | N | THR | A | 588 | −14.923 | 7.322 | 1.077 | 1.00 | 21.57 | N |
| ATOM | 2074 | CA | THR | A | 588 | −14.718 | 8.743 | 1.326 | 1.00 | 25.49 | C |
| ATOM | 2075 | CB | THR | A | 588 | −15.271 | 9.205 | 2.705 | 1.00 | 35.32 | C |
| ATOM | 2076 | OG1 | THR | A | 588 | −14.462 | 8.694 | 3.759 | 1.00 | 46.78 | O |
| ATOM | 2077 | CG2 | THR | A | 588 | −16.687 | 8.707 | 2.879 | 1.00 | 33.61 | C |
| ATOM | 2078 | C | THR | A | 588 | −13.257 | 9.153 | 1.212 | 1.00 | 28.40 | C |
| ATOM | 2079 | O | THR | A | 588 | −12.964 | 10.229 | 0.705 | 1.00 | 26.45 | O |
| ATOM | 2080 | N | VAL | A | 589 | −12.341 | 8.293 | 1.670 | 1.00 | 22.28 | N |
| ATOM | 2081 | CA | VAL | A | 589 | −10.927 | 8.646 | 1.549 | 1.00 | 19.94 | C |
| ATOM | 2082 | CB | VAL | A | 589 | −10.021 | 7.525 | 2.161 | 1.00 | 27.25 | C |
| ATOM | 2083 | CG1 | VAL | A | 589 | −8.539 | 7.770 | 1.790 | 1.00 | 22.69 | C |
| ATOM | 2084 | CG2 | VAL | A | 589 | −10.180 | 7.525 | 3.673 | 1.00 | 24.26 | C |
| ATOM | 2085 | C | VAL | A | 589 | −10.502 | 8.911 | 0.102 | 1.00 | 20.96 | C |
| ATOM | 2086 | O | VAL | A | 589 | −9.788 | 9.888 | −0.178 | 1.00 | 21.66 | O |
| ATOM | 2087 | N | GLU | A | 590 | −10.932 | 8.047 | −0.818 | 1.00 | 23.06 | N |
| ATOM | 2088 | CA | GLU | A | 590 | −10.583 | 8.205 | −2.227 | 1.00 | 22.93 | C |
| ATOM | 2089 | CB | GLU | A | 590 | −11.130 | 7.022 | −3.049 | 1.00 | 24.91 | C |
| ATOM | 2090 | CG | GLU | A | 590 | −10.775 | 7.021 | −4.559 | 1.00 | 27.15 | C |
| ATOM | 2091 | CD | GLU | A | 590 | −11.603 | 8.014 | −5.414 | 1.00 | 31.97 | C |
| ATOM | 2092 | OE1 | GLU | A | 590 | −12.804 | 8.214 | −5.130 | 1.00 | 31.00 | O |
| ATOM | 2093 | OE2 | GLU | A | 590 | −11.056 | 8.562 | −6.399 | 1.00 | 29.57 | O |
| ATOM | 2094 | C | GLU | A | 590 | −11.137 | 9.531 | −2.784 | 1.00 | 25.86 | C |
| ATOM | 2095 | O | GLU | A | 590 | −10.466 | 10.218 | −3.552 | 1.00 | 21.69 | O |
| ATOM | 2096 | N | GLN | A | 591 | −12.362 | 9.875 | −2.394 | 1.00 | 24.00 | N |
| ATOM | 2097 | CA | GLN | A | 591 | −12.972 | 11.131 | −2.856 | 1.00 | 27.84 | C |
| ATOM | 2098 | CB | GLN | A | 591 | −14.426 | 11.197 | −2.398 | 1.00 | 29.39 | C |
| ATOM | 2099 | CG | GLN | A | 591 | −15.298 | 10.136 | −2.993 | 1.00 | 32.75 | C |
| ATOM | 2100 | CD | GLN | A | 591 | −16.754 | 10.334 | −2.629 | 1.00 | 38.78 | C |
| ATOM | 2101 | OE1 | GLN | A | 591 | −17.078 | 11.132 | −1.745 | 1.00 | 43.77 | O |
| ATOM | 2102 | NE2 | GLN | A | 591 | −17.636 | 9.614 | −3.302 | 1.00 | 44.60 | N |
| ATOM | 2103 | C | GLN | A | 591 | −12.240 | 12.361 | −2.354 | 1.00 | 28.08 | C |
| ATOM | 2104 | O | GLN | A | 591 | −12.023 | 13.332 | −3.097 | 1.00 | 27.52 | O |
| ATOM | 2105 | N | ARG | A | 592 | −11.851 | 12.345 | −1.084 | 1.00 | 24.43 | N |
| ATOM | 2106 | CA | ARG | A | 592 | −11.138 | 13.471 | −0.520 | 1.00 | 25.06 | C |
| ATOM | 2107 | CB | ARG | A | 592 | −11.060 | 13.338 | 1.004 | 1.00 | 25.79 | C |
| ATOM | 2108 | CG | ARG | A | 592 | −12.433 | 13.494 | 1.004 | 1.00 | 25.79 | C |
| ATOM | 2109 | CD | ARG | A | 592 | −12.418 | 13.215 | 3.127 | 1.00 | 31.24 | C |
| ATOM | 2110 | NE | ARG | A | 592 | −11.576 | 14.162 | 3.844 | 1.00 | 25.60 | N |
| ATOM | 2111 | CZ | ARG | A | 592 | −11.336 | 14.096 | 5.146 | 1.00 | 25.97 | C |
| ATOM | 2112 | NH1 | ARG | A | 592 | −11.883 | 13.123 | 5.859 | 1.00 | 27.29 | N |
| ATOM | 2113 | NH2 | ARG | A | 592 | −10.551 | 14.996 | 5.720 | 1.00 | 28.63 | N |
| ATOM | 2114 | C | ARG | A | 592 | −9.750 | 13.563 | −1.117 | 1.00 | 25.17 | C |
| ATOM | 2115 | O | ARG | A | 592 | −9.235 | 14.667 | −1.360 | 26.90 | 1.00 | O |
| ATOM | 2116 | N | MET | A | 593 | −9.138 | 12.409 | −1.368 | 1.00 | 23.69 | N |
| ATOM | 2117 | CA | MET | A | 593 | −7.796 | 12.416 | −1.931 | 1.00 | 28.31 | C |
| ATOM | 2118 | CB | MET | A | 593 | −7.192 | 11.000 | −1.892 | 1.00 | 23.46 | C |
| ATOM | 2119 | CG | MET | A | 593 | −5.743 | 10.934 | −2.292 | 1.00 | 23.46 | C |
| ATOM | 2120 | SD | MET | A | 593 | −4.646 | 11.896 | −1.177 | 1.00 | 28.98 | S |
| ATOM | 2121 | CE | MET | A | 593 | −3.866 | 12.944 | −2.359 | 1.00 | 26.58 | C |
| ATOM | 2122 | C | MET | A | 593 | −7.862 | 12.937 | −3.372 | 1.00 | 26.14 | C |
| ATOM | 2123 | O | MET | A | 593 | −6.998 | 13.697 | −3.802 | 1.00 | 28.83 | O |
| ATOM | 2124 | N | ARG | A | 594 | −8.877 | 12.519 | −4.117 | 1.00 | 23.87 | N |
| ATOM | 2125 | CA | ARG | A | 594 | −9.023 | 12.958 | −5.498 | 1.00 | 24.90 | C |
| ATOM | 2126 | CB | ARG | A | 594 | −10.275 | 12.328 | −6.106 | 1.00 | 21.24 | C |
| ATOM | 2127 | CG | ARG | A | 594 | −10.428 | 12.694 | −7.596 | 1.00 | 25.80 | C |
| ATOM | 2128 | CD | ARG | A | 594 | −11.817 | 12.169 | −8.118 | 1.00 | 36.37 | C |
| ATOM | 2129 | NE | ARG | A | 594 | −11.832 | 10.715 | −8.236 | 1.00 | 43.01 | N |
| ATOM | 2130 | CZ | ARG | A | 594 | −11.353 | 10.041 | −9.281 | 1.00 | 44.29 | C |
| ATOM | 2131 | NH1 | ARG | A | 594 | −10.823 | 10.690 | −10.313 | 1.00 | 45.97 | N |
| ATOM | 2132 | NH2 | ARG | A | 594 | −11.387 | 8.716 | −9.289 | 1.00 | 38.74 | N |
| ATOM | 2133 | C | ARG | A | 594 | −9.116 | 14.494 | −5.542 | 1.00 | 28.27 | C |
| ATOM | 2134 | O | ARG | A | 594 | −8.465 | 15.150 | −6.362 | 1.00 | 30.97 | O |
| ATOM | 2135 | N | ALA | A | 595 | −9.911 | 15.050 | −4.637 | 1.00 | 30.31 | N |
| ATOM | 2136 | CA | ALA | A | 595 | −10.118 | 16.495 | −4.530 | 1.00 | 34.21 | C |
| ATOM | 2137 | CB | ALA | A | 595 | −11.224 | 16.787 | −3.496 | 1.00 | 32.96 | C |
| ATOM | 2138 | C | ALA | A | 595 | −8.834 | 17.234 | −4.161 | 1.00 | 36.64 | C |
| ATOM | 2139 | O | ALA | A | 595 | −8.550 | 18.300 | −4.705 | 1.00 | 34.21 | O |
| ATOM | 2140 | N | CYS | A | 596 | −8.057 | 16.664 | −3.237 | 1.00 | 31.92 | N |
| ATOM | 2141 | CA | CYS | A | 596 | −6.803 | 17.277 | −2.826 | 1.00 | 33.56 | C |
| ATOM | 2142 | CB | CYS | A | 596 | −6.169 | 16.505 | −1.655 | 1.00 | 31.86 | C |
| ATOM | 2143 | SG | CYS | A | 596 | −7.012 | 16.710 | −0.074 | 1.00 | 40.76 | S |
| ATOM | 2144 | C | CYS | A | 596 | −5.830 | 17.280 | −3.990 | 1.00 | 32.72 | C |
| ATOM | 2145 | O | CYS | A | 596 | −5.155 | 18.279 | −4.245 | 1.00 | 31.91 | O |
| ATOM | 2146 | N | TYR | A | 597 | −5.747 | 16.150 | −4.686 | 1.00 | 28.25 | N |
| ATOM | 2147 | CA | TYR | A | 597 | −4.832 | 16.036 | −5.814 | 1.00 | 30.48 | C |
| ATOM | 2148 | CB | TYR | A | 597 | −4.881 | 14.615 | −6.389 | 1.00 | 31.14 | C |
| ATOM | 2149 | CG | TYR | A | 597 | −4.225 | 14.464 | −7.742 | 1.00 | 31.11 | C |
| ATOM | 2150 | CD1 | TYR | A | 597 | −2.963 | 15.000 | −7.993 | 1.00 | 32.87 | C |
| ATOM | 2151 | CE1 | TYR | A | 597 | −2.370 | 14.894 | −9.259 | 1.00 | 37.85 | C |
| ATOM | 2152 | CD2 | TYR | A | 597 | −4.877 | 13.806 | −8.780 | 1.00 | 34.93 | C |
| ATOM | 2153 | CE2 | TYR | A | 597 | −4.298 | 13.687 | −10.047 | 1.00 | 41.03 | C |
| ATOM | 2154 | CZ | TYR | A | 597 | −3.048 | 12.420 | −10.281 | 1.00 | 39.13 | C |
| ATOM | 2155 | OH | TYR | A | 597 | −2.505 | 14.165 | −11.543 | 1.00 | 39.42 | O |
| ATOM | 2156 | C | TYR | A | 597 | −5.168 | 17.049 | −6.907 | 1.00 | 34.57 | C |
| ATOM | 2157 | O | TYR | A | 597 | −4.275 | 17.751 | −7.421 | 1.00 | 33.33 | O |
| ATOM | 2158 | N | TYR | A | 598 | −6.446 | 17.106 | −7.261 | 1.00 | 30.49 | N |
| ATOM | 2159 | CA | TYR | A | 598 | −6.921 | 18.019 | −8.298 | 1.00 | 40.96 | C |
| ATOM | 2160 | CB | TYR | A | 598 | −8.427 | 17.833 | −8.532 | 1.00 | 38.84 | C |
| ATOM | 2161 | CG | TYR | A | 598 | −8.800 | 16.590 | −9.316 | 1.00 | 41.02 | C |
| ATOM | 2162 | CD1 | TYR | A | 598 | −10.130 | 16.335 | −9.659 | 1.00 | 49.62 | C |
| ATOM | 2163 | CE1 | TYR | A | 598 | −10.490 | 15.170 | −10.353 | 1.00 | 46.11 | C |
| ATOM | 2164 | CD2 | TYR | A | 598 | −7.838 | 15.651 | −9.691 | 1.00 | 46.14 | C |
| ATOM | 2165 | CE2 | TYR | A | 598 | −8.182 | 14.489 | −10.399 | 1.00 | 45.32 | C |
| ATOM | 2166 | CZ | TYR | A | 598 | −9.511 | 14.252 | −10.703 | 1.00 | 50.82 | C |
| ATOM | 2167 | OH | TYR | A | 598 | −9.857 | 13.082 | −11.342 | 1.00 | 48.64 | O |
| ATOM | 2168 | C | TYR | A | 598 | −6.630 | 19.453 | −7.903 | 1.00 | 44.26 | C |
| ATOM | 2169 | O | TYR | A | 598 | −6.255 | 20.275 | −8.744 | 1.00 | 50.25 | O |
| ATOM | 2170 | N | SER | A | 599 | −6.790 | 19.759 | −6.623 | 1.00 | 36.48 | N |
| ATOM | 2171 | CA | SER | A | 599 | −6.525 | 21.095 | −6.1332 | 1.00 | 42.68 | C |
| ATOM | 2172 | CB | SER | A | 599 | −6.935 | 21.185 | −4.670 | 1.00 | 43.70 | C |
| ATOM | 2173 | OG | SER | A | 599 | −6.732 | 22.487 | −4.171 | 1.00 | 57.23 | O |
| ATOM | 2174 | C | SER | A | 599 | −5.035 | 21.427 | −6.295 | 1.00 | 46.61 | C |
| ATOM | 2175 | O | SER | A | 599 | −4.663 | 22.541 | −6.686 | 1.00 | 38.28 | O |
| ATOM | 2176 | N | LEU | A | 600 | −4.176 | 20.459 | −6.006 | 1.00 | 35.37 | N |
| ATOM | 2177 | CA | LEU | A | 600 | −2.740 | 20.681 | −6.137 | 1.00 | 43.98 | C |
| ATOM | 2178 | CB | LEU | A | 600 | −1.955 | 19.613 | −5.372 | 1.00 | 36.49 | C |
| ATOM | 2179 | CG | LEU | A | 600 | −2.119 | 19.658 | −3.850 | 1.00 | 42.50 | C |
| ATOM | 2180 | CD1 | LEU | A | 600 | −1.522 | 18.409 | −3.229 | 1.00 | 35.50 | C |
| ATOM | 2181 | CD2 | LEU | A | 600 | −1.449 | 20.896 | −3.293 | 1.00 | 45.68 | C |
| ATOM | 2182 | C | LEU | A | 600 | −2.301 | 20.674 | −7.587 | 1.00 | 41.54 | C |
| ATOM | 2183 | O | LEU | A | 600 | −1.381 | 21.406 | −7.965 | 1.00 | 46.25 | O |
| ATOM | 2184 | N | ALA | A | 601 | −2.963 | 19.850 | −8.393 | 1.00 | 38.74 | N |
| ATOM | 2185 | CA | ALA | A | 601 | −2.648 | 19.698 | −9.811 | 1.00 | 47.05 | C |
| ATOM | 2186 | CB | ALA | A | 601 | −3.470 | 18.564 | −10.404 | 1.00 | 45.68 | C |
| ATOM | 2187 | C | ALA | A | 601 | −2.912 | 20.997 | −10.596 | 1.00 | 52.87 | C |
| ATOM | 2188 | O | ALA | A | 601 | −2.239 | 21.306 | −11.556 | 1.00 | 53.37 | O |
| ATOM | 2189 | N | SER | A | 602 | −3.899 | 21.748 | −10.094 | 1.00 | 51.50 | N |
| ATOM | 2190 | CA | SER | A | 602 | −4.266 | 23.024 | −10.691 | 1.00 | 57.64 | C |
| ATOM | 2191 | CB | SER | A | 602 | −5.750 | 23.293 | −10.458 | 1.00 | 57.42 | C |
| ATOM | 2192 | OG | SER | A | 602 | −6.542 | 22.301 | −11.091 | 1.00 | 55.95 | O |
| ATOM | 2193 | C | SER | A | 602 | −3.428 | 24.130 | −10.054 | 1.00 | 59.56 | C |
| ATOM | 2194 | O | SER | A | 602 | −3.790 | 25.302 | −10.103 | 1.00 | 64.84 | O |
| ATOM | 2195 | N | LYS | A | 603 | −2.307 | 23.730 | −9.459 | 1.00 | 63.84 | N |
| ATOM | 2196 | CA | LYS | A | 603 | −1.378 | 24.639 | −8.792 | 1.00 | 65.05 | C |
| ATOM | 2197 | CB | LYS | A | 603 | −0.968 | 25.767 | −9.747 | 1.00 | 65.01 | C |
| ATOM | 2198 | CG | LYS | A | 603 | 0.509 | 26.120 | −9.697 | 1.00 | 65.21 | C |
| ATOM | 2199 | CD | LYS | A | 603 | 0.835 | 27.209 | −10.706 | 1.00 | 65.84 | C |
| ATOM | 2200 | CE | LYS | A | 603 | 2.334 | 27.480 | −10.766 | 1.00 | 67.72 | C |
| ATOM | 2201 | NZ | LYS | A | 603 | 2.656 | 28.634 | −11.659 | 1.00 | 67.84 | N |
| ATOM | 2202 | C | LYS | A | 603 | −2.011 | 25.214 | −7.519 | 1.00 | 65.66 | C |
| ATOM | 2203 | O | LYS | A | 603 | −1.435 | 25.016 | −6.424 | 1.00 | 67.21 | O |
| ATOM | 2204 | OXT | LYS | A | 603 | −3.085 | 25.847 | −7.626 | 1.00 | 69.14 | O |
| TER | 1 | | LYS | A | 603 | | | | | | |
| ATOM | 2205 | CB | PHE | B | 331 | 23.385 | 4.944 | 44.783 | 1.00 | 47.38 | C |
| ATOM | 2206 | CG | PHE | B | 331 | 26.386 | 4.527 | 43.338 | 1.00 | 47.76 | C |
| ATOM | 2207 | CD1 | PHE | B | 331 | 27.574 | 4.465 | 42.621 | 1.00 | 52.74 | C |
| ATOM | 2208 | CD2 | PHE | B | 331 | 25.201 | 4.218 | 42.689 | 1.00 | 47.87 | C |
| ATOM | 2209 | CE1 | PHE | B | 331 | 27.582 | 4.104 | 41.283 | 1.00 | 48.41 | C |
| ATOM | 2210 | CE2 | PHE | B | 331 | 25.196 | 3.855 | 41.347 | 1.00 | 52.09 | C |
| ATOM | 2211 | CZ | PHE | B | 331 | 26.387 | 3.797 | 41.347 | 1.00 | 50.05 | C |
| ATOM | 2212 | C | PHE | B | 331 | 23.230 | 6.878 | 46.363 | 1.00 | 45.38 | C |
| ATOM | 2213 | O | PHE | B | 331 | 27.005 | 6.988 | 47.313 | 1.00 | 49.66 | O |
| ATOM | 2214 | N | PHE | B | 331 | 28.209 | 6.632 | 44.860 | 1.00 | 48.36 | N |
| ATOM | 2215 | CA | PHE | B | 331 | 26.733 | 6.417 | 44.999 | 1.00 | 44.38 | C |
| ATOM | 2216 | N | LEU | B | 332 | 24.933 | 7.156 | 46.441 | 1.00 | 39.16 | N |
| ATOM | 2217 | CA | LEU | B | 332 | 24.306 | 7.623 | 47.678 | 1.00 | 40.71 | C |
| ATOM | 2218 | CB | LEU | B | 332 | 23.601 | 8.967 | 47.426 | 1.00 | 40.95 | C |
| ATOM | 2219 | CG | LEU | B | 332 | 24.412 | 10.129 | 46.828 | 1.00 | 42.67 | C |
| ATOM | 2220 | CD1 | LEU | B | 332 | 23.451 | 11.230 | 46.342 | 1.00 | 38.80 | C |
| ATOM | 2221 | CD2 | LEU | B | 332 | 25.394 | 10.684 | 47.864 | 1.00 | 39.78 | C |
| ATOM | 2222 | C | LEU | B | 332 | 23.276 | 6.592 | 48.138 | 1.00 | 42.07 | C |
| ATOM | 2223 | O | LEU | B | 332 | 22.808 | 5.781 | 47.347 | 1.00 | 39.83 | O |
| ATOM | 2224 | N | LYS | B | 333 | 22.929 | 6.621 | 49.423 | 1.00 | 42.54 | N |
| ATOM | 2225 | CA | LYS | B | 333 | 21.927 | 5.699 | 49.947 | 1.00 | 44.61 | C |
| ATOM | 2226 | CB | LYS | B | 333 | 22.050 | 5.571 | 51.467 | 1.00 | 46.09 | C |
| ATOM | 2227 | CG | LYS | B | 333 | 23.341 | 4.930 | 51.902 | 1.00 | 50.67 | C |
| ATOM | 2228 | CD | LYS | B | 333 | 23.230 | 4.443 | 53.337 | 1.00 | 58.35 | C |
| ATOM | 2229 | CE | LYS | B | 333 | 24.498 | 3.707 | 53.764 | 1.00 | 59.84 | C |
| ATOM | 2230 | NZ | LYS | B | 333 | 24.351 | 3.125 | 55.132 | 1.00 | 62.41 | N |
| ATOM | 2231 | C | LYS | B | 333 | 20.542 | 6.206 | 49.605 | 1.00 | 41.31 | C |
| ATOM | 2232 | O | LYS | B | 333 | 20.214 | 7.357 | 49.888 | 1.00 | 39.53 | O |
| ATOM | 2233 | N | ARG | B | 334 | 19.729 | 5.332 | 49.021 | 1.00 | 36.95 | N |
| ATOM | 2234 | CA | ARG | B | 334 | 18.375 | 5.682 | 48.628 | 1.00 | 43.46 | C |
| ATOM | 2235 | CB | ARG | B | 334 | 17.707 | 4.487 | 47.944 | 1.00 | 44.85 | C |
| ATOM | 2236 | CG | ARG | B | 334 | 16.275 | 4.735 | 47.485 | 1.00 | 43.32 | C |
| ATOM | 2237 | CD | ARG | B | 334 | 16.238 | 5.676 | 46.299 | 1.00 | 38.79 | C |
| ATOM | 2238 | NE | ARG | B | 334 | 14.873 | 5.974 | 45.879 | 1.00 | 37.97 | N |
| ATOM | 2239 | CZ | ARG | B | 334 | 14.074 | 6.819 | 46.509 | 1.00 | 38.57 | C |
| ATOM | 2240 | NH1 | ARG | B | 334 | 14.505 | 7.457 | 47.595 | 1.00 | 36.94 | N |
| ATOM | 2241 | NH2 | ARG | B | 334 | 12.846 | 7.038 | 46.054 | 1.00 | 42.39 | N |
| ATOM | 2242 | C | ARG | B | 334 | 17.547 | 6.103 | 49.839 | 1.00 | 45.57 | C |
| ATOM | 2243 | O | ARG | B | 334 | 16.571 | 6.845 | 49.712 | 1.00 | 41.28 | O |
| ATOM | 2244 | N | ASP | B | 335 | 17.960 | 5.619 | 51.010 | 1.00 | 46.31 | N |
| ATOM | 2245 | CA | ASP | B | 335 | 17.274 | 5.898 | 52.272 | 1.00 | 45.57 | C |
| ATOM | 2246 | CB | ASP | B | 335 | 17.823 | 4.969 | 53.354 | 1.00 | 51.22 | C |
| ATOM | 2247 | CG | ASP | B | 335 | 17.911 | 3.533 | 52.887 | 1.00 | 54.98 | C |
| ATOM | 2248 | OD1 | ASP | B | 335 | 16.856 | 2.869 | 52.816 | 1.00 | 60.53 | O |
| ATOM | 2249 | OD2 | ASP | B | 335 | 19.032 | 3.074 | 52.567 | 1.00 | 62.94 | O |
| ATOM | 2250 | C | ASP | B | 335 | 17.439 | 7.338 | 52.719 | 1.00 | 42.46 | C |
| ATOM | 2251 | O | ASP | B | 335 | 16.647 | 7.840 | 53.525 | 1.00 | 36.68 | O |
| ATOM | 2252 | N | ASN | B | 336 | 18.471 | 8.010 | 52.219 | 1.00 | 29.28 | N |
| ATOM | 2253 | CA | ASN | B | 336 | 18.713 | 9.383 | 52.603 | 1.00 | 31.89 | C |
| ATOM | 2254 | CB | ASN | B | 336 | 20.211 | 9.667 | 52.652 | 1.00 | 33.88 | C |
| ATOM | 2255 | CG | ASN | B | 336 | 20.904 | 8.875 | 53.734 | 1.00 | 49.73 | C |
| ATOM | 2256 | OD1 | ASN | B | 336 | 20.356 | 8.676 | 54.822 | 1.00 | 46.67 | O |
| ATOM | 2257 | ND2 | ASN | B | 336 | 22.124 | 8.437 | 53.457 | 1.00 | 47.83 | N |
| ATOM | 2258 | C | ASN | B | 336 | 18.054 | 10.366 | 51.655 | 1.00 | 27.35 | C |
| ATOM | 2259 | O | ASN | B | 336 | 18.188 | 11.571 | 51.819 | 1.00 | 28.55 | O |
| ATOM | 2260 | N | LEU | B | 337 | 17.341 | 9.834 | 50.679 | 1.00 | 31.14 | N |
| ATOM | 2261 | CA | LEU | B | 337 | 16.687 | 10.667 | 49.677 | 1.00 | 34.02 | C |
| ATOM | 2262 | CB | LEU | B | 337 | 17.174 | 10.263 | 48.277 | 1.00 | 30.37 | C |
| ATOM | 2263 | CG | LEU | B | 337 | 16.623 | 11.013 | 47.049 | 1.00 | 29.39 | C |
| ATOM | 2264 | CD1 | LEU | B | 337 | 17.255 | 12.379 | 46.971 | 1.00 | 29.26 | C |
| ATOM | 2265 | CD2 | LEU | B | 337 | 16.949 | 10.246 | 45.775 | 1.00 | 25.80 | C |
| ATOM | 2266 | C | LEU | B | 337 | 15.163 | 10.583 | 49.728 | 1.00 | 29.91 | C |
| ATOM | 2267 | O | LEU | B | 337 | 14.581 | 9.510 | 49.799 | 1.00 | 30.87 | O |
| ATOM | 2268 | N | LEU | B | 338 | 14.516 | 11.733 | 49.674 | 1.00 | 26.54 | N |
| ATOM | 2269 | CA | LEU | B | 338 | 13.060 | 11.780 | 49.667 | 1.00 | 26.34 | C |
| ATOM | 2270 | CB | LEU | B | 338 | 12.534 | 12.524 | 50.910 | 1.00 | 27.85 | C |
| ATOM | 2271 | CG | LEU | B | 338 | 10.999 | 12.521 | 51.023 | 1.00 | 29.64 | C |
| ATOM | 2272 | CD1 | LEU | B | 338 | 10.508 | 11.097 | 51.215 | 1.00 | 34.52 | C |
| ATOM | 2273 | CD2 | LEU | B | 338 | 10.550 | 13.388 | 52.184 | 1.00 | 27.72 | C |
| ATOM | 2274 | C | LEU | B | 338 | 12.693 | 12.562 | 48.397 | 1.00 | 29.11 | C |
| ATOM | 2275 | O | LEU | B | 338 | 12.941 | 13.762 | 48.321 | 1.00 | 29.08 | O |
| ATOM | 2276 | N | ILE | B | 339 | 12.145 | 11.868 | 47.402 | 1.00 | 29.57 | N |
| ATOM | 2277 | CA | ILE | B | 339 | 11.785 | 12.534 | 46.150 | 1.00 | 28.48 | C |
| ATOM | 2278 | CB | ILE | B | 339 | 11.902 | 11.564 | 44.950 | 1.00 | 32.66 | C |
| ATOM | 2279 | CG2 | ILE | B | 339 | 11.633 | 12.331 | 43.627 | 1.00 | 32.51 | C |
| ATOM | 2280 | CG1 | ILE | B | 339 | 13.286 | 10.909 | 44.961 | 1.00 | 32.18 | C |
| ATOM | 2281 | CD1 | ILE | B | 339 | 13.495 | 9.849 | 43.902 | 1.00 | 41.01 | C |
| ATOM | 2282 | C | ILE | B | 339 | 10.366 | 13.062 | 46.208 | 1.00 | 31.19 | C |
| ATOM | 2283 | O | ILE | B | 339 | 9.431 | 12.309 | 46.466 | 1.00 | 33.54 | O |
| ATOM | 2284 | N | ALA | B | 340 | 10.208 | 14.358 | 45.965 | 1.00 | 28.66 | N |
| ATOM | 2285 | CA | ALA | B | 340 | 8.880 | 14.966 | 45.989 | 1.00 | 34.20 | C |
| ATOM | 2286 | CB | ALA | B | 340 | 8.990 | 16.462 | 46.265 | 1.00 | 32.40 | C |
| ATOM | 2287 | C | ALA | B | 340 | 8.152 | 14.717 | 44.667 | 1.00 | 36.35 | C |
| ATOM | 2288 | O | ALA | B | 340 | 8.775 | 14.336 | 43.663 | 1.00 | 35.42 | O |
| ATOM | 2289 | N | ASP | B | 341 | 6.836 | 14.907 | 44.674 | 1.00 | 33.98 | N |
| ATOM | 2290 | CA | ASP | B | 341 | 6.041 | 14.711 | 43.469 | 1.00 | 39.65 | C |
| ATOM | 2291 | CB | ASP | B | 341 | 4.631 | 14.226 | 43.840 | 1.00 | 45.61 | C |
| ATOM | 2292 | CG | ASP | B | 341 | 3.912 | 13.554 | 42.670 | 1.00 | 57.27 | C |
| ATOM | 2293 | OD1 | ASP | B | 341 | 4.096 | 14.002 | 41.519 | 1.00 | 59.44 | O |
| ATOM | 2294 | OD2 | ASP | B | 341 | 3.149 | 12.582 | 42.901 | 1.00 | 63.51 | O |
| ATOM | 2295 | C | ASP | B | 341 | 5.959 | 16.070 | 42.779 | 1.00 | 44.29 | C |
| ATOM | 2296 | O | ASP | B | 341 | 4.868 | 16.568 | 42.502 | 1.00 | 45.33 | O |
| ATOM | 2297 | N | ILE | B | 342 | 7.119 | 16.671 | 42.514 | 1.00 | 36.54 | N |
| ATOM | 2298 | CA | ILE | B | 342 | 7.202 | 17.981 | 41.879 | 1.00 | 36.97 | C |
| ATOM | 2299 | CB | ILE | B | 342 | 7.661 | 19.043 | 42.896 | 1.00 | 38.63 | C |
| ATOM | 2300 | CG2 | ILE | B | 342 | 7.852 | 20.397 | 42.215 | 1.00 | 42.84 | C |
| ATOM | 2301 | CG1 | ILE | B | 342 | 6.645 | 19.131 | 44.033 | 1.00 | 39.67 | C |
| ATOM | 2302 | CD1 | ILE | B | 342 | 7.093 | 20.032 | 45.166 | 1.00 | 46.12 | C |
| ATOM | 2303 | C | ILE | B | 342 | 8.225 | 17.899 | 40.753 | 1.00 | 37.86 | C |
| ATOM | 2304 | O | ILE | B | 342 | 9.336 | 17.438 | 40.961 | 1.00 | 33.45 | O |
| ATOM | 2305 | N | GLU | B | 343 | 7.840 | 18.335 | 39.561 | 1.00 | 31.27 | N |
| ATOM | 2306 | CA | GLU | B | 343 | 8.736 | 18.281 | 38.415 | 1.00 | 35.65 | C |
| ATOM | 2307 | CB | GLU | B | 343 | 7.994 | 17.733 | 37.194 | 1.00 | 35.13 | C |
| ATOM | 2308 | CG | GLU | B | 343 | 8.842 | 17.680 | 35.940 | 1.00 | 40.36 | C |
| ATOM | 2309 | CD | GLU | B | 343 | 8.204 | 16.874 | 34.824 | 1.00 | 49.77 | C |
| ATOM | 2310 | OE1 | GLU | B | 343 | 8.173 | 15.627 | 34.926 | 1.00 | 48.98 | O |
| ATOM | 2311 | OE2 | GLU | B | 343 | 7.734 | 17.488 | 33.845 | 1.00 | 50.13 | O |
| ATOM | 2312 | C | GLU | B | 343 | 9.287 | 19.661 | 38.122 | 1.00 | 35.92 | C |
| ATOM | 2313 | O | GLU | B | 343 | 8.522 | 20.568 | 37.825 | 1.00 | 33.14 | O |
| ATOM | 2314 | N | LEU | B | 344 | 10.612 | 19.811 | 38.179 | 1.00 | 26.19 | N |
| ATOM | 2315 | CA | LEU | B | 344 | 11.245 | 21.100 | 37.907 | 1.00 | 26.71 | C |
| ATOM | 2316 | CB | LEU | B | 344 | 12.646 | 21.179 | 38.577 | 1.00 | 25.66 | C |
| ATOM | 2317 | CG | LEU | B | 344 | 12.593 | 21.025 | 40.097 | 1.00 | 30.64 | C |
| ATOM | 2318 | CD1 | LEU | B | 344 | 14.017 | 20.874 | 40.644 | 1.00 | 27.22 | C |
| ATOM | 2319 | CD2 | LEU | B | 344 | 11.877 | 22.223 | 40.737 | 1.00 | 33.34 | C |
| ATOM | 2320 | C | LEU | B | 344 | 11.373 | 21.299 | 36.404 | 1.00 | 25.27 | C |
| ATOM | 2321 | O | LEU | B | 344 | 11.313 | 22.428 | 35.912 | 1.00 | 30.00 | O |
| ATOM | 2322 | N | GLY | B | 345 | 11.576 | 20.206 | 35.672 | 1.00 | 24.88 | N |
| ATOM | 2323 | CA | GLY | B | 345 | 11.683 | 20.297 | 34.228 | 1.00 | 29.60 | C |
| ATOM | 2324 | C | GLY | B | 345 | 11.779 | 18.915 | 33.635 | 1.00 | 29.46 | C |
| ATOM | 2325 | O | GLY | B | 345 | 11.720 | 17.929 | 34.336 | 1.00 | 32.49 | O |
| ATOM | 2326 | N | CYS | B | 346 | 11.915 | 18.818 | 32.315 | 1.00 | 27.20 | N |
| ATOM | 2327 | CA | CYS | B | 346 | 12.057 | 17.510 | 31.688 | 1.00 | 32.81 | C |
| ATOM | 2328 | CB | CYS | B | 346 | 10.690 | 16.909 | 31.391 | 1.00 | 41.74 | C |
| ATOM | 2329 | SG | CYS | B | 346 | 9.815 | 17.890 | 30.185 | 1.00 | 44.47 | S |
| ATOM | 2330 | C | CYS | B | 346 | 12.843 | 17.626 | 30.391 | 1.00 | 38.59 | C |
| ATOM | 2331 | O | CYS | B | 346 | 13.184 | 18.728 | 29.963 | 1.00 | 36.63 | O |
| ATOM | 2332 | N | GLY | B | 347 | 13.120 | 16.478 | 29.785 | 1.00 | 36.78 | N |
| ATOM | 2333 | CA | GLY | B | 347 | 13.846 | 16.426 | 28.528 | 1.00 | 36.32 | C |
| ATOM | 2334 | C | GLY | B | 347 | 13.362 | 15.050 | 27.936 | 1.00 | 37.34 | C |
| ATOM | 2335 | O | GLY | B | 347 | 12.888 | 14.252 | 28.505 | 1.00 | 35.37 | O |
| ATOM | 2336 | N | ASN | B | 348 | 14.274 | 14.760 | 26.812 | 1.00 | 38.30 | N |
| ATOM | 2337 | CA | ASN | B | 348 | 14.143 | 13.451 | 26.180 | 1.00 | 41.78 | C |
| ATOM | 2338 | CB | ASN | B | 348 | 14.905 | 13.414 | 24.850 | 1.00 | 50.03 | C |
| ATOM | 2339 | CG | ASN | B | 348 | 14.269 | 14.284 | 23.799 | 1.00 | 55.15 | C |
| ATOM | 2340 | OD1 | ASN | B | 348 | 13.105 | 14.090 | 23.440 | 1.00 | 59.57 | O |
| ATOM | 2341 | ND2 | ASN | B | 348 | 15.028 | 15.256 | 23.289 | 1.00 | 59.33 | N |
| ATOM | 2342 | C | ASN | B | 348 | 14.670 | 12.334 | 27.077 | 1.00 | 39.62 | C |
| ATOM | 2343 | O | ASN | B | 348 | 14.281 | 11.183 | 26.930 | 1.00 | 38.79 | O |
| ATOM | 2344 | N | PHE | B | 349 | 15.565 | 12.683 | 28.002 | 1.00 | 34.25 | N |
| ATOM | 2345 | CA | PHE | B | 349 | 16.144 | 11.700 | 28.915 | 1.00 | 36.33 | C |
| ATOM | 2346 | CB | PHE | B | 349 | 17.408 | 12.277 | 29.580 | 1.00 | 38.59 | C |
| ATOM | 2347 | CG | PHE | B | 349 | 17.140 | 13.471 | 30.459 | 1.00 | 35.63 | C |
| ATOM | 2348 | CD1 | PHE | B | 349 | 16.529 | 13.315 | 31.702 | 1.00 | 37.36 | C |
| ATOM | 2349 | CD2 | PHE | B | 349 | 17.441 | 14.759 | 30.022 | 1.00 | 35.00 | C |
| ATOM | 2350 | CE1 | PHE | B | 349 | 16.216 | 14.431 | 32.496 | 1.00 | 36.08 | C |
| ATOM | 2351 | CE2 | PHE | B | 349 | 17.135 | 15.872 | 30.805 | 1.00 | 39.51 | C |
| ATOM | 2352 | CZ | PHE | B | 349 | 16.518 | 15.707 | 32.047 | 1.00 | 34.21 | C |
| ATOM | 2353 | C | PHE | B | 349 | 15.156 | 11.303 | 30.014 | 1.00 | 34.30 | C |
| ATOM | 2354 | O | PHE | B | 349 | 15.219 | 10.212 | 30.562 | 1.00 | 37.30 | O |
| ATOM | 2355 | N | GLY | B | 350 | 14.263 | 12.220 | 30.358 | 1.00 | 34.85 | N |
| ATOM | 2356 | CA | GLY | B | 350 | 13.316 | 11.949 | 31.422 | 1.00 | 32.42 | C |
| ATOM | 2357 | C | GLY | B | 350 | 12.919 | 13.235 | 32.096 | 1.00 | 33.80 | C |
| ATOM | 2358 | O | GLY | B | 350 | 12.575 | 14.204 | 31.433 | 1.00 | 38.01 | O |
| ATOM | 2359 | N | SER | B | 351 | 12.954 | 13.274 | 33.421 | 1.00 | 34.81 | N |
| ATOM | 2360 | CA | SER | B | 351 | 12.560 | 14.497 | 34.079 | 1.00 | 31.64 | C |
| ATOM | 2361 | CB | SER | B | 351 | 11.114 | 14.395 | 34.536 | 1.00 | 42.24 | C |
| ATOM | 2362 | OG | SER | B | 351 | 10.994 | 13.473 | 35.583 | 1.00 | 38.56 | O |
| ATOM | 2363 | C | SER | B | 351 | 13.461 | 14.859 | 35.246 | 1.00 | 24.48 | C |
| ATOM | 2364 | O | SER | B | 351 | 14.303 | 14.076 | 35.670 | 1.00 | 31.02 | O |
| ATOM | 2365 | N | VAL | B | 352 | 13.276 | 16.064 | 34.751 | 1.00 | 23.21 | N |
| ATOM | 2366 | CA | VAL | B | 352 | 14.069 | 16.551 | 36.869 | 1.00 | 25.51 | C |
| ATOM | 2367 | CB | VAL | B | 352 | 14.776 | 17.865 | 36.521 | 1.00 | 23.36 | C |
| ATOM | 2368 | CG1 | VAL | B | 352 | 15.570 | 18.356 | 37.728 | 1.00 | 28.15 | C |
| ATOM | 2369 | CG2 | VAL | B | 352 | 15.710 | 17.645 | 35.316 | 1.00 | 25.42 | C |
| ATOM | 2370 | C | VAL | B | 352 | 13.051 | 16.809 | 37.964 | 1.00 | 25.74 | C |
| ATOM | 2371 | O | VAL | B | 352 | 12.153 | 17.661 | 37.818 | 1.00 | 26.16 | O |
| ATOM | 2372 | N | ARG | B | 353 | 13.208 | 16.078 | 39.059 | 1.00 | 22.97 | N |
| ATOM | 2373 | CA | ARG | B | 353 | 12.299 | 16.161 | 40.194 | 1.00 | 23.75 | C |
| ATOM | 2374 | CB | ARG | B | 353 | 11.909 | 14.745 | 40.652 | 1.00 | 25.68 | C |
| ATOM | 2375 | CG | ARG | B | 353 | 11.121 | 13.891 | 39.655 | 1.00 | 41.51 | C |
| ATOM | 2376 | CD | ARG | B | 353 | 9.703 | 14.400 | 39.449 | 1.00 | 48.70 | C |
| ATOM | 2377 | NE | ARG | B | 353 | 8.746 | 13.308 | 39.273 | 1.00 | 58.71 | N |
| ATOM | 2378 | CZ | ARG | B | 353 | 8.258 | 12.560 | 40.623 | 1.00 | 59.20 | C |
| ATOM | 2379 | NH1 | ARG | B | 353 | 8.627 | 12.776 | 41.519 | 1.00 | 53.03 | N |
| ATOM | 2380 | NH2 | ARG | B | 353 | 7.404 | 11.579 | 39.992 | 1.00 | 59.16 | N |
| ATOM | 2381 | C | ARG | B | 353 | 12.924 | 16.882 | 41.366 | 1.00 | 29.40 | C |
| ATOM | 2382 | O | ARG | B | 353 | 14.147 | 16.866 | 41.554 | 1.00 | 30.85 | O |
| ATOM | 2383 | N | GLN | B | 354 | 12.086 | 17.506 | 42.178 | 1.00 | 26.67 | N |
| ATOM | 2384 | CA | GLN | B | 354 | 12.580 | 18.193 | 43.361 | 1.00 | 23.26 | C |
| ATOM | 2385 | CB | GLN | B | 354 | 11.648 | 19.339 | 43.751 | 1.00 | 25.56 | C |
| ATOM | 2386 | CG | GLN | B | 354 | 12.166 | 20.151 | 44.923 | 1.00 | 30.39 | C |
| ATOM | 2387 | CD | GLN | B | 354 | 11.363 | 21.415 | 45.160 | 1.00 | 38.39 | C |
| ATOM | 2388 | OE1 | GLN | B | 354 | 10.625 | 21.870 | 44.288 | 1.00 | 35.27 | O |
| ATOM | 2389 | NE2 | GLN | B | 354 | 11.523 | 22.004 | 46.341 | 1.00 | 39.36 | N |
| ATOM | 2390 | C | GLN | B | 354 | 12.589 | 17.139 | 44.470 | 1.00 | 27.58 | C |
| ATOM | 2391 | O | GLN | B | 354 | 11.786 | 16.188 | 44.441 | 1.00 | 27.31 | O |
| ATOM | 2392 | N | GLY | B | 355 | 13.496 | 17.303 | 45.431 | 1.00 | 24.17 | N |
| ATOM | 2393 | CA | GLY | B | 355 | 13.551 | 16.367 | 46.542 | 1.00 | 26.73 | C |
| ATOM | 2394 | C | GLY | B | 355 | 14.388 | 16.954 | 47.667 | 1.00 | 29.41 | C |
| ATOM | 2395 | O | GLY | B | 355 | 14.773 | 18.123 | 47.626 | 1.00 | 24.83 | O |
| ATOM | 2396 | N | VAL | B | 356 | 14.6651 | 16.125 | 48.684 | 1.00 | 29.40 | N |
| ATOM | 2397 | CA | VAL | B | 356 | 15.476 | 16.534 | 49.817 | 1.00 | 27.26 | C |
| ATOM | 2398 | CB | VAL | B | 356 | 14.633 | 16.754 | 51.105 | 1.00 | 32.61 | C |
| ATOM | 2399 | CG1 | VAL | B | 356 | 15.549 | 17.148 | 52.284 | 1.00 | 32.35 | C |
| ATOM | 2400 | CG2 | VAL | B | 356 | 13.611 | 17.822 | 50.855 | 1.00 | 32.47 | C |
| ATOM | 2401 | C | VAL | B | 356 | 16.446 | 15.389 | 50.059 | 1.00 | 21.33 | C |
| ATOM | 2402 | O | VAL | B | 356 | 16.077 | 14.233 | 49.936 | 1.00 | 25.09 | O |
| ATOM | 2403 | N | TYR | B | 357 | 17.690 | 15.724 | 50.382 | 1.00 | 29.52 | N |
| ATOM | 2404 | CA | TYR | B | 357 | 18.705 | 14.706 | 50.643 | 1.00 | 30.43 | C |
| ATOM | 2405 | CB | TYR | B | 357 | 19.852 | 14.784 | 49.631 | 1.00 | 35.80 | C |
| ATOM | 2406 | CG | TYR | B | 357 | 20.948 | 13.782 | 49.918 | 1.00 | 33.13 | C |
| ATOM | 2407 | CD1 | TYR | B | 357 | 20.784 | 12.426 | 49.633 | 1.00 | 34.25 | C |
| ATOM | 2408 | CE1 | TYR | B | 357 | 21.789 | 11.496 | 49.933 | 1.00 | 38.44 | C |
| ATOM | 2409 | CD2 | TYR | B | 357 | 22.135 | 14.192 | 50.510 | 1.00 | 40.34 | C |
| ATOM | 2410 | CE2 | TYR | B | 357 | 23.143 | 13.274 | 50.816 | 1.00 | 39.74 | C |
| ATOM | 2411 | CZ | TYR | B | 357 | 22.964 | 11.935 | 50.524 | 1.00 | 39.72 | C |
| ATOM | 2412 | OH | TYR | B | 357 | 23.971 | 11.048 | 50.819 | 1.00 | 49.57 | O |
| ATOM | 2413 | C | TYR | B | 357 | 19.266 | 14.944 | 52.034 | 1.00 | 33.64 | C |
| ATOM | 2414 | O | TYR | B | 357 | 19.528 | 16.085 | 52.420 | 1.00 | 32.19 | O |
| ATOM | 2415 | N | ARG | B | 358 | 19.461 | 13.863 | 52.777 | 1.00 | 39.19 | N |
| ATOM | 2416 | CA | ARG | B | 358 | 19.975 | 13.990 | 54.133 | 1.00 | 42.57 | C |
| ATOM | 2417 | CB | ARG | B | 358 | 19.248 | 13.003 | 55.061 | 1.00 | 40.77 | C |
| ATOM | 2418 | CG | ARG | B | 358 | 19.743 | 13.027 | 56.509 | 1.00 | 46.82 | C |
| ATOM | 2419 | CD | ARG | B | 358 | 18.942 | 12.054 | 57.364 | 1.00 | 45.02 | C |
| ATOM | 2420 | NE | ARG | B | 358 | 18.989 | 10.695 | 56.828 | 1.00 | 44.71 | N |
| ATOM | 2421 | CZ | ARG | B | 358 | 18.075 | 9.765 | 57.089 | 1.00 | 45.22 | C |
| ATOM | 2422 | NH1 | ARG | B | 358 | 17.048 | 10.048 | 57.875 | 1.00 | 46.71 | N |
| ATOM | 2423 | NH2 | ARG | B | 358 | 18.184 | 8.548 | 56.564 | 1.00 | 47.43 | N |
| ATOM | 2424 | C | ARG | B | 358 | 21.475 | 13.760 | 54.228 | 1.00 | 37.12 | C |
| ATOM | 2425 | O | ARG | B | 358 | 21.957 | 12.680 | 53.901 | 1.00 | 38.27 | O |
| ATOM | 2426 | N | MET | B | 359 | 22.198 | 14.798 | 54.643 | 1.00 | 48.39 | N |
| ATOM | 2427 | CA | MET | B | 359 | 23.641 | 14.713 | 54.854 | 1.00 | 52.85 | C |
| ATOM | 2428 | CB | MET | B | 359 | 24.368 | 15.892 | 54.211 | 1.00 | 56.92 | C |
| ATOM | 2429 | CG | MET | B | 359 | 24.070 | 16.084 | 52.741 | 1.00 | 60.92 | C |
| ATOM | 2430 | SD | MET | B | 359 | 25.113 | 17.339 | 51.987 | 1.00 | 69.39 | S |
| ATOM | 2431 | CE | MET | B | 359 | 24.465 | 18.838 | 52.768 | 1.00 | 66.46 | C |
| ATOM | 2432 | C | MET | B | 359 | 23.745 | 14.820 | 56.371 | 1.00 | 57.53 | C |
| ATOM | 2433 | O | MET | B | 359 | 23.423 | 15.866 | 59.936 | 1.00 | 66.72 | O |
| ATOM | 2434 | N | ARG | B | 360 | 24.163 | 13.741 | 57.024 | 1.00 | 61.83 | N |
| ATOM | 2435 | CA | ARG | B | 360 | 24.269 | 13.706 | 58.488 | 1.00 | 63.23 | C |
| ATOM | 2436 | CB | ARG | B | 360 | 25.166 | 12.537 | 58.925 | 1.00 | 63.83 | C |
| ATOM | 2437 | CG | ARG | B | 360 | 24.590 | 11.170 | 58.552 | 1.00 | 65.58 | C |
| ATOM | 2438 | CD | ARG | B | 360 | 23.197 | 11.000 | 59.158 | 1.00 | 65.50 | C |
| ATOM | 2439 | NE | ARG | B | 360 | 22.294 | 10.220 | 58.310 | 1.00 | 64.72 | N |
| ATOM | 2440 | CZ | ARG | B | 360 | 22.467 | 8.936 | 57.995 | 1.00 | 63.90 | C |
| ATOM | 2441 | NH1 | ARG | B | 360 | 23.523 | 8.261 | 58.451 | 1.00 | 62.73 | N |
| ATOM | 2442 | NH2 | ARG | B | 360 | 21.573 | 8.317 | 57.233 | 1.00 | 62.15 | N |
| ATOM | 2443 | C | ARG | B | 360 | 24.765 | 15.008 | 59.099 | 1.00 | 61.92 | C |
| ATOM | 2444 | O | ARG | B | 360 | 25.971 | 15.181 | 59.320 | 1.00 | 68.74 | O |
| ATOM | 2445 | N | LYS | B | 361 | 23.811 | 15.905 | 59.374 | 1.00 | 58.30 | N |
| ATOM | 2446 | CA | LYS | B | 361 | 24.045 | 17.233 | 59.984 | 1.00 | 55.64 | C |
| ATOM | 2447 | CB | LYS | B | 361 | 25.338 | 17.844 | 59.389 | 1.00 | 60.30 | C |
| ATOM | 2448 | CG | LYS | B | 361 | 25.334 | 18.061 | 57.877 | 1.00 | 60.80 | C |
| ATOM | 2449 | CD | LYS | B | 361 | 26.738 | 18.379 | 57.388 | 1.00 | 63.13 | C |
| ATOM | 2450 | CE | LYS | B | 361 | 27.334 | 19.641 | 57.964 | 1.00 | 63.56 | C |
| ATOM | 2451 | NZ | LYS | B | 361 | 28.715 | 19.918 | 57.453 | 1.00 | 66.04 | N |
| ATOM | 2452 | C | LYS | B | 361 | 22.857 | 18.139 | 59.589 | 1.00 | 60.03 | C |
| ATOM | 2453 | O | LYS | B | 361 | 22.408 | 18.968 | 60.395 | 1.00 | 59.15 | O |
| ATOM | 2454 | N | LYS | B | 362 | 22.353 | 17.971 | 58.365 | 1.00 | 53.10 | N |
| ATOM | 2455 | CA | LYS | B | 362 | 21.225 | 18.756 | 57.878 | 1.00 | 55.57 | C |
| ATOM | 2456 | CB | LYS | B | 362 | 21.661 | 20.202 | 57.665 | 1.00 | 57.57 | C |
| ATOM | 2457 | CG | LYS | B | 362 | 22.780 | 20.355 | 56.656 | 1.00 | 54.04 | C |
| ATOM | 2458 | CD | LYS | B | 362 | 23.226 | 21.799 | 56.608 | 1.00 | 58.13 | C |
| ATOM | 2459 | CE | LYS | B | 362 | 24.111 | 22.049 | 55.408 | 1.00 | 60.20 | C |
| ATOM | 2460 | NZ | LYS | B | 362 | 25.278 | 21.126 | 55.386 | 1.00 | 60.23 | N |
| ATOM | 2461 | C | LYS | B | 362 | 20.659 | 18.192 | 56.571 | 1.00 | 48.43 | C |
| ATOM | 2462 | O | LYS | B | 362 | 21.259 | 17.329 | 55.945 | 1.00 | 47.21 | O |
| ATOM | 2463 | N | GLN | B | 363 | 19.490 | 18.675 | 56.168 | 1.00 | 51.49 | N |
| ATOM | 2464 | CA | GLN | B | 363 | 18.877 | 18.200 | 54.935 | 1.00 | 49.86 | C |
| ATOM | 2465 | CB | GLN | B | 363 | 17.463 | 17.662 | 55.190 | 1.00 | 56.81 | C |
| ATOM | 2466 | CG | GLN | B | 363 | 17.400 | 16.534 | 56.212 | 1.00 | 54.91 | C |
| ATOM | 2467 | CD | GLN | B | 363 | 16.007 | 15.927 | 56.325 | 1.00 | 55.16 | C |
| ATOM | 2468 | OE1 | GLN | B | 363 | 15.000 | 16.600 | 56.098 | 1.00 | 61.00 | O |
| ATOM | 2469 | NE2 | GLN | B | 363 | 15.947 | 14.658 | 56.692 | 1.00 | 58.13 | N |
| ATOM | 2470 | C | GLN | B | 363 | 18.824 | 19.341 | 53.943 | 1.00 | 48.03 | C |
| ATOM | 2471 | O | GLN | B | 363 | 18.369 | 20.435 | 54.261 | 1.00 | 50.07 | O |
| ATOM | 2472 | N | ILE | B | 364 | 19.287 | 19.079 | 52.727 | 1.00 | 39.54 | N |
| ATOM | 2473 | CA | ILE | B | 364 | 19.294 | 20.120 | 51.725 | 1.00 | 40.83 | C |
| ATOM | 2474 | CB | ILE | B | 364 | 20.699 | 20.335 | 51.165 | 1.00 | 45.38 | C |
| ATOM | 2475 | CG2 | ILE | B | 364 | 21.653 | 20.714 | 52.290 | 1.00 | 41.39 | C |
| ATOM | 2476 | CG1 | ILE | B | 364 | 21.174 | 19.071 | 50.475 | 1.00 | 39.09 | C |
| ATOM | 2477 | CD1 | ILE | B | 364 | 22.299 | 19.333 | 49.517 | 1.00 | 48.16 | C |
| ATOM | 2478 | C | ILE | B | 364 | 18.361 | 19.817 | 50.574 | 1.00 | 36.23 | C |
| ATOM | 2479 | O | ILE | B | 364 | 18.086 | 18.656 | 50.268 | 1.00 | 30.72 | O |
| ATOM | 2480 | N | ASP | B | 365 | 17.875 | 20.8700 | 49.934 | 1.00 | 35.72 | N |
| ATOM | 2481 | CA | ASP | B | 365 | 16.983 | 20.700 | 48.797 | 1.00 | 33.63 | C |
| ATOM | 2482 | CB | ASP | B | 365 | 16.300 | 22.025 | 48.483 | 1.00 | 37.07 | C |
| ATOM | 2483 | CG | ASP | B | 365 | 15.425 | 22.506 | 49.621 | 1.00 | 43.21 | C |
| ATOM | 2484 | OD1 | ASP | B | 365 | 14.589 | 21.712 | 50.105 | 1.00 | 44.93 | O |
| ATOM | 2485 | OD2 | ASP | B | 365 | 15.576 | 23.678 | 50.026 | 1.00 | 47.38 | O |
| ATOM | 2486 | C | ASP | B | 365 | 17.825 | 20.269 | 47.590 | 1.00 | 29.49 | C |
| ATOM | 2487 | O | ASP | B | 365 | 18.899 | 20.819 | 47.363 | 1.00 | 32.88 | O |
| ATOM | 2488 | N | VAL | B | 366 | 17.340 | 19.303 | 48.816 | 1.00 | 24.92 | N |
| ATOM | 2489 | CA | VAL | B | 366 | 18.080 | 18.847 | 45.654 | 1.00 | 25.63 | C |
| ATOM | 2490 | CB | VAL | B | 366 | 18.718 | 17.443 | 45.885 | 1.00 | 22.38 | C |
| ATOM | 2491 | CG1 | VAL | B | 366 | 19.793 | 17.506 | 46.993 | 1.00 | 22.29 | C |
| ATOM | 2492 | CG2 | VAL | B | 366 | 17.619 | 16.405 | 46.205 | 1.00 | 24.67 | C |
| ATOM | 2493 | C | VAL | B | 366 | 17.190 | 18.731 | 44.420 | 1.00 | 23.28 | C |
| ATOM | 2494 | O | VAL | B | 366 | 15.974 | 18.745 | 44.522 | 1.00 | 26.31 | O |
| ATOM | 2495 | N | ALA | B | 367 | 17.816 | 18.653 | 43.250 | 1.00 | 23.51 | N |
| ATOM | 2496 | CA | ALA | B | 367 | 17.091 | 18.439 | 42.010 | 1.00 | 26.78 | C |
| ATOM | 2497 | CB | ALA | B | 367 | 19.396 | 19.567 | 40.990 | 1.00 | 25.36 | C |
| ATOM | 2498 | C | ALA | B | 367 | 17.639 | 17.074 | 41.554 | 1.00 | 26.08 | C |
| ATOM | 2499 | O | ALA | B | 367 | 18.843 | 16.821 | 41.602 | 1.00 | 30.40 | O |
| ATOM | 2500 | N | ILE | B | 368 | 16.751 | 16.192 | 41.128 | 1.00 | 25.61 | N |
| ATOM | 2501 | CA | ILE | B | 368 | 17.124 | 14.839 | 40.735 | 1.00 | 25.37 | C |
| ATOM | 2502 | CB | ILE | B | 368 | 16.365 | 13.781 | 41.603 | 1.00 | 28.96 | C |
| ATOM | 2503 | CG2 | ILE | B | 368 | 16.853 | 12.365 | 41.280 | 1.00 | 26.27 | C |
| ATOM | 2504 | CG1 | ILE | B | 368 | 16.580 | 14.082 | 43.092 | 1.00 | 26.82 | C |
| ATOM | 2505 | CD1 | ILE | B | 368 | 15.300 | 14.229 | 43.893 | 1.00 | 35.04 | C |
| ATOM | 2506 | C | ILE | B | 368 | 16.796 | 14.548 | 39.284 | 1.00 | 23.83 | C |
| ATOM | 2507 | O | ILE | B | 368 | 15.638 | 14.575 | 38.895 | 1.00 | 24.17 | O |
| ATOM | 2508 | N | LYS | B | 369 | 17.820 | 14.261 | 38.491 | 1.00 | 25.66 | N |
| ATOM | 2509 | CA | LYS | B | 369 | 17.593 | 13.923 | 37.089 | 1.00 | 26.48 | C |
| ATOM | 2510 | CB | LYS | B | 369 | 18.853 | 14.235 | 36.278 | 1.00 | 26.70 | C |
| ATOM | 2511 | CG | LYS | B | 369 | 18.727 | 13.997 | 34.804 | 1.00 | 32.20 | C |
| ATOM | 2512 | CD | LYS | B | 369 | 20.014 | 14.364 | 34.094 | 1.00 | 31.43 | C |
| ATOM | 2513 | CE | LYS | B | 369 | 20.013 | 13.896 | 32.654 | 1.00 | 37.28 | C |
| ATOM | 2514 | NZ | LYS | B | 369 | 21.184 | 14.385 | 31.848 | 1.00 | 31.40 | N |
| ATOM | 2515 | C | LYS | B | 369 | 17.250 | 12.429 | 37.070 | 1.00 | 30.56 | C |
| ATOM | 2516 | O | LYS | B | 369 | 18.079 | 11.572 | 37.414 | 1.00 | 29.63 | O |
| ATOM | 2517 | N | VAL | B | 370 | 16.016 | 12.112 | 36.703 | 1.00 | 28.76 | N |
| ATOM | 2518 | CA | VAL | B | 370 | 15.553 | 10.730 | 36.690 | 1.00 | 29.89 | C |
| ATOM | 2519 | CB | VAL | B | 370 | 14.192 | 10.594 | 17.410 | 1.00 | 32.92 | C |
| ATOM | 2520 | CG1 | VAL | B | 370 | 13.824 | 9.133 | 37.553 | 1.00 | 38.36 | C |
| ATOM | 2521 | CG2 | VAL | B | 370 | 14.235 | 11.289 | 38.775 | 1.00 | 31.30 | C |
| ATOM | 2522 | C | VAL | B | 370 | 15.391 | 10.222 | 32.265 | 1.00 | 33.31 | C |
| ATOM | 2523 | O | VAL | B | 370 | 14.623 | 10.779 | 34.484 | 1.00 | 33.69 | O |
| ATOM | 2524 | N | LEU | B | 371 | 16.110 | 9.166 | 34.921 | 1.00 | 32.83 | N |
| ATOM | 2525 | CA | LEU | B | 371 | 15.992 | 8.614 | 33.580 | 1.00 | 37.54 | C |
| ATOM | 2526 | CB | LEU | B | 371 | 17.164 | 7.676 | 33.288 | 1.00 | 39.83 | C |
| ATOM | 2527 | CG | LEU | B | 371 | 18.543 | 8.328 | 33.234 | 1.00 | 40.84 | C |
| ATOM | 2528 | CD1 | LEU | B | 371 | 19.589 | 7.261 | 32.948 | 1.00 | 41.39 | C |
| ATOM | 2529 | CD2 | LEU | B | 371 | 18.555 | 9.402 | 32.166 | 1.00 | 39.75 | C |
| ATOM | 2530 | C | LEU | B | 371 | 14.675 | 7.853 | 33.449 | 1.00 | 43.14 | C |
| ATOM | 2531 | O | LEU | B | 371 | 14.307 | 7.075 | 34.339 | 1.00 | 39.21 | O |
| ATOM | 2532 | N | LYS | B | 372 | 13.978 | 8.075 | 32.337 | 1.00 | 41.93 | N |
| ATOM | 2533 | CA | LYS | B | 372 | 12.699 | 7.420 | 32.080 | 1.00 | 47.49 | C |
| ATOM | 2534 | CB | LYS | B | 372 | 12.114 | 7.917 | 30.756 | 1.00 | 49.29 | C |
| ATOM | 2535 | CG | LYS | B | 372 | 13.009 | 7.677 | 29.541 | 1.00 | 52.95 | C |
| ATOM | 2536 | CD | LYS | B | 372 | 12.390 | 8.257 | 28.275 | 1.00 | 54.13 | C |
| ATOM | 2537 | CE | LYS | B | 372 | 13.254 | 7.957 | 27.057 | 1.00 | 57.77 | C |
| ATOM | 2538 | NZ | LYS | B | 372 | 12.655 | 8.487 | 25.796 | 1.00 | 61.32 | N |
| ATOM | 2539 | C | LYS | B | 372 | 12.851 | 5.900 | 32.052 | 1.00 | 52.52 | C |
| ATOM | 2540 | O | LYS | B | 372 | 13.951 | 5.378 | 31.877 | 1.00 | 47.39 | O |
| ATOM | 2541 | N | GLN | B | 373 | 11.740 | 5.193 | 32.238 | 1.00 | 59.30 | N |
| ATOM | 2542 | CA | GLN | B | 373 | 11.755 | 3.733 | 32.231 | 1.00 | 57.74 | C |
| ATOM | 2543 | CB | GLN | B | 373 | 10.418 | 3.197 | 32.762 | 1.00 | 64.02 | C |
| ATOM | 2544 | CG | GLN | B | 373 | 10.377 | 1.685 | 32.981 | 1.00 | 66.26 | C |
| ATOM | 2545 | CD | GLN | B | 373 | 11.342 | 1.215 | 34.063 | 1.00 | 70.74 | C |
| ATOM | 2546 | OE1 | GLN | B | 373 | 12.562 | 1.337 | 33.924 | 1.00 | 69.05 | O |
| ATOM | 2547 | NE | GLN | B | 373 | 10.795 | 0.672 | 35.147 | 1.00 | 69.07 | N |
| ATOM | 2548 | C | GLN | B | 373 | 12.005 | 3.213 | 30.811 | 1.00 | 61.28 | C |
| ATOM | 2549 | O | GLN | B | 373 | 11.441 | 3.728 | 29.840 | 1.00 | 59.36 | O |
| ATOM | 2550 | N | GLY | B | 374 | 12.863 | 2.203 | 30.692 | 1.00 | 60.31 | N |
| ATOM | 2551 | CA | GLY | B | 374 | 13.160 | 1.645 | 29.385 | 1.00 | 62.87 | C |
| ATOM | 2552 | C | GLY | B | 374 | 14.425 | 2.207 | 28.763 | 1.00 | 64.46 | C |
| ATOM | 2553 | O | GLY | B | 374 | 14.792 | 1.838 | 27.643 | 1.00 | 64.42 | O |
| ATOM | 2554 | N | THR | B | 375 | 15.091 | 3.101 | 29.490 | 1.00 | 62.61 | N |
| ATOM | 2555 | CA | THR | B | 375 | 16.326 | 3.722 | 29.019 | 1.00 | 62.15 | C |
| ATOM | 2556 | CB | THR | B | 375 | 16.913 | 4.678 | 30.089 | 1.00 | 61.37 | C |
| ATOM | 2557 | OG1 | THR | B | 375 | 16.039 | 5.799 | 30.268 | 1.00 | 57.85 | O |
| ATOM | 2558 | OG2 | THR | B | 375 | 18.288 | 5.175 | 29.671 | 1.00 | 61.31 | C |
| ATOM | 2559 | C | THR | B | 375 | 17.374 | 2.666 | 28.673 | 1.00 | 60.79 | C |
| ATOM | 2560 | O | THR | B | 375 | 17.728 | 1.835 | 29.507 | 1.00 | 61.50 | O |
| ATOM | 2561 | N | GLU | B | 376 | 17.867 | 2.708 | 27.440 | 1.00 | 61.17 | N |
| ATOM | 2562 | CA | GLU | B | 376 | 18.874 | 1.753 | 26.990 | 1.00 | 63.67 | C |
| ATOM | 2563 | CB | GLU | B | 376 | 19.139 | 1.933 | 25.496 | 1.00 | 65.15 | C |
| ATOM | 2564 | CG | GLU | B | 376 | 20.120 | 0.923 | 24.917 | 1.00 | 71.18 | C |
| ATOM | 2565 | CD | GLU | B | 376 | 20.089 | 0.883 | 23.398 | 1.00 | 73.78 | C |
| ATOM | 2566 | OE1 | GLU | B | 376 | 20.380 | 1.923 | 22.758 | 1.00 | 72.53 | O |
| ATOM | 2567 | OE2 | GLU | B | 376 | 19.769 | −0.193 | 22.843 | 1.00 | 73.05 | O |
| ATOM | 2568 | C | GLU | B | 376 | 20.169 | 1.943 | 27.771 | 1.00 | 65.33 | C |
| ATOM | 2569 | O | GLU | B | 376 | 20.642 | 3.046 | 28.237 | 1.00 | 61.91 | O |
| ATOM | 2570 | N | LYS | B | 377 | 20.940 | 0.868 | 27.910 | 1.00 | 59.58 | N |
| ATOM | 2571 | CA | LYS | B | 377 | 22.196 | 0.927 | 28.644 | 1.00 | 61.29 | C |
| ATOM | 2572 | CB | LYS | B | 377 | 22.896 | −0.432 | 28.608 | 1.00 | 65.21 | C |
| ATOM | 2573 | CG | LYS | B | 377 | 24.222 | −0.454 | 29.356 | 1.00 | 65.59 | C |
| ATOM | 2574 | CD | LYS | B | 377 | 24.611 | −1.872 | 29.736 | 1.00 | 71.26 | C |
| ATOM | 2575 | CE | LYS | B | 377 | 25.867 | −1.896 | 30.599 | 1.00 | 70.47 | C |
| ATOM | 2576 | NZ | LYS | B | 377 | 26.267 | −3.292 | 30.962 | 1.00 | 72.53 | N |
| ATOM | 2577 | C | LYS | B | 377 | 23.131 | 1.993 | 28.095 | 1.00 | 59.78 | C |
| ATOM | 2578 | O | LYS | B | 377 | 23.999 | 2.497 | 28.808 | 1.00 | 56.95 | O |
| ATOM | 2579 | N | ALA | B | 378 | 22.952 | 2.329 | 26.823 | 1.00 | 61.07 | N |
| ATOM | 2580 | CA | ALA | B | 378 | 23.777 | 3.337 | 26.168 | 1.00 | 59.33 | C |
| ATOM | 2581 | CB | ALA | B | 378 | 23.399 | 3.435 | 24.695 | 1.00 | 59.59 | C |
| ATOM | 2582 | C | ALA | B | 378 | 23.624 | 4.703 | 26.836 | 1.00 | 57.38 | C |
| ATOM | 2583 | O | ALA | B | 378 | 24.612 | 5.376 | 27.138 | 1.00 | 56.25 | O |
| ATOM | 2584 | N | ASP | B | 379 | 22.380 | 5.098 | 27.075 | 1.00 | 57.35 | N |
| ATOM | 2585 | CA | ASP | B | 379 | 22.094 | 6.388 | 27.691 | 1.00 | 59.15 | C |
| ATOM | 2586 | CB | ASP | B | 379 | 20.650 | 6.788 | 27.416 | 1.00 | 61.51 | C |
| ATOM | 2587 | CG | ASP | B | 379 | 20.151 | 6.255 | 26.088 | 1.00 | 68.59 | C |
| ATOM | 2588 | OD1 | ASP | B | 379 | 20.739 | 6.601 | 25.035 | 1.00 | 70.20 | O |
| ATOM | 2589 | OD2 | ASP | B | 379 | 19.168 | 5.479 | 26.098 | 1.00 | 70.89 | O |
| ATOM | 2590 | C | ASP | B | 379 | 22.341 | 6.358 | 29.197 | 1.00 | 56.77 | C |
| ATOM | 2591 | O | ASP | B | 379 | 22.596 | 7.392 | 29.811 | 1.00 | 53.33 | O |
| ATOM | 2592 | N | THR | B | 380 | 22.258 | 5.170 | 29.790 | 1.00 | 52.16 | N |
| ATOM | 2593 | CA | THR | B | 380 | 22.513 | 5.019 | 31.215 | 1.00 | 53.38 | C |
| ATOM | 2594 | CB | THR | B | 380 | 22.187 | 3.590 | 31.684 | 1.00 | 53.55 | C |
| ATOM | 2595 | OG1 | THR | B | 380 | 20.768 | 3.391 | 31.641 | 1.00 | 54.68 | O |
| ATOM | 2596 | OG2 | THR | B | 380 | 22.683 | 3.371 | 33.096 | 1.00 | 58.07 | C |
| ATOM | 2597 | C | THR | B | 380 | 23.998 | 5.295 | 31.432 | 1.00 | 52.60 | C |
| ATOM | 2598 | O | THR | B | 380 | 24.390 | 5.980 | 32.374 | 1.00 | 48.13 | O |
| ATOM | 2599 | N | GLU | B | 381 | 24.819 | 4.752 | 30.540 | 1.00 | 46.95 | N |
| ATOM | 2600 | CA | GLU | B | 381 | 26.260 | 4.937 | 30.617 | 1.00 | 49.11 | C |
| ATOM | 2601 | CB | GLU | B | 381 | 26.955 | 4.060 | 29.573 | 1.00 | 54.95 | C |
| ATOM | 2602 | CG | GLU | B | 381 | 28.020 | 3.129 | 30.134 | 1.00 | 58.45 | C |
| ATOM | 2603 | CD | GLU | B | 381 | 27.442 | 1.927 | 30.862 | 1.00 | 65.92 | C |
| ATOM | 2604 | OE1 | GLU | B | 381 | 26.790 | 2.114 | 31.913 | 1.00 | 66.10 | O |
| ATOM | 2605 | OE2 | GLU | B | 381 | 27.644 | 0.788 | 30.377 | 1.00 | 67.00 | O |
| ATOM | 2606 | C | GLU | B | 381 | 26.559 | 6.412 | 30.343 | 1.00 | 45.43 | C |
| ATOM | 2607 | O | GLU | B | 381 | 27.518 | 6.974 | 30.864 | 1.00 | 40.91 | O |
| ATOM | 2608 | N | GLU | B | 382 | 25.716 | 7.029 | 29.525 | 1.00 | 43.34 | N |
| ATOM | 2609 | CA | GLU | B | 382 | 25.871 | 8.435 | 29.164 | 1.00 | 45.73 | C |
| ATOM | 2610 | CB | GLU | B | 382 | 24.842 | 8.795 | 28.099 | 1.00 | 50.57 | C |
| ATOM | 2611 | CG | GLU | B | 382 | 24.988 | 10.184 | 27.524 | 1.00 | 55.43 | C |
| ATOM | 2612 | CD | GLU | B | 382 | 24.064 | 10.410 | 26.336 | 1.00 | 60.92 | C |
| ATOM | 2613 | OE1 | GLU | B | 382 | 22.825 | 10.449 | 26.528 | 1.00 | 67.80 | O |
| ATOM | 2614 | OE2 | GLU | B | 382 | 24.580 | 10.536 | 25.203 | 1.00 | 65.61 | O |
| ATOM | 2615 | C | GLU | B | 382 | 25.696 | 9.331 | 30.394 | 1.00 | 42.57 | C |
| ATOM | 2616 | O | GLU | B | 382 | 26.444 | 10.294 | 30.587 | 1.00 | 36.66 | O |
| ATOM | 2617 | N | MET | B | 383 | 24.700 | 9.017 | 31.217 | 1.00 | 36.27 | N |
| ATOM | 2618 | CA | MET | B | 383 | 24.482 | 9.806 | 31.417 | 1.00 | 36.21 | C |
| ATOM | 2619 | CB | MET | B | 383 | 23.116 | 9.489 | 33.051 | 1.00 | 37.94 | C |
| ATOM | 2620 | CG | MET | B | 383 | 22.686 | 10.580 | 34.040 | 1.00 | 45.05 | C |
| ATOM | 2621 | SD | MET | B | 383 | 21.039 | 10.380 | 34.434 | 1.00 | 49.17 | S |
| ATOM | 2622 | CE | MET | B | 383 | 21.377 | 9.057 | 35.776 | 1.00 | 31.72 | C |
| ATOM | 2623 | C | MET | B | 383 | 25.602 | 9.543 | 33.420 | 1.00 | 32.29 | C |
| ATOM | 2624 | O | MET | B | 383 | 25.912 | 10.389 | 34.245 | 1.00 | 27.76 | O |
| ATOM | 2625 | N | MET | B | 384 | 26.212 | 8.361 | 33.362 | 1.00 | 32.45 | N |
| ATOM | 2626 | CA | MET | B | 384 | 27.310 | 8.076 | 34.276 | 1.00 | 34.42 | C |
| ATOM | 2627 | CB | MET | B | 384 | 27.664 | 6.588 | 34.262 | 1.00 | 38.83 | C |
| ATOM | 2628 | CG | MET | B | 384 | 26.634 | 5.702 | 39.943 | 1.00 | 41.15 | C |
| ATOM | 2629 | SD | MET | B | 384 | 26.295 | 6.179 | 36.672 | 1.00 | 47.37 | S |
| ATOM | 2630 | CE | MET | B | 384 | 27.936 | 6.041 | 37.407 | 1.00 | 46.98 | C |
| ATOM | 2631 | C | MET | B | 384 | 28.529 | 8.911 | 33.896 | 1.00 | 31.48 | C |
| ATOM | 2632 | O | MET | B | 384 | 29.284 | 9.352 | 34.762 | 1.00 | 31.65 | O |
| ATOM | 2633 | N | ARG | B | 385 | 28.727 | 9.133 | 32.603 | 1.00 | 34.67 | N |
| ATOM | 2634 | CA | ARG | B | 385 | 29.848 | 9.962 | 32.172 | 1.00 | 36.33 | C |
| ATOM | 2635 | CB | ARG | B | 385 | 30.011 | 9.926 | 30.652 | 1.00 | 37.82 | C |
| ATOM | 2636 | CG | ARG | B | 385 | 30.687 | 8.667 | 30.129 | 1.00 | 45.45 | C |
| ATOM | 2637 | CD | ARG | B | 385 | 31.105 | 8.837 | 28.665 | 1.00 | 46.60 | C |
| ATOM | 2638 | NE | ARG | B | 385 | 29.969 | 8.752 | 27.756 | 1.00 | 45.85 | N |
| ATOM | 2639 | CZ | ARG | B | 385 | 29.377 | 7.610 | 27.417 | 1.00 | 47.08 | C |
| ATOM | 2640 | NH1 | ARG | B | 385 | 29.823 | 6.459 | 27.913 | 1.00 | 48.61 | N |
| ATOM | 2641 | NH2 | ARG | B | 385 | 28.342 | 7.612 | 26.592 | 1.00 | 46.74 | N |
| ATOM | 2642 | C | ARG | B | 385 | 29.596 | 11.391 | 32.636 | 1.00 | 29.96 | C |
| ATOM | 2643 | O | ARG | B | 385 | 30.520 | 12.098 | 33.021 | 1.00 | 33.63 | O |
| ATOM | 2644 | N | GLU | B | 386 | 28.330 | 11.799 | 32.612 | 1.00 | 30.00 | N |
| ATOM | 2645 | CA | GLU | B | 386 | 27.948 | 13.141 | 33.051 | 1.00 | 29.80 | C |
| ATOM | 2646 | CB | GLU | B | 386 | 26.447 | 13.368 | 32.790 | 1.00 | 27.12 | C |
| ATOM | 2647 | CG | GLU | B | 386 | 25.882 | 14.720 | 33.233 | 1.00 | 32.18 | C |
| ATOM | 2648 | CD | GLU | B | 386 | 24.372 | 14.825 | 32.961 | 1.00 | 35.05 | C |
| ATOM | 2649 | OE1 | GLU | B | 386 | 23.801 | 13.830 | 32.475 | 1.00 | 34.50 | O |
| ATOM | 2650 | OE2 | GLU | B | 386 | 23.756 | 15.890 | 33.251 | 1.00 | 35.81 | O |
| ATOM | 2651 | C | GLU | B | 386 | 28.259 | 13.298 | 34.540 | 1.00 | 28.79 | C |
| ATOM | 2652 | O | GLU | B | 386 | 28.752 | 14.350 | 34.986 | 1.00 | 26.92 | O |
| ATOM | 2653 | N | ALA | B | 387 | 27.967 | 12.253 | 35.305 | 1.00 | 30.29 | N |
| ATOM | 2654 | CA | ALA | B | 387 | 28.223 | 12.295 | 36.736 | 1.00 | 33.27 | C |
| ATOM | 2655 | CB | ALA | B | 387 | 27.644 | 11.044 | 37.421 | 1.00 | 30.95 | C |
| ATOM | 2656 | C | ALA | B | 387 | 29.744 | 12.398 | 36.962 | 1.00 | 36.41 | C |
| ATOM | 2657 | O | ALA | B | 387 | 30.191 | 13.182 | 37.774 | 1.00 | 35.19 | O |
| ATOM | 2658 | N | GLN | B | 388 | 30.518 | 11.614 | 36.219 | 1.00 | 32.50 | N |
| ATOM | 2659 | CA | GLN | B | 388 | 31.984 | 11.654 | 36.367 | 1.00 | 37.92 | C |
| ATOM | 2660 | CB | GLN | B | 388 | 32.647 | 10.664 | 35.387 | 1.00 | 38.99 | C |
| ATOM | 2661 | CG | GLN | B | 388 | 32.269 | 9.211 | 35.625 | 1.00 | 47.85 | C |
| ATOM | 2662 | CD | GLN | B | 388 | 32.810 | 8.279 | 34.546 | 1.00 | 56.30 | C |
| ATOM | 2663 | OE1 | GLN | B | 388 | 34.016 | 8.248 | 34.281 | 1.00 | 56.71 | O |
| ATOM | 2664 | NE2 | GLN | B | 388 | 31.914 | 7.514 | 33.915 | 1.00 | 53.14 | N |
| ATOM | 2665 | C | GLN | B | 388 | 32.546 | 13.060 | 36.134 | 1.00 | 35.03 | C |
| ATOM | 2666 | O | GLN | B | 388 | 33.442 | 13.501 | 36.841 | 1.00 | 32.27 | O |
| ATOM | 2667 | N | ILE | B | 389 | 32.015 | 13.767 | 35.135 | 1.00 | 30.85 | N |
| ATOM | 2668 | CA | ILE | B | 389 | 32.463 | 15.130 | 34.840 | 1.00 | 25.41 | C |
| ATOM | 2669 | CB | ILE | B | 389 | 31.853 | 15.645 | 33.499 | 1.00 | 32.74 | C |
| ATOM | 2670 | CG2 | ILE | B | 389 | 32.051 | 17.159 | 33.363 | 1.00 | 31.69 | C |
| ATOM | 2671 | CG1 | ILE | B | 389 | 32.505 | 14.906 | 32.328 | 1.00 | 38.87 | C |
| ATOM | 2672 | CD1 | ILE | B | 389 | 31.893 | 15.236 | 30.982 | 1.00 | 36.36 | C |
| ATOM | 2673 | C | ILE | B | 389 | 32.072 | 16.074 | 35.949 | 1.00 | 26.93 | C |
| ATOM | 2674 | O | ILE | B | 389 | 32.872 | 16.886 | 36.411 | 1.00 | 30.11 | O |
| ATOM | 2675 | N | MET | B | 390 | 30.832 | 15.978 | 36.403 | 1.00 | 26.48 | N |
| ATOM | 2676 | CA | MET | B | 390 | 30.398 | 16.857 | 37.459 | 1.00 | 24.57 | C |
| ATOM | 2677 | CB | MET | B | 390 | 28.906 | 16.695 | 37.735 | 1.00 | 24.91 | C |
| ATOM | 2678 | CG | MET | B | 390 | 28.002 | 17.248 | 36.638 | 1.00 | 26.56 | C |
| ATOM | 2679 | SD | MET | B | 390 | 26.239 | 17.032 | 37.117 | 1.00 | 30.07 | S |
| ATOM | 2480 | CE | MET | B | 390 | 26.009 | 18.455 | 38.144 | 1.00 | 31.13 | C |
| ATOM | 2681 | C | MET | B | 390 | 31.186 | 16.563 | 38.738 | 1.00 | 30.37 | C |
| ATOM | 2682 | O | MET | B | 390 | 31.506 | 17.484 | 39.487 | 1.00 | 29.10 | O |
| ATOM | 2683 | N | HIS | B | 391 | 31.497 | 15.286 | 38.956 | 1.00 | 31.09 | N |
| ATOM | 2684 | CA | HIS | B | 391 | 32.243 | 14.890 | 40.160 | 1.00 | 37.62 | C |
| ATOM | 2685 | CB | HIS | B | 391 | 32.285 | 13.358 | 40.270 | 1.00 | 42.66 | C |
| ATOM | 2686 | CG | HIS | B | 391 | 32.831 | 12.854 | 41.573 | 1.00 | 50.59 | C |
| ATOM | 2687 | CD2 | HIS | B | 391 | 32.206 | 12.397 | 42.686 | 1.00 | 51.17 | C |
| ATOM | 2688 | ND1 | HIS | B | 391 | 34.182 | 12.815 | 41.850 | 1.00 | 56.54 | N |
| ATOM | 2689 | CE1 | HIS | B | 391 | 34.366 | 12.356 | 43.076 | 1.00 | 56.46 | C |
| ATOM | 2690 | NE2 | HIS | B | 391 | 33.183 | 12.094 | 43.605 | 1.00 | 57.11 | N |
| ATOM | 2692 | C | HIS | B | 391 | 33.661 | 15.454 | 40.155 | 1.00 | 43.32 | C |
| ATOM | 2692 | O | HIS | B | 391 | 34.338 | 15.452 | 41.182 | 1.00 | 47.18 | O |
| ATOM | 2693 | N | GLN | B | 392 | 34.111 | 15.941 | 38.999 | 1.00 | 39.89 | N |
| ATOM | 2694 | CA | GLN | B | 392 | 35.449 | 16.516 | 38.886 | 1.00 | 40.16 | C |
| ATOM | 2695 | CB | GLN | B | 392 | 36.094 | 16.110 | 37.557 | 1.00 | 42.86 | C |
| ATOM | 2696 | CG | GLN | B | 392 | 36.181 | 14.610 | 37.364 | 1.00 | 47.64 | C |
| ATOM | 2697 | CD | GLN | B | 392 | 36.948 | 14.219 | 36.129 | 1.00 | 50.73 | C |
| ATOM | 2698 | OE1 | GLN | B | 392 | 38.161 | 14.408 | 36.058 | 1.00 | 60.38 | O |
| ATOM | 2699 | NE2 | GLN | B | 392 | 36.250 | 13.664 | 35.145 | 1.00 | 48.83 | N |
| ATOM | 2700 | C | GLN | B | 392 | 35.415 | 18.031 | 38.987 | 1.00 | 47.76 | C |
| ATOM | 2701 | O | GLN | B | 392 | 36.444 | 18.689 | 38.850 | 1.00 | 47.76 | O |
| ATOM | 2702 | N | LEU | B | 393 | 34.233 | 18.586 | 39.240 | 1.00 | 45.72 | N |
| ATOM | 2703 | CA | LEU | B | 393 | 34.069 | 20.034 | 39.342 | 1.00 | 45.69 | C |
| ATOM | 2704 | CB | LEU | B | 393 | 32.909 | 20.491 | 38.454 | 1.00 | 44.69 | C |
| ATOM | 2705 | CG | LEU | B | 393 | 32.913 | 20.011 | 37.000 | 1.00 | 46.90 | C |
| ATOM | 2706 | CD1 | LEU | B | 393 | 31.656 | 20.508 | 36.312 | 1.00 | 44.79 | C |
| ATOM | 2707 | CD2 | LEU | B | 393 | 34.153 | 20.507 | 36.281 | 1.00 | 37.05 | C |
| ATOM | 2708 | C | LEU | B | 393 | 33.826 | 20.515 | 40.770 | 1.00 | 45.06 | C |
| ATOM | 2709 | O | LEU | B | 393 | 33.044 | 19.928 | 41.523 | 1.00 | 49.45 | O |
| ATOM | 2710 | N | ASP | B | 394 | 34.481 | 21.603 | 41.151 | 1.00 | 43.03 | N |
| ATOM | 2711 | CA | ASP | B | 394 | 34.315 | 22.133 | 42.495 | 1.00 | 36.00 | C |
| ATOM | 2712 | CB | ASP | B | 394 | 35.432 | 21.600 | 43.403 | 1.00 | 47.00 | C |
| ATOM | 2713 | CG | ASP | B | 394 | 35.292 | 22.063 | 44.844 | 1.00 | 52.54 | C |
| ATOM | 2714 | OD1 | ASP | B | 394 | 36.167 | 21.705 | 45.668 | 1.00 | 55.59 | O |
| ATOM | 2715 | OD2 | ASP | B | 394 | 34.315 | 22.774 | 45.160 | 1.00 | 47.08 | O |
| ATOM | 2716 | C | ASP | B | 394 | 34.381 | 23.640 | 42.427 | 1.00 | 40.50 | C |
| ATOM | 2717 | O | ASP | B | 394 | 35.434 | 24.247 | 42.671 | 1.00 | 38.62 | O |
| ATOM | 2718 | N | ASN | B | 395 | 33.251 | 24.254 | 42.105 | 1.00 | 35.95 | N |
| ATOM | 2719 | CA | ASN | B | 395 | 33.216 | 25.669 | 41.987 | 1.00 | 31.17 | C |
| ATOM | 2720 | CB | ASN | B | 395 | 33.463 | 26.092 | 40.518 | 1.00 | 25.15 | C |
| ATOM | 2721 | CG | ASN | B | 395 | 33.595 | 27.578 | 40.328 | 1.00 | 36.48 | C |
| ATOM | 2722 | OD1 | ASN | B | 395 | 34.673 | 28.158 | 40.523 | 1.00 | 37.23 | O |
| ATOM | 2723 | ND2 | ASN | B | 395 | 32.497 | 28.217 | 39.950 | 1.00 | 25.99 | N |
| ATOM | 2724 | C | ASN | B | 395 | 31.851 | 26.171 | 42.458 | 1.00 | 30.67 | C |
| ATOM | 2725 | O | ASN | B | 395 | 30.821 | 25.528 | 42.197 | 1.00 | 28.65 | O |
| ATOM | 2726 | N | PRO | B | 396 | 31.822 | 27.315 | 43.147 | 1.00 | 27.83 | N |
| ATOM | 2727 | CD | PRO | B | 396 | 32.966 | 28.068 | 43.680 | 1.00 | 37.81 | C |
| ATOM | 2728 | CA | PRO | B | 396 | 30.570 | 27.867 | 43.655 | 1.00 | 26.99 | C |
| ATOM | 2729 | CB | PRO | B | 396 | 31.036 | 29.053 | 44.483 | 1.00 | 35.20 | C |
| ATOM | 2730 | CG | PRO | B | 396 | 32.414 | 28.620 | 44.946 | 1.00 | 41.91 | C |
| ATOM | 2731 | C | PRO | B | 396 | 29.568 | 28.291 | 42.579 | 1.00 | 29.09 | C |
| ATOM | 2732 | O | PRO | B | 396 | 28.402 | 28.428 | 42.865 | 1.00 | 26.93 | O |
| ATOM | 2733 | N | TYR | B | 397 | 30.043 | 28.485 | 41.343 | 1.00 | 24.67 | N |
| ATOM | 2734 | CA | TYR | B | 397 | 29.145 | 28.956 | 40.263 | 1.00 | 24.00 | C |
| ATOM | 2735 | CB | TYR | B | 397 | 29.827 | 30.136 | 39.545 | 1.00 | 23.32 | C |
| ATOM | 2736 | CG | TYR | B | 397 | 30.228 | 31.229 | 40.524 | 1.00 | 26.27 | C |
| ATOM | 2737 | CD1 | TYR | B | 397 | 31.568 | 31.425 | 40.881 | 1.00 | 26.92 | C |
| ATOM | 2738 | CE1 | TYR | B | 397 | 31.929 | 32.379 | 41.823 | 1.00 | 31.33 | C |
| ATOM | 2739 | CD2 | TYR | B | 397 | 29.266 | 32.024 | 41.136 | 1.00 | 25.87 | C |
| ATOM | 2740 | CE2 | TYR | B | 397 | 29.620 | 32.987 | 42.085 | 1.00 | 33.47 | C |
| ATOM | 2741 | CZ | TYR | B | 397 | 30.950 | 33.155 | 42.426 | 1.00 | 34.44 | C |
| ATOM | 2742 | OH | TYR | B | 397 | 31.263 | 34.067 | 43.411 | 1.00 | 37.25 | O |
| ATOM | 2743 | C | TYR | B | 397 | 28.694 | 27.893 | 39.294 | 1.00 | 23.67 | C |
| ATOM | 2744 | O | TYR | B | 397 | 28.286 | 28.165 | 38.135 | 1.00 | 24.69 | O |
| ATOM | 2745 | N | ILE | B | 398 | 28.755 | 26.659 | 39.752 | 1.00 | 21.85 | N |
| ATOM | 2746 | CA | ILE | B | 398 | 28.336 | 25.519 | 38.991 | 1.00 | 26.71 | C |
| ATOM | 2747 | CB | ILE | B | 398 | 29.567 | 24.716 | 38.567 | 1.00 | 34.69 | C |
| ATOM | 2748 | CG2 | ILE | B | 398 | 29.165 | 23.359 | 38.073 | 1.00 | 36.71 | C |
| ATOM | 2749 | CG1 | ILE | B | 398 | 30.332 | 25.502 | 37.498 | 1.00 | 28.39 | C |
| ATOM | 2750 | CD1 | ILE | B | 398 | 31.737 | 25.021 | 37.232 | 1.00 | 39.03 | C |
| ATOM | 2751 | C | ILE | B | 398 | 27.421 | 24.691 | 39.906 | 1.00 | 34.83 | C |
| ATOM | 2752 | O | ILE | B | 398 | 27.640 | 24.636 | 41.120 | 1.00 | 31.60 | O |
| ATOM | 2753 | N | VAL | B | 399 | 26.378 | 24.103 | 39.333 | 1.00 | 26.19 | N |
| ATOM | 2754 | CA | VAL | B | 399 | 25.548 | 23.243 | 40.086 | 1.00 | 29.92 | C |
| ATOM | 2755 | CB | VAL | B | 399 | 24.272 | 22.781 | 39.232 | 1.00 | 30.42 | C |
| ATOM | 2756 | CG1 | VAL | B | 399 | 23.498 | 21.627 | 39.946 | 1.00 | 29.50 | C |
| ATOM | 2757 | CG2 | VAL | B | 399 | 23.360 | 23.960 | 38.970 | 1.00 | 31.26 | C |
| ATOM | 2758 | C | VAL | B | 399 | 26.265 | 22.016 | 40.464 | 1.00 | 34.54 | C |
| ATOM | 2759 | O | VAL | B | 399 | 26.697 | 21.269 | 39.597 | 1.00 | 35.65 | O |
| ATOM | 2760 | N | ARG | B | 400 | 26.430 | 21.805 | 41.763 | 1.00 | 27.76 | N |
| ATOM | 2761 | CA | ARG | B | 400 | 27.227 | 20.683 | 42.244 | 1.00 | 32.53 | C |
| ATOM | 2762 | CB | ARG | B | 400 | 27.744 | 20.994 | 43.658 | 1.00 | 38.09 | C |
| ATOM | 2763 | CG | ARG | B | 400 | 28.606 | 22.262 | 43.787 | 1.00 | 46.29 | C |
| ATOM | 2764 | CD | ARG | B | 400 | 28.970 | 22.541 | 45.262 | 1.00 | 46.35 | C |
| ATOM | 2765 | NE | ARG | B | 400 | 29.833 | 23.711 | 45.437 | 1.00 | 52.96 | N |
| ATOM | 2766 | CZ | ARG | B | 400 | 31.116 | 23.743 | 45.092 | 1.00 | 55.68 | C |
| ATOM | 2767 | NH1 | ARG | B | 400 | 31.675 | 22.666 | 44.557 | 1.00 | 51.73 | N |
| ATOM | 2768 | NH2 | ARG | B | 400 | 31.841 | 24.847 | 45.278 | 1.00 | 52.56 | N |
| ATOM | 2769 | C | ARG | B | 400 | 26.513 | 19.345 | 42.256 | 1.00 | 27.44 | C |
| ATOM | 2770 | O | ARG | B | 400 | 25.313 | 19.254 | 42.507 | 1.00 | 28.50 | O |
| ATOM | 2771 | N | LEU | B | 401 | 27.259 | 18.280 | 42.006 | 1.00 | 27.68 | N |
| ATOM | 2772 | CA | LEU | B | 401 | 26.695 | 16.948 | 42.018 | 1.00 | 24.03 | C |
| ATOM | 2773 | CB | LEU | B | 401 | 27.514 | 16.006 | 41.156 | 1.00 | 32.24 | C |
| ATOM | 2774 | CG | LEU | B | 401 | 27.086 | 14.540 | 41.184 | 1.00 | 29.66 | C |
| ATOM | 2775 | CD1 | LEU | B | 401 | 25.713 | 14.354 | 40.554 | 1.00 | 35.88 | C |
| ATOM | 2776 | CD2 | LEU | B | 401 | 28.132 | 13.737 | 40.425 | 1.00 | 39.27 | C |
| ATOM | 2777 | C | LEU | B | 401 | 26.745 | 16.426 | 43.456 | 1.00 | 35.79 | C |
| ATOM | 2778 | O | LEU | B | 401 | 27.796 | 16.487 | 44.103 | 1.00 | 29.49 | O |
| ATOM | 2779 | N | ILE | B | 402 | 25.611 | 15.945 | 43.957 | 1.00 | 27.95 | N |
| ATOM | 2780 | CA | ILE | B | 402 | 25.581 | 15.368 | 45.299 | 1.00 | 29.27 | C |
| ATOM | 2781 | CB | ILE | B | 402 | 24.154 | 15.383 | 45.922 | 1.00 | 31.13 | C |
| ATOM | 2782 | CG2 | ILE | B | 402 | 24.188 | 14.716 | 47.305 | 1.00 | 33.30 | C |
| ATOM | 2783 | CG1 | ILE | B | 402 | 23.650 | 16.830 | 46.065 | 1.00 | 26.94 | C |
| ATOM | 2784 | CD1 | ILE | B | 402 | 24.616 | 17.763 | 46.743 | 1.00 | 31.79 | C |
| ATOM | 2785 | C | ILE | B | 402 | 26.053 | 13.921 | 45.119 | 1.00 | 32.38 | C |
| ATOM | 2786 | O | ILE | B | 402 | 26.962 | 13.448 | 45.812 | 1.00 | 31.53 | O |
| ATOM | 2787 | N | GLY | B | 403 | 25.469 | 13.226 | 44.157 | 1.00 | 27.83 | N |
| ATOM | 2788 | CA | GLY | B | 403 | 25.870 | 11.853 | 43.900 | 1.00 | 29.46 | C |
| ATOM | 2789 | C | GLY | B | 403 | 24.897 | 11.150 | 42.994 | 1.00 | 29.35 | C |
| ATOM | 2790 | O | GLY | B | 403 | 23.934 | 11.757 | 42.537 | 1.00 | 30.33 | O |
| ATOM | 2791 | N | VAL | B | 404 | 24.145 | 9.878 | 42.717 | 1.00 | 26.15 | N |
| ATOM | 2792 | CA | VAL | B | 404 | 24.281 | 8.084 | 41.865 | 1.00 | 28.12 | C |
| ATOM | 2793 | CB | VAL | B | 404 | 25.106 | 8.273 | 40.844 | 1.00 | 36.04 | C |
| ATOM | 2794 | CG1 | VAL | B | 404 | 24.186 | 7.397 | 39.982 | 1.00 | 40.40 | C |
| ATOM | 2795 | CG2 | VAL | B | 404 | 25.911 | 9.211 | 39.982 | 1.00 | 35.87 | C |
| ATOM | 2796 | C | VAL | B | 404 | 23.519 | 9.211 | 42.755 | 1.00 | 32.26 | C |
| ATOM | 2797 | O | VAL | B | 404 | 24.052 | 7.647 | 43.744 | 1.00 | 31.47 | O |
| ATOM | 2798 | N | CYS | B | 405 | 22.277 | 7.815 | 42.392 | 1.00 | 30.13 | N |
| ATOM | 2799 | CA | CYS | B | 405 | 21.471 | 6.879 | 43.177 | 1.00 | 35.84 | C |
| ATOM | 2800 | CB | CYS | B | 405 | 20.408 | 7.635 | 43.976 | 1.00 | 39.61 | C |
| ATOM | 2810 | SG | CYS | B | 405 | 19.571 | 6.617 | 45.215 | 1.00 | 43.65 | S |
| ATOM | 2802 | C | CYS | B | 405 | 20.815 | 5.849 | 42.258 | 1.00 | 41.06 | C |
| ATOM | 2803 | O | CYS | B | 405 | 20.101 | 6.200 | 41.320 | 1.00 | 38.70 | O |
| ATOM | 2804 | N | GLN | B | 406 | 21.086 | 4.577 | 42.510 | 1.00 | 41.04 | N |
| ATOM | 2805 | CA | GLN | B | 406 | 20.529 | 3.511 | 41.687 | 1.00 | 49.92 | C |
| ATOM | 2806 | CB | GLN | B | 406 | 21.639 | 2.549 | 41.250 | 1.00 | 53.55 | C |
| ATOM | 2807 | CG | GLN | B | 406 | 21.165 | 1.355 | 40.413 | 1.00 | 59.57 | C |
| ATOM | 2808 | CD | GLN | B | 406 | 20.378 | 1.756 | 39.169 | 1.00 | 56.22 | C |
| ATOM | 2809 | OE1 | GLN | B | 406 | 19.205 | 2.123 | 39.246 | 1.00 | 59.42 | O |
| ATOM | 2810 | NE2 | GLN | B | 406 | 21.028 | 1.687 | 38.016 | 1.00 | 59.35 | N |
| ATOM | 2811 | C | GLN | B | 406 | 19.450 | 2.750 | 42.446 | 1.00 | 53.55 | C |
| ATOM | 2812 | O | GLN | B | 406 | 19.746 | 1.894 | 43.273 | 1.00 | 56.70 | O |
| ATOM | 2813 | N | ALA | B | 407 | 18.197 | 3.080 | 42.163 | 1.00 | 56.23 | N |
| ATOM | 2814 | CA | ALA | B | 407 | 17.069 | 2.418 | 42.808 | 1.00 | 60.79 | C |
| ATOM | 2815 | CB | ALA | B | 407 | 16.432 | 3.359 | 41.742 | 1.00 | 58.91 | C |
| ATOM | 2816 | C | ALA | B | 407 | 16.052 | 20.12 | 41.742 | 1.00 | 58.91 | C |
| ATOM | 2817 | O | ALA | B | 407 | 16.364 | 1.212 | 40.856 | 1.00 | 57.00 | O |
| ATOM | 2818 | N | GLU | B | 408 | 14.846 | 2.568 | 41.821 | 1.00 | 55.11 | N |
| ATOM | 2819 | CA | GLU | B | 408 | 13.806 | 2.260 | 40.849 | 1.00 | 57.75 | C |
| ATOM | 2820 | CB | GLU | B | 408 | 12.535 | 3.065 | 41.147 | 1.00 | 61.87 | C |
| ATOM | 2821 | CG | GLU | B | 408 | 12.759 | 4.562 | 41.299 | 1.00 | 61.03 | C |
| ATOM | 2822 | CD | GLU | B | 408 | 12.938 | 4.998 | 42.748 | 1.00 | 61.45 | C |
| ATOM | 2823 | OE1 | GLU | B | 408 | 13.788 | 4.422 | 43.464 | 1.00 | 62.30 | O |
| ATOM | 2824 | OE2 | GLU | B | 408 | 12.228 | 5.933 | 43.169 | 1.00 | 57.96 | O |
| ATOM | 2825 | C | GLU | B | 408 | 14.320 | 2.590 | 39.452 | 1.00 | 58.74 | C |
| ATOM | 2826 | O | GLU | B | 408 | 13.770 | 2.139 | 38.444 | 1.00 | 61.01 | O |
| ATOM | 2827 | N | ALA | B | 409 | 15.383 | 3.387 | 19.408 | 1.00 | 58.64 | N |
| ATOM | 2828 | CA | ALA | B | 409 | 16.017 | 3.788 | 38.159 | 1.00 | 53.23 | C |
| ATOM | 2829 | CB | ALA | B | 409 | 15.054 | 4.621 | 37.327 | 1.00 | 57.75 | C |
| ATOM | 2830 | C | ALA | B | 409 | 17.262 | 4.595 | 38.518 | 1.00 | 48.64 | C |
| ATOM | 2831 | O | ALA | B | 409 | 17.451 | 4.957 | 39.682 | 1.00 | 41.77 | O |
| ATOM | 2832 | N | LEU | B | 410 | 18.113 | 4.859 | 37.527 | 1.00 | 46.67 | N |
| ATOM | 2833 | CA | LEU | B | 410 | 19.348 | 5.606 | 37.757 | 1.00 | 41.56 | C |
| ATOM | 2834 | CB | LEU | B | 410 | 20.326 | 5.370 | 36.607 | 1.00 | 41.30 | C |
| ATOM | 2835 | CG | LEU | B | 410 | 21.745 | 5.849 | 36.893 | 1.00 | 39.49 | C |
| ATOM | 2836 | CD1 | LEU | B | 410 | 22.282 | 5.132 | 38.127 | 1.00 | 40.07 | C |
| ATOM | 2837 | CD2 | LEU | B | 410 | 22.628 | 5.590 | 35.680 | 1.00 | 44.22 | C |
| ATOM | 2838 | C | LEU | B | 410 | 19.024 | 7.087 | 37.877 | 1.00 | 36.27 | C |
| ATOM | 2830 | O | LEU | B | 410 | 18.291 | 7.631 | 37.068 | 1.00 | 32.58 | O |
| ATOM | 2840 | N | MET | B | 411 | 19.579 | 7.740 | 38.888 | 1.00 | 31.99 | N |
| ATOM | 2841 | CA | MET | B | 411 | 19.277 | 9.144 | 39.105 | 1.00 | 28.85 | C |
| ATOM | 2842 | CB | MET | B | 411 | 18.272 | 9.282 | 40.261 | 1.00 | 27.82 | C |
| ATOM | 2843 | CG | MET | B | 411 | 16.908 | 8.467 | 40.120 | 1.00 | 38.03 | C |
| ATOM | 2844 | SD | MET | B | 411 | 15.902 | 8.655 | 41.550 | 1.00 | 39.66 | S |
| ATOM | 2845 | CE | MET | B | 411 | 16.875 | 7.893 | 42.769 | 1.00 | 39.08 | C |
| ATOM | 2846 | C | MET | B | 411 | 20.518 | 9.931 | 39.454 | 1.00 | 34.64 | C |
| ATOM | 2847 | O | MET | B | 411 | 21.415 | 9.412 | 40.110 | 1.00 | 32.86 | O |
| ATOM | 2848 | N | LEU | B | 412 | 20.576 | 11.182 | 39.012 | 1.00 | 27.07 | N |
| ATOM | 2849 | CA | LEU | B | 412 | 21.686 | 12.070 | 39.353 | 1.00 | 24.70 | C |
| ATOM | 2850 | CB | LEU | B | 412 | 22.213 | 12.819 | 38.22 | 1.00 | 31.40 | C |
| ATOM | 2851 | CG | LEU | B | 412 | 23.072 | 12.039 | 37.146 | 1.00 | 42.38 | C |
| ATOM | 2852 | CD1 | LEU | B | 412 | 23.695 | 13.008 | 36.127 | 1.00 | 40.09 | C |
| ATOM | 2853 | CD2 | LEU | B | 412 | 24.156 | 11.325 | 37.940 | 1.00 | 44.83 | C |
| ATOM | 2854 | C | LEU | B | 412 | 21.108 | 13.086 | 40.316 | 1.00 | 32.72 | C |
| ATOM | 2855 | O | LEU | B | 412 | 20.203 | 13.829 | 39.930 | 1.00 | 25.93 | O |
| ATOM | 2856 | N | VAL | B | 413 | 21.632 | 13.118 | 41.552 | 1.00 | 25.13 | N |
| ATOM | 2857 | CA | VAL | B | 413 | 21.179 | 14.048 | 42.591 | 1.00 | 22.67 | C |
| ATOM | 2858 | CB | VAL | B | 413 | 21.169 | 13.374 | 43.997 | 1.00 | 27.42 | C |
| ATOM | 2859 | CG1 | VAL | B | 413 | 20.591 | 14.333 | 45.040 | 1.00 | 22.22 | C |
| ATOM | 2860 | CG2 | VAL | B | 413 | 20.369 | 12.079 | 43.932 | 1.00 | 27.93 | C |
| ATOM | 2861 | C | VAL | B | 413 | 22.099 | 15.245 | 42.627 | 1.00 | 26.70 | C |
| ATOM | 2862 | O | VAL | B | 413 | 23.317 | 15.115 | 42.869 | 1.00 | 28.13 | O |
| ATOM | 2863 | N | MET | B | 414 | 21.503 | 16.417 | 42.414 | 1.00 | 23.91 | N |
| ATOM | 2864 | CA | MET | B | 414 | 22.232 | 17.672 | 42.353 | 1.00 | 23.05 | C |
| ATOM | 2865 | CB | MET | B | 414 | 22.146 | 18.241 | 40.9131 | 1.00 | 26.27 | C |
| ATOM | 2866 | CG | MET | B | 414 | 22.782 | 17.373 | 39.850 | 1.00 | 31.80 | C |
| ATOM | 2867 | SD | MET | B | 414 | 22.187 | 17.909 | 38.179 | 1.00 | 31.87 | S |
| ATOM | 2868 | CE | MET | B | 414 | 20.682 | 16.814 | 38.038 | 1.00 | 34.99 | C |
| ATOM | 2869 | C | MET | B | 414 | 21.683 | 18.729 | 43.269 | 1.00 | 24.82 | C |
| ATOM | 2870 | O | MET | B | 414 | 20.536 | 18.646 | 43.732 | 1.00 | 26.89 | O |
| ATOM | 2871 | N | GLU | B | 415 | 22.475 | 19.767 | 43.508 | 1.00 | 22.82 | N |
| ATOM | 2872 | CA | GLU | B | 415 | 22.008 | 20.867 | 44.330 | 1.00 | 25.46 | C |
| ATOM | 2873 | CB | GLU | B | 415 | 23.089 | 21.939 | 44.500 | 1.00 | 38.09 | C |
| ATOM | 2874 | CG | GLU | B | 415 | 24.381 | 21.466 | 45.121 | 1.00 | 41.76 | C |
| ATOM | 2875 | CD | GLU | B | 415 | 25.406 | 22.600 | 45.191 | 1.00 | 56.15 | C |
| ATOM | 2876 | OE1 | GLU | B | 415 | 25.611 | 23.293 | 44.153 | 1.00 | 43.26 | O |
| ATOM | 2877 | OE2 | GLU | B | 415 | 26.000 | 22.796 | 46.279 | 1.00 | 53.48 | O |
| ATOM | 2878 | C | GLU | B | 415 | 20.848 | 21.511 | 43.581 | 1.00 | 28.34 | C |
| ATOM | 2879 | O | GLU | B | 415 | 20.819 | 21.496 | 42.347 | 1.00 | 27.84 | O |
| ATOM | 2880 | N | MET | B | 416 | 19.903 | 22.079 | 44.327 | 1.00 | 28.24 | N |
| ATOM | 2881 | CA | MET | B | 416 | 18.776 | 22.739 | 43.701 | 1.00 | 27.37 | C |
| ATOM | 2882 | CB | MET | B | 416 | 17.482 | 22.444 | 44.452 | 1.00 | 31.26 | C |
| ATOM | 2883 | CG | MET | B | 416 | 16.245 | 22.889 | 43.658 | 1.00 | 33.79 | C |
| ATOM | 2884 | SD | MET | B | 416 | 14.717 | 22.640 | 44.566 | 1.00 | 40.24 | S |
| ATOM | 2885 | CE | MET | B | 416 | 13.622 | 23.863 | 43.732 | 1.00 | 41.28 | C |
| ATOM | 2886 | C | MET | B | 416 | 18.989 | 24.247 | 43.709 | 1.00 | 32.53 | C |
| ATOM | 2887 | O | MET | B | 416 | 19.462 | 24.797 | 44.695 | 1.00 | 29.31 | O |
| ATOM | 2888 | N | ALA | B | 417 | 18.639 | 24.900 | 42.603 | 1.00 | 29.63 | N |
| ATOM | 2889 | CA | ALA | B | 417 | 18.724 | 26.362 | 42.497 | 1.00 | 32.11 | C |
| ATOM | 2890 | CB | ALA | B | 417 | 19.676 | 26.775 | 41.365 | 1.00 | 31.26 | C |
| ATOM | 2891 | C | ALA | B | 417 | 17.282 | 26.779 | 42.186 | 1.00 | 32.20 | C |
| ATOM | 2892 | O | ALA | B | 417 | 16.903 | 26.933 | 41.017 | 1.00 | 31.90 | O |
| ATOM | 2893 | N | GLY | B | 418 | 16.500 | 26.924 | 43.259 | 1.00 | 34.38 | N |
| ATOM | 2894 | CA | GLY | B | 418 | 15.081 | 27.243 | 43.183 | 1.00 | 36.34 | C |
| ATOM | 2895 | C | GLY | B | 418 | 14.630 | 28.461 | 42.398 | 1.00 | 34.85 | C |
| ATOM | 2896 | O | GLY | B | 418 | 13.447 | 28.598 | 42.082 | 1.00 | 34.44 | O |
| ATOM | 2897 | N | GLY | B | 419 | 15.563 | 29.359 | 42.105 | 1.00 | 28.57 | N |
| ATOM | 2898 | CA | GLY | B | 419 | 15.208 | 30.537 | 41.339 | 1.00 | 30.78 | C |
| ATOM | 2899 | C | GLY | B | 419 | 14.891 | 30.180 | 39.897 | 1.00 | 33.81 | C |
| ATOM | 2900 | O | GLY | B | 419 | 14.245 | 30.944 | 39.202 | 1.00 | 32.35 | O |
| ATOM | 2901 | N | GLY | B | 420 | 15.370 | 29.032 | 39.427 | 1.00 | 30.29 | N |
| ATOM | 2902 | CA | GLY | B | 420 | 15.091 | 28.621 | 38.065 | 1.00 | 27.83 | C |
| ATOM | 2903 | C | GLY | B | 420 | 15.930 | 29.299 | 36.980 | 1.00 | 24.88 | C |
| ATOM | 2904 | O | GLY | B | 420 | 16.843 | 30.054 | 37.303 | 1.00 | 24.54 | O |
| ATOM | 2905 | N | PRO | B | 421 | 15.617 | 29.068 | 35.694 | 1.00 | 26.97 | N |
| ATOM | 2906 | CD | PRO | B | 421 | 14.445 | 28.280 | 35.256 | 1.00 | 26.58 | C |
| ATOM | 2907 | CA | PRO | B | 421 | 16.320 | 29.631 | 34.534 | 1.00 | 26.79 | C |
| ATOM | 2908 | CB | PRO | B | 421 | 15.508 | 29.113 | 33.335 | 1.00 | 31.97 | C |
| ATOM | 2909 | CG | PRO | B | 421 | 14.810 | 27.921 | 33.845 | 1.00 | 32.59 | C |
| ATOM | 2910 | C | PRO | B | 421 | 16.400 | 31.148 | 34.507 | 1.00 | 31.93 | C |
| ATOM | 2911 | O | PRO | B | 421 | 15.430 | 31.851 | 34.801 | 1.00 | 26.82 | O |
| ATOM | 2912 | N | LEU | B | 422 | 17.570 | 31.643 | 34.112 | 1.00 | 24.30 | N |
| ATOM | 2913 | CA | LEU | B | 422 | 17.848 | 33.064 | 34.025 | 1.00 | 25.19 | C |
| ATOM | 2914 | CB | LEU | B | 422 | 19.316 | 33.263 | 33.632 | 1.00 | 26.63 | C |
| ATOM | 2915 | CG | LEU | B | 422 | 19.835 | 34.685 | 33.470 | 1.00 | 27.37 | C |
| ATOM | 2916 | CD1 | LEU | B | 422 | 19.814 | 35.394 | 34.801 | 1.00 | 28.60 | C |
| ATOM | 2917 | CD2 | LEU | B | 422 | 21.251 | 34.623 | 32.917 | 1.00 | 25.74 | C |
| ATOM | 2918 | C | LEU | B | 422 | 16.966 | 33.799 | 33.003 | 1.00 | 22.35 | C |
| ATOM | 2919 | O | LEU | B | 422 | 16.524 | 34.916 | 33.268 | 1.00 | 27.34 | O |
| ATOM | 2920 | N | HIS | B | 423 | 16.753 | 33.207 | 31.835 | 1.00 | 23.59 | N |
| ATOM | 2921 | CA | HIS | B | 423 | 15.944 | 33.884 | 30.829 | 1.00 | 33.17 | C |
| ATOM | 2922 | CB | HIS | B | 423 | 15.899 | 33.099 | 29.518 | 1.00 | 33.79 | C |
| ATOM | 2923 | CG | HIS | B | 423 | 15.322 | 31.725 | 29.650 | 1.00 | 30.44 | C |
| ATOM | 2924 | CD2 | HIS | B | 423 | 15.309 | 30.856 | 30.688 | 1.00 | 33.89 | C |
| ATOM | 2925 | ND1 | HIS | B | 423 | 14.631 | 31.109 | 28.626 | 1.00 | 40.96 | N |
| ATOM | 2926 | CD1 | HIS | B | 423 | 14.222 | 29.919 | 29.029 | 1.00 | 35.92 | C |
| ATOM | 2927 | NE2 | HIS | B | 423 | 14.621 | 29.740 | 30.276 | 1.00 | 37.97 | N |
| ATOM | 2928 | C | HIS | B | 423 | 14.525 | 34.142 | 31.341 | 1.00 | 37.06 | C |
| ATOM | 2929 | O | HIS | B | 423 | 13.989 | 35.236 | 31.153 | 1.00 | 39.39 | O |
| ATOM | 2930 | N | LYS | B | 424 | 13.927 | 33.149 | 31.995 | 1.00 | 28.98 | N |
| ATOM | 2931 | CA | LYS | B | 424 | 12.576 | 33.351 | 32.510 | 1.00 | 31.22 | C |
| ATOM | 2932 | CB | LYS | B | 424 | 11.957 | 32.006 | 32.883 | 1.00 | 28.63 | C |
| ATOM | 2933 | CG | LYS | B | 424 | 11.695 | 31.136 | 31.674 | 1.00 | 29.72 | C |
| ATOM | 2934 | CD | LYS | B | 424 | 11.076 | 29.800 | 32.117 | 1.00 | 42.69 | C |
| ATOM | 2935 | CE | LYS | B | 424 | 10.935 | 28.829 | 30.955 | 1.00 | 47.20 | C |
| ATOM | 2936 | NZ | LYS | B | 424 | 9.949 | 27.753 | 31.294 | 1.00 | 54.05 | N |
| ATOM | 2937 | C | LYS | B | 424 | 12.554 | 34.296 | 33.700 | 1.00 | 33.37 | C |
| ATOM | 2938 | O | LYS | B | 424 | 11.608 | 35.064 | 33.878 | 1.00 | 28.31 | O |
| ATOM | 2939 | N | PHE | B | 425 | 13.602 | 34.269 | 34.519 | 1.00 | 28.20 | N |
| ATOM | 2940 | CA | PHE | B | 425 | 13.660 | 35.137 | 35.681 | 1.00 | 26.95 | C |
| ATOM | 2941 | CB | PHE | B | 425 | 14.890 | 34.802 | 36.540 | 1.00 | 31.21 | C |
| ATOM | 2942 | CG | PHE | B | 425 | 15.042 | 35.680 | 37.747 | 1.00 | 32.76 | C |
| ATOM | 2943 | CD1 | PHE | B | 425 | 14.318 | 35.431 | 38.904 | 1.00 | 41.67 | C |
| ATOM | 2944 | CD2 | PHE | B | 425 | 15.886 | 36.782 | 37.710 | 1.00 | 35.66 | C |
| ATOM | 2945 | CE1 | PHE | B | 425 | 14.429 | 36.272 | 40.011 | 1.00 | 40.79 | C |
| ATOM | 2946 | CE2 | PHE | B | 425 | 16.010 | 37.631 | 38.805 | 1.00 | 43.64 | C |
| ATOM | 2947 | CZ | PHE | B | 425 | 15.277 | 37.377 | 39.962 | 1.00 | 45.58 | C |
| ATOM | 2948 | C | PHE | B | 425 | 13.713 | 36.629 | 35.310 | 1.00 | 28.99 | C |
| ATOM | 2949 | O | PHE | B | 425 | 13.149 | 37.468 | 36.010 | 1.00 | 28.02 | O |
| ATOM | 2950 | N | LEU | B | 426 | 14.379 | 36.961 | 34.212 | 1.00 | 27.54 | N |
| ATOM | 2951 | CA | LEU | B | 426 | 14.522 | 38.373 | 33.838 | 1.00 | 23.94 | C |
| ATOM | 2952 | CB | LEU | B | 426 | 15.838 | 38.577 | 33.072 | 1.00 | 25.46 | C |
| ATOM | 2953 | CG | LEU | B | 426 | 17.102 | 38.323 | 33.905 | 1.00 | 23.52 | C |
| ATOM | 2954 | CD1 | LEU | B | 426 | 18.345 | 38.424 | 33.014 | 1.00 | 25.22 | C |
| ATOM | 2955 | CD2 | LEU | B | 426 | 17.165 | 39.329 | 35.029 | 1.00 | 28.05 | C |
| ATOM | 2956 | C | LEU | B | 426 | 13.368 | 38.959 | 33.019 | 1.00 | 28.74 | C |
| ATOM | 2957 | O | LEU | B | 426 | 13.236 | 40.186 | 32.937 | 1.00 | 26.14 | O |
| ATOM | 2958 | N | VAL | B | 427 | 12.553 | 38.100 | 32.415 | 1.00 | 27.70 | N |
| ATOM | 2959 | CA | VAL | B | 427 | 11.442 | 38.584 | 31.583 | 1.00 | 30.96 | C |
| ATOM | 2960 | CB | VAL | B | 427 | 10.577 | 37.409 | 31.059 | 1.00 | 35.17 | C |
| ATOM | 2961 | CG1 | VAL | B | 427 | 9.323 | 37.939 | 30.343 | 1.00 | 34.67 | C |
| ATOM | 2962 | CG2 | VAL | B | 427 | 11.391 | 36.561 | 30.082 | 1.00 | 31.17 | C |
| ATOM | 2963 | C | VAL | B | 427 | 10.551 | 39.581 | 32.331 | 1.00 | 36.19 | C |
| ATOM | 2964 | O | VAL | B | 427 | 9.984 | 39.269 | 33.386 | 1.00 | 37.99 | O |
| ATOM | 2965 | N | GLY | B | 428 | 10.454 | 40.791 | 31.788 | 1.00 | 44.92 | N |
| ATOM | 2966 | CA | GLY | B | 428 | 9.637 | 41.830 | 32.397 | 1.00 | 39.36 | C |
| ATOM | 2967 | C | GLY | B | 428 | 10.144 | 42.432 | 33.696 | 1.00 | 46.68 | C |
| ATOM | 2968 | A | GLY | B | 428 | 9.384 | 43.091 | 34.410 | 1.00 | 43.86 | O |
| ATOM | 2969 | N | LYS | B | 429 | 11.419 | 42.231 | 34.019 | 1.00 | 36.36 | N |
| ATOM | 2970 | CA | LYS | B | 429 | 11.960 | 42.772 | 35.258 | 1.00 | 41.33 | C |
| ATOM | 2971 | CB | LYS | B | 429 | 12.561 | 41.638 | 36.068 | 1.00 | 45.43 | C |
| ATOM | 2972 | CG | LYS | B | 429 | 11.519 | 40.599 | 36.492 | 1.00 | 48.96 | C |
| ATOM | 2973 | CD | LYS | B | 429 | 11.975 | 39.804 | 37.699 | 1.00 | 53.23 | C |
| ATOM | 2974 | CE | LYS | B | 429 | 11.004 | 38.680 | 38.025 | 1.00 | 52.24 | C |
| ATOM | 2975 | NZ | LYS | B | 429 | 11.552 | 37.819 | 39.108 | 1.00 | 60.84 | N |
| ATOM | 2976 | C | LYS | B | 429 | 12.990 | 43.880 | 35.028 | 1.00 | 42.92 | C |
| ATOM | 2977 | O | LYS | B | 429 | 13.841 | 44.152 | 35.883 | 1.00 | 41.32 | O |
| ATOM | 2978 | N | ARG | B | 430 | 12.889 | 44.535 | 33.878 | 1.00 | 38.04 | N |
| ATOM | 2979 | CA | ARG | B | 430 | 13.824 | 45.954 | 33.532 | 1.00 | 50.47 | C |
| ATOM | 2980 | CB | ARG | B | 430 | 13.541 | 46.091 | 32.116 | 1.00 | 52.47 | C |
| ATOM | 2981 | CG | ARG | B | 430 | 14.669 | 46.918 | 31.520 | 1.00 | 63.31 | C |
| ATOM | 2982 | CD | ARG | B | 430 | 14.548 | 47.014 | 29.999 | 1.00 | 63.30 | C |
| ATOM | 2983 | NE | ARG | B | 430 | 15.857 | 46.896 | 29.351 | 1.00 | 63.29 | N |
| ATOM | 2984 | CZ | ARG | B | 430 | 16.061 | 46.254 | 28.203 | 1.00 | 68.48 | C |
| ATOM | 2985 | NH1 | ARG | B | 430 | 15.043 | 45.680 | 27.573 | 1.00 | 63.45 | N |
| ATOM | 2986 | NH2 | ARG | B | 430 | 17.294 | 46.151 | 27.698 | 1.00 | 66.07 | N |
| ATOM | 2987 | C | ARG | B | 430 | 13.774 | 46.754 | 34.518 | 1.00 | 53.77 | C |
| ATOM | 2988 | O | ARG | B | 430 | 14.727 | 47.516 | 36.643 | 1.00 | 53.09 | O |
| ATOM | 2989 | N | GLU | B | 431 | 12.661 | 46.879 | 35.235 | 1.00 | 54.55 | N |
| ATOM | 2990 | CA | GLU | B | 431 | 12.518 | 47.959 | 36.196 | 1.00 | 55.49 | C |
| ATOM | 2991 | CB | GLU | B | 431 | 11.167 | 48.658 | 36.020 | 1.00 | 57.64 | C |
| ATOM | 2992 | CG | GLU | B | 431 | 11.104 | 49.556 | 34.797 | 1.00 | 61.36 | C |
| ATOM | 2993 | CD | GLU | B | 431 | 9.932 | 50.515 | 34.839 | 1.00 | 66.74 | C |
| ATOM | 2994 | OE1 | GLU | B | 431 | 9.882 | 51.356 | 35.767 | 1.00 | 71.47 | O |
| ATOM | 2995 | OE2 | GLU | B | 431 | 9.062 | 50.433 | 33.946 | 1.00 | 68.60 | O |
| ATOM | 2996 | C | GLU | B | 431 | 12.683 | 47.510 | 37.635 | 1.00 | 56.39 | C |
| ATOM | 2997 | O | GLU | B | 431 | 12.608 | 48.325 | 38.550 | 1.00 | 57.92 | O |
| ATOM | 2998 | N | GLU | B | 432 | 12.910 | 46.218 | 37.844 | 1.00 | 51.06 | N |
| ATOM | 2999 | CA | GLU | B | 432 | 13.091 | 45.720 | 39.194 | 1.00 | 50.31 | C |
| ATOM | 3000 | CB | GLU | B | 432 | 11.992 | 44.713 | 39.554 | 1.00 | 56.46 | C |
| ATOM | 3001 | CG | GLU | B | 432 | 12.301 | 43.274 | 39.217 | 1.00 | 60.14 | C |
| ATOM | 3002 | CD | GLU | B | 432 | 11.319 | 42.313 | 39.866 | 1.00 | 63.83 | C |
| ATOM | 3003 | OE1 | GLU | B | 432 | 10.118 | 42.313 | 39.866 | 1.00 | 63.83 | O |
| ATOM | 3004 | OE2 | GLU | B | 432 | 11.746 | 41.501 | 40.719 | 1.00 | 66.50 | O |
| ATOM | 3005 | C | GLU | B | 432 | 14.464 | 45.083 | 39.364 | 1.00 | 49.21 | C |
| ATOM | 3006 | O | GLU | B | 432 | 14.932 | 44.892 | 40.483 | 1.00 | 47.48 | O |
| ATOM | 3007 | N | ILE | B | 433 | 15.111 | 44.756 | 38.246 | 1.00 | 43.34 | N |
| ATOM | 3008 | CA | ILE | B | 433 | 16.442 | 44.156 | 38.288 | 1.00 | 35.43 | C |
| ATOM | 3009 | CB | ILE | B | 433 | 16.436 | 42.734 | 37.688 | 1.00 | 40.48 | C |
| ATOM | 3010 | CG2 | ILE | B | 433 | 17.842 | 42.125 | 37.754 | 1.00 | 33.02 | C |
| ATOM | 3011 | CG1 | ILE | B | 433 | 15.463 | 41.850 | 38.460 | 1.00 | 39.44 | C |
| ATOM | 3012 | CD1 | ILE | B | 433 | 15.862 | 41.633 | 39.896 | 1.00 | 42.99 | C |
| ATOM | 3013 | C | ILE | B | 433 | 17.335 | 45.049 | 37.446 | 1.00 | 37.58 | C |
| ATOM | 3014 | O | ILE | B | 433 | 17.309 | 44.975 | 36.217 | 1.00 | 33.88 | O |
| ATOM | 3015 | N | PRO | B | 434 | 18.125 | 45.919 | 38.097 | 1.00 | 33.22 | N |
| ATOM | 3016 | CD | PRO | B | 434 | 18.093 | 46.179 | 39.546 | 1.00 | 39.68 | C |
| ATOM | 3017 | CA | PRO | B | 434 | 19.038 | 46.857 | 37.435 | 1.00 | 37.36 | C |
| ATOM | 3018 | CB | PRO | B | 434 | 19.661 | 47.606 | 38.614 | 1.00 | 36.58 | C |
| ATOM | 3019 | CG | PRO | B | 434 | 18.559 | 47.616 | 39.604 | 1.00 | 47.33 | C |
| ATOM | 3020 | C | PRO | B | 434 | 20.083 | 46.163 | 36.578 | 1.00 | 35.02 | C |
| ATOM | 3021 | O | PRO | B | 434 | 20.382 | 44.986 | 36.785 | 1.00 | 29.87 | O |
| ATOM | 3022 | N | VAL | B | 435 | 20.644 | 46.904 | 35.625 | 1.00 | 30.90 | N |
| ATOM | 3023 | CA | VAL | B | 435 | 21.699 | 46.349 | 34.750 | 1.00 | 33.32 | C |
| ATOM | 3024 | CB | VAL | B | 435 | 22.138 | 47.391 | 33.699 | 1.00 | 32.31 | C |
| ATOM | 3025 | CG1 | VAL | B | 435 | 23.255 | 46.802 | 32.839 | 1.00 | 32.23 | C |
| ATOM | 3026 | CG2 | VAL | B | 435 | 20.968 | 47.792 | 32.821 | 1.00 | 38.32 | C |
| ATOM | 3027 | C | VAL | B | 435 | 22.862 | 45.875 | 32.570 | 1.00 | 31.88 | C |
| ATOM | 3028 | O | VAL | B | 435 | 23.504 | 44.873 | 35.218 | 1.00 | 26.54 | O |
| ATOM | 3029 | N | SER | B | 436 | 23.165 | 46.593 | 36.652 | 1.00 | 28.96 | N |
| ATOM | 3030 | CA | SER | B | 436 | 24.278 | 46.225 | 37.519 | 1.00 | 29.85 | C |
| ATOM | 3031 | CB | SER | B | 436 | 24.437 | 47.262 | 38.646 | 1.00 | 33.71 | C |
| ATOM | 3032 | OG | SER | B | 436 | 23.246 | 47.361 | 39.414 | 1.00 | 36.37 | O |
| ATOM | 3033 | C | SER | B | 436 | 24.077 | 44.815 | 38.112 | 1.00 | 30.64 | C |
| ATOM | 3034 | O | SER | B | 436 | 25.039 | 44.069 | 38.281 | 1.00 | 28.51 | O |
| ATOM | 3035 | N | ASN | B | 437 | 22.832 | 44.464 | 38.426 | 1.00 | 25.41 | N |
| ATOM | 3036 | CA | ASN | B | 437 | 22.502 | 43.155 | 38.993 | 1.00 | 26.86 | C |
| ATOM | 3037 | CB | ASN | B | 437 | 21.088 | 43.219 | 39.581 | 1.00 | 29.47 | C |
| ATOM | 3038 | CG | ASN | B | 437 | 20.692 | 41.975 | 40.391 | 1.00 | 32.65 | C |
| ATOM | 3039 | OD1 | ASN | B | 437 | 19.669 | 41.997 | 41.081 | 1.00 | 33.91 | O |
| ATOM | 3040 | ND2 | ASN | B | 437 | 21.466 | 40.891 | 40.294 | 1.00 | 29.62 | N |
| ATOM | 3041 | C | ASN | B | 437 | 22.632 | 42.121 | 37.858 | 1.00 | 26.75 | C |
| ATOM | 3042 | O | ASN | B | 437 | 23.126 | 41.010 | 38.070 | 1.00 | 26.10 | O |
| ATOM | 3043 | N | VAL | B | 438 | 22.220 | 42.483 | 36.648 | 1.00 | 26.29 | N |
| ATOM | 3044 | CA | VAL | B | 438 | 22.390 | 41.546 | 35.548 | 1.00 | 22.79 | C |
| ATOM | 3045 | CB | VAL | B | 438 | 21.761 | 42.073 | 34.250 | 1.00 | 26.63 | C |
| ATOM | 3046 | CG1 | VAL | B | 438 | 22.085 | 41.136 | 33.099 | 1.00 | 25.60 | C |
| ATOM | 3047 | CG2 | VAL | B | 438 | 20.231 | 42.187 | 34.428 | 1.00 | 27.00 | C |
| ATOM | 3048 | C | VAL | B | 438 | 23.896 | 41.313 | 35.343 | 1.00 | 25.06 | C |
| ATOM | 3049 | O | VAL | B | 438 | 24.325 | 40.178 | 35.172 | 1.00 | 22.07 | O |
| ATOM | 3050 | N | ALA | B | 439 | 24.704 | 42.383 | 35.376 | 1.00 | 23.57 | N |
| ATOM | 3051 | CA | ALA | B | 439 | 26.156 | 42.223 | 35.206 | 1.00 | 24.57 | C |
| ATOM | 3052 | CB | ALA | B | 439 | 26.858 | 43.623 | 35.135 | 1.00 | 23.14 | C |
| ATOM | 3053 | C | ALA | B | 439 | 26.775 | 41.369 | 36.326 | 1.00 | 21.99 | C |
| ATOM | 3054 | O | ALA | B | 439 | 27.724 | 40.622 | 36.086 | 1.00 | 23.36 | O |
| ATOM | 3055 | N | GLU | B | 440 | 26.225 | 41.467 | 37.540 | 1.00 | 22.47 | N |
| ATOM | 3056 | CA | GLU | B | 440 | 26.729 | 40.684 | 38.660 | 1.00 | 26.63 | C |
| ATOM | 3057 | CB | GLU | B | 440 | 25.946 | 41.027 | 39.937 | 1.00 | 28.89 | C |
| ATOM | 3058 | CG | GLU | B | 440 | 26.220 | 40.080 | 41.109 | 1.00 | 27.35 | C |
| ATOM | 3059 | CD | GLU | B | 440 | 25.304 | 40.355 | 42.295 | 1.00 | 39.88 | C |
| ATOM | 3060 | OE1 | GLU | B | 440 | 24.142 | 40.766 | 42.075 | 1.00 | 37.76 | O |
| ATOM | 3061 | OE2 | GLU | B | 440 | 25.750 | 40.145 | 43.445 | 1.00 | 37.86 | O |
| ATOM | 3062 | C | GLU | B | 440 | 26.575 | 39.190 | 38.331 | 1.00 | 28.92 | C |
| ATOM | 3063 | O | GLU | B | 440 | 27.503 | 38.411 | 38.506 | 1.00 | 22.24 | O |
| ATOM | 3064 | N | LEU | B | 441 | 25.393 | 38.812 | 37.848 | 1.00 | 22.17 | N |
| ATOM | 3065 | CA | LEU | B | 441 | 25.086 | 37.423 | 37.488 | 1.00 | 22.51 | C |
| ATOM | 3066 | CB | LEU | B | 441 | 23.590 | 37.301 | 37.148 | 1.00 | 22.22 | C |
| ATOM | 3067 | CG | LEU | B | 441 | 22.647 | 37.692 | 38.290 | 1.00 | 26.11 | C |
| ATOM | 3068 | CD1 | LEU | B | 441 | 21.191 | 37.675 | 37.823 | 1.00 | 26.08 | C |
| ATOM | 3069 | CD2 | LEU | B | 441 | 22.853 | 36.702 | 39.443 | 1.00 | 23.20 | C |
| ATOM | 3070 | C | LEU | B | 441 | 25.930 | 36.973 | 36.296 | 1.00 | 20.83 | C |
| ATOM | 3071 | O | LEU | B | 441 | 26.449 | 35.848 | 36.280 | 1.00 | 22.40 | O |
| ATOM | 3072 | N | LEU | B | 442 | 26.062 | 37.829 | 35.290 | 1.00 | 20.51 | N |
| ATOM | 3073 | CA | LEU | B | 442 | 26.893 | 37.442 | 34.147 | 1.00 | 24.46 | C |
| ATOM | 3074 | CB | LEU | B | 442 | 26.822 | 38.487 | 33.038 | 1.00 | 22.94 | C |
| ATOM | 3075 | CG | LEU | B | 442 | 25.484 | 38.527 | 32.291 | 1.00 | 21.56 | C |
| ATOM | 3076 | CD1 | LEU | B | 442 | 25.459 | 39.693 | 31.263 | 1.00 | 24.52 | C |
| ATOM | 3077 | CD2 | LEU | B | 442 | 25.268 | 37.168 | 31.601 | 1.00 | 22.91 | C |
| ATOM | 3078 | C | LEU | B | 442 | 28.346 | 37.244 | 34.590 | 1.00 | 21.94 | C |
| ATOM | 3079 | O | LEU | B | 442 | 29.033 | 36.352 | 34.105 | 1.00 | 22.31 | O |
| ATOM | 3080 | N | HIS | B | 443 | 28.811 | 38.055 | 35.530 | 1.00 | 20.83 | N |
| ATOM | 3081 | CA | HIS | B | 443 | 30.193 | 37.888 | 35.998 | 1.00 | 21.14 | C |
| ATOM | 3002 | CB | HIS | B | 443 | 30.598 | 39.048 | 36.918 | 1.00 | 25.52 | C |
| ATOM | 3083 | CG | HIS | B | 443 | 31.965 | 38.875 | 37.502 | 1.00 | 27.68 | C |
| ATOM | 3084 | CD2 | HIS | B | 443 | 33.143 | 38.507 | 36.932 | 1.00 | 25.68 | C |
| ATOM | 3085 | ND1 | HIS | B | 443 | 32.210 | 38.987 | 38.855 | 1.00 | 26.29 | N |
| ATOM | 3086 | CE1 | HIS | B | 443 | 33.476 | 38.691 | 39.096 | 1.00 | 24.28 | C |
| ATOM | 3087 | NE2 | HIS | B | 443 | 34.064 | 38.394 | 37.947 | 1.00 | 23.65 | N |
| ATOM | 3088 | C | HIS | B | 443 | 30.367 | 36.530 | 36.715 | 1.00 | 22.96 | C |
| ATOM | 3089 | O | HIS | B | 443 | 31.374 | 35.841 | 36.541 | 1.00 | 21.47 | O |
| ATOM | 3090 | N | GLN | B | 444 | 29.370 | 36.130 | 37.498 | 1.00 | 22.87 | N |
| ATOM | 3091 | CA | GLN | B | 444 | 29.429 | 34.843 | 38.177 | 1.00 | 19.38 | C |
| ATOM | 3092 | CB | GLN | B | 444 | 28.203 | 34.688 | 39.080 | 1.00 | 21.91 | C |
| ATOM | 3093 | CG | GLN | B | 444 | 28.254 | 35.697 | 40.221 | 1.00 | 22.66 | C |
| ATOM | 3094 | CD | GLN | B | 444 | 27.013 | 35.648 | 41.058 | 1.00 | 25.66 | C |
| ATOM | 3095 | OE1 | GLN | B | 444 | 25.994 | 35.130 | 40.633 | 1.00 | 27.50 | O |
| ATOM | 3096 | NE2 | GLN | B | 444 | 27.089 | 36.206 | 42.258 | 1.00 | 22.05 | C |
| ATOM | 3097 | C | GLN | B | 444 | 29.483 | 33.735 | 37.149 | 1.00 | 22.05 | C |
| ATOM | 3098 | O | GLN | B | 444 | 30.248 | 32.792 | 37.288 | 1.00 | 23.64 | O |
| ATOM | 3099 | N | VAL | B | 445 | 28.691 | 33.867 | 36.089 | 1.00 | 20.47 | N |
| ATOM | 3100 | CA | VAL | B | 445 | 28.735 | 32.863 | 35.044 | 1.00 | 19.55 | C |
| ATOM | 3101 | CB | VAL | B | 445 | 27.705 | 33.147 | 33.926 | 1.00 | 20.65 | C |
| ATOM | 3102 | CG1 | VAL | B | 445 | 27.876 | 32.121 | 32.784 | 1.00 | 21.65 | C |
| ATOM | 3103 | CG2 | VAL | B | 445 | 26.300 | 33.049 | 34.493 | 1.00 | 21.06 | C |
| ATOM | 3104 | C | VAL | B | 445 | 30.151 | 32.857 | 34.435 | 1.00 | 21.63 | C |
| ATOM | 3105 | O | VAL | B | 445 | 30.682 | 31.792 | 34.150 | 1.00 | 21.53 | O |
| ATOM | 3106 | N | SER | B | 446 | 30.757 | 34.031 | 34.251 | 1.00 | 19.30 | N |
| ATOM | 3107 | CA | SER | B | 446 | 32.108 | 34.047 | 33.663 | 1.00 | 22.00 | C |
| ATOM | 3108 | CB | SER | B | 446 | 32.570 | 35.480 | 33.292 | 1.00 | 19.55 | C |
| ATOM | 3109 | OG | SER | B | 446 | 32.902 | 36.261 | 34.407 | 1.00 | 22.80 | O |
| ATOM | 3110 | C | SER | B | 446 | 33.142 | 33.399 | 34.600 | 1.00 | 22.37 | C |
| ATOM | 3111 | O | SER | B | 446 | 34.109 | 32.812 | 34.131 | 1.00 | 21.37 | O |
| ATOM | 3112 | N | MET | B | 447 | 32.925 | 33.497 | 35.909 | 1.00 | 20.23 | N |
| ATOM | 3113 | CA | MET | B | 447 | 33.860 | 32.859 | 36.846 | 1.00 | 24.29 | C |
| ATOM | 3114 | CB | MET | B | 447 | 33.640 | 33.376 | 38.272 | 1.00 | 23.41 | C |
| ATOM | 3115 | CG | MET | B | 447 | 34.123 | 34.806 | 38.446 | 1.00 | 27.21 | C |
| ATOM | 3116 | SD | MET | B | 447 | 34.087 | 35.327 | 40.218 | 1.00 | 29.84 | S |
| ATOM | 3117 | CE | MET | B | 447 | 32.347 | 35.711 | 40.421 | 1.00 | 28.71 | C |
| ATOM | 3118 | C | MET | B | 447 | 33.698 | 31.342 | 36.767 | 1.00 | 24.03 | C |
| ATOM | 3119 | O | MET | B | 447 | 34.681 | 30.617 | 36.778 | 1.00 | 23.04 | O |
| ATOM | 3120 | N | GLY | B | 448 | 32.460 | 30.858 | 36.669 | 1.00 | 19.35 | N |
| ATOM | 3121 | CA | GLY | B | 448 | 32.276 | 29.423 | 36.546 | 1.00 | 21.93 | C |
| ATOM | 3122 | C | GLY | B | 448 | 32.898 | 28.922 | 35.248 | 1.00 | 28.63 | C |
| ATOM | 3123 | O | GLY | B | 448 | 33.506 | 27.847 | 35.242 | 1.00 | 23.12 | O |
| ATOM | 3124 | N | MET | B | 449 | 32.758 | 29.687 | 34.155 | 1.00 | 21.69 | N |
| ATOM | 3125 | CA | MET | B | 449 | 33.325 | 29.240 | 32.866 | 1.00 | 20.45 | C |
| ATOM | 3126 | CB | MET | B | 449 | 32.714 | 30.027 | 31.692 | 1.00 | 22.80 | C |
| ATOM | 3127 | CG | MET | B | 449 | 31.201 | 29.789 | 31.468 | 1.00 | 21.04 | C |
| ATOM | 3128 | SD | MET | B | 449 | 30.734 | 28.035 | 31.300 | 1.00 | 25.98 | S |
| ATOM | 3129 | CE | MET | B | 449 | 31.656 | 27.593 | 29.836 | 1.00 | 24.75 | C |
| ATOM | 3130 | C | MET | B | 449 | 34.852 | 29.343 | 32.825 | 1.00 | 19.20 | C |
| ATOM | 3131 | O | MET | B | 449 | 35.526 | 28.532 | 32.182 | 1.00 | 23.26 | O |
| ATOM | 3132 | N | LYS | B | 450 | 35.399 | 30.330 | 33.520 | 1.00 | 21.31 | N |
| ATOM | 3133 | CA | LYS | B | 450 | 36.847 | 30.477 | 33.578 | 1.00 | 22.27 | C |
| ATOM | 3134 | CB | LYS | B | 450 | 37.188 | 31.710 | 34.397 | 1.00 | 25.65 | C |
| ATOM | 3135 | CG | LYS | B | 450 | 38.686 | 31.870 | 34.602 | 1.00 | 35.16 | C |
| ATOM | 3136 | CD | LYS | B | 450 | 38.967 | 32.940 | 35.604 | 1.00 | 39.35 | C |
| ATOM | 3137 | CE | LYS | B | 450 | 40.432 | 32.855 | 36.005 | 1.00 | 41.39 | C |
| ATOM | 3138 | NZ | LYS | B | 450 | 40.770 | 33.998 | 36.884 | 1.00 | 50.53 | N |
| ATOM | 3139 | C | LYS | B | 450 | 37.382 | 29.201 | 34.259 | 1.00 | 21.58 | C |
| ATOM | 3140 | O | LYS | B | 450 | 38.395 | 28.634 | 33.863 | 1.00 | 23.54 | O |
| ATOM | 3141 | N | TYR | B | 451 | 36.667 | 28.738 | 35.272 | 1.00 | 22.85 | N |
| ATOM | 3142 | CA | TYR | B | 451 | 37.061 | 27.508 | 35.980 | 1.00 | 26.56 | C |
| ATOM | 3143 | CB | TYR | B | 451 | 36.179 | 27.307 | 37.225 | 1.00 | 31.08 | C |
| ATOM | 3144 | CG | TYR | B | 451 | 36.420 | 25.996 | 37.963 | 1.00 | 26.37 | C |
| ATOM | 3145 | CD1 | TYR | B | 451 | 37.523 | 25.831 | 38.813 | 1.00 | 32.42 | C |
| ATOM | 3146 | CE1 | TYR | B | 451 | 37.719 | 24.612 | 39.503 | 1.00 | 35.50 | C |
| ATOM | 3147 | CD2 | TYR | B | 451 | 35.543 | 24.934 | 37.810 | 1.00 | 29.25 | C |
| ATOM | 3148 | CE2 | TYR | B | 451 | 35.737 | 23.731 | 39.458 | 1.00 | 36.41 | C |
| ATOM | 3149 | CZ | TYR | B | 451 | 36.814 | 23.570 | 39.307 | 1.00 | 36.89 | C |
| ATOM | 3150 | OH | TYR | B | 451 | 36.928 | 22.357 | 39.947 | 1.00 | 35.03 | O |
| ATOM | 3151 | C | TYR | B | 451 | 36.975 | 26.290 | 35.084 | 1.00 | 23.27 | O |
| ATOM | 3152 | O | TYR | B | 451 | 37.904 | 25.484 | 35.035 | 1.00 | 22.89 | O |
| ATOM | 3153 | N | LEU | B | 452 | 35.881 | 26.144 | 34.339 | 1.00 | 21.39 | N |
| ATOM | 3154 | CA | LEU | B | 452 | 35.757 | 25.005 | 33.457 | 1.00 | 21.19 | C |
| ATOM | 3155 | CB | LEU | B | 452 | 34.379 | 24.995 | 32.735 | 1.00 | 25.34 | C |
| ATOM | 3156 | CG | LEU | B | 452 | 33.197 | 24.695 | 33.653 | 1.00 | 31.38 | C |
| ATOM | 3157 | CD1 | LEU | B | 452 | 31.922 | 24.872 | 32.832 | 1.00 | 39.34 | C |
| ATOM | 3158 | CD2 | LEU | B | 452 | 33.275 | 23.271 | 34.196 | 1.00 | 34.35 | C |
| ATOM | 3159 | C | LEU | B | 452 | 36.855 | 24.995 | 32.417 | 1.00 | 23.04 | C |
| ATOM | 3160 | O | LEU | B | 452 | 37.399 | 23.390 | 32.098 | 1.00 | 23.98 | O |
| ATOM | 3161 | N | GLU | B | 453 | 37.178 | 26.179 | 31.894 | 1.00 | 21.93 | N |
| ATOM | 3162 | CA | GLU | B | 453 | 38.215 | 26.298 | 30.881 | 1.00 | 24.62 | C |
| ATOM | 3163 | CB | GLU | B | 453 | 38.283 | 27.747 | 30.398 | 1.00 | 25.46 | C |
| ATOM | 3164 | CG | GLU | B | 453 | 39.415 | 28.050 | 29.406 | 1.00 | 28.82 | C |
| ATOM | 3165 | CD | GLU | B | 453 | 39.405 | 29.510 | 28.934 | 1.00 | 36.37 | C |
| ATOM | 3166 | OE1 | GLU | B | 453 | 40.016 | 30.376 | 29.621 | 1.00 | 33.79 | O |
| ATOM | 3167 | OE2 | GLU | B | 453 | 38.776 | 29.790 | 27.880 | 1.00 | 31.16 | O |
| ATOM | 3168 | C | GLU | B | 453 | 39.585 | 25.865 | 31.454 | 1.00 | 25.13 | C |
| ATOM | 3169 | O | GLU | B | 453 | 40.345 | 25.115 | 30.812 | 1.00 | 25.39 | O |
| ATOM | 3170 | N | GLU | B | 454 | 39.908 | 26.351 | 32.645 | 1.00 | 22.62 | N |
| ATOM | 3171 | CA | GLU | B | 454 | 41.204 | 25.996 | 33.245 | 1.00 | 26.22 | C |
| ATOM | 3172 | CB | GLU | B | 454 | 41.436 | 26.762 | 34.552 | 1.00 | 30.29 | C |
| ATOM | 3173 | CG | GLU | B | 454 | 40.519 | 26.379 | 35.681 | 1.00 | 43.54 | C |
| ATOM | 3174 | CD | GLU | B | 454 | 40.821 | 27.144 | 36.972 | 1.00 | 58.22 | C |
| ATOM | 3175 | OE1 | GLU | B | 454 | 40.838 | 28.404 | 36.941 | 1.00 | 50.28 | O |
| ATOM | 3176 | OE2 | GLU | B | 454 | 41.029 | 26.480 | 38.019 | 1.00 | 56.54 | O |
| ATOM | 3177 | C | GLU | B | 454 | 41.284 | 24.501 | 33.507 | 1.00 | 32.56 | C |
| ATOM | 3178 | O | GLU | B | 454 | 42.368 | 23.927 | 33.489 | 1.00 | 31.49 | O |
| ATOM | 3179 | N | LYS | B | 455 | 40.147 | 23.852 | 33.728 | 1.00 | 26.00 | N |
| ATOM | 3180 | CA | LYS | B | 455 | 40.141 | 22.406 | 33.979 | 1.00 | 27.14 | C |
| ATOM | 3181 | CB | LYS | B | 455 | 39.043 | 22.032 | 34.991 | 1.00 | 26.60 | C |
| ATOM | 3182 | CG | LYS | B | 455 | 39.173 | 22.743 | 36.346 | 1.00 | 29.96 | C |
| ATOM | 3183 | CD | LYS | B | 455 | 40.493 | 22.365 | 37.009 | 1.00 | 36.01 | C |
| ATOM | 3184 | CE | LYS | B | 455 | 40.733 | 23.118 | 38.322 | 1.00 | 40.11 | C |
| ATOM | 3185 | NZ | LYS | B | 455 | 42.067 | 22.747 | 38.909 | 1.00 | 33.10 | N |
| ATOM | 3186 | C | LYS | B | 455 | 39.954 | 21.605 | 32.692 | 1.00 | 26.89 | C |
| ATOM | 3187 | O | LYS | B | 455 | 39.757 | 20.397 | 32.732 | 1.00 | 31.85 | O |
| ATOM | 3188 | N | ASN | B | 456 | 40.047 | 22.280 | 31.551 | 1.00 | 27.82 | N |
| ATOM | 3189 | CA | ASN | B | 456 | 39.901 | 21.640 | 30.245 | 1.00 | 30.31 | C |
| ATOM | 3190 | CB | ASN | B | 456 | 41.089 | 20.707 | 29.967 | 1.00 | 32.92 | C |
| ATOM | 3191 | CG | ASN | B | 456 | 42.384 | 21.456 | 29.907 | 1.00 | 40.95 | C |
| ATOM | 3192 | OD1 | ASN | B | 456 | 42.556 | 22.351 | 29.081 | 1.00 | 37.33 | O |
| ATOM | 3193 | ND2 | ASN | B | 456 | 43.313 | 21.110 | 30.803 | 1.00 | 43.15 | N |
| ATOM | 3194 | C | ASN | B | 456 | 38.602 | 20.891 | 29.992 | 1.00 | 30.21 | C |
| ATOM | 3195 | O | ASN | B | 456 | 38.614 | 19.772 | 39.473 | 1.00 | 28.50 | O |
| ATOM | 3196 | N | PHE | B | 457 | 37.482 | 21.494 | 30.381 | 1.00 | 25.11 | N |
| ATOM | 3197 | CA | PHE | B | 457 | 36.193 | 20.899 | 30.098 | 1.00 | 22.51 | C |
| ATOM | 3198 | CB | PHE | B | 457 | 35.376 | 20.679 | 31.386 | 1.00 | 23.31 | C |
| ATOM | 3179 | CG | PHE | B | 457 | 35.848 | 19.515 | 32.204 | 1.00 | 26.75 | C |
| ATOM | 3200 | CD1 | PHE | B | 457 | 36.692 | 19.706 | 33.302 | 1.00 | 38.60 | C |
| ATOM | 3201 | CD2 | PHE | B | 457 | 35.456 | 18.232 | 31.877 | 1.00 | 29.58 | C |
| ATOM | 3202 | CE1 | PHE | B | 457 | 37.125 | 18.614 | 34.055 | 1.00 | 35.33 | C |
| ATOM | 3203 | CE2 | PHE | B | 457 | 35.887 | 17.140 | 32.624 | 1.00 | 29.78 | C |
| ATOM | 3204 | CZ | PHE | B | 457 | 36.718 | 17.334 | 33.710 | 1.00 | 29.78 | C |
| ATOM | 3205 | C | PHE | B | 457 | 35.435 | 21.903 | 29.198 | 1.00 | 20.08 | C |
| ATOM | 3206 | O | PHE | B | 457 | 35.555 | 23.105 | 29.396 | 1.00 | 24.84 | O |
| ATOM | 3207 | N | VAL | B | 458 | 34.657 | 21.370 | 28.247 | 1.00 | 22.46 | N |
| ATOM | 3208 | CA | VAL | B | 458 | 33.832 | 22.189 | 27.351 | 1.00 | 19.50 | C |
| ATOM | 3209 | CB | VAL | B | 458 | 34.039 | 21.771 | 25.864 | 1.00 | 24.51 | C |
| ATOM | 3210 | CG1 | VAL | B | 458 | 33.044 | 22.521 | 24.946 | 1.00 | 20.39 | C |
| ATOM | 3211 | CG2 | VAL | B | 458 | 35.451 | 22.094 | 25.438 | 1.00 | 26.69 | C |
| ATOM | 3212 | C | VAL | B | 458 | 32.365 | 21.908 | 27.749 | 1.00 | 22.31 | C |
| ATOM | 3213 | O | VAL | B | 458 | 31.956 | 20.779 | 27.818 | 1.00 | 23.69 | O |
| ATOM | 3214 | N | HIS | B | 459 | 31.581 | 22.946 | 27.977 | 1.00 | 21.21 | N |
| ATOM | 3215 | CA | HIS | B | 459 | 30.176 | 22.720 | 28.415 | 1.00 | 20.87 | C |
| ATOM | 3216 | CB | HIS | B | 459 | 29.670 | 23.998 | 29.119 | 1.00 | 26.99 | C |
| ATOM | 3217 | CG | HIS | B | 459 | 28.294 | 23.866 | 29.693 | 1.00 | 26.79 | C |
| ATOM | 3218 | CD2 | HIS | B | 459 | 27.869 | 23.814 | 30.978 | 1.00 | 33.82 | C |
| ATOM | 3219 | ND1 | HIS | B | 459 | 27.163 | 23.762 | 28.912 | 1.00 | 30.62 | N |
| ATOM | 3220 | CE1 | HIS | B | 459 | 26.099 | 23.665 | 29.691 | 1.00 | 28.63 | C |
| ATOM | 3221 | NE2 | HIS | B | 459 | 26.500 | 23.693 | 30.949 | 1.00 | 26.47 | N |
| ATOM | 3222 | C | HIS | B | 459 | 29.279 | 22.376 | 27.225 | 1.00 | 22.74 | C |
| ATOM | 3223 | O | HIS | B | 459 | 28.472 | 21.409 | 27.260 | 1.00 | 26.21 | O |
| ATOM | 3224 | N | ARG | B | 460 | 29.432 | 32.186 | 26.178 | 1.00 | 23.74 | N |
| ATOM | 3225 | CA | ARG | B | 460 | 28.716 | 23.083 | 24.911 | 1.00 | 23.96 | C |
| ATOM | 3226 | CB | ARG | B | 460 | 28.766 | 21.644 | 24.390 | 1.00 | 27.31 | C |
| ATOM | 3227 | CG | ARG | B | 460 | 28.259 | 21.546 | 22.972 | 1.00 | 37.56 | C |
| ATOM | 3228 | CD | ARG | B | 460 | 28.279 | 20.147 | 22.427 | 1.00 | 32.14 | C |
| ATOM | 3229 | NE | ARG | B | 460 | 27.254 | 20.032 | 21.406 | 1.00 | 31.71 | N |
| ATOM | 3230 | CZ | ARG | B | 460 | 27.008 | 18.927 | 20.699 | 1.00 | 39.03 | C |
| ATOM | 3231 | NH1 | ARG | B | 460 | 27.721 | 17.830 | 20.903 | 1.00 | 34.17 | N |
| ATOM | 3232 | NH2 | ARG | B | 460 | 26.040 | 18.924 | 19.793 | 1.00 | 42.19 | N |
| ATOM | 3233 | C | ARG | B | 460 | 27.261 | 23.562 | 24.895 | 1.00 | 30.30 | C |
| ATOM | 3234 | O | ARG | B | 460 | 26.726 | 23.888 | 23.833 | 1.00 | 37.44 | O |
| ATOM | 3235 | N | ASP | B | 461 | 26.628 | 23.648 | 26.054 | 1.00 | 24.84 | N |
| ATOM | 3236 | CA | ASP | B | 461 | 25.230 | 24.057 | 26.087 | 1.00 | 27.22 | C |
| ATOM | 3237 | CB | ASP | B | 461 | 24.389 | 22.843 | 26.547 | 1.00 | 29.93 | C |
| ATOM | 3238 | CG | ASP | B | 461 | 22.893 | 22.954 | 26.185 | 1.00 | 40.58 | C |
| ATOM | 3239 | OD1 | ASP | B | 461 | 22.497 | 23.822 | 25.374 | 1.00 | 47.29 | O |
| ATOM | 3240 | OD2 | ASP | B | 461 | 22.100 | 22.138 | 26.718 | 1.00 | 51.22 | O |
| ATOM | 3241 | C | ASP | B | 461 | 25.039 | 25.281 | 26.998 | 1.00 | 27.48 | C |
| ATOM | 3242 | O | ASP | B | 461 | 24.054 | 25.401 | 27.713 | 1.00 | 25.86 | O |
| ATOM | 3243 | N | LEU | B | 462 | 25.985 | 26.210 | 26.963 | 1.00 | 21.15 | N |
| ATOM | 3244 | CA | LEU | B | 462 | 25.841 | 27.339 | 27.788 | 1.00 | 20.41 | C |
| ATOM | 3245 | CB | LEU | B | 462 | 27.200 | 28.106 | 27.940 | 1.00 | 22.91 | C |
| ATOM | 3246 | CG | LEU | B | 462 | 27.216 | 29.380 | 28.792 | 1.00 | 28.71 | C |
| ATOM | 3247 | CD1 | LEU | B | 462 | 26.774 | 29.008 | 30.203 | 1.00 | 30.96 | C |
| ATOM | 3248 | CD2 | LEU | B | 462 | 28.614 | 30.037 | 28.792 | 1.00 | 27.89 | C |
| ATOM | 3249 | C | LEU | B | 462 | 24.790 | 28.300 | 27.113 | 1.00 | 25.32 | C |
| ATOM | 3250 | O | LEU | B | 462 | 24.913 | 28.640 | 25.958 | 1.00 | 27.45 | O |
| ATOM | 3251 | N | ALA | B | 463 | 23.737 | 28.637 | 27.847 | 1.00 | 20.92 | N |
| ATOM | 3252 | CA | ALA | B | 463 | 22.645 | 29.482 | 27.350 | 1.00 | 20.29 | C |
| ATOM | 3253 | CB | ALA | B | 463 | 21.709 | 28.658 | 26.431 | 1.00 | 17.73 | C |
| ATOM | 3254 | C | ALA | B | 463 | 21.905 | 29.962 | 28.596 | 1.00 | 21.81 | C |
| ATOM | 3255 | O | ALA | B | 463 | 21.978 | 29.319 | 29.655 | 1.00 | 21.64 | O |
| ATOM | 3256 | N | ALA | B | 464 | 21.187 | 31.069 | 28.492 | 1.00 | 19.40 | N |
| ATOM | 3257 | CA | ALA | B | 464 | 20.510 | 31.586 | 29.655 | 1.00 | 21.46 | C |
| ATOM | 3258 | CB | ALA | B | 464 | 19.750 | 32.879 | 29.294 | 1.00 | 20.54 | C |
| ATOM | 3259 | C | ALA | B | 464 | 19.567 | 30.560 | 30.288 | 1.00 | 23.11 | C |
| ATOM | 3260 | O | ALA | B | 464 | 19.399 | 30.567 | 31.499 | 1.00 | 22.21 | O |
| ATOM | 3261 | N | ARG | B | 465 | 18.963 | 29.703 | 29.468 | 1.00 | 22.19 | N |
| ATOM | 3262 | CA | ARG | B | 465 | 18.036 | 28.679 | 29.955 | 1.00 | 25.34 | C |
| ATOM | 3263 | CB | ARG | B | 465 | 17.372 | 27.947 | 28.783 | 1.00 | 22.79 | C |
| ATOM | 3264 | CG | ARG | B | 465 | 18.310 | 27.131 | 27.897 | 1.00 | 21.09 | C |
| ATOM | 3265 | CD | ARG | B | 465 | 17.586 | 26.576 | 26.650 | 1.00 | 28.36 | C |
| ATOM | 3266 | NE | ARG | B | 465 | 18.537 | 25.936 | 25.752 | 1.00 | 33.20 | N |
| ATOM | 3267 | CZ | ARG | B | 465 | 19.230 | 26.568 | 24.801 | 1.00 | 41.11 | C |
| ATOM | 3268 | NH1 | ARG | B | 465 | 19.074 | 27.873 | 24.592 | 1.00 | 34.56 | N |
| ATOM | 3269 | NH2 | ARG | B | 465 | 20.129 | 25.908 | 24.099 | 1.00 | 36.89 | N |
| ATOM | 3270 | C | ARG | B | 465 | 18.746 | 27.650 | 30.840 | 1.00 | 27.21 | C |
| ATOM | 3271 | O | ARG | B | 465 | 18.098 | 26.930 | 31.600 | 1.00 | 25.31 | O |
| ATOM | 3272 | N | ASN | B | 466 | 20.075 | 27.583 | 30.727 | 1.00 | 26.22 | N |
| ATOM | 3273 | CA | ASN | B | 466 | 20.851 | 26.640 | 31.535 | 1.00 | 22.74 | C |
| ATOM | 3274 | CB | ASN | B | 466 | 21.777 | 25.809 | 30.647 | 1.00 | 20.44 | C |
| ATOM | 3275 | CG | ASN | B | 466 | 21.022 | 24.810 | 29.806 | 1.00 | 31.49 | C |
| ATOM | 3276 | OD1 | ASN | B | 466 | 19.963 | 34.316 | 30.217 | 1.00 | 26.09 | O |
| ATOM | 3277 | ND2 | ASN | B | 466 | 21.556 | 24.496 | 28.624 | 1.00 | 25.81 | N |
| ATOM | 3278 | C | ASN | B | 466 | 21.608 | 27.277 | 32.677 | 1.00 | 24.14 | C |
| ATOM | 3279 | O | ASN | B | 466 | 22.379 | 26.617 | 33.372 | 1.00 | 25.61 | O |
| ATOM | 3280 | N | VAL | B | 467 | 21.384 | 28.563 | 32.895 | 1.00 | 19.90 | N |
| ATOM | 3281 | CA | VAL | B | 467 | 21.999 | 29.239 | 34.013 | 1.00 | 18.65 | C |
| ATOM | 3282 | CB | VAL | B | 467 | 22.443 | 30.652 | 33.640 | 1.00 | 21.94 | C |
| ATOM | 3283 | CG1 | VAL | B | 467 | 22.840 | 31.414 | 34.910 | 1.00 | 20.29 | C |
| ATOM | 3284 | CG2 | VAL | B | 467 | 23.617 | 31.414 | 32.634 | 1.00 | 21.43 | C |
| ATOM | 3285 | C | VAL | B | 467 | 20.834 | 29.323 | 35.002 | 1.00 | 25.03 | C |
| ATOM | 3286 | O | VAL | B | 467 | 19.757 | 29.829 | 34.659 | 1.00 | 24.55 | O |
| ATOM | 3287 | N | LEU | B | 468 | 21.047 | 28.821 | 36.212 | 1.00 | 21.56 | N |
| ATOM | 3288 | CA | LEU | B | 468 | 19.972 | 28.813 | 37.219 | 1.00 | 22.81 | C |
| ATOM | 3289 | CB | LEU | B | 468 | 19.738 | 27.385 | 37.734 | 1.00 | 22.41 | C |
| ATOM | 3290 | CG | LEU | B | 468 | 19.493 | 26.291 | 36.695 | 1.00 | 25.80 | C |
| ATOM | 3291 | CD1 | LEU | B | 468 | 19.568 | 24.927 | 37.347 | 1.00 | 27.13 | C |
| ATOM | 3292 | CD2 | LEU | B | 468 | 18.128 | 26.521 | 36.038 | 1.00 | 28.96 | C |
| ATOM | 3293 | C | LEU | B | 468 | 20.287 | 29.707 | 38.394 | 1.00 | 27.22 | C |
| ATOM | 3294 | O | LEU | B | 468 | 21.426 | 29.782 | 38.826 | 1.00 | 26.70 | O |
| ATOM | 3295 | N | LEU | B | 469 | 19.262 | 30.349 | 38.960 | 1.00 | 24.32 | N |
| ATOM | 3296 | CA | LEU | B | 469 | 19.511 | 31.234 | 40.078 | 1.00 | 23.08 | C |
| ATOM | 3297 | CB | LEU | B | 469 | 18.687 | 32.498 | 39.908 | 1.00 | 24.42 | C |
| ATOM | 3298 | CG | LEU | B | 469 | 18.985 | 33.232 | 38.596 | 1.00 | 37.62 | C |
| ATOM | 3299 | CD1 | LEU | B | 469 | 17.999 | 34.336 | 38.412 | 1.00 | 40.80 | C |
| ATOM | 3300 | CD2 | LEU | B | 469 | 20.384 | 33.797 | 38.612 | 1.00 | 33.91 | C |
| ATOM | 3301 | C | LEU | B | 469 | 19.216 | 30.625 | 41.440 | 1.00 | 26.89 | C |
| ATOM | 3302 | O | LEU | B | 469 | 18.162 | 30.024 | 41.643 | 1.00 | 27.22 | O |
| ATOM | 3303 | N | VAL | B | 470 | 20.154 | 30.766 | 42.366 | 1.00 | 27.63 | N |
| ATOM | 3304 | CA | VAL | B | 470 | 19.903 | 30.311 | 43.718 | 1.00 | 30.31 | C |
| ATOM | 3305 | CB | VAL | B | 470 | 21.199 | 30.132 | 44.520 | 1.00 | 33.68 | C |
| ATOM | 3306 | CG1 | VAL | B | 470 | 20.862 | 29.797 | 45.973 | 1.00 | 34.34 | C |
| ATOM | 3307 | CG2 | VAL | B | 470 | 22.027 | 29.002 | 43.912 | 1.00 | 28.72 | C |
| ATOM | 3308 | C | VAL | B | 470 | 19.057 | 31.450 | 44.300 | 1.00 | 31.84 | C |
| ATOM | 3309 | O | VAL | B | 470 | 18.096 | 31.210 | 45.051 | 1.00 | 33.69 | O |
| ATOM | 3310 | N | ASN | B | 471 | 19.407 | 36.682 | 43.930 | 1.00 | 29.94 | N |
| ATOM | 3311 | CA | ASN | B | 471 | 18.688 | 33.903 | 44.335 | 1.00 | 34.01 | C |
| ATOM | 3312 | CB | ASN | B | 471 | 19.139 | 34.394 | 45.723 | 1.00 | 38.51 | C |
| ATOM | 3313 | CG | ASN | B | 471 | 20.631 | 34.554 | 45.824 | 1.00 | 41.61 | C |
| ATOM | 3314 | OD1 | ASN | B | 471 | 21.336 | 33.627 | 46.225 | 1.00 | 62.13 | O |
| ATOM | 3315 | ND2 | ASN | B | 471 | 21.129 | 35.710 | 45.433 | 1.00 | 34.16 | N |
| ATOM | 3316 | C | ASN | B | 471 | 19.009 | 34.971 | 43.283 | 1.00 | 32.05 | C |
| ATOM | 3317 | O | ASN | B | 471 | 19.796 | 34.712 | 42.378 | 1.00 | 29.59 | O |
| ATOM | 3318 | N | ARG | B | 472 | 18.434 | 36.170 | 43.395 | 1.00 | 31.13 | N |
| ATOM | 3319 | CA | ARG | B | 472 | 18.687 | 37.216 | 42.392 | 1.00 | 33.64 | C |
| ATOM | 3320 | CB | ARG | B | 472 | 17.766 | 38.417 | 42.631 | 1.00 | 35.84 | C |
| ATOM | 3321 | CG | ARG | B | 472 | 18.042 | 39.147 | 43.924 | 1.00 | 37.87 | C |
| ATOM | 3322 | CD | ARG | B | 472 | 17.142 | 40.371 | 44.053 | 1.00 | 40.99 | C |
| ATOM | 3323 | NE | ARG | B | 472 | 17.621 | 41.483 | 43.240 | 1.00 | 43.11 | N |
| ATOM | 3324 | CZ | ARG | B | 472 | 16.972 | 42.633 | 43.074 | 1.00 | 48.70 | C |
| ATOM | 3325 | NH1 | ARG | B | 472 | 15.795 | 42.832 | 43.667 | 1.00 | 41.99 | N |
| ATOM | 3326 | NH2 | ARG | B | 472 | 17.503 | 43.593 | 42.334 | 1.00 | 44.48 | N |
| ATOM | 3327 | C | ARG | B | 472 | 20.132 | 37.702 | 42.324 | 1.00 | 24.49 | C |
| ATOM | 3328 | O | ARG | B | 472 | 20.492 | 38.446 | 41.423 | 1.00 | 30.50 | O |
| ATOM | 3329 | N | HIS | B | 473 | 20.952 | 37.294 | 43.288 | 1.00 | 26.54 | N |
| ATOM | 3330 | CA | HIS | B | 473 | 22.340 | 37.700 | 43.324 | 1.00 | 30.93 | C |
| ATOM | 3331 | CB | HIS | B | 473 | 22.628 | 38.717 | 44.614 | 1.00 | 28.14 | C |
| ATOM | 3332 | CG | HIS | B | 473 | 21.888 | 39.817 | 44.673 | 1.00 | 32.49 | C |
| ATOM | 3333 | CD2 | HIS | B | 473 | 20.856 | 40.225 | 45.451 | 1.00 | 40.34 | C |
| ATOM | 3334 | ND1 | HIS | B | 473 | 22.125 | 40.845 | 43.786 | 1.00 | 36.03 | N |
| ATOM | 3335 | CE1 | HIS | B | 473 | 21.266 | 41.825 | 44.008 | 1.00 | 41.10 | C |
| ATOM | 3336 | NE2 | HIS | B | 473 | 20.482 | 41.473 | 45.013 | 1.00 | 42.57 | N |
| ATOM | 3337 | C | HIS | B | 473 | 23.276 | 36.497 | 43.260 | 1.00 | 25.86 | C |
| ATOM | 3338 | O | HIS | B | 473 | 24.472 | 36.628 | 43.573 | 1.00 | 25.18 | O |
| ATOM | 3339 | N | TYR | B | 474 | 22.760 | 35.337 | 42.854 | 1.00 | 26.93 | N |
| ATOM | 3340 | CA | TYR | B | 474 | 23.614 | 34.141 | 42.810 | 1.00 | 28.61 | C |
| ATOM | 3341 | CB | TYR | B | 474 | 23.597 | 33.447 | 44.184 | 1.00 | 26.00 | C |
| ATOM | 3342 | CG | TYR | B | 474 | 24.589 | 32.302 | 44.396 | 1.00 | 29.87 | C |
| ATOM | 3343 | CD1 | TYR | B | 474 | 25.645 | 32.050 | 43.505 | 1.00 | 27.42 | C |
| ATOM | 3344 | CE1 | TYR | B | 474 | 26.563 | 30.995 | 43.747 | 1.00 | 31.28 | C |
| ATOM | 3345 | CD2 | TYR | B | 474 | 24.477 | 31.481 | 45.523 | 1.00 | 34.24 | C |
| ATOM | 3346 | CE2 | TYR | B | 474 | 25.377 | 30.444 | 45.768 | 1.00 | 37.06 | C |
| ATOM | 3347 | CZ | TYR | B | 474 | 26.416 | 30.198 | 44.888 | 1.00 | 35.99 | C |
| ATOM | 3348 | OH | TYR | B | 474 | 27.285 | 29.160 | 45.187 | 1.00 | 32.29 | O |
| ATOM | 3349 | C | TYR | B | 474 | 23.218 | 33.160 | 41.719 | 1.00 | 27.49 | C |
| ATOM | 3350 | O | TYR | B | 474 | 22.237 | 32.413 | 41.862 | 1.00 | 27.72 | O |
| ATOM | 3351 | N | ALA | B | 475 | 24.003 | 33.153 | 40.638 | 1.00 | 21.66 | N |
| ATOM | 3352 | CA | ALA | B | 475 | 23.744 | 32.279 | 39.509 | 1.00 | 25.73 | C |
| ATOM | 3353 | CB | ALA | B | 475 | 23.886 | 33.075 | 38.200 | 1.00 | 27.00 | C |
| ATOM | 3354 | C | ALA | B | 475 | 24.680 | 31.057 | 39.486 | 1.00 | 25.53 | C |
| ATOM | 3355 | O | ALA | B | 475 | 25.809 | 31.113 | 39.970 | 1.00 | 26.97 | 0 |
| ATOM | 3356 | N | LYS | B | 476 | 24.190 | 29.953 | 38.921 | 1.00 | 22.58 | N |
| ATOM | 3357 | CA | LYS | B | 476 | 24.987 | 28.739 | 38.771 | 1.00 | 23.46 | C |
| ATOM | 3358 | CB | LYS | B | 476 | 24.594 | 27.691 | 39.834 | 1.00 | 24.43 | C |
| ATOM | 3359 | CG | LYS | B | 476 | 25.144 | 28.000 | 41.221 | 1.00 | 25.55 | C |
| ATOM | 3360 | CD | LYS | B | 476 | 24.578 | 26.972 | 42.233 | 1.00 | 32.01 | C |
| ATOM | 3361 | CE | LYS | B | 476 | 25.288 | 27.036 | 43.577 | 1.00 | 36.59 | C |
| ATOM | 3362 | NZ | LYS | B | 476 | 26.553 | 26.248 | 43.526 | 1.00 | 37.14 | N |
| ATOM | 3363 | C | LYS | B | 476 | 24.787 | 28.124 | 37.402 | 1.00 | 24.32 | C |
| ATOM | 3364 | O | LYS | B | 476 | 23.658 | 28.084 | 36.876 | 1.00 | 24.88 | O |
| ATOM | 3365 | N | ILE | B | 477 | 25.869 | 27.620 | 36.815 | 1.00 | 21.48 | N |
| ATOM | 3366 | CA | ILE | B | 477 | 25.778 | 26.980 | 35.511 | 1.00 | 23.09 | C |
| ATOM | 3367 | CB | ILE | B | 477 | 27.139 | 26.937 | 34.843 | 1.00 | 20.87 | C |
| ATOM | 3368 | CG2 | ILE | B | 477 | 27.065 | 26.129 | 33.544 | 1.00 | 24.48 | C |
| ATOM | 3369 | CG1 | ILE | B | 477 | 27.644 | 28.370 | 34.642 | 1.00 | 22.01 | C |
| ATOM | 3370 | CD1 | ILE | B | 477 | 29.143 | 28.461 | 34.443 | 1.00 | 23.58 | C |
| ATOM | 3371 | C | ILE | B | 477 | 25.297 | 25.560 | 35.689 | 1.00 | 26.88 | C |
| ATOM | 3372 | O | ILE | B | 477 | 25.794 | 24.860 | 36.565 | 1.00 | 24.82 | O |
| ATOM | 3373 | N | SER | B | 478 | 24.351 | 25.111 | 34.855 | 1.00 | 22.63 | N |
| ATOM | 3374 | CA | SER | B | 478 | 23.876 | 23.743 | 34.992 | 1.00 | 23.59 | C |
| ATOM | 3375 | CB | SER | B | 478 | 22.474 | 23.741 | 35.578 | 1.00 | 30.50 | C |
| ATOM | 3376 | OG | SER | B | 478 | 21.607 | 24.274 | 34.601 | 1.00 | 40.13 | O |
| ATOM | 3377 | C | SER | B | 478 | 23.842 | 22.996 | 33.664 | 1.00 | 27.84 | C |
| ATOM | 3378 | O | SER | B | 478 | 24.253 | 23.528 | 32.616 | 1.00 | 27.42 | O |
| ATOM | 3379 | N | ASP | B | 479 | 23.302 | 21.775 | 33.730 | 1.00 | 23.72 | N |
| ATOM | 3380 | CA | ASP | B | 479 | 23.164 | 20.821 | 32.611 | 1.00 | 25.82 | C |
| ATOM | 3381 | CB | ASP | B | 479 | 22.094 | 21.256 | 31.585 | 1.00 | 24.35 | C |
| ATOM | 3382 | CG | ASP | B | 479 | 21.711 | 20.104 | 30.636 | 1.00 | 34.90 | C |
| ATOM | 3383 | OD1 | ASP | B | 479 | 22.419 | 19.076 | 30.636 | 1.00 | 30.23 | O |
| ATOM | 3384 | OD2 | ASP | B | 479 | 20.703 | 20.214 | 29.899 | 1.00 | 30.91 | O |
| ATOM | 3385 | C | ASP | B | 479 | 24.460 | 20.489 | 31.878 | 1.00 | 24.50 | C |
| ATOM | 3386 | O | ASP | B | 479 | 24.751 | 21.037 | 30.784 | 1.00 | 21.34 | O |
| ATOM | 3387 | N | PHE | B | 480 | 25.197 | 19.529 | 32.444 | 1.00 | 26.62 | N |
| ATOM | 3388 | CA | PHE | B | 480 | 26.464 | 19.062 | 31.863 | 1.00 | 27.44 | C |
| ATOM | 3389 | CB | PHE | B | 480 | 27.474 | 18.812 | 32.993 | 1.00 | 26.01 | C |
| ATOM | 3390 | CG | PHE | B | 480 | 27.893 | 20.075 | 33.696 | 1.00 | 25.53 | C |
| ATOM | 3391 | CD1 | PHE | B | 480 | 29.029 | 20.788 | 33.289 | 1.00 | 37.11 | C |
| ATOM | 3392 | CD2 | PHE | B | 480 | 27.095 | 20.617 | 34.700 | 1.00 | 30.32 | C |
| ATOM | 3393 | CE1 | PHE | B | 480 | 29.346 | 22.023 | 33.870 | 1.00 | 34.94 | C |
| ATOM | 3394 | CE2 | PHE | B | 480 | 27.403 | 21.850 | 35.279 | 1.00 | 31.64 | C |
| ATOM | 3395 | CZ | PHE | B | 480 | 28.520 | 22.555 | 34.866 | 1.00 | 33.32 | C |
| ATOM | 3396 | C | PHE | B | 480 | 26.288 | 17.810 | 30.989 | 1.00 | 23.32 | C |
| ATOM | 3397 | O | PHE | B | 480 | 27.242 | 17.078 | 30.712 | 1.00 | 29.60 | O |
| ATOM | 3398 | N | GLY | B | 481 | 25.059 | 17.586 | 30.533 | 1.00 | 24.87 | N |
| ATOM | 3399 | CA | GLY | B | 481 | 24.743 | 16.444 | 29.690 | 1.00 | 25.16 | C |
| ATOM | 3400 | C | GLY | B | 481 | 25.510 | 16.367 | 28.377 | 1.00 | 32.36 | C |
| ATOM | 3401 | O | GLY | B | 481 | 25.643 | 15.300 | 27.819 | 1.00 | 28.44 | O |
| ATOM | 3402 | N | LEU | B | 482 | 26.017 | 17.491 | 27.876 | 1.00 | 24.22 | N |
| ATOM | 3403 | CA | LEU | B | 482 | 26.778 | 17.474 | 26.617 | 1.00 | 24.32 | C |
| ATOM | 3404 | CB | LEU | B | 482 | 26.169 | 18.473 | 25.622 | 1.00 | 27.61 | C |
| ATOM | 3405 | CG | LEU | B | 482 | 24.767 | 18.111 | 25.133 | 1.00 | 31.09 | C |
| ATOM | 3406 | CD1 | LEU | B | 482 | 24.143 | 19.316 | 24.392 | 1.00 | 37.33 | C |
| ATOM | 3407 | CD2 | LEU | B | 482 | 24.868 | 16.912 | 24.231 | 1.00 | 33.44 | C |
| ATOM | 3408 | C | LEU | B | 482 | 28.299 | 17.843 | 26.833 | 1.00 | 20.28 | C |
| ATOM | 3409 | O | LEU | B | 482 | 28.998 | 18.006 | 25.881 | 1.00 | 27.53 | O |
| ATOM | 3410 | N | SER | B | 483 | 28.607 | 17.986 | 28.093 | 1.00 | 21.03 | N |
| ATOM | 3411 | CA | SER | B | 483 | 29.959 | 18.392 | 28.423 | 1.00 | 17.07 | C |
| ATOM | 3412 | CB | SER | B | 483 | 30.001 | 18.818 | 29.882 | 1.00 | 21.12 | C |
| ATOM | 3413 | OG | SER | B | 483 | 29.143 | 19.933 | 30.053 | 1.00 | 31.09 | O |
| ATOM | 3414 | C | SER | B | 483 | 30.984 | 17.298 | 28.156 | 1.00 | 21.12 | C |
| ATOM | 3415 | O | SER | B | 483 | 30.662 | 16.131 | 28.176 | 1.00 | 23.30 | O |
| ATOM | 3416 | N | LYS | B | 484 | 32.225 | 17.700 | 27.901 | 1.00 | 22.23 | N |
| ATOM | 3417 | CA | LYS | B | 484 | 33.281 | 16.732 | 27.641 | 1.00 | 22.46 | C |
| ATOM | 3418 | CB | LYS | B | 484 | 33.403 | 16.456 | 26.135 | 1.00 | 25.43 | C |
| ATOM | 3419 | CG | LYS | B | 484 | 32.202 | 15.661 | 25.564 | 1.00 | 37.37 | C |
| ATOM | 3420 | CD | LYS | B | 484 | 32.323 | 15.382 | 24.069 | 1.00 | 42.87 | C |
| ATOM | 3421 | CE | LYS | B | 484 | 31.228 | 14.402 | 23.622 | 1.00 | 42.01 | C |
| ATOM | 3422 | NZ | LYS | B | 484 | 29.878 | 14.732 | 24.149 | 1.00 | 57.01 | N |
| ATOM | 3423 | C | LYS | B | 484 | 34.622 | 17.227 | 28.157 | 1.00 | 26.66 | C |
| ATOM | 3424 | O | LYS | B | 484 | 24.870 | 18.426 | 28.229 | 1.00 | 25.66 | O |
| ATOM | 3425 | N | ALA | B | 485 | 35.489 | 16.280 | 28.492 | 1.00 | 22.91 | N |
| ATOM | 3426 | CA | ALA | B | 485 | 36.825 | 16.636 | 28.962 | 1.00 | 21.91 | C |
| ATOM | 3427 | CB | ALA | B | 485 | 37.359 | 15.560 | 29.932 | 1.00 | 32.95 | C |
| ATOM | 3428 | C | ALA | B | 485 | 37.689 | 16.684 | 27.679 | 1.00 | 29.52 | C |
| ATOM | 3429 | O | ALA | B | 485 | 37.552 | 15.827 | 26.804 | 1.00 | 32.44 | O |
| ATOM | 3430 | N | LEU | B | 486 | 38.546 | 17.694 | 27.576 | 1.00 | 24.43 | N |
| ATOM | 3431 | CA | LEU | B | 486 | 39.419 | 17.841 | 26.403 | 1.00 | 19.74 | C |
| ATOM | 3432 | CB | LEU | B | 486 | 39.663 | 19.335 | 26.108 | 1.00 | 28.91 | C |
| ATOM | 3433 | CG | LEU | B | 486 | 38.582 | 20.155 | 25.390 | 1.00 | 28.77 | C |
| ATOM | 3434 | CD1 | LEU | B | 486 | 38.967 | 21.633 | 25.366 | 1.00 | 34.39 | C |
| ATOM | 3435 | CD2 | LEU | B | 486 | 38.408 | 19.629 | 24.006 | 1.00 | 36.84 | C |
| ATOM | 3436 | C | LEU | B | 486 | 40.794 | 17.187 | 26.636 | 1.00 | 31.66 | C |
| ATOM | 3437 | O | LEU | B | 486 | 41.477 | 16.831 | 25.690 | 1.00 | 34.07 | O |
| ATOM | 3438 | N | GLY | B | 487 | 41.203 | 17.066 | 27.897 | 1.00 | 30.25 | N |
| ATOM | 3439 | CA | GLY | B | 487 | 42.510 | 16.493 | 28.177 | 1.00 | 35.90 | C |
| ATOM | 3440 | C | GLY | B | 487 | 43.591 | 17.425 | 27.643 | 1.00 | 35.05 | C |
| ATOM | 3441 | O | GLY | B | 487 | 43.547 | 18.624 | 27.847 | 1.00 | 37.70 | O |
| ATOM | 3442 | N | ALA | B | 488 | 44.540 | 16.853 | 26.919 | 1.00 | 36.40 | N |
| ATOM | 3443 | CA | ALA | B | 488 | 45.644 | 17.630 | 26.364 | 1.00 | 41.44 | C |
| ATOM | 3444 | CB | ALA | B | 488 | 46.841 | 16.711 | 26.100 | 1.00 | 44.00 | C |
| ATOM | 3445 | C | ALA | B | 488 | 45.283 | 18.375 | 25.080 | 1.00 | 40.64 | C |
| ATOM | 3446 | O | ALA | B | 488 | 46.035 | 19.227 | 24.627 | 1.00 | 40.65 | O |
| ATOM | 3447 | N | ASP | B | 489 | 44.125 | 18.068 | 24.506 | 1.00 | 37.22 | N |
| ATOM | 3448 | CA | ASP | B | 489 | 43.706 | 18.723 | 23.255 | 1.00 | 35.43 | C |
| ATOM | 3449 | CB | ASP | B | 489 | 42.740 | 17.811 | 22.497 | 1.00 | 36.96 | C |
| ATOM | 3450 | CG | ASP | B | 489 | 43.331 | 16.450 | 22.240 | 1.00 | 48.65 | C |
| ATOM | 3451 | OD1 | ASP | B | 489 | 44.563 | 16.383 | 22.017 | 1.00 | 51.66 | O |
| ATOM | 3452 | OD2 | ASP | B | 489 | 42.571 | 15.457 | 22.249 | 1.00 | 47.30 | O |
| ATOM | 3453 | C | ASP | B | 489 | 43.062 | 20.090 | 23.440 | 1.00 | 31.19 | C |
| ATOM | 3454 | O | ASP | B | 489 | 42.545 | 20.391 | 24.502 | 1.00 | 31.54 | O |
| ATOM | 3455 | N | ASP | B | 490 | 43.085 | 20.911 | 22.387 | 1.00 | 28.93 | N |
| ATOM | 3456 | CA | ASP | B | 490 | 42.480 | 22.235 | 22.480 | 1.00 | 31.45 | C |
| ATOM | 3457 | CB | ASP | B | 490 | 43.333 | 23.272 | 21.742 | 1.00 | 36.63 | C |
| ATOM | 3458 | CG | ASP | B | 490 | 43.495 | 22.950 | 20.292 | 1.00 | 47.74 | C |
| ATOM | 3459 | OD1 | ASP | B | 490 | 44.554 | 23.298 | 19.706 | 1.00 | 55.99 | O |
| ATOM | 3460 | OD2 | ASP | B | 490 | 42.557 | 22.364 | 19.727 | 1.00 | 54.77 | O |
| ATOM | 3461 | C | ASP | B | 490 | 41.047 | 22.241 | 21.942 | 1.00 | 26.58 | C |
| ATOM | 3462 | O | ASP | B | 490 | 40.347 | 23.252 | 22.028 | 1.00 | 25.36 | O |
| ATOM | 3463 | N | SER | B | 491 | 40.599 | 21.115 | 21.398 | 1.00 | 24.18 | N |
| ATOM | 3464 | CA | SER | B | 491 | 39.215 | 21.008 | 20.901 | 1.00 | 21.95 | C |
| ATOM | 3465 | CB | SER | B | 491 | 39.067 | 21.782 | 19.592 | 1.00 | 33.37 | C |
| ATOM | 3466 | OG | SER | B | 491 | 39.830 | 21.147 | 18.559 | 1.00 | 33.09 | O |
| ATOM | 3467 | C | SER | B | 491 | 38.879 | 19.574 | 20.619 | 1.00 | 29.49 | C |
| ATOM | 3468 | O | SER | B | 491 | 39.754 | 18.698 | 20.726 | 1.00 | 26.31 | O |
| ATOM | 3469 | N | TYR | B | 492 | 37.611 | 19.310 | 20.296 | 1.00 | 23.30 | N |
| ATOM | 3470 | CA | TYR | B | 492 | 37.228 | 17.987 | 19.891 | 1.00 | 24.79 | C |
| ATOM | 3471 | CB | TYR | B | 492 | 36.708 | 17.112 | 21.060 | 1.00 | 24.52 | C |
| ATOM | 3472 | CG | TYR | B | 492 | 35.358 | 17.507 | 21.590 | 1.00 | 26.67 | C |
| ATOM | 3473 | CD1 | TYR | B | 492 | 34.187 | 16.946 | 21.070 | 1.00 | 26.01 | C |
| ATOM | 3474 | CE1 | TYR | B | 492 | 32.928 | 17.362 | 21.510 | 1.00 | 29.14 | C |
| ATOM | 3475 | CD2 | TYR | B | 492 | 35.249 | 18.487 | 22.568 | 1.00 | 24.91 | C |
| ATOM | 3476 | CE2 | TYR | B | 492 | 33.979 | 18.912 | 23.025 | 1.00 | 24.00 | C |
| ATOM | 3477 | CZ | TYR | B | 492 | 32.835 | 18.343 | 22.483 | 1.00 | 26.42 | C |
| ATOM | 3478 | OH | TYR | B | 492 | 31.589 | 18.775 | 22.887 | 1.00 | 26.61 | O |
| ATOM | 3479 | C | TYR | B | 492 | 36.180 | 18.166 | 18.803 | 1.00 | 20.40 | C |
| ATOM | 3480 | O | TYR | B | 492 | 35.632 | 19.255 | 18.622 | 1.00 | 22.94 | O |
| ATOM | 3481 | N | TYR | B | 493 | 35.960 | 17.099 | 18.050 | 1.00 | 20.88 | N |
| ATOM | 3482 | CA | TYR | B | 493 | 34.958 | 17.072 | 16.980 | 1.00 | 19.63 | C |
| ATOM | 3483 | CB | TYR | B | 493 | 35.559 | 16.596 | 15.671 | 1.00 | 21.26 | C |
| ATOM | 3484 | CG | TYR | B | 493 | 36.515 | 17.601 | 15.095 | 1.00 | 24.50 | C |
| ATOM | 3485 | CD1 | TYR | B | 493 | 37.777 | 17.769 | 15.645 | 1.00 | 27.04 | C |
| ATOM | 3486 | CE1 | TYR | B | 493 | 38.652 | 18.713 | 15.144 | 1.00 | 36.13 | C |
| ATOM | 3487 | CD2 | TYR | B | 493 | 36.137 | 18.407 | 14.014 | 1.00 | 26.33 | C |
| ATOM | 3488 | CE2 | TYR | B | 493 | 37.011 | 19.352 | 13.501 | 1.00 | 27.99 | C |
| ATOM | 3489 | CZ | TYR | B | 493 | 38.262 | 19.497 | 14.067 | 1.00 | 32.35 | C |
| ATOM | 3490 | OH | TYR | B | 493 | 39.144 | 20.396 | 13.526 | 1.00 | 33.44 | O |
| ATOM | 3491 | C | TYR | B | 493 | 33.944 | 16.069 | 17.468 | 1.00 | 21.80 | C |
| ATOM | 3492 | O | TYR | B | 493 | 34.293 | 14.925 | 17.800 | 1.00 | 22.36 | O |
| ATOM | 3493 | N | THR | B | 494 | 32.695 | 16.514 | 17.539 | 1.00 | 26.46 | N |
| ATOM | 3494 | CA | THR | B | 494 | 31.623 | 15.700 | 18.095 | 1.00 | 33.93 | C |
| ATOM | 3495 | CB | THR | B | 494 | 30.580 | 16.611 | 18.817 | 1.00 | 33.02 | C |
| ATOM | 3496 | OG1 | THR | B | 494 | 29.647 | 15.794 | 19.538 | 1.00 | 40.25 | O |
| ATOM | 3497 | CG2 | THR | B | 494 | 29.817 | 17.435 | 17.812 | 1.00 | 33.65 | C |
| ATOM | 3498 | C | THR | B | 494 | 30.875 | 14.851 | 17.104 | 1.00 | 36.70 | C |
| ATOM | 3499 | O | THR | B | 494 | 30.757 | 15.179 | 15.921 | 1.00 | 28.58 | O |
| ATOM | 3500 | N | ALA | B | 495 | 30.294 | 13.773 | 17.611 | 1.00 | 44.45 | N |
| ATOM | 3501 | CA | ALA | B | 495 | 29.508 | 12.911 | 16.764 | 1.00 | 49.14 | C |
| ATOM | 3502 | CB | ALA | B | 495 | 29.442 | 11.529 | 17.370 | 1.00 | 47.66 | C |
| ATOM | 3503 | C | ALA | B | 495 | 28.108 | 13.503 | 16.624 | 1.00 | 49.65 | C |
| ATOM | 3504 | O | ALA | B | 495 | 27.651 | 14.233 | 17.498 | 1.00 | 48.99 | O |
| ATOM | 3505 | N | ARG | B | 496 | 27.446 | 13.194 | 15.519 | 1.00 | 53.97 | N |
| ATOM | 3506 | CA | ARG | B | 496 | 26.098 | 13.678 | 15.264 | 1.00 | 57.66 | C |
| ATOM | 3507 | CB | ARG | B | 496 | 25.804 | 13.674 | 13.759 | 1.00 | 60.80 | C |
| ATOM | 3508 | CG | ARG | B | 496 | 25.116 | 14.929 | 13.230 | 1.00 | 65.91 | C |
| ATOM | 3509 | CD | ARG | B | 496 | 23.905 | 15.299 | 14.043 | 1.00 | 66.81 | C |
| ATOM | 3510 | NE | ARG | B | 496 | 23.322 | 16.552 | 13.573 | 1.00 | 66.60 | N |
| ATOM | 3511 | CZ | ARG | B | 496 | 22.295 | 17.160 | 14.156 | 1.00 | 71.04 | C |
| ATOM | 3512 | NH1 | ARG | B | 496 | 21.737 | 16.626 | 15.237 | 1.00 | 70.95 | N |
| ATOM | 3513 | NH2 | ARG | B | 496 | 21.826 | 18.299 | 13.660 | 1.00 | 66.70 | N |
| ATOM | 3514 | C | ARG | B | 496 | 25.106 | 12.741 | 15.964 | 1.00 | 60.28 | C |
| ATOM | 3515 | O | ARG | B | 496 | 25.185 | 11.519 | 15.826 | 1.00 | 58.20 | O |
| ATOM | 3516 | N | SER | B | 497 | 24.183 | 13.316 | 16.721 | 1.00 | 58.58 | N |
| ATOM | 3517 | CA | SER | B | 497 | 23.175 | 12.513 | 17.407 | 1.00 | 60.66 | C |
| ATOM | 3518 | CB | SER | B | 497 | 23.267 | 12.711 | 18.920 | 1.00 | 58.31 | C |
| ATOM | 3519 | OG | SER | B | 497 | 22.882 | 14.026 | 19.276 | 1.00 | 66.25 | O |
| ATOM | 3520 | C | SER | B | 497 | 21.809 | 12.965 | 16.913 | 1.00 | 60.66 | C |
| ATOM | 3521 | O | SER | B | 497 | 21.711 | 13.896 | 16.114 | 1.00 | 59.92 | O |
| ATOM | 3522 | N | ALA | B | 498 | 20.764 | 12.305 | 17.396 | 1.00 | 63.28 | N |
| ATOM | 3523 | CA | ALA | B | 498 | 19.399 | 12.645 | 17.015 | 1.00 | 61.54 | C |
| ATOM | 3524 | CB | ALA | B | 498 | 18.440 | 11.531 | 17.450 | 1.00 | 58.15 | C |
| ATOM | 3525 | C | ALA | B | 498 | 18.995 | 13.963 | 17.671 | 1.00 | 60.15 | C |
| ATOM | 3526 | G | ALA | B | 498 | 19.422 | 14.275 | 18.783 | 1.00 | 59.70 | O |
| ATOM | 3527 | N | GLY | B | 499 | 18.168 | 14.733 | 16.977 | 1.00 | 61.09 | N |
| ATOM | 3528 | CA | GLY | B | 499 | 17.719 | 15.994 | 17.533 | 1.00 | 63.27 | C |
| ATOM | 3529 | C | GLY | B | 499 | 18.325 | 17.184 | 16.826 | 1.00 | 61.70 | C |
| ATOM | 3530 | O | GLY | B | 499 | 19.309 | 17.051 | 16.096 | 1.00 | 63.96 | O |
| ATOM | 3531 | N | LYS | B | 500 | 17.735 | 18.352 | 17.042 | 1.00 | 59.76 | N |
| ATOM | 3532 | CA | LYS | B | 500 | 18.216 | 19.576 | 16.417 | 1.00 | 57.53 | C |
| ATOM | 3533 | CB | LYS | B | 500 | 17.052 | 20.550 | 16.210 | 1.00 | 60.45 | C |
| ATOM | 3534 | CG | LYS | B | 500 | 16.017 | 20.091 | 15.199 | 1.00 | 61.48 | C |
| ATOM | 3535 | CD | LYS | B | 500 | 16.561 | 20.185 | 13.780 | 1.00 | 65.74 | C |
| ATOM | 3536 | CE | LYS | B | 500 | 15.500 | 19.800 | 12.756 | 1.00 | 62.41 | C |
| ATOM | 3537 | NZ | LYS | B | 500 | 16.013 | 19.898 | 11.358 | 1.00 | 67.47 | N |
| ATOM | 3538 | C | LYS | B | 500 | 19.283 | 20.251 | 17.275 | 1.00 | 51.49 | C |
| ATOM | 3539 | O | LYS | B | 500 | 19.282 | 20.137 | 18.495 | 1.00 | 49.86 | O |
| ATOM | 3540 | N | TRP | B | 501 | 20.195 | 20.957 | 16.618 | 1.00 | 45.23 | N |
| ATOM | 3541 | CA | TRP | B | 501 | 21.253 | 21.680 | 17.325 | 1.00 | 46.44 | C |
| ATOM | 3542 | CB | TRP | B | 501 | 22.530 | 21.673 | 16.483 | 1.00 | 40.73 | C |
| ATOM | 3543 | CG | TRP | B | 501 | 23.332 | 20.423 | 16.584 | 1.00 | 38.73 | C |
| ATOM | 3544 | CD2 | TRP | B | 501 | 24.704 | 20.258 | 16.199 | 1.00 | 35.99 | C |
| ATOM | 3545 | CE2 | TRP | B | 501 | 25.085 | 18.943 | 16.562 | 1.00 | 39.61 | C |
| ATOM | 3546 | CE3 | TRP | B | 501 | 25.651 | 21.095 | 15.586 | 1.00 | 39.41 | C |
| ATOM | 3547 | CD1 | TRP | B | 501 | 22.942 | 19.235 | 17.133 | 1.00 | 40.88 | C |
| ATOM | 3548 | NE1 | TRP | B | 501 | 23.992 | 18.339 | 17.130 | 1.00 | 40.09 | N |
| ATOM | 3549 | CZ3 | TRP | B | 501 | 26.375 | 18.448 | 16.337 | 1.00 | 35.77 | C |
| ATOM | 3550 | CZ3 | TRP | B | 501 | 26.942 | 20.597 | 15.361 | 1.00 | 34.28 | C |
| ATOM | 3551 | CH2 | TRP | B | 501 | 27.286 | 19.287 | 15.739 | 1.00 | 40.15 | C |
| ATOM | 3552 | C | TRP | B | 501 | 20.795 | 23.126 | 17.545 | 1.00 | 39.13 | C |
| ATOM | 3553 | O | TRP | B | 501 | 20.193 | 23.722 | 16.648 | 1.00 | 42.06 | O |
| ATOM | 3554 | N | PRO | B | 502 | 21.060 | 23.703 | 18.736 | 1.00 | 35.62 | N |
| ATOM | 3555 | CD | PRO | B | 502 | 21.732 | 23.060 | 19.884 | 1.00 | 36.26 | C |
| ATOM | 3556 | CA | PRO | B | 502 | 20.675 | 25.094 | 19.056 | 1.00 | 38.57 | C |
| ATOM | 3557 | CB | PRO | B | 502 | 20.841 | 25.158 | 20.569 | 1.00 | 36.44 | C |
| ATOM | 3558 | CG | PRO | B | 502 | 22.021 | 24.232 | 20.803 | 1.00 | 42.17 | C |
| ATOM | 3559 | C | PRO | B | 502 | 21.650 | 26.019 | 18.328 | 1.00 | 34.08 | C |
| ATOM | 3560 | O | PRO | B | 502 | 22.528 | 26.650 | 18.941 | 1.00 | 27.50 | O |
| ATOM | 3561 | N | LEU | B | 503 | 21.487 | 26.069 | 17.011 | 1.00 | 27.87 | N |
| ATOM | 3562 | CA | LEU | B | 503 | 22.377 | 26.847 | 16.147 | 1.00 | 30.99 | C |
| ATOM | 3563 | CB | LEU | B | 503 | 21.846 | 26.834 | 14.706 | 1.00 | 35.93 | C |
| ATOM | 3564 | CG | LEU | B | 503 | 21.555 | 25.457 | 14.117 | 1.00 | 38.86 | C |
| ATOM | 3565 | CD1 | LEU | B | 503 | 20.943 | 25.607 | 12.720 | 1.00 | 46.43 | C |
| ATOM | 3566 | CD2 | LEU | B | 503 | 22.833 | 24.656 | 14.046 | 1.00 | 42.19 | C |
| ATOM | 3567 | C | LEU | B | 503 | 22.603 | 28.287 | 16.564 | 1.00 | 24.64 | C |
| ATOM | 3568 | O | LEU | B | 503 | 23.707 | 28.826 | 16.364 | 1.00 | 24.20 | O |
| ATOM | 3569 | N | LYS | B | 504 | 21.586 | 28.914 | 17.154 | 1.00 | 24.76 | N |
| ATOM | 3570 | CA | LYS | B | 504 | 21.712 | 30.313 | 17.542 | 1.00 | 26.03 | C |
| ATOM | 3571 | CB | LYS | B | 504 | 20.353 | 30.890 | 17.939 | 1.00 | 29.28 | C |
| ATOM | 3572 | CG | LYS | B | 504 | 19.400 | 31.000 | 16.729 | 1.00 | 31.16 | C |
| ATOM | 3573 | CD | LYS | B | 504 | 18.070 | 31.656 | 17.108 | 1.00 | 33.06 | C |
| ATOM | 3574 | CE | LYS | B | 504 | 17.121 | 31.755 | 15.913 | 1.00 | 34.32 | C |
| ATOM | 3575 | NZ | LYS | B | 504 | 15.941 | 32.606 | 16.264 | 1.00 | 37.82 | N |
| ATOM | 3576 | C | LYS | B | 504 | 22.726 | 30.522 | 18.657 | 1.00 | 28.80 | C |
| ATOM | 3577 | O | LYS | B | 504 | 23.130 | 31.663 | 18.884 | 1.00 | 20.01 | O |
| ATOM | 3578 | N | TRP | B | 505 | 23.141 | 29.497 | 19.338 | 1.00 | 23.22 | N |
| ATOM | 3579 | CA | TRP | B | 505 | 24.134 | 29.656 | 20.408 | 1.00 | 19.35 | C |
| ATOM | 3580 | CB | TRP | B | 505 | 23.681 | 28.883 | 21.665 | 1.00 | 25.81 | C |
| ATOM | 3581 | CG | TRP | B | 505 | 22.680 | 29.620 | 22.472 | 1.00 | 24.36 | C |
| ATOM | 3582 | CD2 | TRP | B | 505 | 21.277 | 29.724 | 22.215 | 1.00 | 22.43 | C |
| ATOM | 3583 | CE2 | TRP | B | 505 | 20.739 | 30.586 | 23.194 | 1.00 | 23.82 | C |
| ATOM | 3584 | CE3 | TRP | B | 505 | 20.415 | 30.389 | 21.251 | 1.00 | 27.42 | C |
| ATOM | 3585 | CD1 | TRP | B | 505 | 22.934 | 30.389 | 23.567 | 1.00 | 18.37 | C |
| ATOM | 3586 | NE1 | TRP | B | 505 | 21.771 | 30.980 | 24.005 | 1.00 | 23.48 | N |
| ATOM | 3587 | CZ2 | TRP | B | 505 | 19.371 | 30.916 | 23.245 | 1.00 | 31.39 | C |
| ATOM | 3588 | CZ3 | TRP | B | 505 | 19.051 | 29.496 | 21.299 | 1.00 | 28.93 | C |
| ATOM | 3589 | CH2 | TRP | B | 505 | 18.547 | 30.365 | 22.296 | 1.00 | 25.25 | C |
| ATOM | 3590 | C | TRP | B | 505 | 25.497 | 29.098 | 19.954 | 1.00 | 19.69 | C |
| ATOM | 3591 | O | TRP | B | 505 | 26.496 | 29.233 | 20.663 | 1.00 | 22.22 O | |
| ATOM | 3592 | N | TYR | B | 506 | 25.546 | 28.513 | 18.767 | 1.00 | 21.76 | N |
| ATOM | 3593 | CA | TYR | B | 506 | 26.794 | 27.885 | 18.285 | 1.00 | 22.94 | C |
| ATOM | 3594 | CB | TYR | B | 506 | 26.426 | 26.615 | 17.488 | 1.00 | 21.82 | C |
| ATOM | 3595 | CG | TYR | B | 506 | 26.093 | 25.389 | 18.339 | 1.00 | 23.08 | C |
| ATOM | 3596 | CD1 | TYR | B | 506 | 25.885 | 25.496 | 19.710 | 1.00 | 25.11 | C |
| ATOM | 3597 | CE1 | TYR | B | 506 | 25.559 | 24.358 | 20.489 | 1.00 | 34.90 | C |
| ATOM | 3598 | CD2 | TYR | B | 506 | 25.981 | 24.124 | 17.751 | 1.00 | 29.64 | C |
| ATOM | 3599 | CE2 | TYR | B | 506 | 25.669 | 22.993 | 18.524 | 1.00 | 32.44 | C |
| ATOM | 3600 | CZ | TYR | B | 506 | 25.454 | 23.117 | 19.880 | 1.00 | 32.19 | C |
| ATOM | 3601 | OH | TYR | B | 506 | 25.086 | 22.008 | 20.619 | 1.00 | 37.37 | O |
| ATOM | 3602 | C | TYR | B | 506 | 27.745 | 28.757 | 17.434 | 1.00 | 28.60 | C |
| ATOM | 3603 | O | TYR | B | 506 | 27.299 | 29.447 | 16.524 | 1.00 | 25.58 | O |
| ATOM | 3604 | N | ALA | B | 507 | 29.048 | 28.703 | 17.731 | 1.00 | 23.48 | N |
| ATOM | 3605 | CA | ALA | B | 507 | 30.061 | 29.440 | 16.980 | 1.00 | 22.63 | C |
| ATOM | 3606 | CB | ALA | B | 507 | 31.443 | 29.298 | 17.674 | 1.00 | 19.24 | C |
| ATOM | 3607 | C | ALA | B | 507 | 30.122 | 28.845 | 15.567 | 1.00 | 24.54 | C |
| ATOM | 3608 | O | ALA | B | 507 | 29.709 | 27.709 | 15.343 | 1.00 | 23.92 | O |
| ATOM | 3609 | N | PRO | B | 508 | 30.659 | 29.597 | 14.603 | 1.00 | 22.52 | N |
| ATOM | 3610 | CD | PRO | B | 508 | 31.136 | 30.985 | 14.747 | 1.00 | 24.20 | C |
| ATOM | 3611 | CA | PRO | B | 508 | 30.772 | 29.135 | 13.212 | 1.00 | 23.99 | C |
| ATOM | 3612 | CB | PRO | B | 508 | 31.546 | 30.265 | 12.529 | 1.00 | 28.71 | C |
| ATOM | 3613 | CG | PRO | B | 508 | 31.113 | 31.477 | 13.328 | 1.00 | 35.43 | C |
| ATOM | 3614 | C | PRO | B | 508 | 31.452 | 27.787 | 13.007 | 1.00 | 23.80 | C |
| ATOM | 3615 | O | PRO | B | 508 | 30.960 | 26.964 | 12.231 | 1.00 | 23.69 | O |
| ATOM | 3616 | N | GLU | B | 509 | 32.576 | 27.568 | 13.695 | 1.00 | 22.83 | N |
| ATOM | 3617 | CA | GLU | B | 509 | 33.316 | 26.315 | 13.564 | 1.00 | 26.16 | C |
| ATOM | 3618 | CB | GLU | B | 509 | 34.689 | 26.390 | 14.268 | 1.00 | 26.11 | C |
| ATOM | 3619 | CG | GLU | B | 509 | 34.622 | 26.429 | 15.792 | 1.00 | 25.28 | C |
| ATOM | 3620 | CD | GLU | B | 509 | 34.538 | 27.843 | 16.341 | 1.00 | 30.45 | C |
| ATOM | 3621 | OE1 | GLU | B | 509 | 34.270 | 28.798 | 15.561 | 1.00 | 24.15 | O |
| ATOM | 3622 | OE2 | GLU | B | 509 | 34.716 | 27.989 | 17.568 | 1.00 | 25.31 | O |
| ATOM | 3623 | C | GLU | B | 509 | 32.512 | 25.128 | 14.098 | 1.00 | 22.82 | C |
| ATOM | 3624 | O | GLU | B | 509 | 32.751 | 23.992 | 13.686 | 1.00 | 25.61 | O |
| ATOM | 3625 | N | CYS | B | 510 | 31.571 | 25.389 | 15.010 | 1.00 | 20.67 | N |
| ATOM | 3626 | CA | CYS | B | 510 | 30.726 | 24.323 | 15.536 | 1.00 | 17.87 | C |
| ATOM | 3627 | CB | CYS | B | 510 | 29.867 | 24.838 | 16.704 | 1.00 | 17.65 | C |
| ATOM | 3628 | SG | CYS | B | 510 | 30.852 | 25.448 | 18.085 | 1.00 | 24.98 | S |
| ATOM | 3629 | C | CYS | B | 510 | 29.772 | 23.849 | 14.428 | 1.00 | 20.96 | C |
| ATOM | 3630 | O | CYS | B | 510 | 29.602 | 22.676 | 14.188 | 1.00 | 25.35 | O |
| ATOM | 3631 | N | ILE | B | 511 | 29.149 | 24.803 | 13.763 | 1.00 | 21.34 | N |
| ATOM | 3632 | CA | ILE | B | 511 | 28.188 | 24.473 | 12.713 | 1.00 | 26.63 | C |
| ATOM | 3633 | CB | ILE | B | 511 | 27.286 | 25.707 | 12.388 | 1.00 | 27.47 | C |
| ATOM | 3634 | CG2 | ILE | B | 511 | 26.264 | 25.345 | 11.289 | 1.00 | 33.99 | C |
| ATOM | 3635 | CG1 | ILE | B | 511 | 26.574 | 26.153 | 13.671 | 1.00 | 26.92 | C |
| ATOM | 3636 | CD1 | ILE | B | 511 | 25.895 | 27.516 | 13.597 | 1.00 | 31.85 | C |
| ATOM | 3637 | C | ILE | B | 511 | 28.871 | 23.984 | 11.446 | 1.00 | 30.34 | C |
| ATOM | 3638 | O | ILE | B | 511 | 28.434 | 22.987 | 10.848 | 1.00 | 29.71 | O |
| ATOM | 3639 | N | ASN | B | 512 | 29.968 | 24.623 | 11.058 | 1.00 | 25.07 | N |
| ATOM | 3640 | CA | ASN | B | 512 | 30.615 | 24.223 | 9.825 | 1.00 | 25.52 | C |
| ATOM | 3641 | CB | ASN | B | 512 | 31.416 | 25.376 | 9.236 | 1.00 | 23.08 | C |
| ATOM | 3642 | CG | ASN | B | 512 | 30.539 | 26.354 | 8.817 | 1.00 | 31.89 | C |
| ATOM | 3643 | OD1 | ASN | B | 512 | 29.466 | 26.336 | 8.268 | 1.00 | 35.21 | O |
| ATOM | 3644 | ND2 | ASN | B | 512 | 31.000 | 27.747 | 9.072 | 1.00 | 40.49 | N |
| ATOM | 3645 | C | ASN | B | 512 | 31.518 | 23.014 | 9.970 | 1.00 | 30.37 | C |
| ATOM | 3646 | O | ASN | B | 512 | 31.633 | 22.224 | 9.045 | 1.00 | 30.30 | O |
| ATOM | 3647 | N | PHE | B | 513 | 32.178 | 22.874 | 11.109 | 1.00 | 23.05 | N |
| ATOM | 3648 | CA | PHE | B | 513 | 33.084 | 21.744 | 11.273 | 1.00 | 21.06 | C |
| ATOM | 3649 | CB | PHE | B | 513 | 34.513 | 22.245 | 11.303 | 1.00 | 21.42 | C |
| ATOM | 3650 | CG | PHE | B | 513 | 34.924 | 22.945 | 10.031 | 1.00 | 32.74 | C |
| ATOM | 3651 | CD1 | PHE | B | 513 | 35.063 | 24.326 | 10.005 | 1.00 | 34.47 | C |
| ATOM | 3652 | CD2 | PHE | B | 513 | 35.120 | 22.216 | 8.858 | 1.00 | 32.93 | C |
| ATOM | 3653 | CE1 | PHE | B | 513 | 35.393 | 24.986 | 8.805 | 1.00 | 39.39 | C |
| ATOM | 3654 | CE2 | PHE | B | 513 | 35.448 | 22.871 | 7.664 | 1.00 | 40.01 | C |
| ATOM | 3655 | CZ | PHE | B | 513 | 35.581 | 24.253 | 7.645 | 1.00 | 36.34 | C |
| ATOM | 3656 | C | PHE | B | 513 | 32.856 | 20.836 | 12.477 | 1.00 | 24.61 | C |
| ATOM | 3657 | O | PHE | B | 513 | 33.687 | 19.968 | 12.741 | 1.00 | 25.10 | O |
| ATOM | 3658 | N | ARG | B | 514 | 31.758 | 21.060 | 13.207 | 1.00 | 20.51 | N |
| ATOM | 3659 | CA | ARG | B | 514 | 31.407 | 20.266 | 14.381 | 1.00 | 21.86 | C |
| ATOM | 3660 | CB | ARG | B | 514 | 31.062 | 18.815 | 13.944 | 1.00 | 21.50 | C |
| ATOM | 3661 | CG | ARG | B | 514 | 29.938 | 17.415 | 12.935 | 1.00 | 22.64 | C |
| ATOM | 3662 | CD | ARG | B | 514 | 29.437 | 17.550 | 12.570 | 1.00 | 25.89 | N |
| ATOM | 3663 | NE | ARG | B | 514 | 28.284 | 17.550 | 11.668 | 1.00 | 25.50 | N |
| ATOM | 3664 | CZ | ARG | B | 514 | 27.531 | 16.537 | 11.265 | 1.00 | 27.87 | C |
| ATOM | 3665 | NH1 | ARG | B | 514 | 27.803 | 15.306 | 11.672 | 1.00 | 28.30 | N |
| ATOM | 3666 | NH2 | ARG | B | 514 | 26.476 | 16.760 | 10.478 | 1.00 | 28.78 | N |
| ATOM | 3667 | C | ARG | B | 514 | 32.560 | 20.260 | 15.395 | 1.00 | 21.80 | C |
| ATOM | 3668 | O | ARG | B | 514 | 32.735 | 19.299 | 16.141 | 1.00 | 23.13 | O |
| ATOM | 3669 | N | LYS | B | 515 | 33.288 | 21.373 | 15.438 | 1.00 | 23.39 | N |
| ATOM | 3670 | CA | LYS | B | 515 | 34.442 | 21.512 | 16.323 | 1.00 | 21.15 | C |
| ATOM | 3671 | CB | LYS | B | 515 | 35.551 | 22.203 | 15.532 | 1.00 | 22.48 | C |
| ATOM | 3672 | CG | LYS | B | 515 | 36.848 | 22.399 | 16.309 | 1.00 | 23.37 | C |
| ATOM | 3673 | CD | LYS | B | 515 | 37.848 | 23.195 | 16.490 | 1.00 | 30.48 | C |
| ATOM | 3674 | CE | LYS | B | 515 | 38.992 | 23.662 | 16.372 | 1.00 | 37.06 | C |
| ATOM | 3675 | NZ | LYS | B | 515 | 39.892 | 34.621 | 15.659 | 1.00 | 43.43 | N |
| ATOM | 3676 | C | LYS | B | 515 | 34.100 | 22.315 | 17.597 | 1.00 | 22.11 | C |
| ATOM | 3677 | O | LYS | B | 515 | 33.704 | 23.477 | 17.503 | 1.00 | 20.32 | O |
| ATOM | 3678 | N | PHE | B | 516 | 34.302 | 21.712 | 18.773 | 1.00 | 22.31 | N |
| ATOM | 3679 | CA | PHE | B | 516 | 33.987 | 22.381 | 20.045 | 1.00 | 19.14 | C |
| ATOM | 3680 | CB | PHE | B | 516 | 32.903 | 21.592 | 20.788 | 1.00 | 20.60 | C |
| ATOM | 3681 | CG | PHE | B | 516 | 31.585 | 21.555 | 20.037 | 1.00 | 21.15 | C |
| ATOM | 3682 | CD1 | PHE | B | 516 | 31.411 | 20.683 | 18.963 | 1.00 | 22.16 | C |
| ATOM | 3683 | CD2 | PHE | B | 516 | 30.559 | 22.439 | 20.371 | 1.00 | 25.22 | C |
| ATOM | 3684 | CE1 | PHE | B | 516 | 30.223 | 20.683 | 18.229 | 1.00 | 25.00 | C |
| ATOM | 3685 | CE2 | PHE | B | 516 | 29.374 | 22.450 | 19.629 | 1.00 | 23.08 | C |
| ATOM | 3686 | CZ | PHE | B | 516 | 29.209 | 21.565 | 18.561 | 1.00 | 27.70 | C |
| ATOM | 3687 | C | PHE | B | 516 | 35.214 | 22.578 | 20.911 | 1.00 | 19.07 | C |
| ATOM | 3688 | O | PHE | B | 516 | 36.127 | 21.732 | 20.908 | 1.00 | 21.35 | O |
| ATOM | 3689 | N | SER | B | 517 | 35.206 | 23.667 | 21.684 | 1.00 | 19.27 | N |
| ATOM | 3690 | CA | SER | B | 517 | 36.366 | 24.059 | 22.496 | 1.00 | 26.23 | C |
| ATOM | 3691 | CB | SER | B | 517 | 37.385 | 24.718 | 21.542 | 1.00 | 24.44 | C |
| ATOM | 3692 | OG | SER | B | 517 | 36.770 | 25.861 | 20.910 | 1.00 | 22.44 | O |
| ATOM | 3693 | C | SER | B | 517 | 35.935 | 25.102 | 23.528 | 1.00 | 21.53 | C |
| ATOM | 3694 | O | SER | B | 517 | 34.792 | 25.509 | 23.523 | 1.00 | 20.37 | O |
| ATOM | 3695 | N | SER | B | 518 | 36.836 | 25.554 | 24.404 | 1.00 | 19.72 | N |
| ATOM | 3696 | CA | SER | B | 518 | 36.435 | 26.590 | 25.329 | 1.00 | 20.34 | C |
| ATOM | 3697 | CB | SER | B | 518 | 37.555 | 26.890 | 26.341 | 1.00 | 27.31 | C |
| ATOM | 3698 | OG | SER | B | 518 | 37.592 | 25.809 | 27.275 | 1.00 | 32.37 | O |
| ATOM | 3699 | C | SER | B | 518 | 36.039 | 27.842 | 24.551 | 1.00 | 21.23 | C |
| ATOM | 3700 | O | SER | B | 518 | 35.165 | 28.589 | 24.977 | 1.00 | 23.54 | O |
| ATOM | 3701 | N | ARG | B | 519 | 36.689 | 28.055 | 23.421 | 1.00 | 21.32 | N |
| ATOM | 3702 | CA | ARG | B | 519 | 36.347 | 29.205 | 22.610 | 1.00 | 18.97 | C |
| ATOM | 3703 | CB | ARG | B | 519 | 37.414 | 29.420 | 21.545 | 1.00 | 21.22 | C |
| ATOM | 3704 | CG | ARG | B | 519 | 38.585 | 30.196 | 22.148 | 1.00 | 26.38 | C |
| ATOM | 3705 | CD | ARG | B | 519 | 39.753 | 30.191 | 21.208 | 1.00 | 35.68 | C |
| ATOM | 3706 | NE | ARG | B | 519 | 40.851 | 30.943 | 21.789 | 1.00 | 42.06 | N |
| ATOM | 3707 | CZ | ARG | B | 519 | 42.082 | 30.952 | 21.288 | 1.00 | 46.57 | C |
| ATOM | 3708 | NH1 | ARG | B | 519 | 42.367 | 30.246 | 20.195 | 1.00 | 43.72 | N |
| ATOM | 3709 | NH2 | ARG | B | 519 | 43.032 | 31.653 | 21.892 | 1.00 | 49.64 | N |
| ATOM | 3710 | C | ARG | B | 519 | 34.964 | 29.121 | 21.987 | 1.00 | 19.81 | C |
| ATOM | 3711 | O | ARG | B | 519 | 34.363 | 30.171 | 21.729 | 1.00 | 21.27 | O |
| ATOM | 3712 | N | SER | B | 520 | 34.460 | 27.909 | 21.718 | 1.00 | 18.58 | N |
| ATOM | 3713 | CA | SER | B | 520 | 33.075 | 27.864 | 21.213 | 1.00 | 21.84 | C |
| ATOM | 3714 | CB | SER | B | 520 | 32.742 | 26.535 | 20.503 | 1.00 | 20.81 | C |
| ATOM | 3715 | OG | SER | B | 520 | 32.908 | 25.379 | 21.297 | 1.00 | 22.92 | O |
| ATOM | 3716 | C | SER | B | 520 | 32.158 | 28.162 | 22.437 | 1.00 | 23.07 | C |
| ATOM | 3717 | O | SER | B | 520 | 31.091 | 28.760 | 22.290 | 1.00 | 21.17 | O |
| ATOM | 3718 | N | ASP | B | 521 | 32.597 | 27.810 | 23.649 | 1.00 | 19.80 | N |
| ATOM | 3719 | CA | ASP | B | 521 | 31.824 | 28.119 | 24.877 | 1.00 | 21.41 | C |
| ATOM | 3720 | CB | ASP | B | 521 | 32.462 | 27.523 | 26.150 | 1.00 | 28.78 | C |
| ATOM | 3721 | CG | ASP | B | 521 | 32.050 | 26.058 | 26.409 | 1.00 | 22.85 | C |
| ATOM | 3722 | OD1 | ASP | B | 521 | 31.099 | 25.558 | 25.763 | 1.00 | 22.20 | O |
| ATOM | 3723 | OD2 | ASP | B | 521 | 32.683 | 25.426 | 27.306 | 1.00 | 22.04 | O |
| ATOM | 3724 | C | ASP | B | 521 | 31.827 | 29.656 | 25.044 | 1.00 | 17.40 | C |
| ATOM | 3725 | O | ASP | B | 521 | 30.822 | 30.257 | 25.477 | 1.00 | 20.22 | O |
| ATOM | 3726 | N | VAL | B | 522 | 32.944 | 30.299 | 24.673 | 1.00 | 16.61 | N |
| ATOM | 3727 | CA | VAL | B | 522 | 32.990 | 31.785 | 24.758 | 1.00 | 15.75 | C |
| ATOM | 3728 | CB | VAL | B | 522 | 34.397 | 32.326 | 24.385 | 1.00 | 18.63 | C |
| ATOM | 3729 | CG1 | VAL | B | 522 | 34.392 | 33.849 | 24.190 | 1.00 | 20.55 | C |
| ATOM | 3730 | CG2 | VAL | B | 522 | 35.377 | 31.957 | 25.537 | 1.00 | 18.25 | C |
| ATOM | 3731 | C | VAL | B | 522 | 31.927 | 32.402 | 23.834 | 1.00 | 16.27 | C |
| ATOM | 3732 | O | VAL | B | 522 | 31.243 | 33.352 | 24.221 | 1.00 | 18.90 | O |
| ATOM | 3733 | N | TRP | B | 523 | 31.783 | 31.848 | 22.634 | 1.00 | 17.66 | N |
| ATOM | 3734 | CA | TRP | B | 523 | 30.758 | 32.371 | 21.708 | 1.00 | 16.82 | C |
| ATOM | 3735 | CB | TRP | B | 523 | 30.805 | 31.584 | 20.386 | 1.00 | 15.58 | C |
| ATOM | 3736 | CG | TRP | B | 523 | 29.813 | 32.106 | 19.335 | 1.00 | 17.41 | C |
| ATOM | 3737 | CD2 | TRP | B | 523 | 30.129 | 32.847 | 18.145 | 1.00 | 19.38 | C |
| ATOM | 3738 | CE2 | TRP | B | 523 | 28.904 | 33.164 | 17.516 | 1.00 | 20.03 | C |
| ATOM | 3739 | CE3 | TRP | B | 523 | 31.332 | 33.280 | 17.546 | 1.00 | 18.44 | C |
| ATOM | 3740 | CD1 | TRP | B | 523 | 28.448 | 31.999 | 19.375 | 1.00 | 22.97 | C |
| ATOM | 3741 | NE1 | TRP | B | 523 | 27.891 | 32.641 | 18.280 | 1.00 | 18.65 | N |
| ATOM | 3742 | CZ2 | TRP | B | 523 | 28.839 | 33.895 | 16.322 | 1.00 | 21.60 | C |
| ATOM | 3743 | CZ3 | TRP | B | 523 | 31.263 | 34.010 | 16.359 | 1.00 | 19.75 | C |
| ATOM | 3744 | CH2 | TRP | B | 523 | 30.030 | 34.313 | 15.728 | 1.00 | 19.91 | C |
| ATOM | 3745 | C | TRP | B | 523 | 29.373 | 32.241 | 22.358 | 1.00 | 18.12 | C |
| ATOM | 3746 | O | TRP | B | 523 | 28.573 | 33.189 | 22.321 | 1.00 | 19.38 | O |
| ATOM | 3747 | N | SER | B | 524 | 29.088 | 31.082 | 22.956 | 1.00 | 17.71 | N |
| ATOM | 3748 | CA | SER | B | 524 | 27.791 | 30.853 | 23.643 | 1.00 | 22.69 | C |
| ATOM | 3749 | CB | SER | B | 524 | 27.740 | 29.419 | 24.213 | 1.00 | 21.28 | C |
| ATOM | 3750 | OG | SER | B | 524 | 27.817 | 28.467 | 23.185 | 1.00 | 31.98 | O |
| ATOM | 3751 | C | SER | B | 524 | 27.579 | 31.829 | 24.792 | 1.00 | 22.41 | C |
| ATOM | 3752 | O | SER | B | 524 | 26.464 | 32.331 | 25.014 | 1.00 | 18.61 | O |
| ATOM | 3753 | N | TYR | B | 525 | 28.644 | 32.099 | 25.546 | 1.00 | 22.85 | N |
| ATOM | 3754 | CA | TYR | B | 525 | 28.619 | 33.051 | 26.640 | 1.00 | 21.01 | C |
| ATOM | 3755 | CB | TYR | B | 525 | 30.040 | 33.201 | 27.268 | 1.00 | 19.16 | C |
| ATOM | 3756 | CG | TYR | B | 525 | 30.046 | 34.181 | 28.409 | 1.00 | 20.09 | C |
| ATOM | 3757 | CD1 | TYR | B | 525 | 29.456 | 33.865 | 26.638 | 1.00 | 21.83 | C |
| ATOM | 3758 | CE1 | TYR | B | 525 | 29.411 | 34.806 | 30.692 | 1.00 | 20.51 | C |
| ATOM | 3759 | CD2 | TYR | B | 525 | 30.602 | 35.461 | 28.251 | 1.00 | 19.44 | C |
| ATOM | 3760 | CE2 | TYR | B | 525 | 30.553 | 36.400 | 29.286 | 1.00 | 21.12 | C |
| ATOM | 3761 | CZ | TYR | B | 525 | 29.956 | 36.066 | 30.501 | 1.00 | 26.32 | C |
| ATOM | 3762 | OH | TYR | B | 525 | 29.911 | 36.999 | 31.518 | 1.00 | 24.81 | O |
| ATOM | 3763 | C | TYR | B | 525 | 28.159 | 34.407 | 26.098 | 1.00 | 20.91 | C |
| ATOM | 3764 | O | TYR | B | 525 | 27.344 | 35.092 | 26.718 | 1.00 | 20.39 | O |
| ATOM | 3765 | N | GLY | B | 526 | 28.711 | 34.811 | 24.950 | 1.00 | 18.42 | N |
| ATOM | 3766 | CA | GLY | B | 526 | 28.306 | 36.086 | 24.357 | 1.00 | 19.09 | C |
| ATOM | 3767 | C | GLY | B | 526 | 26.803 | 36.116 | 34.081 | 1.00 | 16.71 | C |
| ATOM | 3768 | O | GLY | B | 526 | 26.135 | 37.118 | 24.371 | 1.00 | 19.95 | O |
| ATOM | 3769 | N | VAL | B | 527 | 26.271 | 35.039 | 23.506 | 1.00 | 19.63 | N |
| ATOM | 3770 | CA | VAL | B | 527 | 24.821 | 34.980 | 23.240 | 1.00 | 17.92 | C |
| ATOM | 3771 | CB | VAL | B | 527 | 24.470 | 33.705 | 22.461 | 1.00 | 18.12 | C |
| ATOM | 3772 | CG1 | VAL | B | 527 | 22.943 | 33.645 | 22.145 | 1.00 | 19.94 | C |
| ATOM | 3773 | CG2 | VAL | B | 527 | 25.270 | 33.697 | 21.122 | 1.00 | 19.40 | C |
| ATOM | 3774 | C | VAL | B | 527 | 24.082 | 35.026 | 24.600 | 1.00 | 20.57 | C |
| ATOM | 3775 | O | VAL | B | 527 | 23.063 | 35.686 | 24.735 | 1.00 | 21.39 | O |
| ATOM | 3776 | N | THR | B | 528 | 24.633 | 34.377 | 25.619 | 1.00 | 20.54 | N |
| ATOM | 3777 | CA | THR | B | 528 | 24.010 | 34.397 | 26.949 | 1.00 | 18.80 | C |
| ATOM | 3778 | CB | THR | B | 528 | 24.768 | 33.458 | 27.906 | 1.00 | 20.59 | C |
| ATOM | 3779 | OG1 | THR | B | 528 | 24.673 | 32.114 | 27.395 | 1.00 | 21.50 | O |
| ATOM | 3780 | CG2 | THR | B | 528 | 24.169 | 33.499 | 29.287 | 1.00 | 22.95 | C |
| ATOM | 3781 | C | THR | B | 528 | 23.981 | 35.821 | 27.507 | 1.00 | 25.29 | C |
| ATOM | 3782 | O | THR | B | 528 | 22.972 | 36.235 | 28.084 | 1.00 | 19.03 | O |
| ATOM | 3783 | N | MET | B | 529 | 25.083 | 36.560 | 27.337 | 1.00 | 21.53 | N |
| ATOM | 3784 | CA | MET | B | 529 | 25.147 | 37.955 | 27.789 | 1.00 | 20.49 | C |
| ATOM | 3785 | CB | MET | B | 529 | 26.479 | 38.621 | 37.360 | 1.00 | 20.90 | C |
| ATOM | 3786 | CG | MET | B | 529 | 27.726 | 38.072 | 28.092 | 1.00 | 22.65 | C |
| ATOM | 3787 | SD | MET | B | 529 | 29.178 | 39.101 | 27.591 | 1.00 | 24.81 | S |
| ATOM | 3788 | CE | MET | B | 529 | 28.792 | 40.693 | 28.291 | 1.00 | 22.12 | C |
| ATOM | 3789 | C | MET | B | 529 | 24.013 | 38.740 | 27.103 | 1.00 | 19.53 | C |
| ATOM | 3790 | O | MET | B | 529 | 23.331 | 39.551 | 27.739 | 1.00 | 20.14 | O |
| ATOM | 3791 | N | TRP | B | 530 | 23.843 | 38.505 | 25.802 | 1.00 | 20.92 | N |
| ATOM | 3792 | CA | TRP | B | 530 | 22.800 | 39.234 | 25.035 | 1.00 | 19.60 | C |
| ATOM | 3793 | CB | TRP | B | 530 | 22.848 | 38.845 | 23.556 | 1.00 | 20.03 | C |
| ATOM | 3794 | CG | TRP | B | 530 | 21.996 | 39.725 | 22.693 | 1.00 | 18.41 | C |
| ATOM | 3795 | CD2 | TRP | B | 530 | 20.610 | 39.533 | 22.395 | 1.00 | 19.07 | C |
| ATOM | 3796 | CE2 | TRP | B | 530 | 20.191 | 40.635 | 21.590 | 1.00 | 20.94 | C |
| ATOM | 3797 | CE3 | TRP | B | 530 | 19.678 | 38.543 | 22.726 | 1.00 | 22.34 | C |
| ATOM | 3798 | CD1 | TRP | B | 530 | 22.373 | 40.909 | 22.072 | 1.00 | 18.66 | C |
| ATOM | 3799 | NE1 | TRP | B | 530 | 21.276 | 41.460 | 21.399 | 1.00 | 21.22 | N |
| ATOM | 3800 | CZ2 | TRP | B | 530 | 18.866 | 40.762 | 21.116 | 1.00 | 26.05 | C |
| ATOM | 3801 | CZ3 | TRP | B | 530 | 18.369 | 38.673 | 22.254 | 1.00 | 23.03 | C |
| ATOM | 3802 | CH2 | TRP | B | 530 | 17.978 | 39.770 | 21.462 | 1.00 | 27.41 | C |
| ATOM | 3803 | C | TRP | B | 530 | 21.403 | 38.947 | 25.567 | 1.00 | 21.14 | C |
| ATOM | 3804 | O | TRP | B | 530 | 20.607 | 39.880 | 25.807 | 1.00 | 22.19 | O |
| ATOM | 3805 | N | GLU | B | 531 | 21.101 | 37.665 | 25.754 | 1.00 | 21.34 | N |
| ATOM | 3806 | CA | GLU | B | 531 | 19.799 | 37.234 | 26.288 | 1.00 | 22.58 | C |
| ATOM | 3807 | CB | GLU | B | 531 | 19.785 | 35.725 | 26.566 | 1.00 | 23.47 | C |
| ATOM | 3808 | CG | GLU | B | 531 | 19.941 | 34.786 | 25.384 | 1.00 | 26.14 | C |
| ATOM | 3809 | CD | GLU | B | 531 | 19.873 | 33.318 | 25.844 | 1.00 | 26.77 | C |
| ATOM | 3810 | OE1 | GLU | B | 531 | 20.953 | 32.715 | 26.078 | 1.00 | 22.88 | O |
| ATOM | 3811 | OE2 | GLU | B | 531 | 18.731 | 32.788 | 25.983 | 1.00 | 27.11 | O |
| ATOM | 3812 | C | GLU | B | 531 | 19.548 | 37.897 | 27.617 | 1.00 | 20.02 | C |
| ATOM | 3813 | O | GLU | B | 531 | 18.432 | 38.384 | 27.895 | 1.00 | 23.77 | O |
| ATOM | 3814 | N | ALA | B | 532 | 20.565 | 37.903 | 28.478 | 1.00 | 20.17 | N |
| ATOM | 3815 | CA | ALA | B | 532 | 20.408 | 38.497 | 29.795 | 1.00 | 18.65 | C |
| ATOM | 3816 | CB | ALA | B | 532 | 21.617 | 38.155 | 30.711 | 1.00 | 20.90 | C |
| ATOM | 3817 | C | ALA | B | 532 | 20.204 | 39.994 | 29.776 | 1.00 | 24.11 | C |
| ATOM | 3818 | O | ALA | B | 532 | 19.290 | 40.506 | 30.425 | 1.00 | 20.67 | O |
| ATOM | 3819 | N | LEU | B | 533 | 21.062 | 40.701 | 29.045 | 1.00 | 24.27 | N |
| ATOM | 3820 | CA | LEU | B | 533 | 20.980 | 42.163 | 28.975 | 1.00 | 28.04 | C |
| ATOM | 3821 | CB | LEU | B | 533 | 22.282 | 42.737 | 28.376 | 1.00 | 22.81 | C |
| ATOM | 3822 | CG | LEU | B | 533 | 23.472 | 42.615 | 29.345 | 1.00 | 21.62 | C |
| ATOM | 3823 | CD1 | LEU | B | 533 | 24.801 | 42.869 | 28.608 | 1.00 | 29.89 | C |
| ATOM | 3824 | CD2 | LEU | B | 533 | 23.309 | 43.620 | 30.508 | 1.00 | 25.39 | C |
| ATOM | 3825 | C | LEU | B | 533 | 19.745 | 42.629 | 28.194 | 1.00 | 26.38 | C |
| ATOM | 3826 | O | LEU | B | 533 | 19.352 | 43.810 | 28.275 | 1.00 | 22.57 | O |
| ATOM | 3827 | N | SER | B | 534 | 19.122 | 41.702 | 27.464 | 1.00 | 21.44 | N |
| ATOM | 3828 | CA | SER | B | 534 | 17.888 | 41.991 | 26.714 | 1.00 | 23.07 | C |
| ATOM | 3829 | CB | SER | B | 534 | 17.862 | 41.187 | 25.249 | 1.00 | 21.95 | C |
| ATOM | 3830 | OG | SER | B | 534 | 18.842 | 41.679 | 25.534 | 1.00 | 31.02 | O |
| ATOM | 3831 | C | SER | B | 534 | 16.709 | 41.560 | 27.562 | 1.00 | 24.51 | C |
| ATOM | 3832 | O | SER | B | 534 | 15.571 | 41.538 | 27.106 | 1.00 | 27.12 | O |
| ATOM | 3833 | N | TYR | B | 535 | 17.000 | 41.199 | 28.803 | 1.00 | 25.92 | N |
| ATOM | 3834 | CA | TYR | B | 535 | 15.990 | 40.738 | 39.739 | 1.00 | 28.32 | C |
| ATOM | 3835 | CB | TYR | B | 535 | 15.102 | 41.910 | 30.198 | 1.00 | 28.47 | C |
| ATOM | 3836 | CG | TYR | B | 535 | 15.858 | 42.811 | 31.144 | 1.00 | 29.31 | C |
| ATOM | 3837 | CD1 | TYR | B | 535 | 16.777 | 43.7333 | 30.652 | 1.00 | 28.02 | C |
| ATOM | 3838 | CE1 | TYR | B | 535 | 17.563 | 44.492 | 31.511 | 1.00 | 29.55 | C |
| ATOM | 3839 | CD2 | TYR | B | 535 | 15.736 | 42.679 | 32.526 | 1.00 | 28.97 | C |
| ATOM | 3840 | CE2 | TYR | B | 535 | 16.514 | 43.434 | 33.395 | 1.00 | 30.17 | C |
| ATOM | 3841 | CZ | TYR | B | 535 | 17.431 | 44.341 | 32.874 | 1.00 | 22.54 | C |
| ATOM | 3842 | OH | TYR | B | 535 | 18.185 | 45.117 | 33.713 | 1.00 | 29.23 | O |
| ATOM | 3843 | C | TYR | B | 535 | 15.137 | 39.579 | 29.224 | 1.00 | 30.50 | C |
| ATOM | 3844 | O | TYR | B | 535 | 13.918 | 39.637 | 29.245 | 1.00 | 29.15 | O |
| ATOM | 3845 | N | GLY | B | 536 | 15.805 | 38.525 | 28.754 | 1.00 | 25.34 | N |
| ATOM | 3846 | CA | GLY | B | 536 | 15.090 | 37.334 | 28.313 | 1.00 | 26.66 | C |
| ATOM | 3847 | C | GLY | B | 536 | 14.563 | 37.236 | 26.907 | 1.00 | 28.90 | C |
| ATOM | 3848 | O | GLY | B | 536 | 13.831 | 36.301 | 26.583 | 1.00 | 34.84 | O |
| ATOM | 3849 | N | GLN | B | 537 | 14.940 | 38.177 | 26.044 | 1.00 | 30.40 | N |
| ATOM | 3850 | CA | GLN | B | 537 | 14.504 | 38.138 | 24.658 | 1.00 | 28.41 | C |
| ATOM | 3851 | CB | GLN | B | 537 | 14.840 | 39.463 | 23.973 | 1.00 | 32.82 | C |
| ATOM | 3852 | CG | GLN | B | 537 | 13.837 | 40.571 | 24.309 | 1.00 | 43.11 | C |
| ATOM | 3853 | CD | GLN | B | 537 | 14.217 | 41.920 | 23.731 | 1.00 | 47.65 | C |
| ATOM | 3854 | OE1 | GLN | B | 537 | 14.599 | 42.029 | 22.562 | 1.00 | 54.72 | O |
| ATOM | 3855 | NE2 | GLN | B | 537 | 14.108 | 42.961 | 24.547 | 1.00 | 50.31 | N |
| ATOM | 3856 | C | GLN | B | 537 | 15.175 | 36.979 | 23.918 | 1.00 | 30.53 | C |
| ATOM | 3857 | O | GLN | B | 537 | 16.238 | 36.507 | 24.326 | 1.00 | 25.81 | O |
| ATOM | 3858 | N | LYS | B | 538 | 14.550 | 36.515 | 22.832 | 1.00 | 26.52 | N |
| ATOM | 3859 | CA | LYS | B | 538 | 15.124 | 35.249 | 22.060 | 1.00 | 26.82 | C |
| ATOM | 3860 | CB | LYS | B | 538 | 14.036 | 34.673 | 21.292 | 1.00 | 33.23 | C |
| ATOM | 3861 | CG | LYS | B | 538 | 13.077 | 33.922 | 22.201 | 1.00 | 43.54 | C |
| ATOM | 3862 | CD | LYS | B | 538 | 12.031 | 33.160 | 21.393 | 1.00 | 44.85 | C |
| ATOM | 3863 | CE | LYS | B | 538 | 11.278 | 32.173 | 22.282 | 1.00 | 50.77 | C |
| ATOM | 3864 | NZ | LYS | B | 538 | 10.275 | 31.376 | 21.506 | 1.00 | 55.00 | C |
| ATOM | 3865 | C | LYS | B | 538 | 16.142 | 35.971 | 21.055 | 1.00 | 28.86 | C |
| ATOM | 3866 | O | LYS | B | 538 | 15.915 | 37.005 | 20.425 | 1.00 | 29.11 | O |
| ATOM | 3867 | N | PRO | B | 539 | 17.289 | 35.290 | 20.917 | 1.00 | 25.45 | N |
| ATOM | 3868 | CD | PRO | B | 539 | 17.826 | 34.185 | 21.727 | 1.00 | 22.65 | C |
| ATOM | 3869 | CA | PRO | B | 539 | 18.285 | 35.763 | 19.952 | 1.00 | 24.40 | C |
| ATOM | 3870 | CB | PRO | B | 539 | 19.518 | 34.912 | 20.276 | 1.00 | 28.27 | C |
| ATOM | 3871 | CG | PRO | B | 539 | 18.920 | 33.647 | 20.824 | 1.00 | 33.25 | C |
| ATOM | 3872 | C | PRO | B | 539 | 17.828 | 35.548 | 18.507 | 1.00 | 28.17 | C |
| ATOM | 3873 | O | PRO | B | 539 | 17.136 | 34.584 | 18.201 | 1.00 | 25.84 | O |
| ATOM | 3874 | N | TYR | B | 540 | 18.229 | 36.458 | 17.630 | 1.00 | 22.91 | N |
| ATOM | 3875 | CA | TYR | B | 540 | 17.918 | 36.423 | 16.206 | 1.00 | 26.70 | C |
| ATOM | 3876 | CB | TYR | B | 540 | 18.740 | 35.333 | 15.518 | 1.00 | 23.59 | C |
| ATOM | 3877 | CG | TYR | B | 540 | 20.240 | 35.373 | 15.839 | 1.00 | 29.92 | C |
| ATOM | 3878 | CD1 | TYR | B | 540 | 21.094 | 36.237 | 15.163 | 1.00 | 24.88 | C |
| ATOM | 3879 | CE1 | TYR | B | 540 | 22.470 | 36.264 | 15.447 | 1.00 | 24.04 | C |
| ATOM | 3880 | CD2 | TYR | B | 540 | 20.773 | 34.542 | 16.821 | 1.00 | 28.81 | C |
| ATOM | 3881 | CE2 | TYR | B | 540 | 22.153 | 34.568 | 17.142 | 1.00 | 22.99 | C |
| ATOM | 3882 | CZ | TYR | B | 540 | 22.988 | 35.424 | 16.443 | 1.00 | 20.60 | C |
| ATOM | 3883 | OH | TYR | B | 540 | 24.327 | 35.447 | 16.724 | 1.00 | 23.56 | O |
| ATOM | 3884 | C | TYR | B | 540 | 16.435 | 36.134 | 16.017 | 1.00 | 25.23 | C |
| ATOM | 3885 | O | TYR | B | 540 | 16.063 | 35.273 | 15.224 | 1.00 | 28.39 | O |
| ATOM | 3886 | N | LYS | B | 541 | 15.637 | 36.889 | 16.769 | 1.00 | 28.66 | N |
| ATOM | 3887 | CA | LYS | B | 541 | 14.186 | 36.785 | 16.823 | 1.00 | 40.37 | C |
| ATOM | 3888 | CB | LYS | B | 541 | 13.642 | 38.072 | 17.450 | 1.00 | 43.79 | C |
| ATOM | 3888 | CG | LYS | B | 541 | 12.245 | 37.962 | 18.030 | 1.00 | 55.46 | C |
| ATOM | 3890 | CD | LYS | B | 541 | 11.876 | 39.211 | 18.856 | 1.00 | 55.84 | C |
| ATOM | 3891 | CE | LYS | B | 541 | 12.786 | 39.383 | 20.081 | 1.00 | 60.14 | C |
| ATOM | 3892 | NZ | LYS | B | 541 | 12.345 | 40.490 | 20.993 | 1.00 | 56.25 | N |
| ATOM | 3893 | C | LYS | B | 541 | 13.502 | 36.524 | 15.480 | 1.00 | 43.30 | C |
| ATOM | 3894 | O | LYS | B | 541 | 12.731 | 35.578 | 15.337 | 1.00 | 43.84 | O |
| ATOM | 3895 | N | LYS | B | 542 | 13.791 | 37.349 | 14.493 | 1.00 | 43.89 | N |
| ATOM | 3896 | CA | LYS | B | 542 | 13.147 | 37.178 | 13.200 | 1.00 | 50.62 | C |
| ATOM | 3897 | CB | LYS | B | 542 | 12.893 | 38.547 | 12.575 | 1.00 | 51.21 | C |
| ATOM | 3898 | CG | LYS | B | 542 | 14.153 | 39.355 | 12.339 | 1.00 | 56.73 | C |
| ATOM | 3899 | CD | LYS | B | 542 | 13.849 | 40.636 | 11.571 | 1.00 | 61.48 | C |
| ATOM | 3900 | CE | LYS | B | 542 | 15.135 | 41.350 | 11.161 | 1.00 | 60.94 | C |
| ATOM | 3901 | NZ | LYS | B | 542 | 14.892 | 42.611 | 10.395 | 1.00 | 64.23 | C |
| ATOM | 3902 | C | LYS | B | 542 | 13.921 | 36.309 | 12.221 | 1.00 | 56.90 | C |
| ATOM | 3903 | O | LYS | B | 542 | 13.821 | 36.518 | 11.008 | 1.00 | 59.68 | O |
| ATOM | 3904 | N | MET | B | 543 | 14.663 | 35.322 | 12.724 | 1.00 | 41.97 | N |
| ATOM | 3905 | CA | MET | B | 543 | 15.443 | 34.453 | 11.844 | 1.00 | 45.64 | C |
| ATOM | 3906 | CB | MET | B | 543 | 16.952 | 34.817 | 11.907 | 1.00 | 43.71 | C |
| ATOM | 3907 | CG | MET | B | 543 | 17.348 | 35.994 | 11.066 | 1.00 | 53.65 | C |
| ATOM | 3908 | SD | MET | B | 543 | 19.100 | 36.330 | 11.367 | 1.00 | 42.99 | S |
| ATOM | 3909 | CE | MET | B | 543 | 18.961 | 37.898 | 12.209 | 1.00 | 45.52 | C |
| ATOM | 3910 | C | MET | B | 543 | 15.332 | 32.979 | 12.173 | 1.00 | 47.61 | C |
| ATOM | 3911 | O | MET | B | 543 | 15.075 | 32.599 | 13.318 | 1.00 | 48.77 | O |
| ATOM | 3912 | N | LYS | B | 544 | 15.562 | 32.152 | 11.157 | 1.00 | 48.07 | N |
| ATOM | 3913 | CA | LYS | B | 544 | 15.522 | 30.704 | 11.301 | 1.00 | 54.78 | C |
| ATOM | 3914 | CB | LYS | B | 544 | 14.693 | 30.079 | 10.181 | 1.00 | 55.48 | C |
| ATOM | 3915 | CG | LYS | B | 544 | 15.303 | 30.244 | 8.792 | 1.00 | 64.05 | C |
| ATOM | 3916 | CD | LYS | B | 544 | 14.493 | 29.491 | 7.739 | 1.00 | 64.50 | C |
| ATOM | 3917 | CE | LYS | B | 544 | 15.167 | 29.517 | 6.367 | 1.00 | 65.75 | C |
| ATOM | 3918 | NZ | LYS | B | 544 | 14.420 | 28.692 | 5.363 | 1.00 | 63.25 | N |
| ATOM | 3919 | C | LYS | B | 544 | 16.953 | 30.168 | 11.231 | 1.00 | 56.68 | C |
| ATOM | 3920 | O | LYS | B | 544 | 17.908 | 30.904 | 11.473 | 1.00 | 54.87 | O |
| ATOM | 3921 | N | GLY | B | 545 | 17.092 | 28.890 | 10.885 | 1.00 | 54.19 | N |
| ATOM | 3922 | CA | GLY | B | 545 | 18.408 | 28.280 | 10.805 | 1.00 | 54.15 | C |
| ATOM | 3923 | C | GLY | B | 545 | 19.352 | 28.912 | 9.797 | 1.00 | 50.65 | C |
| ATOM | 3924 | O | GLY | B | 545 | 20.219 | 29.694 | 10.174 | 1.00 | 52.75 | O |
| ATOM | 3925 | N | PRO | B | 546 | 19.213 | 28.585 | 8.504 | 1.00 | 50.84 | N |
| ATOM | 3926 | CD | PRO | B | 546 | 18.223 | 27.634 | 7.965 | 1.00 | 54.61 | C |
| ATOM | 3927 | CA | PRO | B | 546 | 20.056 | 29.110 | 7.423 | 1.00 | 51.43 | C |
| ATOM | 3928 | CB | PRO | B | 546 | 19.341 | 28.613 | 6.164 | 1.00 | 52.58 | C |
| ATOM | 3929 | CG | PRO | B | 546 | 18.809 | 27.294 | 6.609 | 1.00 | 51.61 | C |
| ATOM | 3930 | C | PRO | B | 546 | 20.241 | 30.624 | 7.416 | 1.00 | 45.67 | C |
| ATOM | 3931 | O | PRO | B | 546 | 21.274 | 31.128 | 6.963 | 1.00 | 40.66 | O |
| ATOM | 3932 | N | GLU | B | 547 | 19.234 | 31.343 | 7.903 | 1.00 | 40.50 | N |
| ATOM | 3933 | CA | GLU | B | 547 | 19.294 | 32.796 | 7.953 | 1.00 | 44.42 | C |
| ATOM | 3934 | CB | GLU | B | 547 | 17.963 | 33.376 | 8.410 | 1.00 | 44.41 | C |
| ATOM | 3935 | CG | GLU | B | 547 | 16.811 | 33.093 | 7.475 | 1.00 | 59.26 | C |
| ATOM | 3936 | CD | GLU | B | 547 | 15.538 | 33.763 | 7.927 | 1.00 | 56.22 | C |
| ATOM | 3937 | OE1 | GLU | B | 547 | 15.479 | 35.013 | 7.898 | 1.00 | 62.09 | O |
| ATOM | 3938 | OE2 | GLU | B | 547 | 14.603 | 33.037 | 8.319 | 1.00 | 59.62 | O |
| ATOM | 3939 | C | GLU | B | 547 | 20.391 | 33.282 | 8.902 | 1.00 | 35.91 | C |
| ATOM | 3940 | O | GLU | B | 547 | 21.149 | 34.172 | 8.555 | 1.00 | 34.83 | O |
| ATOM | 3941 | N | VAL | B | 548 | 20.460 | 32.694 | 10.094 | 1.00 | 36.33 | N |
| ATOM | 3942 | CA | VAL | B | 548 | 21.473 | 33.108 | 11.080 | 1.00 | 26.71 | C |
| ATOM | 3943 | CB | VAL | B | 548 | 21.252 | 32.396 | 12.420 | 1.00 | 35.48 | C |
| ATOM | 3944 | CG1 | VAL | B | 548 | 22.285 | 32.872 | 13.448 | 1.00 | 32.82 | C |
| ATOM | 3945 | CG2 | VAL | B | 548 | 19.847 | 32.688 | 12.917 | 1.00 | 40.22 | C |
| ATOM | 3946 | C | VAL | B | 548 | 22.890 | 32.843 | 10.591 | 1.00 | 31.26 | C |
| ATOM | 3947 | O | VAL | B | 548 | 23.762 | 33.696 | 10.708 | 1.00 | 33.92 | O |
| ATOM | 3948 | N | MET | B | 549 | 23.125 | 31.663 | 10.027 | 1.00 | 31.21 | N |
| ATOM | 3949 | CA | MET | B | 549 | 24.439 | 31.316 | 9.511 | 1.00 | 35.46 | C |
| ATOM | 3950 | CB | MET | B | 549 | 24.397 | 29.909 | 8.908 | 1.00 | 40.89 | C |
| ATOM | 3951 | CG | MET | B | 549 | 25.766 | 29.271 | 8.799 | 1.00 | 51.71 | C |
| ATOM | 3952 | SD | MET | B | 549 | 26.636 | 29.238 | 10.404 | 1.00 | 72.82 | S |
| ATOM | 3953 | CE | MET | B | 549 | 27.806 | 30.616 | 10.266 | 1.00 | 56.71 | C |
| ATOM | 3954 | C | MET | B | 549 | 24.918 | 32.325 | 8.445 | 1.00 | 31.51 | C |
| ATOM | 3955 | O | MET | B | 549 | 26.050 | 32.788 | 8.479 | 1.00 | 31.13 | O |
| ATOM | 3956 | N | ALA | B | 550 | 24.042 | 32.645 | 7.501 | 1.00 | 32.16 | N |
| ATOM | 3957 | CA | ALA | B | 550 | 24.373 | 33.588 | 6.431 | 1.00 | 33.32 | C |
| ATOM | 3958 | CB | ALA | B | 550 | 23.206 | 33.653 | 5.428 | 1.00 | 37.58 | C |
| ATOM | 3959 | C | ALA | B | 550 | 24.656 | 34.979 | 7.014 | 1.00 | 25.47 | C |
| ATOM | 3960 | O | ALA | B | 550 | 25.568 | 35.680 | 6.578 | 1.00 | 29.24 | O |
| ATOM | 3961 | N | PHE | B | 551 | 23.857 | 35.360 | 8.007 | 1.00 | 25.23 | N |
| ATOM | 3962 | CA | PHE | B | 551 | 23.975 | 36.647 | 8.706 | 1.00 | 28.55 | C |
| ATOM | 3963 | CB | PHE | B | 551 | 22.852 | 36.714 | 9.769 | 1.00 | 20.31 | C |
| ATOM | 3964 | CG | PHE | B | 551 | 22.783 | 38.000 | 10.543 | 1.00 | 29.64 | C |
| ATOM | 3965 | CD1 | PHE | B | 551 | 22.226 | 39.144 | 9.973 | 1.00 | 28.55 | C |
| ATOM | 3966 | CD2 | PHE | B | 551 | 23.209 | 38.052 | 11.876 | 1.00 | 29.88 | C |
| ATOM | 3967 | CE1 | PHE | B | 551 | 22.081 | 40.319 | 10.716 | 1.00 | 26.70 | C |
| ATOM | 3968 | CE2 | PHE | B | 551 | 23.066 | 39.232 | 12.636 | 1.00 | 26.29 | C |
| ATOM | 3969 | CZ | PHE | B | 551 | 22.498 | 40.362 | 12.050 | 1.00 | 31.05 | C |
| ATOM | 3970 | C | PHE | B | 551 | 25.354 | 36.743 | 9.361 | 1.00 | 26.40 | C |
| ATOM | 3971 | O | PHE | B | 551 | 26.084 | 37.721 | 9.188 | 1.00 | 22.68 | O |
| ATOM | 3972 | N | ILE | B | 552 | 25.717 | 35.720 | 10.123 | 1.00 | 23.43 | N |
| ATOM | 3973 | CA | ILE | B | 552 | 27.022 | 35.707 | 10.778 | 1.00 | 21.41 | C |
| ATOM | 3974 | CB | ILE | B | 552 | 27.135 | 34.460 | 11.681 | 1.00 | 26.93 | C |
| ATOM | 3975 | CG2 | ILE | B | 552 | 28.553 | 34.320 | 12.206 | 1.00 | 28.00 | C |
| ATOM | 3976 | CG1 | ILE | B | 552 | 26.046 | 34.533 | 12.764 | 1.00 | 27.40 | C |
| ATOM | 3977 | CD1 | ILE | B | 552 | 26.179 | 35.709 | 13.768 | 1.00 | 29.73 | C |
| ATOM | 3978 | C | ILE | B | 552 | 28.146 | 35.706 | 9.737 | 1.00 | 22.04 | C |
| ATOM | 3979 | O | ILE | B | 552 | 29.139 | 36.428 | 9.871 | 1.00 | 25.44 | O |
| ATOM | 3980 | N | GLU | B | 553 | 27.988 | 34.910 | 8.684 | 1.00 | 25.85 | N |
| ATOM | 3981 | CA | GLU | B | 553 | 29.004 | 34.842 | 7.630 | 1.00 | 30.95 | C |
| ATOM | 3982 | CB | GLU | B | 553 | 28.613 | 33.796 | 6.580 | 1.00 | 40.19 | C |
| ATOM | 3983 | CG | GLU | B | 553 | 28.654 | 32.360 | 7.092 | 1.00 | 48.42 | C |
| ATOM | 3984 | CD | GLU | B | 553 | 30.068 | 31.858 | 7.339 | 1.00 | 49.16 | C |
| ATOM | 3985 | OE1 | GLU | B | 553 | 30.212 | 30.786 | 7.970 | 1.00 | 54.92 | O |
| ATOM | 3986 | OE2 | GLU | B | 553 | 31.030 | 32.525 | 6.896 | 1.00 | 56.67 | O |
| ATOM | 3987 | C | GLU | B | 553 | 29.220 | 36.206 | 6.947 | 1.00 | 29.01 | C |
| ATOM | 3988 | O | GLU | B | 553 | 30.331 | 36.527 | 6.525 | 1.00 | 34.32 | O |
| ATOM | 3989 | N | GLN | B | 554 | 28.152 | 36.988 | 6.835 | 1.00 | 30.24 | N |
| ATOM | 3990 | CA | GLN | B | 554 | 28.214 | 38.324 | 6.240 | 1.00 | 33.67 | C |
| ATOM | 3991 | CB | GLN | B | 554 | 26.806 | 38.870 | 6.010 | 1.00 | 35.02 | C |
| ATOM | 3992 | CG | GLN | B | 554 | 26.026 | 38.224 | 4.891 | 1.00 | 47.06 | C |
| ATOM | 3993 | CD | GLN | B | 554 | 24.620 | 38.761 | 4.858 | 1.00 | 45.80 | C |
| ATOM | 3994 | OE1 | GLN | B | 554 | 24.410 | 39.970 | 5.004 | 1.00 | 52.95 | O |
| ATOM | 3995 | NE2 | GLN | B | 554 | 23.647 | 37.874 | 4.669 | 1.00 | 52.85 | N |
| ATOM | 3996 | C | GLN | B | 554 | 28.940 | 39.308 | 7.154 | 1.00 | 35.79 | C |
| ATOM | 3997 | O | GLN | B | 554 | 29.176 | 40.452 | 6.772 | 1.00 | 29.12 | O |
| ATOM | 3998 | N | GLY | B | 555 | 29.274 | 38.871 | 8.366 | 1.00 | 26.81 | N |
| ATOM | 3999 | CA | GLY | B | 555 | 29.960 | 39.765 | 9.284 | 1.00 | 29.43 | C |
| ATOM | 4000 | C | GLY | B | 555 | 29.025 | 40.644 | 10.119 | 1.00 | 27.00 | C |
| ATOM | 4001 | O | GLY | B | 555 | 29.447 | 41.672 | 10.660 | 1.00 | 26.99 | O |
| ATOM | 4002 | N | LYS | B | 556 | 27.759 | 40.257 | 10.231 | 1.00 | 20.65 | N |
| ATOM | 4003 | CA | LYS | B | 556 | 26.826 | 41.024 | 11.045 | 1.00 | 21.47 | C |
| ATOM | 4004 | CB | LYS | B | 556 | 25.489 | 41.198 | 10.315 | 1.00 | 24.59 | C |
| ATOM | 4005 | CG | LYS | B | 556 | 25.631 | 41.916 | 8.959 | 1.00 | 24.98 | C |
| ATOM | 4006 | CD | LYS | B | 556 | 24.280 | 42.165 | 8.338 | 1.00 | 24.97 | C |
| ATOM | 4007 | CE | LYS | B | 556 | 24.429 | 42.911 | 6.990 | 1.00 | 31.95 | C |
| ATOM | 4008 | NZ | LYS | B | 556 | 23.084 | 43.238 | 6.391 | 1.00 | 32.23 | N |
| ATOM | 4009 | C | LYS | B | 556 | 26.580 | 40.283 | 12.364 | 1.00 | 22.25 | C |
| ATOM | 4010 | O | LYS | B | 556 | 26.701 | 39.057 | 12.431 | 1.00 | 21.17 | O |
| ATOM | 4011 | N | ARG | B | 557 | 26.208 | 41.049 | 13.384 | 1.00 | 20.82 | N |
| ATOM | 4012 | CA | ARG | B | 557 | 25.950 | 40.496 | 14.709 | 1.00 | 22.17 | C |
| ATOM | 4013 | CB | ARG | B | 557 | 27.164 | 40.778 | 15.629 | 1.00 | 19.79 | C |
| ATOM | 4014 | CG | ARG | B | 557 | 28.487 | 40.159 | 15.124 | 1.00 | 21.41 | C |
| ATOM | 4015 | CD | ARG | B | 557 | 28.495 | 38.618 | 15.195 | 1.00 | 19.59 | C |
| ATOM | 4016 | NE | ARG | B | 557 | 29.796 | 38.029 | 14.814 | 1.00 | 20.25 | N |
| ATOM | 4017 | CZ | ARG | B | 557 | 30.161 | 37.681 | 13.579 | 1.00 | 23.23 | C |
| ATOM | 4018 | NH1 | ARG | B | 557 | 29.329 | 37.849 | 12.534 | 1.00 | 21.34 | N |
| ATOM | 4019 | NH2 | ARG | B | 557 | 31.354 | 37.117 | 13.379 | 1.00 | 18.85 | N |
| ATOM | 4020 | C | ARG | B | 557 | 24.699 | 41.113 | 15.311 | 1.00 | 25.58 | C |
| ATOM | 4021 | O | ARG | B | 557 | 24.163 | 42.122 | 14.787 | 1.00 | 20.60 | O |
| ATOM | 4022 | N | MET | B | 558 | 24.211 | 40.520 | 16.405 | 1.00 | 23.12 | N |
| ATOM | 4023 | CA | MET | B | 558 | 23.031 | 41.057 | 17.061 | 1.00 | 23.08 | C |
| ATOM | 4024 | CB | MET | B | 558 | 22.577 | 40.150 | 18.226 | 1.00 | 21.21 | C |
| ATOM | 4025 | CG | MET | B | 558 | 21.929 | 38.861 | 17.759 | 1.00 | 23.06 | C |
| ATOM | 4026 | SD | MET | B | 558 | 21.172 | 37.972 | 19.155 | 1.00 | 25.40 | S |
| ATOM | 4027 | CE | MET | B | 558 | 22.589 | 37.274 | 19.959 | 1.00 | 20.17 | C |
| ATOM | 4028 | C | MET | B | 558 | 23.299 | 42.458 | 17.588 | 1.00 | 26.20 | C |
| ATOM | 4029 | O | MET | B | 558 | 24.416 | 42.787 | 18.026 | 1.00 | 22.21 | O |
| ATOM | 4030 | N | GLU | B | 559 | 22.262 | 43.287 | 17.554 | 1.00 | 22.33 | N |
| ATOM | 4031 | CA | GLU | B | 559 | 22.354 | 44.649 | 18.023 | 1.00 | 25.04 | C |
| ATOM | 4032 | CB | GLU | B | 559 | 21.047 | 45.392 | 17.711 | 1.00 | 33.85 | C |
| ATOM | 4033 | CG | GLU | B | 559 | 19.765 | 44.572 | 17.953 | 1.00 | 44.82 | C |
| ATOM | 4034 | CD | GLU | B | 559 | 19.514 | 43.500 | 16.868 | 1.00 | 56.27 | C |
| ATOM | 4035 | OE1 | GLU | B | 559 | 19.120 | 43.868 | 15.733 | 1.00 | 60.02 | O |
| ATOM | 4036 | OE2 | GLU | B | 559 | 19.710 | 42.290 | 17.149 | 1.00 | 44.19 | O |
| ATOM | 4037 | C | GLU | B | 559 | 22.646 | 44.778 | 19.513 | 1.00 | 25.59 | C |
| ATOM | 4038 | O | GLU | B | 559 | 22.362 | 43.880 | 20.302 | 1.00 | 29.33 | O |
| ATOM | 4039 | N | CYS | B | 560 | 23.210 | 45.913 | 19.897 | 1.00 | 22.19 | N |
| ATOM | 4040 | CA | CYS | B | 560 | 23.471 | 46.170 | 21.306 | 1.00 | 25.34 | C |
| ATOM | 4041 | CB | CYS | B | 560 | 24.313 | 47.440 | 21.454 | 1.00 | 26.43 | C |
| ATOM | 4042 | SG | CYS | B | 560 | 24.611 | 47.893 | 23.164 | 1.00 | 30.43 | S |
| ATOM | 4043 | C | CYS | B | 560 | 22.108 | 46.336 | 22.013 | 1.00 | 32.06 | C |
| ATOM | 4044 | O | CYS | B | 560 | 21.262 | 47.124 | 21.570 | 1.00 | 29.35 | O |
| ATOM | 4045 | N | PRO | B | 561 | 21.855 | 45.564 | 23.092 | 1.00 | 30.63 | N |
| ATOM | 4046 | CD | PRO | B | 561 | 22.644 | 44.426 | 23.611 | 1.00 | 28.65 | C |
| ATOM | 4047 | CA | PRO | B | 561 | 20.570 | 45.686 | 23.803 | 1.00 | 31.69 | C |
| ATOM | 4048 | CB | PRO | B | 561 | 20.727 | 44.723 | 24.987 | 1.00 | 28.11 | C |
| ATOM | 4049 | CG | PRO | B | 561 | 21.605 | 43.638 | 24.418 | 1.00 | 25.66 | C |
| ATOM | 4050 | C | PRO | B | 561 | 20.348 | 47.111 | 24.298 | 1.00 | 28.57 | C |
| ATOM | 4051 | O | PRO | B | 561 | 21.292 | 47.850 | 24.542 | 1.00 | 26.62 | O |
| ATOM | 4052 | N | PRO | B | 562 | 19.092 | 47.519 | 24.459 | 1.00 | 37.07 | N |
| ATOM | 4053 | CD | PRO | B | 562 | 17.852 | 46.840 | 24.046 | 1.00 | 39.32 | C |
| ATOM | 4054 | CA | PRO | B | 562 | 18.821 | 48.880 | 24.939 | 1.00 | 38.49 | C |
| ATOM | 4055 | CB | PRO | B | 562 | 17.294 | 48.942 | 24.948 | 1.00 | 42.66 | C |
| ATOM | 4056 | CG | PRO | B | 562 | 16.920 | 48.002 | 23.833 | 1.00 | 48.28 | C |
| ATOM | 4057 | C | PRO | B | 562 | 19.419 | 49.086 | 26.336 | 1.00 | 38.16 | C |
| ATOM | 4058 | O | PRO | B | 562 | 19.329 | 48.207 | 27.193 | 1.00 | 38.50 | O |
| ATOM | 4059 | N | GLU | B | 563 | 20.042 | 50.236 | 26.568 | 1.00 | 37.38 | N |
| ATOM | 4060 | CA | GLU | B | 563 | 20.623 | 50.530 | 27.881 | 1.00 | 43.37 | C |
| ATOM | 4061 | CB | GLU | B | 563 | 19.564 | 50.341 | 28.981 | 1.00 | 49.78 | C |
| ATOM | 4062 | CG | GLU | B | 563 | 18.309 | 51.190 | 28.805 | 1.00 | 53.84 | C |
| ATOM | 4063 | CD | GLU | B | 563 | 17.088 | 50.587 | 29.496 | 1.00 | 61.69 | C |
| ATOM | 4064 | OE1 | GLU | B | 563 | 17.133 | 50.389 | 30.734 | 1.00 | 64.80 | O |
| ATOM | 4065 | OE2 | GLU | B | 563 | 16.085 | 50.307 | 27.795 | 1.00 | 62.44 | O |
| ATOM | 4066 | C | GLU | B | 563 | 21.873 | 49.714 | 28.228 | 1.00 | 42.92 | C |
| ATOM | 4067 | O | GLU | B | 563 | 22.433 | 49.860 | 29.314 | 1.00 | 46.04 | O |
| ATOM | 4068 | N | CYS | B | 564 | 22.312 | 48.832 | 27.332 | 1.00 | 40.16 | N |
| ATOM | 4069 | CA | CYS | B | 564 | 23.527 | 48.070 | 27.589 | 1.00 | 35.91 | C |
| ATOM | 4070 | CB | CYS | B | 564 | 23.614 | 46.843 | 26.662 | 1.00 | 35.94 | C |
| ATOM | 4071 | SG | CYS | B | 564 | 25.157 | 45.907 | 26.802 | 1.00 | 37.39 | S |
| ATOM | 4072 | C | CYS | B | 564 | 24.706 | 49.006 | 27.324 | 1.00 | 37.21 | C |
| ATOM | 4073 | O | CYS | B | 564 | 24.832 | 49.559 | 26.231 | 1.00 | 36.78 | O |
| ATOM | 4074 | N | PRO | B | 565 | 25.580 | 49.200 | 28.324 | 1.00 | 35.01 | N |
| ATOM | 4075 | CD | PRO | B | 565 | 25.531 | 48.573 | 29.655 | 1.00 | 39.96 | C |
| ATOM | 4076 | CA | PRO | B | 565 | 26.753 | 50.072 | 28.200 | 1.00 | 32.47 | C |
| ATOM | 4077 | CB | PRO | B | 565 | 27.382 | 50.015 | 29.589 | 1.00 | 40.02 | C |
| ATOM | 4078 | CG | PRO | B | 565 | 26.960 | 48.666 | 30.098 | 1.00 | 43.81 | C |
| ATOM | 4079 | C | PRO | B | 565 | 27.709 | 49.607 | 27.127 | 1.00 | 35.02 | C |
| ATOM | 4080 | O | PRO | B | 565 | 27.860 | 48.403 | 26.893 | 1.00 | 34.15 | O |
| ATOM | 4081 | N | PRO | B | 566 | 28.396 | 50.558 | 26.475 | 1.00 | 33.68 | N |
| ATOM | 4082 | CD | PRO | B | 566 | 28.297 | 52.010 | 26.727 | 1.00 | 34.27 | C |
| ATOM | 4083 | CA | PRO | B | 566 | 29.360 | 50.272 | 25.408 | 1.00 | 30.53 | C |
| ATOM | 4084 | CB | PRO | B | 566 | 29.908 | 51.647 | 25.137 | 1.00 | 31.64 | C |
| ATOM | 4085 | CG | PRO | B | 566 | 28.908 | 52.607 | 25.481 | 1.00 | 31.51 | C |
| ATOM | 4086 | C | PRO | B | 566 | 30.412 | 49.223 | 25.792 | 1.00 | 31.72 | C |
| ATOM | 4087 | O | PRO | B | 566 | 30.757 | 48.379 | 24.978 | 1.00 | 28.48 | O |
| ATOM | 4088 | N | GLU | B | 567 | 30.920 | 49.285 | 27.028 | 1.00 | 27.33 | N |
| ATOM | 4089 | CA | GLU | B | 567 | 31.960 | 48.345 | 27.472 | 1.00 | 33.27 | C |
| ATOM | 4090 | CB | GLU | B | 567 | 32.451 | 48.701 | 28.894 | 1.00 | 36.30 | C |
| ATOM | 4091 | CG | GLU | B | 567 | 32.553 | 50.189 | 29.192 | 1.00 | 49.34 | C |
| ATOM | 4092 | CD | GLU | B | 567 | 31.195 | 50.835 | 29.391 | 1.00 | 50.90 | C |
| ATOM | 4093 | OE1 | GLU | B | 567 | 30.471 | 50.431 | 30.330 | 1.00 | 63.92 | O |
| ATOM | 4094 | OE2 | GLU | B | 567 | 30.851 | 51.744 | 28.612 | 1.00 | 47.96 | O |
| ATOM | 4095 | C | GLU | B | 567 | 31.440 | 46.908 | 27.485 | 1.00 | 26.60 | C |
| ATOM | 4096 | O | GLU | B | 567 | 32.143 | 45.964 | 27.089 | 1.00 | 25.31 | O |
| ATOM | 4097 | N | LEU | B | 568 | 30.208 | 46.747 | 27.948 | 1.00 | 25.52 | N |
| ATOM | 4098 | CA | LEU | B | 568 | 29.590 | 45.442 | 28.017 | 1.00 | 28.35 | C |
| ATOM | 4099 | CB | LEU | B | 568 | 28.312 | 45.549 | 28.851 | 1.00 | 31.11 | C |
| ATOM | 4100 | CG | LEU | B | 568 | 27.993 | 44.399 | 29.788 | 1.00 | 31.00 | C |
| ATOM | 4101 | CD1 | LEU | B | 568 | 29.277 | 43.796 | 30.349 | 1.00 | 35.96 | C |
| ATOM | 4102 | CD2 | LEU | B | 568 | 27.101 | 44.939 | 30.912 | 1.00 | 25.70 | C |
| ATOM | 4103 | C | LEU | B | 568 | 29.289 | 44.939 | 30.912 | 1.00 | 25.70 | C |
| ATOM | 4104 | O | LEU | B | 568 | 29.498 | 43.738 | 26.341 | 1.00 | 22.86 | O |
| ATOM | 4105 | N | TYR | B | 569 | 28.793 | 45.791 | 25.733 | 1.00 | 23.22 | N |
| ATOM | 4106 | CA | TYR | B | 569 | 28.538 | 45.310 | 24.383 | 1.00 | 22.95 | C |
| ATOM | 4107 | CB | TYR | B | 569 | 27.765 | 46.361 | 23.542 | 1.00 | 21.93 | C |
| ATOM | 4108 | CG | TYR | B | 569 | 27.412 | 45.828 | 22.170 | 1.00 | 21.83 | C |
| ATOM | 4109 | CD1 | TYR | B | 569 | 26.583 | 44.721 | 22.022 | 1.00 | 21.17 | C |
| ATOM | 4110 | CE1 | TYR | B | 569 | 26.304 | 44.169 | 20.757 | 1.00 | 22.67 | C |
| ATOM | 4111 | CD2 | TYR | B | 569 | 27.966 | 46.396 | 21.006 | 1.00 | 28.53 | C |
| ATOM | 4112 | CE2 | TYR | B | 569 | 27.703 | 45.853 | 19.739 | 1.00 | 27.28 | C |
| ATOM | 4113 | CZ | TYR | B | 569 | 26.881 | 44.746 | 19.622 | 1.00 | 28.33 | C |
| ATOM | 4114 | OH | TYR | B | 569 | 26.664 | 44.202 | 18.384 | 1.00 | 25.54 | O |
| ATOM | 4115 | C | TYR | B | 569 | 29.856 | 44.948 | 23.683 | 1.00 | 19.58 | C |
| ATOM | 4116 | O | TYR | B | 569 | 29.909 | 43.976 | 22.908 | 1.00 | 23.18 | O |
| ATOM | 4117 | N | ALA | B | 570 | 30.927 | 45.704 | 23.943 | 1.00 | 23.62 | N |
| ATOM | 4118 | CA | ALA | B | 570 | 32.206 | 45.415 | 23.296 | 1.00 | 24.27 | C |
| ATOM | 4119 | CB | ALA | B | 570 | 32.287 | 46.413 | 23.739 | 1.00 | 26.06 | C |
| ATOM | 4120 | C | ALA | B | 570 | 32.670 | 43.993 | 23.615 | 1.00 | 24.88 | C |
| ATOM | 4121 | O | ALA | B | 570 | 33.226 | 43.307 | 22.768 | 1.00 | 23.64 | O |
| ATOM | 4122 | N | LEU | B | 571 | 32.443 | 43.587 | 24.856 | 1.00 | 23.04 | N |
| ATOM | 4123 | CA | LEU | B | 571 | 32.844 | 42.259 | 25.336 | 1.00 | 21.51 | C |
| ATOM | 4124 | CB | LEU | B | 571 | 32.668 | 42.201 | 26.866 | 1.00 | 23.34 | C |
| ATOM | 4125 | CG | LEU | B | 571 | 32.296 | 40.815 | 27.465 | 1.00 | 22.60 | C |
| ATOM | 4126 | CD1 | LEU | B | 571 | 34.295 | 40.301 | 27.028 | 1.00 | 24.30 | C |
| ATOM | 4127 | CD2 | LEU | B | 571 | 32.820 | 40.914 | 28.981 | 1.00 | 24.39 | C |
| ATOM | 4128 | C | LEU | B | 571 | 31.988 | 41.197 | 24.676 | 1.00 | 16.75 | C |
| ATOM | 4129 | O | LEU | B | 571 | 32.472 | 40.189 | 24.163 | 1.00 | 20.66 | O |
| ATOM | 4130 | N | MET | B | 572 | 30.687 | 41.4277 | 24.715 | 1.00 | 20.93 | N |
| ATOM | 4131 | CA | MET | B | 572 | 29.728 | 40.535 | 24.102 | 1.00 | 20.96 | C |
| ATOM | 4132 | CB | MET | B | 572 | 28.356 | 41.204 | 24.223 | 1.00 | 28.64 | C |
| ATOM | 4133 | CG | MET | B | 572 | 27.271 | 40.510 | 23.502 | 1.00 | 30.16 | C |
| ATOM | 4134 | SD | MET | B | 572 | 25.729 | 41.414 | 23.691 | 1.00 | 24.19 | S |
| ATOM | 4135 | CE | MET | B | 572 | 25.754 | 41.989 | 25.454 | 1.00 | 22.91 | C |
| ATOM | 4136 | C | MET | B | 572 | 30.117 | 40.358 | 22.625 | 1.00 | 20.33 | C |
| ATOM | 4137 | O | MET | B | 572 | 30.204 | 39.235 | 22.101 | 1.00 | 20.73 | O |
| ATOM | 4138 | N | SER | B | 573 | 30.373 | 41.470 | 21.941 | 1.00 | 18.391 | N |
| ATOM | 4139 | CA | SER | B | 573 | 30.735 | 41.412 | 20.528 | 1.00 | 21.31 | C |
| ATOM | 4140 | CB | SER | B | 573 | 30.747 | 42.839 | 19.939 | 1.00 | 25.35 | C |
| ATOM | 4141 | OG | SER | B | 573 | 31.143 | 42.855 | 18.581 | 1.00 | 27.38 | O |
| ATOM | 4142 | C | SER | B | 573 | 32.073 | 40.693 | 20.296 | 1.00 | 23.59 | C |
| ATOM | 4143 | O | SER | B | 573 | 32.231 | 39.962 | 19.324 | 1.00 | 21.82 | O |
| ATOM | 4144 | N | ASP | B | 574 | 33.043 | 40.889 | 21.176 | 1.00 | 22.93 | N |
| ATOM | 4145 | CA | ASP | B | 574 | 34.322 | 40.196 | 21.019 | 1.00 | 21.19 | C |
| ATOM | 4146 | CB | ASP | B | 574 | 35.363 | 40.684 | 22.039 | 1.00 | 21.91 | C |
| ATOM | 4147 | CG | ASP | B | 574 | 35.914 | 42.071 | 21.707 | 1.00 | 25.96 | C |
| ATOM | 4148 | OD1 | ASP | B | 574 | 35.752 | 42.553 | 20.567 | 1.00 | 23.74 | O |
| ATOM | 4149 | OD2 | ASP | B | 574 | 36.547 | 42.667 | 22.604 | 1.00 | 26.12 | O |
| ATOM | 4150 | C | ASP | B | 574 | 34.156 | 38.667 | 21.166 | 1.00 | 22.88 | C |
| ATOM | 4151 | O | ASP | B | 574 | 34.942 | 37.915 | 20.585 | 1.00 | 23.63 | O |
| ATOM | 4152 | N | CYS | B | 575 | 33.161 | 38.218 | 21.938 | 1.00 | 19.59 | N |
| ATOM | 4153 | CA | CYS | B | 575 | 32.906 | 36.778 | 22.097 | 1.00 | 20.12 | C |
| ATOM | 4154 | CB | CYS | B | 575 | 31.822 | 36.522 | 23.163 | 1.00 | 22.35 | C |
| ATOM | 4155 | SG | CYS | B | 575 | 32.353 | 36.912 | 24.873 | 1.00 | 23.15 | S |
| ATOM | 4156 | C | CYS | B | 575 | 32.426 | 36.205 | 20.772 | 1.00 | 22.05 | C |
| ATOM | 4157 | O | CYS | B | 575 | 32.442 | 34.991 | 20.572 | 1.00 | 19.70 | O |
| ATOM | 4158 | N | TRP | B | 576 | 32.001 | 37.096 | 19.877 | 1.00 | 20.34 | N |
| ATOM | 4159 | CA | TRP | B | 576 | 31.514 | 36.699 | 18.556 | 1.00 | 19.05 | C |
| ATOM | 4160 | CB | TRP | B | 576 | 30.184 | 37.393 | 18.208 | 1.00 | 19.93 | C |
| ATOM | 4161 | CG | TRP | B | 576 | 29.089 | 37.172 | 19.205 | 1.00 | 22.10 | C |
| ATOM | 4162 | CD2 | TRP | B | 576 | 28.077 | 38.106 | 19.558 | 1.00 | 17.49 | C |
| ATOM | 4163 | CE2 | TRP | B | 576 | 27.263 | 27.485 | 20.547 | 1.00 | 18.66 | C |
| ATOM | 4164 | CE3 | TRP | B | 576 | 27.763 | 39.414 | 19.127 | 1.00 | 16.82 | C |
| ATOM | 4165 | CD1 | TRP | B | 576 | 28.868 | 36.045 | 19.956 | 1.00 | 20.31 | C |
| ATOM | 4166 | NE1 | TRP | B | 576 | 27.764 | 36.225 | 20.770 | 1.00 | 19.04 | N |
| ATOM | 4167 | CZ2 | TRP | B | 576 | 26.158 | 38.130 | 21.122 | 1.00 | 19.31 | C |
| ATOM | 4168 | CZ3 | TRP | B | 576 | 26.653 | 40.059 | 19.692 | 1.00 | 19.50 | C |
| ATOM | 4169 | CH2 | TRP | B | 576 | 25.861 | 39.418 | 20.684 | 1.00 | 19.35 | C |
| ATOM | 4170 | C | TRP | B | 576 | 32.493 | 36.958 | 17.405 | 1.00 | 22.30 | C |
| ATOM | 4171 | O | TRP | B | 576 | 32.062 | 37.191 | 16.262 | 1.00 | 20.73 | O |
| ATOM | 4172 | N | ILE | B | 577 | 33.792 | 36.942 | 17.714 | 1.00 | 19.99 | N |
| ATOM | 4173 | CA | ILE | B | 577 | 34.826 | 37.103 | 16.689 | 1.00 | 21.32 | C |
| ATOM | 4174 | CB | ILE | B | 577 | 36.215 | 37.309 | 17.338 | 1.00 | 20.92 | C |
| ATOM | 4175 | CG2 | ILE | B | 577 | 37.351 | 36.977 | 16.323 | 1.00 | 19.24 | C |
| ATOM | 4176 | CG1 | ILE | B | 577 | 36.318 | 38.764 | 17.847 | 1.00 | 26.98 | C |
| ATOM | 4177 | CD1 | ILE | B | 577 | 37.577 | 39.046 | 18.674 | 1.00 | 31.70 | C |
| ATOM | 4178 | C | ILE | B | 577 | 34.774 | 35.798 | 15.891 | 1.00 | 22.46 | C |
| ATOM | 4179 | O | ILE | B | 577 | 34.789 | 34.707 | 16.468 | 1.00 | 22.10 | O |
| ATOM | 4180 | N | TYR | B | 578 | 34.673 | 35.914 | 14.571 | 1.00 | 20.33 | N |
| ATOM | 4181 | CA | TYR | B | 578 | 34.532 | 34.751 | 13.715 | 1.00 | 24.28 | C |
| ATOM | 4182 | CB | TYR | B | 578 | 34.387 | 35.200 | 12.248 | 1.00 | 24.92 | C |
| ATOM | 4183 | CG | TYR | B | 578 | 33.925 | 34.103 | 11.307 | 1.00 | 25.66 | C |
| ATOM | 4184 | CD1 | TYR | B | 578 | 32.574 | 33.963 | 10.977 | 1.00 | 29.87 | C |
| ATOM | 4185 | CE1 | TYR | B | 578 | 32.139 | 32.918 | 10.134 | 1.00 | 32.04 | C |
| ATOM | 4186 | CD2 | TYR | B | 578 | 34.831 | 33.173 | 10.770 | 1.00 | 31.85 | C |
| ATOM | 4187 | CE2 | TYR | B | 578 | 34.397 | 32.126 | 9.938 | 1.00 | 29.55 | C |
| ATOM | 4188 | CZ | TYR | B | 578 | 33.047 | 32.009 | 9.628 | 1.00 | 31.34 | C |
| ATOM | 4189 | OH | TYR | B | 578 | 32.596 | 30.972 | 8.824 | 1.00 | 31.73 | O |
| ATOM | 4190 | C | TYR | B | 578 | 35.648 | 33.715 | 13.814 | 1.00 | 29.60 | C |
| ATOM | 4191 | O | TYR | B | 578 | 35.377 | 32.529 | 14.014 | 1.00 | 21.86 | O |
| ATOM | 4192 | N | LYS | B | 579 | 36.887 | 34.175 | 13.614 | 1.00 | 24.49 | N |
| ATOM | 4193 | CA | LYS | B | 579 | 38.069 | 33.335 | 13.631 | 1.00 | 27.75 | C |
| ATOM | 4194 | CB | LYS | B | 579 | 39.301 | 34.121 | 13.145 | 1.00 | 30.85 | C |
| ATOM | 4195 | CG | LYS | B | 579 | 39.212 | 34.623 | 11.695 | 1.00 | 39.76 | C |
| ATOM | 4196 | CD | LYS | B | 579 | 40.550 | 35.234 | 11.213 | 1.00 | 43.80 | C |
| ATOM | 4197 | CE | LYS | B | 579 | 40.481 | 35.643 | 9.735 | 1.00 | 52.66 | C |
| ATOM | 4198 | NZ | LYS | B | 579 | 41.684 | 36.457 | 9.382 | 1.00 | 53.08 | N |
| ATOM | 4199 | C | LYS | B | 579 | 38.346 | 32.811 | 15.021 | 1.00 | 28.50 | C |
| ATOM | 4200 | O | LYS | B | 579 | 38.585 | 33.568 | 15.942 | 1.00 | 27.27 | O |
| ATOM | 4201 | N | TRP | B | 580 | 38.335 | 31.495 | 15.128 | 1.00 | 27.56 | N |
| ATOM | 4202 | CA | TRP | B | 580 | 38.574 | 30.806 | 16.385 | 1.00 | 33.99 | C |
| ATOM | 4203 | CB | TRP | B | 580 | 38.649 | 29.310 | 16.079 | 1.00 | 33.38 | C |
| ATOM | 4204 | CG | TRP | B | 580 | 38.850 | 28.472 | 17.259 | 1.00 | 38.62 | C |
| ATOM | 4205 | CD2 | TRP | B | 580 | 40.063 | 27.818 | 17.639 | 1.00 | 38.34 | C |
| ATOM | 4206 | CE2 | TRP | B | 580 | 39.793 | 27.097 | 18.830 | 1.00 | 42.10 | C |
| ATOM | 4207 | CE3 | TRP | B | 580 | 41.354 | 27.768 | 17.091 | 1.00 | 43.21 | C |
| ATOM | 4208 | CD1 | TRP | B | 580 | 37.920 | 28.145 | 18.199 | 1.00 | 40.63 | C |
| ATOM | 4209 | NE1 | TRP | B | 580 | 38.479 | 27.312 | 19.150 | 1.00 | 37.48 | N |
| ATOM | 4210 | CZ2 | TRP | B | 580 | 40.767 | 26.33 | 19.484 | 1.00 | 42.20 | C |
| ATOM | 4211 | CZ3 | TRP | B | 580 | 42.328 | 27.004 | 17.742 | 1.00 | 44.42 | C |
| ATOM | 4212 | CH2 | TRP | B | 580 | 42.025 | 26.297 | 18.927 | 1.00 | 49.85 | C |
| ATOM | 4213 | C | TRP | B | 580 | 39.862 | 31.267 | 17.077 | 1.00 | 29.68 | C |
| ATOM | 4214 | O | TRP | B | 580 | 39.862 | 31.591 | 18.262 | 1.00 | 30.01 | O |
| ATOM | 4215 | N | GLU | B | 581 | 40.962 | 31.306 | 16.336 | 1.00 | 27.48 | N |
| ATOM | 4216 | CA | GLU | B | 581 | 42.236 | 31.710 | 16.917 | 1.00 | 27.95 | C |
| ATOM | 4217 | CB | GLU | B | 581 | 43.349 | 31.535 | 15.875 | 1.00 | 41.30 | C |
| ATOM | 4218 | CG | GLU | B | 581 | 43.080 | 30.409 | 14.878 | 1.00 | 45.98 | C |
| ATOM | 4219 | CD | GLU | B | 581 | 42.292 | 20.894 | 13.672 | 1.00 | 53.35 | C |
| ATOM | 4220 | OE1 | GLU | B | 581 | 42.833 | 31.742 | 12.919 | 1.00 | 61.50 | O |
| ATOM | 4221 | OE2 | GLU | B | 581 | 41.144 | 30.422 | 13.466 | 1.00 | 44.26 | O |
| ATOM | 4222 | C | GLU | B | 581 | 42.269 | 33.141 | 17.458 | 1.00 | 31.33 | C |
| ATOM | 4223 | O | GLU | B | 581 | 43.070 | 33.449 | 18.327 | 1.00 | 33.50 | O |
| ATOM | 4224 | N | ASP | B | 582 | 41.386 | 34.006 | 16.961 | 1.00 | 28.16 | N |
| ATOM | 4225 | CA | ASP | B | 582 | 41.341 | 35.407 | 17.389 | 1.00 | 24.75 | C |
| ATOM | 4226 | CB | ASP | B | 582 | 41.073 | 36.298 | 16.169 | 1.00 | 29.45 | C |
| ATOM | 4227 | CG | ASP | B | 582 | 42.179 | 36.201 | 15.131 | 1.00 | 39.01 | C |
| ATOM | 4228 | OD1 | ASP | B | 582 | 43.301 | 35.821 | 15.524 | 1.00 | 35.67 | O |
| ATOM | 4229 | OD2 | ASP | B | 582 | 41.937 | 36.495 | 13.937 | 1.00 | 38.00 | O |
| ATOM | 4230 | C | ASP | B | 582 | 40.307 | 35.726 | 18.478 | 1.00 | 26.77 | C |
| ATOM | 4231 | O | ASP | B | 582 | 40.250 | 36.848 | 18.993 | 1.00 | 25.92 | O |
| ATOM | 4232 | N | ARG | B | 583 | 39.487 | 34.744 | 18.816 | 1.00 | 27.16 | N |
| ATOM | 4233 | CA | ARG | B | 583 | 38.448 | 34.959 | 19.818 | 1.00 | 29.93 | C |
| ATOM | 4234 | CB | ARG | B | 583 | 37.324 | 33.921 | 19.611 | 1.00 | 23.20 | C |
| ATOM | 4235 | CG | ARG | B | 583 | 35.974 | 34.196 | 20.334 | 1.00 | 20.53 | C |
| ATOM | 4236 | CD | ARG | B | 583 | 34.993 | 33.051 | 20.050 | 1.00 | 17.55 | C |
| ATOM | 4237 | NE | ARG | B | 583 | 34.872 | 32.798 | 18.608 | 1.00 | 17.49 | N |
| ATOM | 4238 | CZ | ARG | B | 583 | 34.658 | 31.610 | 18.055 | 1.00 | 22.33 | C |
| ATOM | 4239 | NH1 | ARG | B | 583 | 34.528 | 30.522 | 18.811 | 1.00 | 20.02 | N |
| ATOM | 4240 | NH2 | ARG | B | 583 | 34.618 | 31.501 | 16.721 | 1.00 | 21.31 | N |
| ATOM | 4241 | C | ARG | B | 583 | 39.101 | 34.827 | 21.203 | 1.00 | 26.43 | C |
| ATOM | 4242 | O | ARG | B | 583 | 39.964 | 33.975 | 21.417 | 1.00 | 24.85 | O |
| ATOM | 4243 | N | PRO | B | 584 | 38.694 | 35.679 | 22.159 | 1.00 | 23.83 | N |
| ATOM | 4244 | CD | PRO | B | 584 | 37.731 | 36.791 | 22.028 | 1.00 | 23.73 | C |
| ATOM | 4245 | CA | PRO | B | 584 | 39.252 | 35.638 | 23.516 | 1.00 | 25.03 | C |
| ATOM | 4246 | CB | PRO | B | 584 | 38.660 | 36.877 | 24.178 | 1.00 | 25.59 | C |
| ATOM | 4247 | CG | PRO | B | 584 | 37.346 | 37.053 | 23.469 | 1.00 | 24.23 | C |
| ATOM | 4248 | C | PRO | B | 584 | 38.890 | 34.383 | 24.302 | 1.00 | 25.66 | C |
| ATOM | 4249 | O | PRO | B | 584 | 37.896 | 33.722 | 23.987 | 1.00 | 22.86 | O |
| ATOM | 4250 | N | ASP | B | 585 | 39.714 | 34.082 | 25.314 | 1.00 | 20.92 | N |
| ATOM | 4251 | CA | ASP | B | 585 | 39.492 | 32.946 | 26.205 | 1.00 | 23.08 | C |
| ATOM | 4252 | CB | ASP | B | 585 | 40.833 | 32.346 | 26.685 | 1.00 | 32.60 | C |
| ATOM | 4253 | CG | ASP | B | 585 | 41.670 | 31.806 | 25.541 | 1.00 | 38.55 | C |
| ATOM | 4254 | OD1 | ASP | B | 585 | 41.153 | 31.012 | 24.727 | 1.00 | 48.57 | O |
| ATOM | 4255 | OD2 | ASP | B | 585 | 42.865 | 32.180 | 25.469 | 1.00 | 55.39 | O |
| ATOM | 4256 | C | ASP | B | 585 | 38.763 | 33.456 | 27.448 | 1.00 | 20.51 | C |
| ATOM | 4257 | O | ASP | B | 585 | 38.686 | 34.667 | 27.668 | 1.00 | 23.27 | O |
| ATOM | 4258 | N | PHE | B | 586 | 38.260 | 32.542 | 28.286 | 1.00 | 21.45 | N |
| ATOM | 4259 | CA | PHE | B | 586 | 37.538 | 32.967 | 29.485 | 1.00 | 26.29 | C |
| ATOM | 4260 | CB | PHE | B | 586 | 36.795 | 31.794 | 30.151 | 1.00 | 22.81 | C |
| ATOM | 4261 | CG | PHE | B | 586 | 35.482 | 31.470 | 29.484 | 1.00 | 22.63 | C |
| ATOM | 4262 | CD1 | PHE | B | 586 | 34.406 | 32.367 | 29.542 | 1.00 | 24.53 | C |
| ATOM | 4263 | CD2 | PHE | B | 586 | 35.323 | 30.268 | 27.785 | 1.00 | 26.40 | C |
| ATOM | 4264 | CE1 | PHE | B | 586 | 33.179 | 32.049 | 28.910 | 1.00 | 18.28 | C |
| ATOM | 4265 | CE2 | PHE | B | 586 | 34.112 | 29.961 | 28.161 | 1.00 | 24.68 | C |
| ATOM | 4266 | CZ | PHE | B | 586 | 33.048 | 30.847 | 28.231 | 1.00 | 19.51 | C |
| ATOM | 4267 | C | PHE | B | 586 | 38.440 | 33.673 | 30.486 | 1.00 | 24.38 | C |
| ATOM | 4268 | O | PHE | B | 586 | 37.983 | 34.473 | 31.267 | 1.00 | 23.72 | O |
| ATOM | 4269 | N | LEU | B | 587 | 39.741 | 33.395 | 30.440 | 1.00 | 27.00 | N |
| ATOM | 4270 | CA | LEU | B | 587 | 40.650 | 34.114 | 31.337 | 1.00 | 24.91 | C |
| ATOM | 4271 | CB | LEU | B | 587 | 42.093 | 33.688 | 31.039 | 1.00 | 32.67 | C |
| ATOM | 4272 | CG | LEU | B | 587 | 43.216 | 34.188 | 31.943 | 1.00 | 43.71 | C |
| ATOM | 4273 | CD1 | LEU | B | 587 | 43.348 | 35.672 | 31.784 | 1.00 | 43.71 | C |
| ATOM | 4274 | CD2 | LEU | B | 587 | 43.926 | 33.834 | 33.380 | 1.00 | 40.03 | C |
| ATOM | 4275 | C | LEU | B | 587 | 40.455 | 35.627 | 31.103 | 1.00 | 23.16 | C |
| ATOM | 4276 | O | LEU | B | 587 | 40.225 | 36.374 | 32.063 | 1.00 | 26.74 | O |
| ATOM | 4277 | N | THR | B | 588 | 40.494 | 36.088 | 29.852 | 1.00 | 23.64 | N |
| ATOM | 4278 | CA | THR | B | 588 | 40.294 | 37.516 | 29.607 | 1.00 | 22.82 | C |
| ATOM | 4279 | CB | THR | B | 588 | 40.852 | 37.982 | 28.299 | 1.00 | 33.41 | C |
| ATOM | 4280 | OG1 | THR | B | 588 | 40.043 | 37.479 | 27.172 | 1.00 | 46.39 | O |
| ATOM | 4281 | CG2 | THR | B | 588 | 42.266 | 37.477 | 28.056 | 1.00 | 31.62 | C |
| ATOM | 4282 | C | THR | B | 588 | 38.832 | 37.929 | 29.711 | 1.00 | 22.83 | C |
| ATOM | 4283 | O | THR | B | 588 | 38.539 | 39.006 | 30.205 | 1.00 | 27.35 | O |
| ATOM | 4284 | N | VAL | B | 589 | 37.915 | 37.069 | 29.258 | 1.00 | 23.23 | N |
| ATOM | 4285 | CA | VAL | B | 589 | 36.502 | 37.418 | 29.370 | 1.00 | 23.31 | C |
| ATOM | 4286 | CB | VAL | B | 589 | 35.601 | 36.298 | 28.759 | 1.00 | 25.23 | C |
| ATOM | 4287 | CG1 | VAL | B | 589 | 34.128 | 36.521 | 29.142 | 1.00 | 24.91 | C |
| ATOM | 4288 | CG2 | VAL | B | 589 | 35.751 | 36.315 | 27.238 | 1.00 | 24.02 | C |
| ATOM | 4289 | C | VAL | B | 589 | 36.082 | 37.688 | 30.816 | 1.00 | 21.77 | C |
| ATOM | 4290 | O | VAL | B | 589 | 35.384 | 38.666 | 31.097 | 1.00 | 21.67 | O |
| ATOM | 4291 | N | GLU | B | 590 | 36.503 | 36.817 | 31.736 | 1.00 | 24.09 | N |
| ATOM | 4292 | CA | GLU | B | 590 | 36.158 | 36.986 | 33.149 | 1.00 | 23.44 | C |
| ATOM | 4293 | CB | GLU | B | 590 | 36.700 | 35.792 | 33.980 | 1.00 | 23.66 | C |
| ATOM | 4294 | CG | GLU | B | 590 | 36.346 | 35.806 | 35.489 | 1.00 | 28.31 | C |
| ATOM | 4295 | CD | GLU | B | 590 | 37.173 | 36.793 | 36.342 | 1.00 | 30.31 | C |
| ATOM | 4296 | OE1 | GLU | B | 590 | 38.375 | 36.990 | 36.060 | 1.00 | 30.33 | O |
| ATOM | 4297 | OE2 | GLU | B | 590 | 36.623 | 37.345 | 37.325 | 1.00 | 30.51 | O |
| ATOM | 4298 | C | GLU | B | 590 | 36.722 | 38.300 | 33.698 | 1.00 | 24.63 | C |
| ATOM | 4299 | O | GLU | B | 590 | 36.060 | 38.995 | 34.469 | 1.00 | 21.17 | O |
| ATOM | 4300 | N | GLN | B | 591 | 37.948 | 38.645 | 33.304 | 1.00 | 23.83 | N |
| ATOM | 4301 | CA | GLN | B | 591 | 38.550 | 39.896 | 33.775 | 1.00 | 25.38 | C |
| ATOM | 4302 | CB | GLN | B | 591 | 40.005 | 39.980 | 33.324 | 1.00 | 27.76 | C |
| ATOM | 4303 | CG | GLN | B | 591 | 40.877 | 38.896 | 33.902 | 1.00 | 36.74 | C |
| ATOM | 4304 | CD | GLN | B | 591 | 42.333 | 39.101 | 33.554 | 1.00 | 43.26 | C |
| ATOM | 4305 | OE1 | GLN | B | 591 | 42.669 | 39.908 | 32.677 | 1.00 | 44.86 | O |
| ATOM | 4306 | NE2 | GLN | B | 591 | 43.206 | 38.370 | 34.230 | 1.00 | 44.68 | N |
| ATOM | 4307 | C | GLN | B | 591 | 37.818 | 41.132 | 33.279 | 1.00 | 25.08 | C |
| ATOM | 4308 | O | GLN | B | 591 | 37.616 | 42.102 | 34.019 | 1.00 | 26.57 | O |
| ATOM | 4309 | N | ARG | B | 592 | 37.423 | 41.110 | 32.012 | 1.00 | 23.67 | N |
| ATOM | 4310 | CA | ARG | B | 592 | 36.712 | 42.239 | 31.437 | 1.00 | 25.81 | C |
| ATOM | 4311 | CB | ARG | B | 592 | 36.631 | 42.087 | 29.917 | 1.00 | 23.30 | C |
| ATOM | 4312 | CG | ARG | B | 592 | 38.018 | 42.247 | 29.276 | 1.00 | 31.02 | C |
| ATOM | 4313 | CD | ARG | B | 592 | 38.006 | 41.980 | 27.792 | 1.00 | 31.50 | C |
| ATOM | 4314 | NE | ARG | B | 592 | 37.163 | 42.935 | 27.079 | 1.00 | 25.46 | N |
| ATOM | 4315 | CZ | ARG | B | 592 | 36.913 | 42.861 | 25.781 | 1.00 | 24.58 | C |
| ATOM | 4316 | NH1 | ARG | B | 592 | 37.446 | 41.880 | 25.069 | 1.00 | 25.78 | N |
| ATOM | 4317 | NH2 | ARG | B | 592 | 36.127 | 43.760 | 25.206 | 1.00 | 26.82 | N |
| ATOM | 4318 | C | ARG | B | 592 | 35.326 | 42.323 | 32.039 | 1.00 | 25.35 | C |
| ATOM | 4319 | O | ARG | B | 592 | 34.803 | 43.415 | 32.284 | 1.00 | 25.98 | O |
| ATOM | 4320 | N | MET | B | 593 | 34.722 | 41.165 | 32.293 | 1.00 | 23.15 | N |
| ATOM | 4321 | CA | MET | B | 593 | 33.380 | 41.176 | 32.867 | 1.00 | 24.63 | C |
| ATOM | 4322 | CB | MET | B | 593 | 32.774 | 39.764 | 32.822 | 1.00 | 22.51 | C |
| ATOM | 4323 | CG | MET | B | 593 | 31.324 | 39.697 | 33.218 | 1.00 | 22.94 | C |
| ATOM | 4324 | SD | MET | B | 593 | 30.230 | 10.679 | 32.086 | 1.00 | 29.24 | S |
| ATOM | 4325 | CE | MET | B | 593 | 29.463 | 41.710 | 33.269 | 1.00 | 27.63 | C |
| ATOM | 4326 | C | MET | B | 593 | 33.442 | 41.705 | 34.297 | 1.00 | 25.96 | C |
| ATOM | 4327 | O | MET | B | 593 | 32.582 | 42.480 | 34.719 | 1.00 | 28.48 | O |
| ATOM | 4328 | N | ARG | B | 594 | 34.459 | 41.296 | 35.044 | 1.00 | 23.96 | N |
| ATOM | 4329 | CA | ARG | B | 594 | 34.605 | 41.743 | 36.417 | 1.00 | 24.96 | C |
| ATOM | 4330 | CB | ARG | B | 594 | 35.849 | 41.106 | 37.042 | 1.00 | 23.17 | C |
| ATOM | 4331 | CG | ARG | B | 594 | 36.061 | 41.480 | 38.522 | 1.00 | 25.85 | C |
| ATOM | 4332 | CD | ARG | B | 594 | 37.401 | 40.946 | 39.044 | 1.00 | 37.38 | C |
| ATOM | 4333 | NE | ARG | B | 594 | 37.415 | 39.490 | 39.157 | 1.00 | 42.96 | N |
| ATOM | 4334 | CZ | ARG | B | 594 | 36.932 | 38.814 | 40.201 | 1.00 | 43.14 | C |
| ATOM | 4335 | NH1 | ARG | B | 594 | 36.400 | 39.464 | 41.232 | 1.00 | 47.77 | N |
| ATOM | 4336 | NH2 | ARG | B | 594 | 36.966 | 37.488 | 40.206 | 1.00 | 41.23 | N |
| ATOM | 4337 | C | ARG | B | 594 | 34.707 | 43.271 | 36.461 | 1.00 | 27.20 | C |
| ATOM | 4338 | O | ARG | B | 594 | 34.071 | 43.930 | 37.291 | 1.00 | 29.00 | O |
| ATOM | 4339 | N | ALA | B | 595 | 35.499 | 43.827 | 35.548 | 1.00 | 29.98 | N |
| ATOM | 4340 | CA | ALA | B | 595 | 35.709 | 45.271 | 35.455 | 1.00 | 32.90 | C |
| ATOM | 4341 | CB | ALA | B | 595 | 36.814 | 45.570 | 34.424 | 1.00 | 33.21 | C |
| ATOM | 4342 | C | ALA | B | 595 | 34.423 | 46.010 | 35.087 | 1.00 | 33.52 | C |
| ATOM | 4343 | O | ALA | B | 595 | 34.139 | 47.075 | 35.627 | 1.00 | 31.72 | O |
| ATOM | 4344 | N | CYS | B | 596 | 33.646 | 45.439 | 34.166 | 1.00 | 31.09 | N |
| ATOM | 4345 | CA | CYS | B | 596 | 32.389 | 46.044 | 33.750 | 1.00 | 32.73 | C |
| ATOM | 4346 | CB | CYS | B | 596 | 31.768 | 45.264 | 32.585 | 1.00 | 31.31 | C |
| ATOM | 4347 | SG | CYS | B | 596 | 32.614 | 45.490 | 30.999 | 1.00 | 41.27 | S |
| ATOM | 4348 | C | CYS | B | 596 | 31.412 | 46.053 | 34.907 | 1.00 | 33.26 | C |
| ATOM | 4349 | O | CYS | B | 596 | 30.741 | 47.055 | 35.158 | 1.00 | 31.15 | O |
| ATOM | 4350 | N | TYR | B | 597 | 31.326 | 44.925 | 35.604 | 1.00 | 28.78 | N |
| ATOM | 4351 | CA | TYR | B | 597 | 30.415 | 44.811 | 36.736 | 1.00 | 29.03 | C |
| ATOM | 4352 | CB | TYR | B | 597 | 30.465 | 43.384 | 37.311 | 1.00 | 30.58 | C |
| ATOM | 4353 | CG | TYR | B | 597 | 29.811 | 43.233 | 38.665 | 1.00 | 31.39 | C |
| ATOM | 4354 | CD1 | TYR | B | 597 | 28.548 | 43.773 | 38.917 | 1.00 | 33.23 | C |
| ATOM | 4355 | CE1 | TYR | B | 597 | 27.957 | 43.673 | 40.186 | 1.00 | 36.25 | C |
| ATOM | 4356 | CD2 | TYR | B | 597 | 30.465 | 42.579 | 39.702 | 1.00 | 36.29 | C |
| ATOM | 4357 | CE2 | TYR | B | 597 | 29.884 | 42.466 | 40.977 | 1.00 | 42.36 | C |
| ATOM | 4358 | CZ | TYR | B | 597 | 28.634 | 43.021 | 41.210 | 1.00 | 40.23 | C |
| ATOM | 4359 | OH | TYR | B | 597 | 28.091 | 42.957 | 42.474 | 1.00 | 40.28 | O |
| ATOM | 4360 | C | TYR | B | 597 | 30.758 | 45.825 | 37.827 | 1.00 | 32.53 | C |
| ATOM | 4361 | O | TYR | B | 597 | 29.871 | 46.537 | 38.335 | 1.00 | 33.59 | O |
| ATOM | 4362 | N | TYR | B | 598 | 32.036 | 45.883 | 38.179 | 1.00 | 30.77 | N |
| ATOM | 4363 | CA | TYR | B | 598 | 32.508 | 46.793 | 39.218 | 1.00 | 38.97 | C |
| ATOM | 4364 | CB | TYR | B | 598 | 34.012 | 46.604 | 39.460 | 1.00 | 39.06 | C |
| ATOM | 4365 | CG | TYR | B | 598 | 34.385 | 45.365 | 40.244 | 1.00 | 40.81 | C |
| ATOM | 4366 | CD1 | TYR | B | 598 | 35.714 | 45.111 | 40.590 | 1.00 | 48.71 | C |
| ATOM | 4367 | CE1 | TYR | B | 598 | 36.075 | 43.943 | 41.281 | 1.00 | 48.51 | C |
| ATOM | 4368 | CD2 | TYR | B | 598 | 33.424 | 44.423 | 40.612 | 1.00 | 47.38 | C |
| ATOM | 4369 | CE2 | TYR | B | 598 | 33.771 | 43.259 | 41.298 | 1.00 | 46.97 | C |
| ATOM | 4370 | CZ | TYR | B | 598 | 35.098 | 43.024 | 41.626 | 1.00 | 49.34 | C |
| ATOM | 4371 | OH | TYR | B | 598 | 32.445 | 41.854 | 42.264 | 1.00 | 48.67 | O |
| ATOM | 4372 | C | TYR | B | 598 | 32.227 | 48.230 | 38.823 | 1.00 | 43.37 | C |
| ATOM | 4373 | O | TYR | B | 598 | 31.873 | 49.057 | 39.667 | 1.00 | 48.95 | O |
| ATOM | 4374 | N | SER | B | 599 | 32.379 | 48.535 | 37.541 | 1.00 | 37.43 | N |
| ATOM | 4375 | CA | SER | B | 599 | 32.122 | 49.872 | 37.055 | 1.00 | 43.16 | C |
| ATOM | 4376 | CB | SER | B | 599 | 32.529 | 49.961 | 35.590 | 1.00 | 43.45 | C |
| ATOM | 4377 | OG | SER | B | 599 | 32.332 | 51.262 | 35.090 | 1.00 | 53.97 | O |
| ATOM | 4378 | C | SER | B | 599 | 30.636 | 50.205 | 37.223 | 1.00 | 45.93 | C |
| ATOM | 4379 | O | SER | B | 599 | 30.271 | 51.316 | 37.617 | 1.00 | 40.00 | O |
| ATOM | 4380 | N | LEU | B | 600 | 29.773 | 49.240 | 36.935 | 1.00 | 39.11 | N |
| ATOM | 4381 | CA | LEU | B | 600 | 28.337 | 49.462 | 37.065 | 1.00 | 44.57 | C |
| ATOM | 4382 | CB | LEU | B | 600 | 27.552 | 48.395 | 36.295 | 1.00 | 38.25 | C |
| ATOM | 4383 | CG | LEU | B | 600 | 27.711 | 48.411 | 34.770 | 1.00 | 42.03 | C |
| ATOM | 4384 | CD1 | LEU | B | 600 | 27.111 | 47.196 | 34.147 | 1.00 | 37.50 | C |
| ATOM | 4385 | CD2 | LEU | B | 600 | 27.039 | 49.683 | 34.214 | 1.00 | 45.85 | C |
| ATOM | 4386 | C | LEU | B | 600 | 27.897 | 49.456 | 38.516 | 1.00 | 43.73 | C |
| ATOM | 4387 | O | LEU | B | 600 | 26.980 | 50.191 | 38.892 | 1.00 | 46.87 | O |
| ATOM | 4388 | N | ALA | B | 601 | 28.556 | 48.628 | 39.323 | 1.00 | 40.69 | N |
| ATOM | 4389 | CA | ALA | B | 601 | 28.242 | 48.478 | 40.741 | 1.00 | 47.37 | C |
| ATOM | 4390 | CB | ALA | B | 601 | 29.065 | 47.346 | 41.335 | 1.00 | 46.75 | C |
| ATOM | 4391 | C | ALA | B | 601 | 28.506 | 49.775 | 41.498 | 1.00 | 52.65 | C |
| ATOM | 4392 | O | ALA | B | 601 | 27.834 | 50.082 | 42.483 | 1.00 | 53.31 | O |
| ATOM | 4393 | N | SER | B | 602 | 29.492 | 50.527 | 41.021 | 1.00 | 52.09 | N |
| ATOM | 4394 | CA | SER | B | 602 | 29.860 | 51.804 | 41.617 | 1.00 | 57.33 | C |
| ATOM | 4395 | CB | SER | B | 602 | 31.346 | 52.069 | 41.381 | 1.00 | 58.94 | C |
| ATOM | 4396 | OG | SER | B | 602 | 32.141 | 51.082 | 42.020 | 1.00 | 59.82 | O |
| ATOM | 4397 | C | SER | B | 602 | 29.024 | 52.912 | 40.981 | 1.00 | 59.94 | C |
| ATOM | 4398 | O | SER | B | 602 | 29.388 | 54.086 | 41.029 | 1.00 | 64.66 | O |
| ATOM | 4399 | N | LYS | B | 603 | 27.900 | 52.515 | 40.389 | 1.00 | 62.33 | N |
| ATOM | 4400 | CA | LYS | B | 603 | 26.974 | 53.425 | 39.720 | 1.00 | 64.02 | C |
| ATOM | 4401 | CB | LYS | B | 603 | 26.569 | 54.555 | 40.673 | 1.00 | 64.16 | C |
| ATOM | 4402 | CG | LYS | B | 603 | 25.091 | 54.908 | 40.626 | 1.00 | 65.22 | C |
| ATOM | 4403 | CD | LYS | B | 603 | 24.767 | 55.995 | 41.635 | 1.00 | 64.28 | C |
| ATOM | 4404 | CE | LYS | B | 603 | 23.270 | 56.270 | 41.693 | 1.00 | 67.89 | C |
| ATOM | 4405 | NZ | LYS | B | 603 | 22.949 | 57.422 | 42.589 | 1.00 | 67.06 | N |
| ATOM | 4406 | C | LYS | B | 603 | 27.609 | 53.996 | 38.446 | 1.00 | 65.47 | C |
| ATOM | 4407 | O | LYS | B | 603 | 27.032 | 53.798 | 37.352 | 1.00 | 66.51 | O |
| ATOM | 4408 | OXT | LYS | B | 603 | 28.682 | 54.625 | 38.551 | 1.00 | 68.34 | O |
| TER | 1 | | LYS | B | 603 | | | | | | |
| ATOM | 4409 | O4 | STU | C | 995 | 10.701 | −7.072 | −3.088 | 1.00 | 26.93 | O |
| ATOM | 4410 | C25 | STU | C | 995 | 11.180 | −5.863 | −3.690 | 1.00 | 23.34 | C |
| ATOM | 4411 | C24 | STU | C | 995 | 10.859 | −4.664 | −2.680 | 1.00 | 29.45 | C |
| ATOM | 4412 | C23 | STU | C | 995 | 10.138 | −5.127 | −1.364 | 1.00 | 22.89 | C |
| ATOM | 4413 | C22 | STU | C | 995 | 8.878 | −5.986 | −1.697 | 1.00 | 25.16 | C |
| ATOM | 4414 | C21 | STU | C | 995 | 9.461 | −7.275 | −2.433 | 1.00 | 26.44 | C |
| ATOM | 4415 | C26 | STU | C | 995 | 9.777 | −8.334 | −1.360 | 1.00 | 25.18 | C |
| ATOM | 4416 | N2 | STU | C | 995 | 8.422 | −7.719 | −3.422 | 1.00 | 23.09 | N |
| ATOM | 4417 | C18 | STU | C | 995 | 8.412 | −7.138 | −4.747 | 1.00 | 21.71 | C |
| ATOM | 4418 | C19 | STU | C | 995 | 9.290 | −6.274 | −6.405 | 1.00 | 24.08 | C |
| ATOM | 4419 | C6 | STU | C | 995 | 9.064 | −5.832 | −6.736 | 1.00 | 22.57 | C |
| ATOM | 4420 | C7 | STU | C | 995 | 7.866 | −6.422 | −7.405 | 1.00 | 25.97 | C |
| ATOM | 4421 | C10 | STU | C | 995 | 7.050 | −7.293 | −6.788 | 1.00 | 23.03 | C |
| ATOM | 4422 | C11 | STU | C | 995 | 7.275 | −7.726 | −5.438 | 1.00 | 20.70 | C |
| ATOM | 4423 | C12 | STU | C | 995 | 6.603 | −8.591 | −4.523 | 1.00 | 24.72 | C |
| ATOM | 4424 | C17 | STU | C | 995 | 7.298 | −8.588 | −3.264 | 1.00 | 25.22 | C |
| ATOM | 4425 | C16 | STU | C | 995 | 6.820 | −9.373 | −2.193 | 1.00 | 26.63 | C |
| ATOM | 4426 | C15 | STU | C | 995 | 5.671 | −10.131 | −2.193 | 1.00 | 26.63 | C |
| ATOM | 4427 | C14 | STU | C | 995 | 4.957 | −10.136 | −3.585 | 1.00 | 27.59 | C |
| ATOM | 4428 | C13 | STU | C | 995 | 5.443 | −9.364 | −4.643 | 1.00 | 24.26 | C |
| ATOM | 4429 | C9 | STU | C | 995 | 5.906 | −7.701 | −7.728 | 1.00 | 24.26 | C |
| ATOM | 4430 | N1 | STU | C | 995 | 6.283 | −6.914 | −8.894 | 1.00 | 26.80 | N |
| ATOM | 4431 | C8 | STU | C | 995 | 7.390 | −6.161 | −8.794 | 1.00 | 24.42 | C |
| ATOM | 4432 | O5 | STU | C | 995 | 7.870 | −5.431 | −9.638 | 1.00 | 25.22 | O |
| ATOM | 4433 | C5 | STU | C | 995 | 10.084 | −4.899 | −7.090 | 1.00 | 21.94 | C |
| ATOM | 4434 | C20 | STU | C | 995 | 10.927 | −4.761 | −5.943 | 1.00 | 28.12 | C |
| ATOM | 4435 | C1 | STU | C | 995 | 12.071 | −3.894 | −5.922 | 1.00 | 27.73 | C |
| ATOM | 4436 | C2 | STU | C | 995 | 12.350 | −3.175 | −7.097 | 1.00 | 30.27 | C |
| ATOM | 4437 | C3 | STU | C | 995 | 11.500 | −3.313 | −8.263 | 1.00 | 28.96 | C |
| ATOM | 4438 | C4 | STU | C | 995 | 10.403 | −4.161 | −8.243 | 1.00 | 26.89 | C |
| ATOM | 4439 | N3 | STU | C | 995 | 10.433 | −5.565 | −4.940 | 1.00 | 23.33 | N |
| ATOM | 4440 | O6 | STU | C | 995 | 8.116 | −5.181 | −2.652 | 1.00 | 24.62 | O |
| ATOM | 4441 | C27 | STU | C | 995 | 6.704 | −5.144 | −2.385 | 1.00 | 24.32 | C |
| ATOM | 4442 | N4 | STU | C | 995 | 9.777 | −3.833 | −0.560 | 1.00 | 24.88 | N |
| ATOM | 4443 | C28 | STU | C | 995 | 9.296 | −4.199 | 0.820 | 1.00 | 26.60 | C |
| TER | 1 | | STU | C | 995 | | | | | | |
| ATOM | 4444 | O4 | STU | D | 996 | 14.841 | 21.718 | 34.032 | 1.00 | 26.13 | O |
| ATOM | 4445 | C25 | STU | D | 996 | 14.373 | 22.920 | 34.632 | 1.00 | 22.93 | C |
| ATOM | 4446 | C24 | STU | D | 996 | 14.706 | 24.128 | 33.610 | 1.00 | 28.10 | C |
| ATOM | 4447 | C23 | STU | D | 996 | 15.417 | 23.644 | 32.288 | 1.00 | 24.94 | C |
| ATOM | 4448 | C22 | STU | D | 996 | 16.665 | 22.776 | 32.631 | 1.00 | 23.21 | C |
| ATOM | 4449 | C21 | STU | D | 996 | 16.072 | 21.495 | 33.377 | 1.00 | 27.43 | C |
| ATOM | 4450 | C26 | STU | D | 996 | 15.756 | 20.434 | 32.323 | 1.00 | 26.04 | C |
| ATOM | 4451 | N2 | STU | D | 996 | 17.133 | 21.077 | 32.323 | 1.00 | 21.43 | N |
| ATOM | 4452 | C18 | STU | D | 996 | 17.120 | 21.637 | 35.688 | 1.00 | 20.69 | C |
| ATOM | 4453 | C19 | STU | D | 996 | 16.275 | 22.524 | 36.330 | 1.00 | 23.53 | C |
| ATOM | 4454 | C6 | STU | D | 996 | 16.487 | 22.957 | 37.663 | 1.00 | 22.38 | C |
| ATOM | 4455 | C7 | STU | D | 996 | 17.682 | 22.366 | 38.337 | 1.00 | 26.78 | C |
| ATOM | 4456 | C10 | STU | D | 996 | 18.504 | 21.518 | 37.720 | 1.00 | 22.89 | C |
| ATOM | 4457 | C11 | STU | D | 996 | 18.270 | 21.070 | 36.370 | 1.00 | 19.86 | C |
| ATOM | 4458 | C12 | STU | D | 996 | 18.952 | 20.227 | 35.457 | 1.00 | 22.51 | C |
| ATOM | 4459 | C17 | STU | D | 996 | 18.237 | 20.202 | 34.206 | 1.00 | 25.41 | C |
| ATOM | 4460 | C16 | STU | D | 996 | 18.719 | 19.424 | 33.133 | 1.00 | 26.19 | C |
| ATOM | 4461 | C15 | STU | D | 996 | 19.869 | 18.670 | 33.275 | 1.00 | 25.48 | C |
| ATOM | 4462 | C14 | STU | D | 996 | 20.600 | 18.678 | 34.521 | 1.00 | 25.48 | C |
| ATOM | 4463 | C13 | STU | D | 996 | 20.112 | 19.449 | 35.580 | 1.00 | 23.07 | C |
| ATOM | 4464 | C9 | STU | D | 996 | 19.647 | 21.104 | 38.659 | 1.00 | 23.46 | C |
| ATOM | 4465 | N1 | STU | D | 996 | 19.276 | 21.887 | 39.826 | 1.00 | 25.16 | N |
| ATOM | 4466 | C8 | STU | D | 996 | 18.166 | 22.640 | 39.728 | 1.00 | 22.97 | C |
| ATOM | 4467 | O5 | STU | D | 996 | 17.688 | 23.365 | 40.576 | 1.00 | 23.93 | O |
| ATOM | 4468 | C5 | STU | D | 996 | 15.472 | 23.870 | 38.015 | 1.00 | 23.02 | C |
| ATOM | 4469 | C20 | STU | D | 996 | 14.631 | 24.010 | 36.865 | 1.00 | 26.41 | C |
| ATOM | 4470 | C1 | STU | D | 996 | 13.476 | 24.883 | 36.850 | 1.00 | 25.65 | C |
| ATOM | 4471 | C2 | STU | D | 996 | 13.208 | 25.595 | 38.019 | 1.00 | 30.69 | C |
| ATOM | 4472 | C3 | STU | D | 996 | 14.057 | 25.459 | 39.181 | 1.00 | 27.27 | C |
| ATOM | 4473 | C4 | STU | D | 996 | 15.157 | 24.612 | 39.158 | 1.00 | 26.75 | C |
| ATOM | 4474 | N3 | STU | D | 996 | 15.112 | 23.212 | 35.878 | 1.00 | 22.92 | N |
| ATOM | 4475 | O6 | STU | D | 996 | 17.429 | 23.573 | 33.576 | 1.00 | 23.16 | O |
| ATOM | 4476 | C27 | STU | D | 996 | 18.832 | 23.642 | 33.286 | 1.00 | 24.37 | C |
| ATOM | 4477 | N4 | STU | D | 996 | 15.802 | 24.935 | 31.463 | 1.00 | 23.94 | N |
| ATOM | 4478 | C28 | STU | D | 996 | 16.275 | 24.547 | 30.090 | 1.00 | 24.01 | C |
| TER | 1 | | STU | D | 996 | | | | | | |
| ATOM | 4479 | OH2 | TIP | S | 1 | 0.421 | 4.204 | 13.673 | 1.00 | 22.10 | O |
| ATOM | 4480 | OH2 | TIP | S | 2 | 0.033 | 9.184 | 13.707 | 1.00 | 21.18 | O |
| ATOM | 4481 | OH2 | TIP | S | 3 | 11.945 | −2.213 | 0.210 | 1.00 | 22.87 | O |
| ATOM | 4482 | OH2 | TIP | S | 4 | 25.132 | 33.017 | 17.239 | 1.00 | 22.45 | O |
| ATOM | 4483 | OH2 | TIP | S | 5 | 25.496 | 37.992 | 17.154 | 1.00 | 20.79 | O |
| ATOM | 4484 | OH2 | TIP | S | 6 | −12.049 | −13.944 | 12.451 | 1.00 | 22.33 | O |
| ATOM | 4485 | OH2 | TIP | S | 7 | 14.395 | 8.858 | 57.889 | 1.00 | 29.61 | O |
| ATOM | 4486 | OH2 | TIP | S | 8 | 13.627 | 26.578 | 30.735 | 1.00 | 22.89 | O |
| ATOM | 4487 | OH2 | TIP | S | 9 | 11.059 | −19.955 | −26.913 | 1.00 | 28.80 | O |
| ATOM | 4488 | OH2 | TIP | S | 10 | 29.070 | 27.910 | 20.630 | 1.00 | 25.77 | O |
| ATOM | 4489 | OH2 | TIP | S | 11 | −3.520 | −0.809 | 10.277 | 1.00 | 25.06 | O |
| ATOM | 4490 | OH2 | TIP | S | 12 | 18.345 | 30.239 | 26.486 | 1.00 | 23.39 | O |
| ATOM | 4491 | OH2 | TIP | S | 13 | 1.869 | 14.815 | 18.471 | 1.00 | 23.60 | O |
| ATOM | 4492 | OH2 | TIP | S | 14 | 35.075 | 25.727 | 25.589 | 1.00 | 28.87 | O |
| ATOM | 4493 | OH2 | TIP | S | 15 | 7.227 | 1.385 | 4.419 | 1.00 | 22.68 | O |
| ATOM | 4494 | OH2 | TIP | S | 16 | −9.560 | −3.026 | 2.397 | 1.00 | 27.48 | O |
| ATOM | 4495 | OH2 | TIP | S | 17 | −5.464 | −3.857 | 7.924 | 1.00 | 23.68 | O |
| ATOM | 4496 | OH2 | TIP | S | 18 | −6.496 | 8.745 | 20.202 | 1.00 | 23.82 | O |
| ATOM | 4497 | OH2 | TIP | S | 19 | −2.873 | −2.292 | 5.603 | 1.00 | 23.16 | O |
| ATOM | 4498 | OH2 | TIP | S | 20 | −11.803 | 8.285 | 18.254 | 1.00 | 24.29 | O |
| ATOM | 4499 | OH2 | TIP | S | 21 | 28.416 | 26.453 | 25.290 | 1.00 | 23.25 | O |
| ATOM | 4500 | OH2 | TIP | S | 22 | 37.391 | 37.035 | 12.669 | 1.00 | 26.06 | O |
| ATOM | 4501 | OH2 | TIP | S | 23 | −17.573 | −7.098 | 4.343 | 1.00 | 27.79 | O |
| ATOM | 4502 | OH2 | TIP | S | 24 | 32.073 | 37.514 | 10.731 | 1.00 | 24.94 | O |
| ATOM | 4503 | OH2 | TIP | S | 25 | 13.480 | 31.449 | 36.563 | 1.00 | 25.71 | O |
| ATOM | 4504 | OH2 | TIP | S | 26 | −14.437 | 9.362 | 17.730 | 1.00 | 32.36 | O |
| ATOM | 4505 | OH2 | TIP | S | 27 | 35.416 | 25.457 | 18.662 | 1.00 | 25.28 | O |
| ATOM | 4506 | OH2 | TIP | S | 28 | 31.029 | 24.811 | 22.942 | 1.00 | 25.60 | O |
| ATOM | 4507 | OH2 | TIP | S | 29 | −9.872 | −3.294 | 12.263 | 1.00 | 24.74 | O |
| ATOM | 4508 | OH2 | TIP | S | 30 | 26.467 | 9.752 | 17.607 | 1.00 | 27.17 | O |
| ATOM | 4509 | OH2 | TIP | S | 31 | 6.790 | −6.857 | 1.076 | 1.00 | 30.39 | O |
| ATOM | 4510 | OH2 | TIP | S | 32 | 40.010 | 38.095 | 13.209 | 1.00 | 33.16 | O |
| ATOM | 4511 | OH2 | TIP | S | 33 | 34.735 | 13.425 | 28.194 | 1.00 | 29.80 | O |
| ATOM | 4512 | OH2 | TIP | S | 34 | −9.205 | −15.364 | 2.764 | 1.00 | 26.99 | O |
| ATOM | 4513 | OH2 | TIP | S | 35 | −0.980 | −18.986 | 13.270 | 1.00 | 26.43 | O |
| ATOM | 4514 | OH2 | TIP | S | 36 | 24.213 | 18.114 | 34.694 | 1.00 | 29.43 | O |
| ATOM | 4515 | OH2 | TIP | S | 37 | 1.416 | −10.764 | −3.798 | 1.00 | 29.81 | O |
| ATOM | 4516 | OH2 | TIP | S | 38 | 43.144 | 21.699 | 26.548 | 1.00 | 30.26 | O |
| ATOM | 4517 | OH2 | TIP | S | 39 | −14.848 | 7.171 | −3.854 | 1.00 | 30.65 | O |
| ATOM | 4518 | OH2 | TIP | S | 40 | 39.180 | 42.936 | 36.141 | 1.00 | 31.31 | O |
| ATOM | 4519 | OH2 | TIP | S | 41 | 40.406 | 35.919 | 34.748 | 1.00 | 30.00 | O |
| ATOM | 4520 | OH2 | TIP | S | 42 | 0.420 | 2.218 | 15.485 | 1.00 | 29.77 | O |
| ATOM | 4521 | OH2 | TIP | S | 43 | 35.271 | 29.926 | 13.265 | 1.00 | 31.24 | O |
| ATOM | 4522 | OH2 | TIP | S | 44 | 12.095 | 2.647 | −5.592 | 1.00 | 26.76 | O |
| ATOM | 4523 | OH2 | TIP | S | 45 | −9.718 | 1.172 | 17.653 | 1.00 | 30.07 | O |
| ATOM | 4524 | OH2 | TIP | S | 46 | −3.404 | 14.594 | 13.732 | 1.00 | 30.39 | O |
| ATOM | 4525 | OH2 | TIP | S | 47 | 18.726 | 21.922 | 29.867 | 1.00 | 32.07 | O |
| ATOM | 4526 | OH2 | TIP | S | 48 | 13.555 | 7.866 | 51.920 | 1.00 | 39.23 | O |
| ATOM | 4527 | OH2 | TIP | S | 49 | 28.975 | 43.404 | 17.200 | 1.00 | 29.02 | O |
| ATOM | 4528 | OH2 | TIP | S | 50 | −13.569 | 14.156 | −5.246 | 1.00 | 33.70 | O |
| ATOM | 4528 | OH2 | TIP | S | 51 | 26.355 | 20.255 | 28.832 | 1.00 | 29.58 | O |
| ATOM | 4530 | OH2 | TIP | S | 52 | 9.161 | 10.323 | 16.585 | 1.00 | 35.40 | O |
| ATOM | 4531 | OH2 | TIP | S | 53 | −0.757 | −8.626 | 2.106 | 1.00 | 29.81 | O |
| ATOM | 4532 | OH2 | TIP | S | 54 | −14.070 | −4.107 | 6.620 | 1.00 | 27.24 | O |
| ATOM | 4533 | OH2 | TIP | S | 55 | 11.946 | −20.893 | −21.008 | 1.00 | 43.13 | O |
| ATOM | 4534 | OH2 | TIP | S | 56 | 25.189 | 30.969 | 15.454 | 1.00 | 29.79 | O |
| ATOM | 4535 | OH2 | TIP | S | 57 | 36.813 | 31.260 | 38.289 | 1.00 | 31.14 | O |
| ATOM | 4536 | OH2 | TIP | S | 58 | −11.290 | 2.441 | −7.391 | 1.00 | 30.15 | O |
| ATOM | 4537 | OH2 | TIP | S | 59 | 9.218 | 10.129 | 12.339 | 1.00 | 32.37 | O |
| ATOM | 4538 | OH2 | TIP | S | 60 | 16.469 | 39.110 | 14.335 | 1.00 | 36.98 | O |
| ATOM | 4539 | OH2 | TIP | S | 61 | 39.600 | 24.658 | 24.347 | 1.00 | 28.57 | O |
| ATOM | 4540 | OH2 | TIP | S | 62 | 40.886 | 19.814 | 39.523 | 1.00 | 31.30 | O |
| ATOM | 4541 | OH2 | TIP | S | 63 | 28.663 | 25.506 | 21.907 | 1.00 | 28.05 | O |
| ATOM | 4542 | OH2 | TIP | S | 64 | 16.334 | 39.034 | 18.587 | 1.00 | 30.97 | O |
| ATOM | 4543 | OH2 | TIP | S | 65 | −4.453 | −10.056 | −10.600 | 1.00 | 35.01 | O |
| ATOM | 4544 | OH2 | TIP | S | 66 | 30.644 | 48.610 | 22.338 | 1.00 | 34.83 | O |
| ATOM | 4545 | OH2 | TIP | S | 67 | 29.080 | 17.008 | 23.184 | 1.00 | 42.53 | O |
| ATOM | 4546 | OH2 | TIP | S | 68 | 20.437 | −8.985 | −8.586 | 1.00 | 30.66 | O |
| ATOM | 4547 | OH2 | TIP | S | 69 | −3.136 | −3.317 | 8.937 | 1.00 | 26.92 | O |
| ATOM | 4548 | OH2 | TIP | S | 70 | −3.528 | −11.754 | 7.661 | 1.00 | 42.36 | O |
| ATOM | 4549 | OH2 | TIP | S | 71 | −14.052 | 9.990 | −6.784 | 1.00 | 36.91 | O |
| ATOM | 4550 | OH2 | TIP | S | 72 | 13.280 | 20.389 | 48.150 | 1.00 | 37.12 | O |
| ATOM | 4551 | OH2 | TIP | S | 73 | 39.598 | 27.291 | 23.321 | 1.00 | 29.86 | O |
| ATOM | 4552 | OH2 | TIP | S | 74 | −14.000 | −1.419 | 7.609 | 1.00 | 29.52 | O |
| ATOM | 4553 | OH2 | TIP | S | 75 | 12.287 | −8.370 | −17.212 | 1.00 | 38.16 | O |
| ATOM | 4554 | OH2 | TIP | S | 76 | 0.127 | −2.062 | 7.336 | 1.00 | 31.92 | O |
| ATOM | 4555 | OH2 | TIP | S | 77 | 39.657 | 38.747 | 37.688 | 1.00 | 35.97 | O |
| ATOM | 4556 | OH2 | TIP | S | 78 | 6.921 | 10.871 | 13.240 | 1.00 | 39.95 | O |
| ATOM | 4557 | OH2 | TIP | S | 79 | 3.173 | 20.739 | −5.925 | 1.00 | 36.12 | O |
| ATOM | 4558 | OH2 | TIP | S | 80 | 2.499 | −2.849 | 7.192 | 1.00 | 37.12 | O |
| ATOM | 4559 | OH2 | TIP | S | 81 | −5.087 | 20.010 | 8.585 | 1.00 | 36.89 | O |
| ATOM | 4560 | OH2 | TIP | S | 82 | 25.391 | 26.672 | 23.460 | 1.00 | 34.04 | O |
| ATOM | 4561 | OH2 | TIP | S | 83 | 23.029 | 25.932 | 23.668 | 1.00 | 37.18 | O |
| ATOM | 4562 | OH2 | TIP | S | 84 | 13.382 | −6.941 | −0.286 | 1.00 | 40.85 | O |
| ATOM | 4563 | OH2 | TIP | S | 85 | 27.715 | 12.569 | 29.074 | 1.00 | 44.29 | O |
| ATOM | 4564 | OH2 | TIP | S | 86 | 29.990 | 18.665 | 41.491 | 1.00 | 37.50 | O |
| ATOM | 4565 | OH2 | TIP | S | 87 | 22.380 | 47.448 | 36.934 | 1.00 | 39.98 | O |
| ATOM | 4566 | OH2 | TIP | S | 88 | 18.662 | 39.652 | 17.714 | 1.00 | 39.70 | O |
| ATOM | 4567 | OH2 | TIP | S | 89 | 40.100 | 17.469 | 30.708 | 1.00 | 36.01 | O |
| ATOM | 4568 | OH2 | TIP | S | 90 | −14.239 | 11.528 | 5.631 | 1.00 | 40.89 | O |
| ATOM | 4569 | OH2 | TIP | S | 91 | 15.281 | 18.931 | 57.001 | 1.00 | 45.15 | O |
| ATOM | 4570 | OH2 | TIP | S | 92 | 15.691 | −4.194 | −6.244 | 1.00 | 46.89 | O |
| ATOM | 4571 | OH2 | TIP | S | 93 | −5.712 | −9.326 | 5.564 | 1.00 | 28.37 | O |
| ATOM | 4572 | OH2 | TIP | S | 94 | 12.167 | 21.855 | 31.241 | 1.00 | 41.68 | O |
| ATOM | 4573 | OH2 | TIP | S | 95 | −3.343 | −17.247 | 16.589 | 1.00 | 36.78 | O |
| ATOM | 4574 | OH2 | TIP | S | 96 | 11.156 | 23.711 | 33.209 | 1.00 | 35.57 | O |
| ATOM | 4575 | OH2 | TIP | S | 97 | 25.545 | 16.245 | 18.621 | 1.00 | 42.42 | O |
| ATOM | 4576 | OH2 | TIP | S | 98 | 40.918 | 18.920 | 34.722 | 1.00 | 45.15 | O |
| ATOM | 4577 | OH2 | TIP | S | 99 | 11.862 | 37.569 | 22.369 | 1.00 | 39.52 | O |
| ATOM | 4578 | OH2 | TIP | S | 100 | 15.658 | 8.162 | −3.968 | 1.00 | 36.43 | O |
| ATOM | 4579 | OH2 | TIP | S | 101 | 9.902 | 24.553 | 37.142 | 1.00 | 45.39 | O |
| ATOM | 4580 | OH2 | TIP | S | 102 | 6.314 | −5.317 | −19.652 | 1.00 | 42.39 | O |
| ATOM | 4581 | OH2 | TIP | S | 103 | 41.337 | 39.304 | 18.249 | 1.00 | 43.68 | O |
| ATOM | 4582 | OH2 | TIP | S | 104 | 1.800 | −8.520 | −5.551 | 1.00 | 35.38 | O |
| ATOM | 4583 | OH2 | TIP | S | 105 | −2.162 | −16.185 | 1.874 | 1.00 | 43.33 | O |
| ATOM | 4584 | OH2 | TIP | S | 106 | 39.849 | 40.296 | 25.274 | 1.00 | 39.96 | O |
| ATOM | 4585 | OH2 | TIP | S | 107 | 17.812 | 14.377 | 26.537 | 1.00 | 49.82 | O |
| ATOM | 4586 | OH2 | TIP | S | 108 | 20.087 | 16.149 | 58.833 | 1.00 | 41.90 | O |
| ATOM | 4587 | OH2 | TIP | S | 109 | 23.681 | 20.283 | 36.450 | 1.00 | 34.15 | O |
| ATOM | 4588 | OH2 | TIP | S | 110 | 31.232 | 19.396 | 25.383 | 1.00 | 31.15 | O |
| ATOM | 4589 | OH2 | TIP | S | 111 | 9.926 | 36.917 | 34.935 | 1.00 | 39.68 | O |
| ATOM | 4590 | OH2 | TIP | S | 112 | 13.742 | 8.657 | 8.468 | 1.00 | 37.13 | O |
| ATOM | 4591 | OH2 | TIP | S | 113 | 14.444 | −5.155 | −2.332 | 1.00 | 34.36 | O |
| ATOM | 4592 | OH2 | TIP | S | 114 | −14.505 | −11.350 | 0.205 | 1.00 | 38.40 | O |
| ATOM | 4593 | OH2 | TIP | S | 115 | −14.458 | 0.142 | 5.499 | 1.00 | 35.12 | O |
| ATOM | 4594 | OH2 | TIP | S | 116 | −0.021 | −12.439 | 12.153 | 1.00 | 43.53 | O |
| ATOM | 4595 | OH2 | TIP | S | 117 | 28.916 | 11.485 | 14.387 | 1.00 | 40.27 | O |
| ATOM | 4596 | OH2 | TIP | S | 118 | −4.527 | 10.289 | −9.542 | 1.00 | 45.18 | O |
| ATOM | 4597 | OH2 | TIP | S | 119 | 1.974 | 14.755 | −11.306 | 1.00 | 47.77 | O |
| ATOM | 4598 | OH2 | TIP | S | 120 | −15.749 | 10.668 | 12.672 | 1.00 | 45.53 | O |
| ATOM | 4599 | OH2 | TIP | S | 121 | −11.214 | −2.226 | −11.345 | 1.00 | 46.10 | O |
| ATOM | 4600 | OH2 | TIP | S | 122 | 36.682 | 45.523 | 28.569 | 1.00 | 37.78 | O |
| ATOM | 4601 | OH2 | TIP | S | 123 | 19.320 | 23.480 | 50.684 | 1.00 | 40.47 | O |
| ATOM | 4602 | OH2 | TIP | S | 124 | −15.435 | −9.919 | −3.811 | 1.00 | 41.21 | O |
| ATOM | 4603 | OH2 | TIP | S | 125 | 38.588 | 26.717 | 13.793 | 1.00 | 44.52 | O |
| ATOM | 4604 | OH2 | TIP | S | 126 | 7.685 | −14.424 | 4.306 | 1.00 | 51.58 | O |
| ATOM | 4605 | OH2 | TIP | S | 127 | 36.734 | 26.561 | 42.263 | 1.00 | 44.35 | O |
| ATOM | 4606 | OH2 | TIP | S | 128 | 23.012 | 18.699 | 64.427 | 1.00 | 55.43 | O |
| ATOM | 4607 | OH2 | TIP | S | 129 | −11.079 | 16.753 | 2.337 | 1.00 | 36.15 | O |
| ATOM | 4608 | OH2 | TIP | S | 130 | 32.804 | 36.247 | 8.025 | 1.00 | 46.53 | O |
| ATOM | 4609 | OH2 | TIP | S | 131 | 5.452 | −12.666 | −27.857 | 1.00 | 46.53 | O |
| ATOM | 4610 | OH2 | TIP | S | 132 | 5.777 | −5.213 | 4.878 | 1.00 | 52.94 | O |
| ATOM | 4611 | OH2 | TIP | S | 133 | 17.902 | 43.584 | 22.88 | 1.00 | 38.79 | O |
| ATOM | 4612 | OH2 | TIP | S | 134 | 41.529 | 41.359 | 30.489 | 1.00 | 45.08 | O |
| ATOM | 4613 | OH2 | TIP | S | 135 | 19.433 | 49.428 | 35.071 | 1.00 | 36.73 | O |
| ATOM | 4614 | OH2 | TIP | S | 136 | 40.681 | 17.939 | 17.861 | 1.00 | 52.60 | O |
| ATOM | 4615 | OH2 | TIP | S | 137 | 7.694 | 14.779 | 8.016 | 1.00 | 38.37 | O |
| ATOM | 4616 | OH2 | TIP | S | 138 | 43.631 | 21.569 | 35.087 | 1.00 | 42.92 | O |
| ATOM | 4617 | OH2 | TIP | S | 139 | −14.096 | −4.522 | 2.725 | 1.00 | 38.87 | O |
| ATOM | 4618 | OH2 | TIP | S | 140 | 39.647 | 24.357 | 28.138 | 1.00 | 37.44 | O |
| ATOM | 4619 | OH2 | TIP | S | 141 | 34.792 | 45.810 | 26.786 | 1.00 | 33.97 | O |
| ATOM | 4620 | OH2 | TIP | S | 142 | 35.678 | 30.939 | 40.741 | 1.00 | 50.18 | O |
| ATOM | 4621 | OH2 | TIP | S | 143 | 1.690 | −5.943 | 7.932 | 1.00 | 46.04 | O |
| ATOM | 4622 | OH2 | TIP | S | 144 | 16.446 | 8.323 | 29.203 | 1.00 | 43.29 | O |
| ATOM | 4623 | OH2 | TIP | S | 145 | 4.806 | −6.139 | −16.268 | 1.00 | 46.89 | O |
| ATOM | 4624 | OH2 | TIP | S | 146 | 9.706 | 7.514 | −13.847 | 1.00 | 40.39 | O |
| ATOM | 4625 | OH2 | TIP | S | 147 | −2.757 | −8.476 | 20.990 | 1.00 | 44.56 | O |
| ATOM | 4626 | OH2 | TIP | S | 148 | 19.924 | 51.030 | 32.912 | 1.00 | 56.09 | O |
| ATOM | 4627 | OH2 | TIP | S | 149 | −9.256 | 17.012 | 4.187 | 1.00 | 37.40 | O |
| ATOM | 4628 | OH2 | TIP | S | 150 | 13.798 | 12.721 | 1.801 | 1.00 | 41.15 | O |
| ATOM | 4629 | OH2 | TIP | S | 151 | −16.062 | 12.522 | 0.503 | 1.00 | 45.91 | O |
| ATOM | 4630 | OH2 | TIP | S | 152 | 43.042 | 24.814 | 30.118 | 1.00 | 45.63 | O |
| ATOM | 4631 | OH2 | TIP | S | 153 | 32.777 | 49.012 | 32.108 | 1.00 | 49.84 | O |
| ATOM | 4632 | OH2 | TIP | S | 154 | 35.924 | 45.614 | 31.001 | 1.00 | 42.83 | O |
| ATOM | 4633 | OH2 | TIP | S | 155 | −4.339 | −23.117 | −0.551 | 1.00 | 49.33 | O |
| ATOM | 4634 | OH2 | TIP | S | 156 | 19.827 | 23.697 | 25.905 | 1.00 | 53.14 | O |
| ATOM | 4635 | OH2 | TIP | S | 157 | 37.820 | 44.919 | 22.145 | 1.00 | 44.38 | O |
| ATOM | 4636 | OH2 | TIP | S | 158 | 40.593 | 29.995 | 32.300 | 1.00 | 31.52 | O |
| ATOM | 4637 | OH2 | TIP | S | 159 | −15.055 | 1.168 | −1.359 | 1.00 | 30.62 | O |
| ATOM | 4638 | OH2 | TIP | S | 160 | −16.412 | 7.003 | 5.433 | 1.00 | 32.94 | O |
| ATOM | 4639 | OH2 | TIP | S | 161 | 15.985 | 29.616 | 25.353 | 1.00 | 32.94 | O |
| ATOM | 4640 | OH2 | TIP | S | 162 | 42.076 | 35.832 | 25.453 | 1.00 | 35.51 | O |
| ATOM | 4641 | OH2 | TIP | S | 163 | 10.339 | −9.751 | −25.987 | 1.00 | 46.65 | O |
| ATOM | 4642 | OH2 | TIP | S | 164 | 34.356 | 38.780 | 10.727 | 1.00 | 36.90 | O |
| ATOM | 4643 | OH2 | TIP | S | 165 | 9.618 | 0.824 | 5.500 | 1.00 | 35.38 | O |
| ATOM | 4644 | OH2 | TIP | S | 166 | 26.940 | 9.884 | 20.217 | 1.00 | 38.88 | O |
| ATOM | 4645 | OH2 | TIP | S | 167 | −7.023 | 7.631 | 22.982 | 1.00 | 45.13 | O |
| ATOM | 4646 | OH2 | TIP | S | 168 | −17.523 | −3.768 | 0.764 | 1.00 | 47.38 | O |
| ATOM | 4647 | OH2 | TIP | S | 169 | 34.610 | 28.019 | 11.023 | 1.00 | 41.98 | O |
| ATOM | 4648 | OH2 | TIP | S | 170 | 38.500 | 22.241 | 10.888 | 1.00 | 49.86 | O |
| ATOM | 4649 | OH2 | TIP | S | 171 | 39.773 | 16.186 | 22.302 | 1.00 | 42.81 | O |
| ATOM | 4650 | OH2 | TIP | S | 172 | 39.007 | 29.639 | 38.711 | 1.00 | 40.01 | O |
| ATOM | 4651 | OH2 | TIP | S | 173 | −3.162 | −13.972 | 1.056 | 1.00 | 37.80 | O |
| ATOM | 4652 | OH2 | TIP | S | 174 | 15.866 | 36.292 | 44.854 | 1.00 | 41.03 | O |
| ATOM | 4653 | OH2 | TIP | S | 175 | 28.716 | 14.934 | 29.871 | 1.00 | 40.86 | O |
| ATOM | 4654 | OH2 | TIP | S | 176 | 12.877 | 7.462 | 40.598 | 1.00 | 50.68 | O |
| ATOM | 4655 | OH2 | TIP | S | 177 | 6.133 | 20.704 | −4.054 | 1.00 | 39.16 | O |
| ATOM | 4656 | OH2 | TIP | S | 178 | 39.969 | 28.942 | 25.391 | 1.00 | 38.15 | O |
| ATOM | 4657 | OH2 | TIP | S | 179 | −13.449 | 0.920 | −7.668 | 1.00 | 44.30 | O |
| ATOM | 4658 | OH2 | TIP | S | 180 | 1.907 | −19.850 | −13.019 | 1.00 | 46.61 | O |
| ATOM | 4659 | OH2 | TIP | S | 181 | −11.803 | 7.776 | 20.873 | 1.00 | 44.08 | O |
| ATOM | 4660 | OH2 | TIP | S | 182 | 15.943 | 16.779 | 25.867 | 1.00 | 50.71 | O |
| ATOM | 4661 | OH2 | TIP | S | 183 | 11.887 | 41.459 | 29.150 | 1.00 | 40.31 | O |
| ATOM | 4662 | OH2 | TIP | S | 184 | −12.916 | −6.604 | 20.001 | 1.00 | 48.89 | O |
| ATOM | 4663 | OH2 | TIP | S | 185 | 37.386 | 36.717 | 10.040 | 1.00 | 47.31 | O |
| ATOM | 4664 | OH2 | TIP | S | 186 | 2.834 | 18.279 | 41.221 | 1.00 | 54.64 | O |
| ATOM | 4665 | OH2 | TIP | S | 187 | −9.066 | −0.690 | 19.812 | 1.00 | 43.53 | O |
| ATOM | 4666 | OH2 | TIP | S | 188 | 16.454 | 33.980 | 25.756 | 1.00 | 38.52 | O |
| ATOM | 4667 | OH2 | TIP | S | 189 | 23.851 | 22.769 | 22.843 | 1.00 | 48.45 | O |
| ATOM | 4668 | OH2 | TIP | S | 190 | −14.255 | −12.614 | 8.773 | 1.00 | 46.74 | O |
| ATOM | 4669 | OH2 | TIP | S | 191 | −5.249 | −12.948 | −12.695 | 1.00 | 53.90 | O |
| ATOM | 4670 | OH2 | TIP | S | 192 | 9.039 | −20.611 | 1.774 | 1.00 | 42.77 | O |
| ATOM | 4671 | OH2 | TIP | S | 193 | −5.304 | −5.892 | −10.918 | 1.00 | 47.95 | O |
| ATOM | 4672 | OH2 | TIP | S | 194 | 9.516 | −11.723 | 5.016 | 1.00 | 49.40 | O |
| ATOM | 4673 | OH2 | TIP | S | 195 | 20.372 | 36.265 | 6.690 | 1.00 | 42.55 | O |
| ATOM | 4674 | OH2 | TIP | S | 196 | 9.099 | 5.271 | 5.043 | 1.00 | 40.29 | O |
| ATOM | 4675 | OH2 | TIP | S | 197 | 28.290 | 20.438 | 9.887 | 1.00 | 44.18 | O |
| ATOM | 4676 | OH2 | TIP | S | 198 | 41.403 | 24.242 | 26.360 | 1.00 | 41.99 | O |
| ATOM | 4677 | OH2 | TIP | S | 199 | 17.555 | −7.280 | −3.972 | 1.00 | 45.72 | O |
| ATOM | 4678 | OH2 | TIP | S | 200 | 11.643 | 26.387 | 33.558 | 1.00 | 44.67 | O |
| ATOM | 4679 | OH2 | TIP | S | 201 | −13.080 | −2.043 | 17.068 | 1.00 | 48.89 | O |
| ATOM | 4680 | OH2 | TIP | S | 202 | 21.406 | 41.180 | 6.510 | 1.00 | 47.83 | O |
| ATOM | 4681 | OH2 | TIP | S | 203 | −3.647 | −14.349 | 4.372 | 1.00 | 45.03 | O |
| ATOM | 4682 | OH2 | TIP | S | 204 | 1.975 | −16.444 | 0.528 | 1.00 | 46.05 | O |
| ATOM | 4683 | OH2 | TIP | S | 205 | 29.352 | 38.318 | 42.802 | 1.00 | 39.18 | O |
| ATOM | 4684 | OH2 | TIP | S | 206 | 23.716 | 43.491 | 42.218 | 1.00 | 52.21 | O |
| ATOM | 4685 | OH2 | TIP | S | 207 | 37.832 | 29.664 | 12.655 | 1.00 | 40.31 | O |
| ATOM | 4686 | OH2 | TIP | S | 208 | 4.964 | 17.602 | 2.226 | 1.00 | 49.67 | O |
| ATOM | 4687 | OH2 | TIP | S | 209 | 39.168 | 40.024 | 22.519 | 1.00 | 48.01 | O |
| ATOM | 4688 | OH2 | TIP | S | 210 | 13.868 | −18.464 | −3.462 | 1.00 | 45.96 | O |
| ATOM | 4689 | OH2 | TIP | S | 211 | −15.848 | −4.527 | 4.594 | 1.00 | 44.75 | O |
| ATOM | 4690 | OH2 | TIP | S | 212 | −10.372 | 16.898 | −0.055 | 1.00 | 41.74 | O |
| ATOM | 4691 | OH2 | TIP | S | 213 | −3.769 | 9.428 | −11.859 | 1.00 | 41.64 | O |
| ATOM | 4692 | OH2 | TIP | S | 214 | −12.248 | 16.128 | 8.702 | 1.00 | 42.97 | O |
| ATOM | 4693 | OH2 | TIP | S | 215 | −10.705 | −1.226 | −13.945 | 1.00 | 46.83 | O |
| ATOM | 4694 | OH2 | TIP | S | 216 | 22.402 | 4.106 | 44.758 | 1.00 | 42.90 | O |
| ATOM | 4695 | OH2 | TIP | S | 217 | −17.355 | 1.265 | 1.310 | 1.00 | 49.40 | O |
| ATOM | 4696 | OH2 | TIP | S | 218 | 42.477 | 37.616 | 23.340 | 1.00 | 42.07 | O |
| ATOM | 4697 | OH2 | TIP | S | 219 | −16.918 | 8.816 | 7.795 | 1.00 | 46.92 | O |
| ATOM | 4698 | OH2 | TIP | S | 220 | 27.500 | 8.859 | 43.884 | 1.00 | 48.46 | O |
| ATOM | 4699 | OH2 | TIP | S | 221 | 30.021 | 39.029 | 40.447 | 1.00 | 44.55 | O |
| ATOM | 4700 | OH2 | TIP | S | 222 | 39.391 | 18.850 | 37.190 | 1.00 | 52.09 | O |
| ATOM | 4701 | OH2 | TIP | S | 223 | 4.533 | −18.856 | 1.922 | 1.00 | 50.69 | O |
| ATOM | 4702 | OH2 | TIP | S | 224 | 29.183 | 14.389 | 26.673 | 1.00 | 47.97 | O |
| ATOM | 4703 | OH2 | TIP | S | 225 | −18.903 | −8.822 | 10.919 | 1.00 | 49.49 | O |
| ATOM | 4704 | OH2 | TIP | S | 226 | 29.080 | 14.271 | 21.460 | 1.00 | 41.94 | O |
| ATOM | 4705 | OH2 | TIP | S | 227 | −12.418 | 15.936 | −7.420 | 1.00 | 49.12 | O |
| ATOM | 4706 | OH2 | TIP | S | 228 | 40.908 | 38.798 | 10.820 | 1.00 | 48.24 | O |
| ATOM | 4707 | OH2 | TIP | S | 229 | 20.873 | 22.585 | 47.103 | 1.00 | 50.53 | O |
| ATOM | 4708 | OH2 | TIP | S | 230 | 21.831 | −10.033 | −6.237 | 1.00 | 53.05 | O |
| ATOM | 4709 | OH2 | TIP | S | 231 | 29.823 | 5.586 | 31.413 | 1.00 | 52.07 | O |
| ATOM | 4710 | OH2 | TIP | S | 232 | 44.472 | 19.890 | 19.952 | 1.00 | 49.75 | O |
| ATOM | 4711 | OH2 | TIP | S | 233 | −14.590 | −13.479 | 12.084 | 1.00 | 44.70 | O |
| ATOM | 4712 | OH2 | TIP | S | 234 | 36.315 | 29.168 | 8.598 | 1.00 | 49.27 | O |
| ATOM | 4713 | OH2 | TIP | S | 235 | −3.492 | −14.586 | 9.423 | 1.00 | 44.24 | O |
| ATOM | 4714 | OH2 | TIP | S | 236 | 37.931 | 44.730 | 38.212 | 1.00 | 52.70 | O |
| ATOM | 4715 | OH2 | TIP | S | 237 | −13.540 | 11.244 | 8.565 | 1.00 | 48.15 | O |
| ATOM | 4716 | OH2 | TIP | S | 238 | −15.408 | 10.000 | 20.145 | 1.00 | 50.07 | O |
| ATOM | 4717 | OH2 | TIP | S | 239 | 20.743 | 2.608 | 48.382 | 1.00 | 49.86 | O |
| ATOM | 4718 | OH2 | TIP | S | 240 | 23.597 | 12.310 | 30.357 | 1.00 | 48.72 | O |
| ATOM | 4719 | OH2 | TIP | S | 241 | −5.755 | −8.661 | −12.899 | 1.00 | 51.10 | O |
| ATOM | 4720 | OH2 | TIP | S | 242 | 40.644 | 44.705 | 34.483 | 1.00 | 51.24 | O |
| ATOM | 4721 | OH2 | TIP | S | 243 | 32.880 | 11.239 | 31.811 | 1.00 | 48.34 | O |
| ATOM | 4722 | OH2 | TIP | S | 244 | −7.454 | −17.415 | −0.915 | 1.00 | 45.03 | O |
| ATOM | 4723 | OH2 | TIP | S | 245 | 13.922 | −2.398 | −2.504 | 1.00 | 46.83 | O |
| ATOM | 4724 | OH2 | TIP | S | 246 | 0.782 | 12.427 | −14.770 | 1.00 | 48.57 | O |
| ATOM | 4725 | OH2 | TIP | S | 247 | −15.049 | 15.812 | −3.622 | 1.00 | 51.01 | O |
| ATOM | 4726 | OH2 | TIP | S | 248 | 4.404 | 15.313 | 18.746 | 1.00 | 47.05 | O |
| ATOM | 4727 | OH2 | TIP | S | 249 | 31.402 | 20.217 | 43.758 | 1.00 | 52.57 | O |
| ATOM | 4728 | OH2 | TIP | S | 250 | −10.062 | 2.073 | −9.809 | 1.00 | 50.33 | O |
| ATOM | 4729 | OH2 | TIP | S | 251 | 11.650 | −0.830 | 4.614 | 1.00 | 50.82 | O |
| ATOM | 4730 | OH2 | TIP | S | 252 | −1.232 | −15.435 | 4.563 | 1.00 | 50.12 | O |
| ATOM | 4731 | OH2 | TIP | S | 253 | −12.330 | 0.786 | 18.255 | 1.00 | 43.46 | O |
| ATOM | 4732 | OH2 | TIP | S | 254 | 36.403 | 27.682 | 44.769 | 1.00 | 51.60 | O |
| ATOM | 4733 | OH2 | TIP | S | 255 | −18.273 | 6.055 | 3.212 | 1.00 | 51.88 | O |
| ATOM | 4734 | OH2 | TIP | S | 256 | −13.609 | 16.893 | −0.632 | 1.00 | 50.05 | O |
| ATOM | 4735 | OH2 | TIP | S | 257 | 14.001 | 15.235 | 0.255 | 1.00 | 48.29 | O |
| ATOM | 4736 | OH2 | TIP | S | 258 | 10.461 | 2.911 | 6.999 | 1.00 | 51.70 | O |
| ATOM | 4737 | OH2 | TIP | S | 259 | 11.740 | 10.308 | 34.387 | 1.00 | 49.96 | O |
| ATOM | 4738 | OH2 | TIP | S | 260 | −11.901 | 5.018 | −8.016 | 1.00 | 50.95 | O |
| ATOM | 4739 | OH2 | TIP | S | 261 | −10.492 | 19.957 | −5.817 | 1.00 | 45.58 | O |
| TER | 1 | | TIP | S | 261 | | | | | | |
| END | | | | | | | | | | | |

TABLE 2
Crystal Data and X-ray data statistics

| | |
|---|---|
| Number of crystals | 1 |
| Space group | P1 (primitive triclinic) |
| Unit cell dimensions | a = 35.77 b = 57.56 c = 80.20 Å |
| | α = 68.97 β = 89.83 γ = 89.95° |
| Number of monomers/a.u. | 2 |
| Packing coefficient | 2.05 Å³/Da |
| Solvent content | 38% |
| Resolution range | 50-1.9 Å |
| Number of observations | 386926 |
| Number of reject observations | 24465 |
| Number of unique reflections | 46855 |
| Mosaicity | 0.817 |
| Overall | |
| Data redundancy | 8.25 |
| Data completeness | 97.0% |
| <I/σ (I)> | 20.6 |
| R_{merge} | 0.049 |
| Highest resolution shell | |
| Resolution range | 1.97-1.90 Å |
| Completeness for shell | 96.5% |
| R_{merge} for shell | 0.175 |
| Reflections with I ≧ 3σ(I) | 86.7% |

TABLE 3
Refinement Statistics

| Data used in refinement | |
|---|---|
| resolution range | 19.26-1.90 Å |
| intensity cutoff (Sigma(F)) | 0.0 |
| number of reflections | 45463 |
| completeness (working + test set) | 96.7% |
| test set | 5.0% |
| Fit to data used in refinement | |
| overall R_{cryst} | 0.182 |
| overall R_{free} | 0.209 |
| Fit in the highest resolution bin | |
| resolution range | 2.02-1.90 Å |
| bin completeness (working + test set) | 93.7% |
| bin R_{cryst} | 0.195 |
| bin R_{free} | 0.241 |
| Number of non-hydrogen atoms | |
| protein atoms | 4478 |
| ligand (2 molecules staurosporin) | 70 |
| waters | 261 |
| Overall B value from Wilson plot | 24.1 Å² |
| Overall mean B value | 35.0 Å² |
| Cross-validated estimated coordinate error (low res. | |
| cutoff: 5.0 Å) | |
| from Luzzati plot | 0.22 Å |
| from σ_{A} | 0.11 Å |
| Rms deviations from ideal values | |
| bond lengths | 0.011 Å |
| bond angles | 1.3° |
| dihedral angles | 21.4° |
| improper angles | 0.91° |

TABLE 4
List of Contacts Between Catalytic Domain of ZAP-70 and Staurosporine

| atom in protein | | | atom in staurosporine | | | distance (A) |
|---|---|---|---|---|---|---|
| LEU | 344 | O | STU | C25 | 3.4 | |
| GLY | 345 | CA | STU | O4 | 3.5 | |
| CYS | 346 | O | STU | C26 | 3.9 | |
| GLY | 347 | N | STU | C26 | 5.4 | |
| PHE | 349 | CZ | STU | C16 | 4.5 | |
| VAL | 352 | CG2 | STU | C17 | 3.8 | |
| ALA | 367 | CB | STU | N1 | 3.2 | |
| LYS | 369 | CD | STU | C14 | 4.3 | |
| GLU | 386 | OE2 | STU | C14 | 4.4 | |
| VAL | 399 | CG1 | STU | C9 | 4.1 | |
| MET | 414 | CE | STU | C13 | 3.6 | |
| GLU | 415 | O | STU | N1 | 3.0 | H-bridge |
| MET | 416 | CA | STU | O5 | 3.4 | |
| ALA | 417 | N | STU | O5 | 2.7 | H-bridge |
| GLY | 418 | O | STU | C3 | 4.3 | |
| GLY | 419 | C | STU | C3 | 4.8 | |
| GLY | 420 | CA | STU | C3 | 3.5 | |
| PRO | 421 | CG | STU | C24 | 3.8 | |
| HIS | 423 | NE2 | STU | N4 | 5.1 | through Solv3 |
| Lys | 424 | NZ | STU | N4 | 6.4 | through Solv3 |
| ARG | 465 | O | STU | N4 | 3.0 | H-bridge |
| ASN | 466 | OD1 | STU | C27 | 3.4 | |
| LEU | 468 | CD1 | STU | C7 | 3.3 | |
| SER | 478 | OG | STU | C27 | 3.1 | Alt. Pos. |
| ASP | 479 | CG | STU | C15 | 3.5 | |

1. A crystal of the ZAP-70 kinase comprising the catalytic domain of ZAP-70 kinase with a unit cell dimension of a=35.77±5 Ångstroms, b=57.56±5 Ångstroms, c=80.20±5 Ångstroms; α=68.97±5 degrees, β=89.83±5 degrees, γ=89.95±5 degrees.

2. A crystal of the ZAP-70 kinase comprising the catalytic domain of ZAP-70 kinase wherein said catalytic domain has a three-dimensional structure comprising the atomic structure coordinates of Table 1.

3. A crystal of claim 1 wherein the catalytic domain of ZAP-70 kinase comprises the sequence of SEQ ID. No. 2, fragment or a homologue thereof.

4. A crystal of claim 3 wherein the catalytic domain of ZAP-70 kinase comprises at least the ATP-binding site.

5. A crystal of claim 1 bound to at least one ligand or low molecular weight compound.

6. A computer readable medium comprising data storage material encoded with computer readable data wherein said data comprises the atomic coordinates of Table 1 comprising the catalytic domain of ZAP-70 kinase.

7. A method for making a crystal of a ZAP-70 kinase comprising the steps of:
(i) purification of the full-length ZAP-70 kinase of SEQ ID No.1
(ii) proteolytic domain definition
(iii) expression of the full-length ZAP-70 kinase of SEQ ID No.1 flanked by protease recognition sequences to facilitate proteolytic release of the desired domain of ZAP-70
(iv) expression of the full-length ZAP-70 kinase of step (iii) in a suitable host cell
(v) controlled proteolysis of the desired domain at protease recognition sites
(vi) rapid purification of the desired ZAP-70 domain.

8. A method according to claim 7 wherein the domain comprises the catalytic domain of ZAP-70 kinase of SEQ ID No.2, fragment or a homologue thereof.

9. A method according to claim 7 wherein the catalytic domain of ZAP-70, fragment or homologue thereof is bound to at least one ligand or low molecule weight chemical compound at any step prior to crystallization.

10. A method of determining the three-dimensional structure of the catalytic domain of ZAP-70 comprising:
(i) crystallization of ZAP-70 kinase comprising the catalytic domain of ZAP-70 (SEQ ID No.2), fragment or homologue thereof
(ii) utilizing the atomic coordinates of Table 1 in whole or in part to determine the three dimensional structure of the catalytic domain of ZAP-70, fragment or homologue thereof.

11. A method for determining the three-dimensional structure of a complex comprising the catalytic domain of ZAP-70 kinase (SEQ ID No.2), fragment or homologue thereof bound to at least one ligand comprising:
(i) obtaining x-ray diffraction data for crystals of the complex
(ii) utilizing the atomic coordinates of Table 1 in whole or in part to define the three-dimensional structure of the complex.

12. A method of identifying a ligand or low molecular weight compound that binds to the catalytic domain of ZAP-70 kinase comprising the steps of:
(i) using the three dimensional structure of the catalytic domain of ZAP-70 kinase derived in whole or in part from the set of atomic coordinates in Table 1 to select a potential ligand or low molecular weight compound that binds to the catalytic domain of ZAP-70
(ii) selecting those ligands or low molecular weight compounds that bind to the catalytic domain of ZAP-70.

13. A method of identifying a ligand or low molecular weight compound that binds to the catalytic domain of ZAP-70 kinase according to claim 11 wherein the catalytic domain of ZAP-70 kinase comprises at least the ATP-binding site of said domain.

14. A method of claim 12 for use in selecting ligands which inhibit the biological activity of ZAP-70 kinase.

15. A method of designing a ligand or low molecular weight compound capable of binding to ZAP-70 catalytic domain comprising:
(i) using the atomic coordinates of Table 1 in whole or in part to determine the three dimensional structure of ZAP-70 catalytic domain
(ii) probing the said catalytic domain of ZAP-70 with a candidate ligands or low molecular weight compounds to determine which bind to the catalytic domain of ZAP-70
(iii) selecting those ligands or low molecular weight compounds which bind to the catalytic domain of ZAP-70
(iv) modifying those ligands or low molecular weight compounds which bind to maximize physical binding properties such as Volubility, affinity, specificity or potency.

16. A method according to claim 15 wherein the candidate ligands or low molecular weight compounds are screened in silica.

17. A method according to claims 15 for use in designing ligands which inhibit the biological activity of ZAP-70 kinase.

18. A pharmaceutical composition comprising a ligand identified by the methods of claim 12 for use of treatment of diseases and conditions involving T-cell and lymphocyte activation.


SEQUENCE LISTING (6 sequences)

SEQ ID NO: 1 - protein, 619 aa, Homo sapiens
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 2 - protein, 322 aa, Homo sapiens
RITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 3 - DNA, 74 bp, Unknown
  Oligo MG474
cagatggatacacccctgagccagcactggaagttctgttccaggggccccgcataacgtccccagacaaaccg

SEQ ID NO: 4 - DNA, 20 bp, Unknown
  Oligo RS366
acaacgcacagaatctagcg

SEQ ID NO: 5 - DNA, 74 bp, Unknown
  Oligo MG475
cacactcccagcccacccatccacgctggaagttctgttccaggggcccttgactcatcctcagagacgaatcg

SEQ ID NO: 6 - DNA, 57 bp, Unknown
  Oligo MG479
gctcgaattctcaatgatgatgatgatgatgggcacaggcagcctcagccttctgtg